SEQUENCE LISTING (170 sequences)

SEQ ID NO: 1 - protein, 4 aa, synthetic construct
GFLG

SEQ ID NO: 2 - protein, 4 aa, synthetic construct
KPLR

SEQ ID NO: 3 - protein, 4 aa, synthetic construct
YLLL

SEQ ID NO: 4 - protein, 4 aa, synthetic construct
SPLR

SEQ ID NO: 5 - protein, 8 aa, synthetic construct
KPLRYLLL

SEQ ID NO: 6 - skipped

SEQ ID NO: 7 - protein, 4 aa, synthetic construct
SLVL

SEQ ID NO: 8 - skipped

SEQ ID NO: 9 - protein, 4 aa, synthetic construct
ELVR

SEQ ID NO: 10 - protein, 4 aa, synthetic construct
SPVR

SEQ ID NO: 11 - protein, 4 aa, synthetic construct
SLVR

SEQ ID NO: 12 - protein, 4 aa, synthetic construct
KPVR

SEQ ID NO: 13 - skipped

SEQ ID NO: 14 - protein, 4 aa, synthetic construct
ELVL

SEQ ID NO: 15-16 - skipped (2 entries)

SEQ ID NO: 17 - protein, 6 aa, synthetic construct
GGKLVR

SEQ ID NO: 18 - protein, 6 aa, synthetic construct
GGKPLR

SEQ ID NO: 19 - protein, 6 aa, synthetic construct
GGSLVR

SEQ ID NO: 20 - protein, 6 aa, synthetic construct
  MOD_RES 6: note = Citrulline
GGSLVX

SEQ ID NO: 21 - protein, 6 aa, synthetic construct
  MOD_RES 6: note = Citrulline
GGSPVX

SEQ ID NO: 22 - protein, 6 aa, synthetic construct
GGSLVL

SEQ ID NO: 23 - protein, 6 aa, synthetic construct
GGELVR

SEQ ID NO: 24 - protein, 6 aa, synthetic construct
GGELVL

SEQ ID NO: 25 - protein, 5 aa, synthetic construct
GSLVR

SEQ ID NO: 26 - protein, 5 aa, synthetic construct
  MOD_RES 5: note = Citrulline
GSLVX

SEQ ID NO: 27 - protein, 5 aa, synthetic construct
  MOD_RES 5: note = Citrulline
GKPVX

SEQ ID NO: 28 - protein, 5 aa, synthetic construct
GKPVR

SEQ ID NO: 29 - protein, 5 aa, synthetic construct
GSLVL

SEQ ID NO: 30 - protein, 5 aa, synthetic construct
GELVL

SEQ ID NO: 31 - skipped

SEQ ID NO: 32 - protein, 12 aa, synthetic construct
DDDDDDDDDDDD

SEQ ID NO: 33 - protein, 11 aa, synthetic construct
DDDDDDDDDDD

SEQ ID NO: 34 - protein, 10 aa, synthetic construct
DDDDDDDDDD

SEQ ID NO: 35 - protein, 9 aa, synthetic construct
DDDDDDDDD

SEQ ID NO: 36 - protein, 8 aa, synthetic construct
DDDDDDDD

SEQ ID NO: 37 - protein, 7 aa, synthetic construct
DDDDDDD

SEQ ID NO: 38 - protein, 6 aa, synthetic construct
DDDDDD

SEQ ID NO: 39 - protein, 5 aa, synthetic construct
DDDDD

SEQ ID NO: 40 - protein, 4 aa, synthetic construct
DDDD

SEQ ID NO: 41 - protein, 12 aa, synthetic construct
KKKKKKKKKKKK

SEQ ID NO: 42 - protein, 11 aa, synthetic construct
KKKKKKKKKKK

SEQ ID NO: 43 - protein, 10 aa, synthetic construct
KKKKKKKKKK

SEQ ID NO: 44 - protein, 9 aa, synthetic construct
KKKKKKKKK

SEQ ID NO: 45 - protein, 8 aa, synthetic construct
KKKKKKKK

SEQ ID NO: 46 - protein, 7 aa, synthetic construct
KKKKKKK

SEQ ID NO: 47 - protein, 6 aa, synthetic construct
KKKKKK

SEQ ID NO: 48 - protein, 5 aa, synthetic construct
KKKKK

SEQ ID NO: 49 - protein, 4 aa, synthetic construct
KKKK

SEQ ID NO: 50 - protein, 4 aa, synthetic construct
KDKD

SEQ ID NO: 51 - protein, 6 aa, synthetic construct
KDKDKD

SEQ ID NO: 52 - protein, 5 aa, synthetic construct
KSKSK

SEQ ID NO: 53 - protein, 7 aa, synthetic construct
KSKSKSK

SEQ ID NO: 54 - protein, 5 aa, synthetic construct
DSDSD

SEQ ID NO: 55 - protein, 7 aa, synthetic construct
DSDSDSD

SEQ ID NO: 56 - protein, 29 aa, synthetic construct
MHQKRTAMFQDPQERPRKLPQLCTELQTT

SEQ ID NO: 57 - protein, 29 aa, synthetic construct
PRKLPQLCTELQTTIHDIILECVYCKQQL

SEQ ID NO: 58 - protein, 29 aa, synthetic construct
HDIILECVYCKQQLLRREVYDFAFRDLCI

SEQ ID NO: 59 - protein, 29 aa, synthetic construct
RREVYDFAFRDLCIVYRDGNPYAVCDKCL

SEQ ID NO: 60 - protein, 29 aa, synthetic construct
YRDGNPYAVCDKCLKFYSKISEYRHYCYS

SEQ ID NO: 61 - protein, 29 aa, synthetic construct
FYSKISEYRHYCYSLYGTTLEQQYNKPLC

SEQ ID NO: 62 - protein, 29 aa, synthetic construct
YGTTLEQQYNKPLCDLLIRCINCQKPLCP

SEQ ID NO: 63 - protein, 29 aa, synthetic construct
LLIRCINCQKPLCPEEKQRHLDKKQRFHN

SEQ ID NO: 64 - protein, 28 aa, synthetic construct
EKQRHLDKKQRFHNIRGRWTGRCMSCCR

SEQ ID NO: 65 - protein, 24 aa, synthetic construct
IRGRWTGRCMSCCRSSRTRRETQL

SEQ ID NO: 66 - protein, 27 aa, synthetic construct
MHGDTPTLHEYMLDLQPETTDLYCYEQ

SEQ ID NO: 67 - protein, 25 aa, synthetic construct
DLQPETTDLYCYEQLNDSSEEEDEI

SEQ ID NO: 68 - protein, 25 aa, synthetic construct
YEQLNDSSEEEDEIDGPAGQAEPDR

SEQ ID NO: 69 - protein, 27 aa, synthetic construct
DEIDGPAGQAEPDRAHYNIVTFCCKCD

SEQ ID NO: 70 - protein, 32 aa, synthetic construct
RAHYNIVTFCCKCDSTLRLCVQSTHVDIRTLE

SEQ ID NO: 71 - protein, 32 aa, synthetic construct
LCVQSTHVDIRTLEDLLMGTLGIVCPICSQKP

SEQ ID NO: 72 - protein, 20 aa, synthetic construct
QLYQTCKAAGTCPSDVIPKI

SEQ ID NO: 73 - protein, 29 aa, synthetic construct
  SITE 1: note = Nle
  SITE 8: note = Nle
  MOD_RES 23: note = Abu
XHQKRTAXFQDPQERPRKLPQLXTELQTT

SEQ ID NO: 74 - protein, 29 aa, synthetic construct
  SITE 1: note = Nle
  SITE 8: note = Nle
XHQKRTAXFQDPQERPRKLPQLCTELQTT

SEQ ID NO: 75 - protein, 29 aa, synthetic construct
  MOD_RES 23: note = Abu
MHQKRTAMFQDPQERPRKLPQLXTELQTT

SEQ ID NO: 76 - protein, 29 aa, synthetic construct
  MOD_RES 8: note = Abu
  MOD_RES 22: note = Abu
  MOD_RES 25: note = Abu
PRKLPQLXTELQTTIHDIILEXVYXKQQL

SEQ ID NO: 77 - protein, 29 aa, synthetic construct
  MOD_RES 7: note = Abu
  MOD_RES 10: note = Abu
  MOD_RES 28: note = Abu
HDIILEXVYXKQQLLRREVYDFAFRDLXI

SEQ ID NO: 78 - protein, 29 aa, synthetic construct
  MOD_RES 13: note = Abu
  MOD_RES 25: note = Abu
  MOD_RES 28: note = Abu
RREVYDFAFRDLXIVYRDGNPYAVXDKXL

SEQ ID NO: 79 - protein, 29 aa, synthetic construct
  MOD_RES 10: note = Abu
  MOD_RES 13: note = Abu
  MOD_RES 27: note = Abu
YRDGNPYAVXDKXLKFYSKISEYRHYXYS

SEQ ID NO: 80 - protein, 29 aa, synthetic construct
  MOD_RES 12: note = Abu
  MOD_RES 29: note = Abu
FYSKISEYRHYXYSLYGTTLEQQYNKPLX

SEQ ID NO: 81 - protein, 28 aa, synthetic construct
  MOD_RES 19: note = Abu
  MOD_RES 22: note = Abu
  MOD_RES 27: note = Abu
YGTTLEQQYNKPLDLLIRXINXQKPLXP

SEQ ID NO: 82 - protein, 29 aa, synthetic construct
  MOD_RES 5: note = Abu
  MOD_RES 8: note = Abu
  MOD_RES 13: note = Abu
LLIRXINXQKPLXPEEKQRHLDKKQRFHN

SEQ ID NO: 83 - protein, 28 aa, synthetic construct
  SITE 24: note = Nle
EKQRHLDKKQRFHNIRGRWTGRCXSCCR

SEQ ID NO: 84 - protein, 28 aa, synthetic construct
  MOD_RES 23: note = Abu
  SITE 24: note = Nle
  MOD_RES 26: note = Abu
  MOD_RES 27: note = Abu
EKQRHLDKKQRFHNIRGRWTGRXXSXXR

SEQ ID NO: 85 - protein, 28 aa, synthetic construct
  MOD_RES 23: note = Abu
  MOD_RES 26: note = Abu
  MOD_RES 27: note = Abu
EKQRHLDKKQRFHNIRGRWTGRXMSXXR

SEQ ID NO: 86 - protein, 24 aa, synthetic construct
  SITE 10: note = Nle
IRGRWTGRCXSCCRSSRTRRETQL

SEQ ID NO: 87 - protein, 24 aa, synthetic construct
  SITE 10: note = Nle
  MOD_RES 9: note = Abu
  MOD_RES 12: note = Abu
  MOD_RES 13: note = Abu
IRGRWTGRXXSXXRSSRTRRETQL

SEQ ID NO: 88 - protein, 24 aa, synthetic construct
  MOD_RES 9: note = Abu
  MOD_RES 12: note = Abu
  MOD_RES 13: note = Abu
IRGRWTGRXMSXXRSSRTRRETQL

SEQ ID NO: 89 - protein, 27 aa, synthetic construct
  SITE 1: note = Nle
  SITE 12: note = Nle
XHGDTPTLHEYXLDLQPETTDLYCYEQ

SEQ ID NO: 90 - protein, 27 aa, synthetic construct
  MOD_RES 1: note = Nle
  SITE 12: note = Nle
  MOD_RES 24: note = Abu
XHGDTPTLHEYXLDLQPETTDLYXYEQ

SEQ ID NO: 91 - protein, 27 aa, synthetic construct
  SITE 1: note = Nle
  SITE 12: note = Nle
XHGDTPTLHEYXLDLQPETTDLYMYEQ

SEQ ID NO: 92 - protein, 25 aa, synthetic construct
  MOD_RES 11: note = Abu
DLQPETTDLYXYEQLNDSSEEEDEI

SEQ ID NO: 93 - protein, 25 aa, unidentified
  source 1..25: note = Human papillomavirus
YEQLNDSSEEEDEIDGPAGQAEPDR

SEQ ID NO: 94 - protein, 27 aa, synthetic construct
  MOD_RES 23: note = Abu
  MOD_RES 24: note = Abu
  MOD_RES 26: note = Abu
DEIDGPAGQAEPDRAHYNIVTFXXKXD

SEQ ID NO: 95 - protein, 32 aa, synthetic construct
  MOD_RES 10: note = Abu
  MOD_RES 11: note = Abu
  MOD_RES 13: note = Abu
  MOD_RES 20: note = Abu
RAHYNIVTFXXKXDSTLRLXVQSTHVDIRTLE

SEQ ID NO: 96 - protein, 32 aa, synthetic construct
  SITE 18: note = Nle
LCVQSTHVDIRTLEDLLXGTLGIVCPICSQKP

SEQ ID NO: 97 - protein, 32 aa, synthetic construct
  MOD_RES 2: note = Abu
  SITE 18: note = Nle
  MOD_RES 25: note = Abu
  MOD_RES 28: note = Abu
LXVQSTHVDIRTLEDLLXGTLGIVXPIXSQKP

SEQ ID NO: 98 - protein, 32 aa, synthetic construct
  MOD_RES 2: note = Abu
  MOD_RES 25: note = Abu
  MOD_RES 28: note = Abu
LXVQSTHVDIRTLEDLLMGTLGIVXPIXSQKP

SEQ ID NO: 99 - protein, 20 aa, synthetic construct
  MOD_RES 6: note = Abu
  MOD_RES 12: note = Abu
QLYQTXKAAGTXPSDVIPKI

SEQ ID NO: 100 - protein, 29 aa, synthetic construct
ALQAIELQLTLETIYNSQYSNEKWTLQDV

SEQ ID NO: 101 - protein, 29 aa, synthetic construct
NSQYSNEKWTLQDVSLEVYLTAPTGCIKK

SEQ ID NO: 102 - protein, 29 aa, synthetic construct
SVTVVEGQVDYYGLYYVHEGIRTYFVQFK

SEQ ID NO: 103 - protein, 32 aa, synthetic construct
LKGDANTLKCLRYRFKKHCTLYTAVSSTWHWT

SEQ ID NO: 104 - protein, 29 aa, synthetic construct
KHKSAIVTLTYDSEWQRDQFLSQVKIPKT

SEQ ID NO: 105 - protein, 29 aa, synthetic construct
MHQKRTAMFQDPQERPRKLPQLCTELQTT

SEQ ID NO: 106 - protein, 29 aa, synthetic construct
PRKLPQLCTELQTTIHDIILECVYCKQQL

SEQ ID NO: 107 - protein, 29 aa, synthetic construct
HDIILECVYCKQQLLRREVYDFAFRDLCI

SEQ ID NO: 108 - protein, 29 aa, synthetic construct
RREVYDFAFRDLCIVYRDGNPYAVCDKCL

SEQ ID NO: 109 - protein, 29 aa, synthetic construct
YRDGNPYAVCDKCLKFYSKISEYRHYCYS

SEQ ID NO: 110 - protein, 29 aa, synthetic construct
FYSKISEYRHYCYSLYGTTLEQQYNKPLC

SEQ ID NO: 111 - protein, 29 aa, synthetic construct
YGTTLEQQYNKPLCDLLIRCINCQKPLCP

SEQ ID NO: 112 - protein, 31 aa, synthetic construct
CPEEKQRHLDKKQRFHNIRGRWTGRCMSCCR

SEQ ID NO: 113 - protein, 27 aa, synthetic construct
MHGDTPTLHEYMLDLQPETTDLYCYEQ

SEQ ID NO: 114 - protein, 29 aa, synthetic construct
AGQAEPDRAHYNIVTFCCKCDSTLRLCVQ

SEQ ID NO: 115 - protein, 32 aa, synthetic construct; identical to SEQ ID NO: 71

SEQ ID NO: 116 - protein, 29 aa, synthetic construct
  MOD_RES 26: note = Abu
NSQYSNEKWTLQDVSLEVYLTAPTGXIKK

SEQ ID NO: 117 - protein, 32 aa, synthetic construct
  MOD_RES 10: note = Abu
  MOD_RES 19: note = Abu
LKGDANTLKXLRYRFKKHXTLYTAVSSTWHWT

SEQ ID NO: 118 - protein, 29 aa, synthetic construct
  SITE 1: note = Nle
  SITE 8: note = Nle
  MOD_RES 23: note = Abu
XHQKRTAXFQDPQERPRKLPQLXTELQTT

SEQ ID NO: 119 - protein, 29 aa, synthetic construct
  MOD_RES 8: note = Abu
  MOD_RES 22: note = Abu
  MOD_RES 25: note = Abu
PRKLPQLXTELQTTIHDIILEXVYXKQQL

SEQ ID NO: 120 - protein, 29 aa, synthetic construct
  MOD_RES 7: note = Abu
  MOD_RES 10: note = Abu
  MOD_RES 28: note = Abu
HDIILEXVYXKQQLLRREVYDFAFRDLXI

SEQ ID NO: 121 - protein, 29 aa, synthetic construct
  MOD_RES 13: note = Abu
  MOD_RES 25: note = Abu
  MOD_RES 28: note = Abu
RREVYDFAFRDLXIVYRDGNPYAVXDKXL

SEQ ID NO: 122 - protein, 29 aa, synthetic construct
  MOD_RES 10: note = Abu
  MOD_RES 13: note = Abu
  MOD_RES 27: note = Abu
YRDGNPYAVXDKXLKFYSKISEYRHYXYS

SEQ ID NO: 123 - protein, 29 aa, synthetic construct
  MOD_RES 12: note = Abu
  MOD_RES 29: note = Abu
FYSKISEYRHYXYSLYGTTLEQQYNKPLX

SEQ ID NO: 124 - protein, 29 aa, synthetic construct
  MOD_RES 14: note = Abu
  MOD_RES 20: note = Abu
  MOD_RES 23: note = Abu
  MOD_RES 28: note = Abu
YGTTLEQQYNKPLXDLLIRXINXQKPLXP

SEQ ID NO: 125 - protein, 31 aa, synthetic construct
  MOD_RES 1: note = Abu
  MOD_RES 26: note = Abu
  SITE 27: note = Nle
  MOD_RES 29: note = Abu
  MOD_RES 30: note = Abu
XPEEKQRHLDKKQRFHNIRGRWTGRXXSXXR

SEQ ID NO: 126 - protein, 27 aa, synthetic construct
  SITE 1: note = Nle
  SITE 12: note = Nle
  MOD_RES 24: note = Abu
XHGDTPTLHEYXLDLQPETTDLYXYEQ

SEQ ID NO: 127 - protein, 29 aa, synthetic construct
  MOD_RES 17: note = Abu
  MOD_RES 18: note = Abu
  MOD_RES 20: note = Abu
  MOD_RES 27: note = Abu
AGQAEPDRAHYNIVTFXXKXDSTLRLXVQ

SEQ ID NO: 128 - protein, 32 aa, synthetic construct; identical to SEQ ID NO: 97
  MOD_RES 2: note = Abu
  SITE 18: note = Nle
  MOD_RES 25: note = Abu
  MOD_RES 28: note = Abu

SEQ ID NO: 129 - protein, 244 aa, synthetic construct
APLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 130 - protein, 35 aa, synthetic construct
CGGVLVHPQWVLTAAHCIRNKSVILLGRHSLFHPE

SEQ ID NO: 131 - protein, 35 aa, synthetic construct
SLFHPEDTGQVFQVSHSFPHPLYDMSLLKNRFLRP

SEQ ID NO: 132 - protein, 29 aa, synthetic construct
PCALPERPSLYTKVVHYRKWIKDTIVANP

SEQ ID NO: 133 - protein, 364 aa, synthetic construct
FFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 134 - protein, 35 aa, synthetic construct
RTLMSAMTNLAALFPPEGVSIWNPILLWQPIPVHT

SEQ ID NO: 135 - protein, 35 aa, synthetic construct
PILLWQPIPVHTVPLSEDQLLYLPFRNCPRFQELE

SEQ ID NO: 136 - protein, 35 aa, synthetic construct
ATEDTMTKLRELSELSLLSLYGIHKQKEKSRLQGG

SEQ ID NO: 137 - protein, 35 aa, synthetic construct
LQGGVLVNEILNHMKRATQIPSYKKLIMYSAHDTT

SEQ ID NO: 138 - protein, 35 aa, synthetic construct
MALDVYNGLLPPYASCHLTELYFEKGEYFVEMYYR

SEQ ID NO: 139 - protein, 35 aa, synthetic construct
YFEKGEYFVEMYYRNETQHEPYPLMLPGCSPSCPL

SEQ ID NO: 140 - protein, 338 aa, synthetic construct
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVLIFKSILFLPCLRKKILKIRHGWEDVTKINKTEICSQL

SEQ ID NO: 141 - protein, 35 aa, synthetic construct
LFPQWHLPIKIAAIIASLTFLYTLLREVIHPLATS

SEQ ID NO: 142 - protein, 35 aa, synthetic construct
YTLLREVIHPLATSHQQYFYKIPILVINKVLPMVS

SEQ ID NO: 143 - protein, 38 aa, synthetic construct
RKQFGLLSFFFAVLHAIYSLSYPMRRSYRYKLLNWAYQ

SEQ ID NO: 144 - protein, 35 aa, synthetic construct
EDAWIEHDVWRMEIYVSLGIVGLAILALLAVTSIP

SEQ ID NO: 145 - protein, 35 aa, synthetic construct
LAVTSIPSVSDSLTWREFHYIQSKLGIVSLLLGTI

SEQ ID NO: 146 - protein, 35 aa, synthetic construct
DIKQFVWYTPPTFMIAVFLPIVLIFKSILFLPCLR

SEQ ID NO: 147 - protein, 389 aa, synthetic construct
SSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 148 - protein, 35 aa, synthetic construct
SPTSSASSFSSSAPFLASAVSAQPPLPDQCPALCE

SEQ ID NO: 149 - protein, 35 aa, synthetic construct
RNLTEVPTDLPAYVRNLFLTGNQLAVLPAGAFARR

SEQ ID NO: 150 - protein, 35 aa, synthetic construct
ALQGLRRLELASNHFLYLPRDVLAQLPSLRHLDLS

SEQ ID NO: 151 - protein, 35 aa, synthetic construct
LSNNSLVSLTYVSFRNLTHLESLHLEDNALKVLHN

SEQ ID NO: 152 - protein, 35 aa, synthetic construct
DCDPILPPSLQTSYVFLGIVLALIGAIFLLVLYLN

SEQ ID NO: 153 - protein, 20 aa, synthetic construct
  CARBOHYD 4: note = O-linked glycosylation at threonine
  CARBOHYD 5: note = O-linked glycosylation at serine
  CARBOHYD 9: note = O-linked glycosylation at threonine
  CARBOHYD 15: note = O-linked glycosylation at serine
  CARBOHYD 16: note = O-linked glycosylation at threonine
HGVTSAPDTRPAPGSTAPPA

SEQ ID NO: 154 - protein, 20 aa, synthetic construct
  CARBOHYD 2: note = O-linked glycosylation at threonine
  CARBOHYD 8: note = O-linked glycosylation at serine
  CARBOHYD 9: note = O-linked glycosylation at threonine
  CARBOHYD 17: note = O-linked glycosylation at threonine
  CARBOHYD 18: note = O-linked glycosylation at serine
DTRPAPGSTAPPAHGVTSAP

SEQ ID NO: 155 - protein, 27 aa, synthetic construct
  CARBOHYD 2: note = O-linked glycosylation at serine
  CARBOHYD 22: note = O-linked glycosylation at serine
  CARBOHYD 12: note = O-linked glycosylation at serine
  CARBOHYD 3: note = O-linked glycosylation at threonine
  CARBOHYD 11: note = O-linked glycosylation at threonine
  CARBOHYD 16: note = O-linked glycosylation at threonine
  CARBOHYD 23: note = O-linked glycosylation at threonine
GSTAPPAHGVTSAPDTRPAPGSTAPPA

SEQ ID NO: 156 - protein, 12 aa, synthetic construct
  CARBOHYD 4: note = O-linked glycosylation at serine
  CARBOHYD 3: note = O-linked glycosylation at threonine
  CARBOHYD 8: note = O-linked glycosylation at threonine
GVTSAPDTRPAP

SEQ ID NO: 157 - protein, 12 aa, synthetic construct
  CARBOHYD 4: note = O-linked glycosylation at threonine
  CARBOHYD 11: note = O-linked glycosylation at threonine
  CARBOHYD 10: note = O-linked glycosylation at serine
APDTRPAPGSTA

SEQ ID NO: 158 - protein, 14 aa, synthetic construct
  CARBOHYD 3: note = O-linked glycosylation at threonine
  CARBOHYD 11: note = O-linked glycosylation at threonine
  CARBOHYD 2: note = O-linked glycosylation at serine
  CARBOHYD 12: note = O-linked glycosylation at serine
GSTAPPAHGVTSAP

SEQ ID NO: 159 - protein, 6 aa, synthetic construct
  CARBOHYD 2: note = O-linked glycosylation at threonine
DTRPAP

SEQ ID NO: 160 - protein, 5 aa, synthetic construct
  CARBOHYD 2: note = O-linked glycosylation at serine
  CARBOHYD 3: note = O-linked glycosylation at threonine
GSTAP

SEQ ID NO: 161 - protein, 25 aa, synthetic construct
MTEYKLVVVGACGVGKSALTIQLIQ

SEQ ID NO: 162 - protein, 42 aa, unidentified
  MOD_RES 36: note = Azido-lysine-dibenzylcyclooctyne
  MOD_RES 37: note = Amino-hexanoic acid
  MOD_RES 38: note = Glutamic acid residue is covalently linked to TLR-7/8 agonist drug
  MOD_RES 40: note = Glutamic acid residue is covalently linked to TLR-7/8 agonist drug
  MOD_RES 42: note = Glutamic acid residue is covalently linked to TLR-7/8 agonist drug
VZKHKSAIVTLTYDSEWQRDQFLSQVKIPKTSPVZKXEWEWE

SEQ ID NO: 163 - protein, 48 aa, unidentified
  MOD_RES 42: note = Azido-lysine-dibenzylcyclooctyne
  MOD_RES 43: note = Amino-hexanoic acid
  MOD_RES 48: note = Glutamic acid residue is covalently linked to TLR-7/8 agonist drug
  MOD_RES 46: note = Glutamic acid residue is covalently linked to TLR-7/8 agonist drug
  MOD_RES 44: note = Glutamic acid residue is covalently linked to TLR-7/8 agonist drug
VZCGGVLVHPQWVLTAAHCIRNKSVILLGRHSLFHPESPVZKXEWEWE

SEQ ID NO: 164 - protein, 38 aa, unidentified
  MOD_RES 32: note = Azido-lysine-dibenzylcyclooctyne
  MOD_RES 33: note = Amino-hexanoic acid
  MOD_RES 38: note = Glutamic acid residue is covalently linked to TLR-7/8 agonist drug
  MOD_RES 36: note = Glutamic acid residue is covalently linked to TLR-7/8 agonist drug
  MOD_RES 34: note = Glutamic acid residue is covalently linked to TLR-7/8 agonist drug
VZMTEYKLVVVGACGVGKSALTIQLIQSPVZKXEWEWE

SEQ ID NO: 165 - protein, 6 aa, unidentified
  MOD_RES 1: note = Amino-hexanoic acid
  MOD_RES 6: note = Glutamic acid residue is covalently linked to TLR-7/8 agonist drug
  MOD_RES 4: note = Glutamic acid residue is covalently linked to TLR-7/8 agonist drug
  MOD_RES 2: note = Glutamic acid residue is covalently linked to TLR-7/8 agonist drug
XEWEWE

SEQ ID NO: 166 - protein, 13 aa, unidentified
AKFVAAWTLKAAA

SEQ ID NO: 167 - protein, 24 aa, unidentified
  MOD_RES 22: note = Azido-lysine-dibenzylcyclooctyne
  MOD_RES 23: note = The Glutamic acid of Glu-Lys sequence is linked to C-terminus of the Linker Azido-lysine-dibenzylcyclooctyne (Lys(N3-DBCO)
SLVRAKFVAAWTLKAAASLVRKEK

SEQ ID NO: 168 - protein, 5 aa, synthetic construct
  CARBOHYD 2: note = O-linked glycosylation at threonine
  CARBOHYD 3: note = O-linked glycosylation at serine
VTSAP

SEQ ID NO: 169 - protein, 4 aa, synthetic construct
  source 1..4: note = KS residues can be repeated up to eighteen times
KSKS

SEQ ID NO: 170 - protein, 4 aa, synthetic construct
  source 1..4: note = DS residues can be repeated up to eighteen times
DSDS

(19) United States
(12) Patent Application Publication
(10) Pub. No.: US 2026/0091097 A1
(43) Pub. Date: Apr. 2, 2026

(54) COMBINATION TREATMENT REGIMES FOR TREATING CANCER

(71) Applicants: Barinthus Biotherapeutics North America, Inc., Germantown, MD (US); The United States of America, As Represented by The Secretary, Department of Health and Human Servic, Bethesda, MD (US); The Chancellor, Masters and Scholars of The University of Oxford, Oxford (GB)

(72) Inventors: Geoffrey Martin Lynn, Germantown, MD (US); Christopher Martin O'Brien Garliss, Baltimore, MD (US); Andrew Scott Ishizuka, Washington, DC (US); Faezzah Baharom, San Mateo, CA (US); Robert Alan Seder, Bethesda, MD (US); Ramiro Andrei Ramirez-Valdez, Boston, MA (US)

(21) Appl. No.: 19/183,635

(22) Filed: Apr. 18, 2025

Related U.S. Application Data
(63) Continuation of application No. PCT/US2023/077765, filed on Oct. 25, 2023.
(60) Provisional application No. 63/380,929, filed on Oct. 25, 2022.

(51) Int. Cl. A61K 39/00; A61K 9/00; A61K 35/17; A61K 39/385; A61K 40/11; A61K 40/31; A61K 47/60; A61P 35/00; C07K 14/705; C12N 15/86

(52) U.S. Cl. CPC A61K 39/001102; A61K 9/0019; A61K 35/17; A61K 39/385; A61K 40/11; A61K 40/31; A61K 47/60; A61P 35/00; C07K 14/70517; C12N 15/86; A61K 2039/54; A61K 2039/545; A61K 2039/572; A61K 2039/6031; A61K 2039/627; C12N 2710/10334

(57) ABSTRACT

The present disclosure relates to methods of treating cancer in subjects by a two-part treatment regime comprising a first treatment that provides antigen-specific CD4+ and/or CD8+ T cells in the subject and a second treatment, administered after a time interval, that induces systemic and/or tumor-specific inflammation in the subject.

### CROSS-REFERENCE TO RELATED APPLICATIONS

This application is a continuation of PCT/US2023/077765, filed Oct. 25, 2023, which claims the benefit of, and priority to, U.S. Provisional Application No. 63/380,929, filed on Oct. 25, 2022, the disclosure of each of which is hereby incorporated by reference in its entirety.

### GOVERNMENT RIGHTS

This invention was created in the performance of a Cooperative Research and Development Agreement with the National Institutes of Health, an Agency of the Department of Health and Human Services. The Government of the United States has certain rights in this invention.

### FIELD OF DISCLOSURE

The present disclosure relates to methods of treating cancer in subjects by a two-part treatment regime comprising a first treatment that provides antigen-specific CD4+ and/or CD8+ T cells in the subject and a second treatment, administered after a time interval, that induces systemic and/or tumor-specific inflammation in the subject.

### REFERENCE TO SEQUENCE LISTING

The instant application contains a Sequence Listing which has been submitted electronically in XML format and is hereby incorporated by reference in its entirety. Said XML file, created on Dec. 9, 2025, is named BARI-101USWOC1.xml and is 209,574 bytes in size.

### BACKGROUND OF THE DISCLOSURE

A key tenet of cancer immunotherapy is to harness the patient's own immune system to mediate tumor regression. For example, one goal of therapeutic cancer vaccines delivering tumor antigens is to promote tumor regression by inducing antigen-specific T cells in the subject. Although promising, clinical outcomes and immune responses measured in cancer vaccine trials have been limited in late-stage patients thus far, indicating that several challenges need to be addressed to improve the efficacy of such vaccines.

### SUMMARY OF THE DISCLOSURE

As described herein, the inventors have found that by modifying the route of vaccination, i.e., from subcutaneous to intravenous or intramuscular, one can alter the type of antigen-specific T cells generated, which may impact tumor regression. For instance, the Examples 1-8 herein describe administration of peptide antigen conjugate vaccine called “SNP” of a formula C-E1-A-E2-U-H-D, in which C is a solubilizing block (S) that is charged at physiological pH (e.g. 7.4), A is a peptide antigen, E1 and E2 are N-terminal and C-terminal extensions, U is a linker, H is a hydrophobic block, and D is a drug, in this case a TLR 7/8 agonist drug, and wherein the dash “-” indicates a covalent linkage. For example, this SNP peptide antigen conjugate vaccine incorporating a Toll-like receptor 7/8 agonist (TLR 7/8 agonist) delivered subcutaneously (SNP-SC) generated more terminally-differentiated CD8+ T cells, compared to one administered intravenously (SNP-IV), which generated more stem-cell like CD8+ T cells. Thus, SNP-IV was able to control tumor growth more effectively than SNP-SC despite SNP-SC generating a high level of CD8+ T cells.

Results herein further show that SNP-IV mediated tumor regression through two distinct innate and adaptive immune mechanisms, both inducing antigen-specific CD8+ T cells and also inducing tumor specific and/or systemic inflammation characterized by activation of IFN type I (IFN-I) signaling. Furthermore, optimal cancer vaccine regimens may require both priming of tumor-specific CD8+ T cells followed by inducing systemic and/or tumor-specific inflammation characterized by increased IFN-I signaling. Accordingly, the present disclosure relates to methods of treating cancer in subjects by a two-part treatment regime comprising a first treatment that provides antigen-specific CD4+ and/or CD8+ T cells in the subject and a second treatment, administered after a time interval, that induces systemic and/or tumor-specific inflammation in the subject.

For example, as described in Examples 1-8 herein, the SNP self-assembling peptide antigen conjugate nanoparticle vaccine that links tumor antigen peptides to an immunostimulant such as a Toll-like receptor 7/8 agonist (SNP-7/8a) was administered to tumor-bearing mice either twice subcutaneously (SNP-SC) or twice intravenously (SNP-IV) with a time lag of several days between first and second administrations. Both routes generated antigen-specific CD8⁺ T cells that infiltrated tumors. However, only SNP-IV mediated tumor regression, which was found to be dependent on systemic type I interferon (IFN-I) signaling at the time of the second administration. Single cell RNA-sequencing revealed that intratumoral monocytes expressing an immunoregulatory gene signature (Chil3, Anxa2, Wfdc17) were reduced after the second SNP-IV administration. In humans, the Chil3⁺ monocyte gene signature is enriched in CD16⁻ monocytes and associated with worse outcomes. As disclosed herein, the generation of tumor-specific CD8⁺ T cells combined with remodeling of the tumor microenvironment (TME) is a promising approach for tumor immunotherapy. Further experiments herein showed that a combination of a first treatment of SNP-IM or SNP-IV followed by a second treatment of SNP-IV also mediated tumor regression and increased IFN-I signaling at the time of the second administration. As further described in Example 9 and subsequent Examples herein, a combination of SNP-IM or SNP-IV followed by IV administration of a polynucleotide ChAdOx1 vaccine, optionally encoding a tumor antigen, also mediated tumor regression.

Thus, in some embodiments, the present disclosure encompasses methods of treating cancer in a subject, inter alia, comprising (a) administering a first treatment that provides antigen-specific CD4+ and/or CD8+ T cells in the subject, and (b) following a time interval (T), administering a second treatment that induces systemic and/or tumor-specific inflammation in the subject. In some cases, the second treatment is administered intravenously. In some cases, the first treatment is administered intravenously, intraperitoneally, intramuscularly, subcutaneously or intradermally. In some cases, neither the first nor the second treatment is administered subcutaneously (SC). In some cases, the first treatment is administered either intramuscularly or intravenously (by IM or by IV) while the second is administered IV. In some cases, the first treatment comprises a vaccine that delivers an tumor antigen to the subject, while the second treatment comprises an immunostimulant.

In some cases, both the first and second treatments comprise a vaccine delivering an tumor antigen as well as an immunostimulant, in some cases with the first treatment administered by either IM or IV and the second treatment by IV. In some cases, the first treatment vaccine is a peptide antigen conjugate administered IM or IV and the second treatment is a further peptide antigen conjugate, further comprising an immunostimulant or administered in conjunction with an immunostimulant, and which is administered by IV. In some cases, the peptide antigen constructs used for the first and second treatments are the same, or comprise the same peptide antigen. In other cases, there are structural differences in the constructs based on their intended route of administration (e.g., IM and IV).

For example, the disclosure herein encompasses methods of treating cancer in a subject comprising (a) administering a first treatment that provides antigen-specific CD4+ and/or CD8+ T cells in the subject, and (b) following a time interval (T), administering a second treatment that induces systemic and/or tumor-specific inflammation in the subject, wherein the second treatment comprises an immunostimulant administered intravenously. In some cases, the first treatment comprises a peptide antigen conjugate vaccine of formula S-[E1]-A-[E2]-[U]-H [D] or of formula PEG-[E1]-A-[E2]-[U]-H [D], wherein:
A is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage.

In some cases, the drug molecule D, if present, is covalently linked to the hydrophobic block H, and thus the peptide antigen conjugate has the formula S-[E1]-A-[E2]-[U]-H-[D] or PEG-[E1]-A-[E2]-[U]-H-[D]. In some cases, S is a charged molecule C that comprises one or more functional groups that are charged at physiological pH. In some cases, D may be an immunostimulant drug, or a chemotherapy drug. In some cases, the first treatment is administered by IM or IV. In some cases, if administered IV, a PEG-comprising peptide antigen conjugate is employed. In some cases, the second treatment comprises the PEG-[E1]-A-[E2]-[U]-H [D](e.g, PEG-[E1]-A-[E2]-[U]-H-[D]) administered IV in conjunction with or comprising an immunostimulant drug D.

In some cases, methods of treating cancer in a subject herein comprise (a) administering a first treatment by IM or IV that provides antigen-specific CD4+ and/or CD8+ T cells in the subject, and (b) following a time interval (T), administering a second treatment by IV that induces systemic and/or tumor-specific inflammation in the subject, wherein the first treatment comprises a peptide antigen conjugate vaccine of formula S-[E1]-A-[E2]-[U]-H [D] or of formula PEG-[E1]-A-[E2]-[U]-H [D], with the components as defined above and that may or may not comprise an immunostimulant administered in conjunction with or as part of the vaccine conjugate, and wherein as a second treatment comprises a polynucleotide vaccine, such as a chimp adenovirus (ChAdOx) vaccine (e.g., ChAdOx1), which optionally encodes an tumor antigen, and optionally further proteins, and which optionally encodes an immunostimulant protein or is administered in conjunction with an immunostimulant drug.

In further cases, other types of vaccines or immunotherapies that provide antigen-specific CD4+ and/or CD8+ T cells in the subject are administered as a first treatment, such as, for example, a polynucleotide vaccine or adaptive cell therapy, while an immunostimulant is administered as a second treatment that is able to induce IFN-I signaling. In some cases, an increase in IFN-I signaling may be observed based on increased IL12 or IFN-alpha in the subject, such as in a blood sample from the subject.

Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims. The accompanying drawings, which are incorporated in and constitute a part of this specification, illustrate certain embodiments and together with the description, serve to explain certain principles described herein.

### BRIEF DESCRIPTION OF THE DRAWINGS

FIGS. 1A-1J show that tumor-specific CD8+ T cells generated by SNP-SC controlled tumor growth when followed by IV adjuvant delivery. As described above, the “SNP” is a peptide-antigen conjugate vaccine of a formula C-E1-A-E2-U-H-D, in which C is a solubilizing block (S) that is charged at physiological pH (e.g. 7.4), A is a peptide antigen, E1 and E2 are N-terminal and C-terminal extensions, U is a linker, H is a hydrophobic block, and D is a TLR 7/8 agonist drug, and wherein the dash “-” indicates a covalent linkage. SC indicates subcutaneously administered SNP while IM and IV indicates intramuscularly or intravenously administered SNP. FIG. 1A shows a schematic of therapeutic study design. Mice were implanted with MC38 and treated with SNP-7/8a (Reps1) on day 7 and day 14 together with CPI. FIG. 1B shows tumor growth following treatment with SNP-IV prime and boost (circle), SNP-SC prime and boost (square) or SNP-SC prime and SNP-IV boost (diamond) (n=10). Statistics were assessed by two-way ANOVA. FIG. 1C shows survival curves following treatment or in untreated mice (dashed lines) (n=30). Statistics were assessed by log-rank test. FIG. 1D shows tumor growth following treatment with SNP-SC prime with Reps1 (solid line with circles) or an irrelevant antigen (dashed line with squares) (n=10). Statistics were assessed by two-way ANOVA. FIG. 1E shows tumor growth following treatment SNP-SC prime (Reps1) followed by SNP-IV boost containing an irrelevant antigen (diamonds) or polyIC:LC (triangles) (n=10). Statistics were assessed by two-way ANOVA. FIG. 1F shows survival curves following SNP-IV boost containing an irrelevant antigen (circles) or polyIC:LC (squares) or untreated (dashed lines) (n=30). Statistics were assessed by log-rank test. FIG. 1G shows flow cytometry analysis of blood stained with tetramer and CD44 antibody (concatenated, n=10). FIG. 1H shows bar graphs summarizing the frequency of tetramer⁺ CD8 T cells in blood after treatment (n=30). Statistics were assessed by Kruskal Wallis test. FIG. 1I shows heatmaps representing the median MFI of PD-1, Tim-3 and NKG2A on tetramer⁺ CD8⁺ T cells in blood (n=10) and spleens (n=5) on day 21. FIG. 1J shows histograms summarizing the MFI PD-1, Tim-3 and NKG2A and CD39 on CD8⁺ T cells in tumors (n=5) on day 21.

FIGS. 2A-2J show that NeoAg⁺ CD8⁺ T cells generated by SNP-SC controlled tumor growth when followed by IV adjuvant delivery. FIGS. 2A-2J relate to FIGS. 1A-1J. FIG. 2A shows TC-1 tumor growth following treatment with SNP-IV prime and boost (SNP-IV; SNP-IV), SNP-SC prime and boost (SNP-SC; SNP-SC), SNP-SC prime and SNP-IV boost (SNP-SC′ SNP-IV) or SNP-IV boost with irrelevant antigen (SNP-SC; SNP-IV (Irrelevant Ag)) (n=8). Statistics were assessed by two-way ANOVA. FIG. 2B shows bar graphs summarizing the frequency of tetramer⁺ CD8 T cells in blood after treatment with SNP-7/8a containing E6 antigen (n=5-8). Statistics were assessed by Kruskal Wallis test. FIG. 2C shows tumor growth following treatment with SNP-IV prime and boost with Reps1 (solid line) or irrelevant antigen (dashed line) (n=10). Statistics were assessed by two-way Anova. FIG. 2D shows survival curves following treatment with SNP-IV containing irrelevant antigen (filled circles) or in untreated mice (open circles) (n=10). Statistics were assessed by log-rank test. FIG. 2S shows bar graphs summarizing the frequency of tetramer⁺ CD8 T cells in blood after treatment (n=10). FIGS. 2F and 2G show bar graphs summarizing the frequency of tetramer⁺ CD8 T cells in spleen (FIG. 2D) and tumor (FIG. 2E) after treatment (n=5). FIG. 2H shows bar graphs summarizing the median MFI of PD-1, TIM-3 and NKG2A of tetramer⁺ CD8 T cells in blood (n=10) and spleen (n=5) after treatment. FIG. 2I shows flow cytometry analysis of tetramer⁺ CD8 T cells in the tumor after boosting with SNP-IV containing Trp1, an irrelevant antigen. FIG. 2H shows histograms showing the expression of CD39, NKG2A, PD-1 and Tim-3 on Reps1l (left) or Trp1⁺ (right) CD8⁺ T cells.

FIGS. 3A-3I show that SNP-IV but not SNP-SC resulted in intratumoral vaccine distribution and DC maturation. FIG. 3A shows in vivo imaging of mice following vaccination with fluorescently-labeled SNP-7/8a (n=4). FIG. 3B shows fluorescence radiant efficiency over time after fluorescently-labeled SNP-SC or SNP-IV gating on tumor as the region of interest (ROI) (n=4). FIG. 3C shows harvested tumor (top) and tumor-draining LNs (bottom) after SNP-IV or SNP-SC over time (n=2). FIG. 3D shows flow cytometry analysis of fluorescently-labeled SNP-7/8a and CD80 in tumor (top) and tumor-draining LN (n=4). FIG. 3E shows bar graphs summarizing the frequency of myeloid cell populations of total vaccine⁺ cells in tumor (left) or tumor-draining LN (right) (n=4). FIG. 3F shows measurements of cytokines IFNα (left) and IL-12 (right) in sera of mice after SNP-SC or SNP-IV at 8 nmol and 32 nmol (n=3). FIG. 3G shows bar graphs summarizing the numbers of cDC1 in spleen (left), tumor (middle) and tumor-draining LN (right) of mice that were untreated (“1”) or treated with SNP-IV prime and boost (“2”), SNP-SC prime and boost (“3”) or SNP-SC prime followed by SNP-IV boost (“4”) (n=4-6). FIG. 3H shows flow cytometry analysis of cDC1s 24 h after SNP-SC or SNP-IV vaccination in the spleen (top) and tumor (bottom) (n=6). FIG. 3I shows heatmaps representing the median MFI of CD86 after SNP-IV prime and boost (“2”), SNP-SC prime and boost (“3”) or SNP-SC prime followed by SNP-IV boost (“4”) (n=6).

FIGS. 4A-4I show that SNP-IV but not SNP-SC resulted in intratumoral vaccine distribution and DC maturation. FIGS. 4A-4I relate to FIGS. 3A-3I. Harvested spleens after SNP-IV or SNP-SC over time (n=2). FIG. 4B shows bar graphs summarizing the frequency of myeloid cell populations of total vaccine⁺ cells in the spleen (n=4). FIG. 4C shows a UMAP of live, non-tumor lineage cells identified 6 clusters of myeloid cells. Histograms show the expression of phenotypic markers expressed by cDC1, cDC2, monocytes and macrophages. FIG. 4D shows a UMAP focused on cDCs. Histograms show the expression of CCR7, CD80, CD86 and MHCII, highly expressed by subgroup ‘B’. FIG. 4E shows pie charts summarizing the proportion of cDC1-A and cDC1-B (left) or cDC2-A and cDC2-B (n=5). FIG. 4F shows a UMAP focused on monocytes. Histograms show the expression of CCR7, CD80, CD86 and MHCII, highly expressed by subgroup ‘C’ (monocyte-derived DC, moDC) but not subgroup ‘A’ (myeloid-derived suppressor cell, MDSC). FIG. 4G shows pie charts summarizing the proportion of Mono-A (MDSC), Mono-B and Mono-C (MDSC) after treatment (n=5). FIG. 4H shows a graph summarizing the log fold change of moDC/MDSC after treatment with SNP-SC or SNP-IV (n=5). Statistics were assessed by one-way ANOVA. FIG. 4I shows median MFI of CD86 on cDC1s in the spleen and tumor-draining LNs after treatment (n=3). Statistics were assessed by one-way ANOVA.

FIGS. 5A-5G show scRNA-seq of tumors revealed that intratumoral Chil3⁺ monocytes were significantly reduced after SNP-IV. FIG. 5A shows a schematic of therapeutic study design. Mice (n=3) were implanted with MC38 and treated with SNP-7/8a (Reps1) on day 7 and day 14 together with CPI. Spleens and tumors were harvested on day 15. scRNA-seq was performed on flow sorted myeloid cells. FIG. 5B shows a UMAP of total monocytes, macrophages and DCs identified as 9 metaclusters in spleen and tumor on day 15. FIG. 5C shows a dot plot of canonical markers identifying specific DC, monocyte and macrophage subsets. FIG. 5D shows a bar graph showing proportions of individual metaclusters identified in spleen or tumor. FIG. 5E shows feature plots highlighting individual genes C1qb, Plin2, Ace and Chil3 used to annotate monocyte/macrophage clusters. FIG. 5F shows UMAPs of tumor MNP in untreated mice or mice treated with SNP-SC prime followed by SNP-SC boost, SNP-IV (Reps1) boost or SNP-IV (irrelevant antigen) boost. FIG. 5G shows bar graphs summarizing frequencies of individual metaclusters in SNP-SC (SC), SNP-IV (IV (Reps1)), or SNP-IV (irrelevant antigen; IV (Irr)) boosted animals. Statistics were assessed by one-way ANOVA.

FIGS. 6A-6I show scRNA-seq of tumors revealed that intratumoral Chil3⁺ monocytes were significantly reduced after SNP-IV. FIGS. 6A-6I are related to FIGS. 5A-5G. FIG. 6A shows a UMAP showing original clusters identified by Seurat after filtering out contaminating lymphocytes and granulocytes. FIG. 6B shows generation of 9 metaclusters based on hierarchical ordering. FIG. 6C shows a density plot identifying stable states within UMAP clusters. FIG. 6D shows UMAPs of clusters separated by tissue: spleen (left panel) or tumor (right panel). FIG. 6E shows a bar graph showing proportion of cell cycle genes in each metacluster. FIG. 6F shows violin plots visualize expression of specific genes used to annotate monocyte/macrophage clusters. FIG. 6H shows bar graphs showing frequencies of pDC, cDC1 and cDC2 in tumors based on scRNA-seq data collected on day 15. FIG. 6H shows UMAPs of splenic MNP in untreated mice or mice treated with SNP-SC prime followed by SNP-SC boost, SNP-IV (Reps1) boost or SNP-IV (irrelevant antigen) boost. FIG. 6I shows bar graphs showing frequencies of mregDC, pDC, cDC1, cDC2 and monocyte/macrophages in spleens based on scRNA-seq data collected on day 15.

FIGS. 7A-7G show that Chil3⁺ monocytes expressed immunoregulatory gene signature while Plin2⁺ macrophages expressed interferon-related gene signature. FIG. 7A shows downstream analyses focused on monocyte/macrophage (MoMac) populations. FIG. 7B shows bar graphs showing the number of genes downregulated or upregulated by monocyte/macrophage populations following SNP-7/8a boost compared to untreated controls. FIG. 7C shows a volcano plot comparing significantly (P value<0.05) upregulated (fold change>0.25, right panel) or downregulated (fold change<0.25, left panel) genes within tumor macrophages in SNP-IV treated animals compared to untreated. FIG. 7D shows violin plots highlighting top DEGs related to Plin2+ macrophages (top) and Chil3+ monocytes (bottom). FIG. 7E shows a dot plot highlighting top pathways upregulated (up arrow) or downregulated (down arrow) in SNP-SC or SNP-IV treated groups compared to untreated. FIG. 7F shows flow cytometry plots showing identification of Chil3+ monocytes in tumors 24 h after boosting with SNP-IV compared to untreated animals (concatenated, n=3). FIG. 7G shows a bar graph summarizing the frequency of Chil3+ monocytes in tumors 24 h after boosting with SNP-IV compared to untreated animals (n=3).

FIGS. 8A-8C show that Chil3⁺ monocytes expressed immunoregulatory gene signature while Plin2⁺ macrophages expressed interferon-related gene signature. FIGS. 8A-8C are related to FIGS. 7A-7G. FIG. 8A shows a heatmap of top ten differentially expressed genes (DEGs) of each monocyte/macrophage population. FIG. 8B shows a dot plot highlighting top pathways identified by Metascape. FIG. 8C shows violin plots highlighting genes encoding cell surface receptors to distinguish Plin2⁺ macrophages Chil3⁺ monocytes and Ace⁺ monocytes by flow cytometry.

FIGS. 9A-9I show that interferon alpha is required for mediating anti-tumor efficacy after SNP-IV treatment. FIG. 9A shows a schematic of therapeutic study design. Mice were implanted with MC38 and treated with SNP-7/8a (Reps1) on day 7 and SNP-7/8a (Irrelevant antigen) on day 14 together with CPI. Blocking antibodies against IFNAR (MAR1-5A3) were given on day 13 (500 μg) and day 15 (200 μg). FIG. 9B shows measurements of IFNα in sera of mice after SNP-IV boost with isotype control or IFNAR blocking antibody (n=3-6). Statistics were assessed by Kruskal Wallis test. FIG. 9C shows tumor growth following treatment with SNP-SC prime followed by SNP-IV with isotype control (SC→IV) or IFNAR blocking antibody (SC→IV+αIFNAR Ab) (n=8). Statistics were assessed by two-way ANOVA. FIG. 9D shows survival curves following treatment with SNP-SC prime followed by SNP-IV with isotype control (SC→IV) or IFNAR blocking antibody (SC→IV+αIFNAR Ab) (n=8). Statistics were assessed by log-rank test. FIG. 9E shows bar graphs summarizing the frequency of tetramer+ CD8 T cells in blood after treatment (n=8). Statistics were assessed by Kruskal Wallis test. FIG. 9F shows measurements of cytokines and chemokines in sera of mice after SNP-IV boost with isotype control or IFNAR blocking antibody (n=3-6). Statistics were assessed by Mann-Whitney test. FIG. 9G shows heatmaps representing the median MFI of CD80, CD86 and CCR7 on cDC1s in the spleen, tumor-draining LNs and tumors after treatment (n=5). FIG. 9H shows flow cytometry plots showing identification of “Chil3+ monocytes” in tumors 24 h after boosting with SNP-IV with isotype control (SC→IV) or IFNAR blocking antibody (SC→IV+αIFNAR Ab) (concatenated, n=4). FIG. 9I shows a graph summarizing the frequency of “Chil3+ monocytes” in tumors of untreated animals or 24 h after boosting with SNP-IV with isotype control (SC→IV) or IFNAR blocking antibody (SC→IV+αIFNAR Ab) (n=4). Statistics were assessed by Mann-Whitney test.

FIGS. 10A-10G show that interferon alpha is required for mediating anti-tumor efficacy after SNP-IV treatment. FIGS. 10A-10G are related to FIGS. 9A-9I. FIG. 10A shows a heatmap of cytokines and chemokines analyzed by Luminex on sera collected 24 h after SNP-IV boosting in mice that were treated with isotype control (“1”) or IFNAR blocking antibodies (“2”) compared to untreated animals (“3”) (n=10). FIG. 10B shows bar graphs summarizing the median MFI of CD80, CD86 and CCR7 in the spleen (top row), tumor-draining lymph node (middle row) or tumor (bottom row) 24 h after SNP-IV in mice treated with isotype control (SNP-IV) or IFNAR blocking antibodies (SNP-IV+αIFNAR Ab) (n=10). FIG. 10C shows a schematic of therapeutic study design. zDC-DTR bone marrow chimeric mice were implanted with MC38 and treated with SNP-7/8a (Reps1) on day 7 and day 14 together with CPI. Mice were not given diphtheria toxin (solid) or given DT via IP (open square) or IT (open triangle) injection on days 13, 15 and 17. FIG. 10D shows tumor growth following treatment with SNP-SC prime followed by SNP-IV (grey lines) (n=5) with (open symbols) or without (closed symbols) DT injection in zDC-DTR mice. Statistics were assessed by two-way ANOVA. FIG. 10E shows TC-1 tumor growth following treatment with SNP-SC prime (E6 antigen) followed by SNP-IV (Irrelevant antigen) with isotype control (SC→IV) or IFNAR blocking antibody (SNP→IV+αIFNAR Ab) (n=8). Statistics were assessed by two-way ANOVA. FIG. 10F shows bar graphs summarizing the frequency of tetramer⁺ CD8 T cells in blood (n=5-8). Statistics were assessed by Kruskal Wallis test. FIG. 10G shows bar graphs summarizing the frequency of “Chil3⁺ monocytes” in TC-1 tumors of untreated animals (untreated) or 24 h after boosting with SNP-IV with isotype control (SNP-IV) or IFNAR blocking antibody (SNP-IV+αIFNAR Ab) (n=4).

FIGS. 11A-11C show Chil3⁺ monocyte markers in human tumor-associated myeloid cells. FIGS. 11A-11C are related to FIGS. 12A-12F. FIG. 11A shows the average expression of a gene set of human orthologs of Chil3⁺ monocyte markers expressed in the MoMac-VERSE dataset (referred to as the huChil3⁺ geneset). FIG. 11B shows macrophages and monocytes from Peng et al. (top row) and Kim et al. (middle row) and Zheng et al. (bottom row). Left panel: UMAP reduction and unbiased cluster assignment (resolution: 0.3, cluster membership indicated by color). Middle panel: Score for the huChil3 geneset in cells of each cluster. Right panel: Distribution of log FC between the indicated cluster and all other macrophages/monocytes in the dataset for all genes (left) and huChil3 genes (right). FIG. 11C shows survival curves across all TCGA patients (left panel), low grade glioma (middle panel) and clear cell renal cell carcinoma (right panel). Patients were stratified as high- or low-expression cohorts based on the median xCell monocyte geneset (Aran et al. 2017).

FIGS. 12A-F show that Chil3+ monocyte markers in human tumor-associated myeloid cells. FIG. 12A shows UMAP representation of macrophages and monocytes in the MoMac-VERSE (Mulder et al. Immunity 2021) filtered to contain cancer studies sequenced with 10× technology. FIG. 12B shows violin plots comparing the scores for huChil3 between monocytes and macrophages from (B). Statistics were assessed by Wilcoxon Rank Sum test (***, P<0.0001). FIG. 12C shows median score (y-axis) of huChil3 in each dataset of the MoMac-VERSE (dots) for each of the macrophage/monocyte subsets (x-axis). Mean±SD across studies represented as circles and lines, respectively. Statistics were assessed by one-way Anova (P<0.0001). Adjusted P value (Tukey's HSD test)<0.1 comparing #8 with any other cluster. FIG. 12D shows a heatmap showing a hierarchical clustering of median scores for huChil3 in each dataset and cluster (z-scored per dataset). FIG. 12E shows scores (y-axis) for huChil3 in bulk RNA-seq samples (small dots) from sorted populations (x-axis) of 364 individual tumors across 12 cancer types (Combes et al. Cell 2022). Large dots indicate median in each group. FIG. 12F shows survival curves across all TCGA (left), low grade glioma (middle) and clear cell renal cell carcinoma (right). Patients (n=8,911) were stratified as high- or low-expression cohorts based on median huChil3 geneset scores. Statistics were assessed by log-rank test.

FIGS. 13A-13C show figures related to the ChAdOx1 vectors. FIG. 13A shows the neoantigen cassette structure encoded in ChAdOx1 vectors. Mouse invariant chain (1) at the N-terminus followed by a spacer sequence (2)—to prevent formation of chimeric epitopes, and a pentamer repeat of the neoantigen-spacer (3). FIG. 13B shows exemplary staining of Reps1-tetramer+ CD8 T cells in IM and IV vaccinated mice 2 weeks post vaccination. FIG. 13C shows exemplary IFNγ and TNFα staining following peptide restimulation in both vaccinated and unvaccinated mice.

FIGS. 14A-14J show that ChAdOx1 vaccination elicits durable, higher magnitude, and more terminally differentiated CD8 T cells responses than IM vaccination. FIG. 14A shows a schematic of vaccination and sampling schedule for standard immunogenicity study. Mice are primed with ChAdOx1 and sampled 2 weeks and 16 weeks post vaccination to assess antigen specific CD8 T cell responses. FIG. 14B shows antigen specific CD8 T cell response measured 2 weeks post vaccination by tetramer staining blood, spleen, liver and popliteal lymph node samples. FIG. 14C shows gating strategy for SLECs/MPECs (left panel). Frequency of tetramer+ cells that falls into each of the SLEC/MPEC categories (right panel). FIG. 14D shows frequency of IFNγ and TNFα co-producers following peptide restimulation detected by flow cytometry. FIG. 14E shows pie charts showing proportion of cytokine producing cells that are polyfunctional or monofunctional. FIG. 14F shows gating strategy of Tim-3/PD-1 on representative flow plot of stimulated CD8 T cells (left). Proportion of IFNγ+ cells that express Tim-3 and/or PD-1 (right). FIG. 14G shows immunogenicity 16 weeks after either IV or IM ChAdOx1 vaccination, measured by tetramer staining in blood and spleen. FIG. 14H shows frequency of IFNγ and/or TNFα producing CD8 T cells measured by flow cytometry following peptide restimulation of splenocytes collected 16 weeks post vaccination. FIG. 14I shows MFI of PD-1 on tetramer+ CD8 T cells in the spleen 16 weeks post vaccination. FIG. 14J shows proportion of tetramer+ CD8 T cells in the spleen 16 weeks post vaccination that fall into each of the SLEC/MPEC categories. Statistics: Data represented as mean±SD, Mann-Whitney test.

FIGS. 15A-C show that interval between IV SNP prime and either IM or IV ChAdOx1 boost does not affect Reps1-specific CD8 T cell response magnitude. FIG. 15A shows a schematic for the interval vaccination study. Mice are primed 4, 2, or 1 week prior to boosting with IV or IM ChAdOx1. Some mice receive only IV or IM ChAdOx1 and work as a benchmark for heterologous prime boost. Mice are bled to collect PBMCs for tetramer staining to quantify the Reps1-antigen specific response at the time of ChAdOx1 vaccination and then 1-, 2-, 4-, 8-, and 16-weeks post vaccination. The legend corresponds to the interval, whereas the shape legend indicates the route of ChAdOx1 administration. All SNP vaccinations were given IV. FIG. 15B shows kinetics of Reps1-specific CD8 T cell responses over the course of the study. FIG. 15B shows a comparison of the effect of route on magnitude of the Reps1-specific CD8 T cell response 2 weeks post boost, matched by interval. Statistics: data represented as mean±SD. (FIG. 15C) Mann-Whitney test.

FIGS. 16A-16I show that intravenous heterologous prime boost elicits high magnitude T cell responses that protect mice from MC38 tumor challenge. FIG. 16A shows prophylactic study vaccination and sampling schedule, legend for entire figure. Mice are primed and boosted 2 weeks apart, and then challenged with tumor cells 2 weeks post boost. At the time of tumor challenge, mice are also bled to assess T cell responses and given 1 dose of αPD-L1. Some mice also received αCD80 antibody 3 days and 1 day prior to tumor challenge. FIG. 16B shows a legend for panels 16B-16C. FIG. 16B shows magnitude of Reps1-specific CD8 T cell responses in blood at the time of tumor challenge, measured by tetramer staining blood from groups that received IM ChAdOx1 and control groups. FIG. 16C shows survival of IM ChAdOx1 vaccinated mice and IV SNP positive control mice. FIG. 16D shows a legend for panels 16D-16F. FIG. 16D shows magnitude of Reps1-specific CD8 T cell responses in blood at the time of tumor challenge, measured by tetramer staining blood from groups that received IV ChAdOx1 and control groups. FIG. 16E shows survival curve following tumor implantation for IV ChAdOx1 groups and IV SNP positive control mice. FIG. 16F shows correlation of Reps-1 tetramer specific CD8 T cell response at time of challenge and tumor volume 23 days after implantation. FIG. 16G shows legend for panels 16G-16I. FIG. 16G shows CD8 T cell count in blood at time of challenge. FIG. 16H shows average tumor growth curves following MC38 tumor challenge in IV heterologous prime boost group with and without CD8 T cell depletion. FIG. 16I shows survival curve for IV heterologous prime boost group with and without CD8 T cell depletion. Statistics: (FIGS. 16B, 16D, 16G) Data represented as mean±SD, Kruskal-Wallis test with Dunn's correction for multiple comparisons. (FIGS. 16C, 16E, 16I) Mantel-Cox Log-rank test, compared to naïve mice unless otherwise indicated. (FIG. 16F) Spearman's Rank correlation, line of best fit. (FIG. 16H) Two-way ANOVA with Bonferroni correction for multiple comparisons.

FIGS. 17A-17G show individual tumor growth curves for prophylactic study groups receiving the indicated vaccinations. 30 days post tumor implantation marked with dotted line for comparison.

FIGS. 18A-18J show IV ChAdOx1 vaccination promotes tumor regression when used as part of a heterologous prime boost vaccination strategy. FIG. 18A shows a schematic of therapeutic study design. Mice were implanted with MC38 and vaccinated on day 7 and day 14 with the vaccinations indicated in the legend. Mice received 3 doses of αPD-L1 administered weekly beginning on day 14. Blood, spleens, and tumors were harvested on day 21 to assess Reps1-specific CD8 T cell responses. FIG. 18B shows legend for FIGS. 18B-18D. Average tumor growth curves for the IV heterologous prime boost group compared to the positive control (IV SNP given twice) and IV ChAdOx1 prime alone. FIG. 18C shows survival curves that relate to FIGS. 18B and 18D. FIG. 18D shows magnitude of Reps1-specific CD8 T cell responses in blood at day 21, measured by tetramer staining blood. FIG. 18E shows legend for FIGS. 18E-18G. Average tumor growth curves for the IV heterologous prime boost group compared to antigen-free vaccination controls. Either SNP does not contain the Reps1 antigen (3) or ChAdOx1 does not contain the Reps1 antigen (2). FIG. 18F shows survival curves that relate to FIGS. 18E and 18G. FIG. 18G shows magnitude of Reps1-specific CD8 T cell responses in blood at day 21, measured by tetramer staining blood. FIG. 18H shows legend for FIGS. 18H-18J. Number of Reps1-specific CD8 T cells per mg of tumor tissue processed in groups with equivalent efficacy. FIGS. 18I-18J show MFI of PD-1 (FIG. 18I) or Tim-3 (FIG. 18J) on Reps1-specific CD8 T cells in the tumor at day 21. Statistics: (FIGS. 18B, 28E) Two-way ANOVA with Bonferroni correction for multiple comparisons, p-values compared to naïve mice. (FIGS. 18C, 18F) Mantel-Cox Log-rank test, groups compared as indicated in figure by paired legend color-matched circles. (FIGS. 18D, 18G, 18H, 18I, 18J) Data represented as mean±SD, Kruskal-Wallis test with Dunn's correction for multiple comparisons.

FIGS. 19A-19F show that tumor control in the therapeutic setting is dependent on CD8 T cells. FIG. 19A show a schematic of therapeutic study design. Mice were implanted with MC38 and vaccinated on day 7 and day 14 with the IV vaccinations indicated in the legend. Mice received 3 doses of αPD-L1 administered weekly beginning on day 14. Mice received 3 doses of a CD8 T cell depleting antibody before and after the boost vaccination. Spleens and tdLN were harvested on day 15 to assess effectiveness of CD8 depletion, and blood was collected on day 21 for the same purpose. FIGS. 19B, 19C, and 19D show CD8 T cell counts in the (FIG. 19B) spleen and (FIG. 19C) tdLN 1 day post-boost, and also in (FIG. 19D) blood 1 week post boost. FIG. 19E shows average tumor growth curves for the IV heterologous prime boost group with and without CD8 T cell depletion. FIG. 19F shows survival curves for the IV heterologous prime boost group with and without CD8 T cell depletion. statistics: Data represented as mean±SD (FIGS. 19B, 19C, 19D) Mann-Whitney. (FIG. 19E) Two way ANOVA with Bonferroni correction for multiple comparisons. (FIG. 19F) Mantel-Cox Log-rank test.

FIGS. 20A-20E show that ChAdOx1 vaccination activates STING to elicit transient systemic release of IFNα, which is required for priming CD8 T cell responses. FIG. 20A shows groups of mice were vaccinated in a staggered fashion with IV ChAdOx1 or IV SNP either 72, 24 or 6 hours prior to bleeding. FIGS. 20B-20D show cytokine measurements in serum 6, 24, and 72 hours after IV vaccination. FIG. 20B: IFNα. FIG. 20C: IP-10. FIG. 20D: IL-12p70. FIG. 20E shows antigen-specific CD8 T cell response 2 weeks post IV ChAdOx1 vaccination measured by tetramer staining in WT, STING KO, IFNα receptor KO, and IL-12 KO mice. Statistics: Data represented as mean±standard deviation. (FIGS. 20B, 20C, 20D) Two way ANOVA with Bonferroni correction for multiple comparisons. (FIG. 20E) Kruskal-Wallis test with Dunn's correction for multiple comparisons.

FIGS. 21A-21K show interferon alpha is required for mediating anti-tumor efficacy after ChAdOx1-IV treatment. FIG. 21A shows a schematic of therapeutic study design. Mice were implanted with MC38 and vaccinated on day 7 and day 14 with the vaccinations indicated in the legend. Mice received 3 doses of αPD-L1 administered weekly beginning on day 14. Some groups received saturating doses of IFNα receptor blocking antibody one day prior to and one day after the boost vaccination, as indicated in the legend. Blood, spleens, and tumors were harvested on day 21 to assess Reps1-specific CD8 T cell responses. FIGS. 21B and 21D show average tumor growth curves for the heterologous prime boost vaccinations with either the (FIG. 21B) Reps-1 encoding ChAdOx1 or (FIG. 21D) empty ChAdOx1. FIGS. 21C and 21E show survival curve for the heterologous prime boost vaccination groups with either the (FIG. 21C) Reps-1 encoding ChAdOx1 or (FIG. 21E) empty ChAdOx1. FIG. 21F show magnitude of the Reps1-specific CD8 T cell response 1 week post boost vaccination measured by tetramer staining PBMCs. FIG. 21G show heatmap plot of the average amount of a subset of cytokines assayed by Luminex present in serum 6 hours post boost. Scale is relative to the range for each individual cytokine. FIG. 21H show number of cDC1s per mg of tumor found 1 day post boost. FIG. 21I show number of cDC1s in the tdLN 1 day post boost. FIGS. 21J and 21K show expression of the maturation and migration marker (FIG. 21J) CCR7 and activation marker (FIG. 21K) CD86. Statistics: (FIGS. 21B, 21D) Two way ANOVA with Bonferroni correction for multiple comparisons, p-values compared to naïve mice unless otherwise indicated. (FIGS. 21C, 21E) Mantel-Cox Log-rank test. (FIGS. 21F, 21H, 21I, 21J, 21K) Data represented as mean±SD, Mann-Whitney test.

FIGS. 22A-22D show that tumor control in the therapeutic setting is dependent on CD8 T cells. FIG. 22A shows a schematic of therapeutic study design. Mice were implanted with B16-F10 Adpgk and vaccinated on day 7 and day 14 with the IV vaccinations indicated in the legend. Mice received 3 doses of αPD-L1 administered weekly beginning on day 14. Some groups received saturating doses of IFNα receptor blocking antibody one day prior to and one day after the boost vaccination, as indicated in the legend. FIG. 22B shows average tumor growth curves for the IV heterologous prime boost group with and without IFNAR1 blockade

FIG. 22C shows survival of the IV heterologous prime boost group with and without IFNAR1 blockade. FIG. 22D shows Adpgk-specific CD8 T cell response measured by tetramer staining one week post IV ChAdOx1 boost with or without IFNAR1 blockade.

FIGS. 23A-23F show production of pro-inflammatory systemic cytokines and activation of monocytes in the tumor is dependent on Type I IFNs. FIG. 23A show a schematic of therapeutic study design carried out as described in FIGS. 25A-25E. FIGS. 23A-23F show cytokines detected in the serum 6 hours post boost vaccination (FIG. 23B) IFNγ (FIG. 23C) TNFα (FIG. 23D) CXCL-9 (FIG. 23E) CXCL-10 (FIG. 23F) IL-6. Statistics: Data represented as mean±standard deviation (FIGS. 23B-23F) Mann-Whitney test to compare within each vaccine strategy the effect of IFNα receptor blockade.

FIGS. 24A-24T show IV ChAdOx1 elicits Type I IFNs that increase the ratio of pro-inflammatory to anti-inflammatory monocytes at the tumor site. FIG. 24A shows schematic of therapeutic study design. Mice were implanted with MC38 and vaccinated with IV SNP twice or IV heterologous prime boost utilizing the antigen-encoding ChAdOx1. Spleens and tumors were harvested on 1 day post boost vaccination. Myeloid cells were sorted by FACS and used for scRNA sequencing. FIG. 24B shows UMAP visualization of scRNA sequencing data from spleen and tumor isolated monocytes, macrophages and DCs. Classified according to their metaclusters identity. FIG. 24C shows dot plot of canonical markers identifying specific DC, monocyte and macrophage subsets. FIG. 24D shows correlation matrix of the metaclusters identified in the present study and the metaclusters identified in a published data set (Baharom et. al. 2022). FIG. 24E shows UMAPs of tumor MNPs separated by treatment group. FIG. 24F shows bar graphs summarize frequencies of monocyte and macrophage metaclusters for each animal across different treatment groups in the tumor. FIG. 24G shows heatmap of gene set score analysis results focused on mono/mac subsets. FIG. 24H shows heatmap of gene set score analysis results focused on DC subsets. FIGS. 24I-24Q show bar graphs of DC, mono, or mac metacluster frequencies in the tumor of mice in each treatment group. FIG. 24R shows violin plots of 2 differentially expressed genes (H2-2a, Ly6A) between the Chil-3 monocytes and the remaining mono/mac metaclusters. FIG. 24S shows exemplary staining of Chil-3 monocytes and activated monocytes found in the tumor of different treatment groups 1 day post boost. FIG. 24T shows ratio of activated monocytes to Chil-3 monocytes found in the tumor 1 day post boost.

FIGS. 25A-25E relate to clustering of myeloid cells. FIG. 25A show original clustering of scRNA-seq data visualized by UMAP dimensionality reduction. FIG. 25B show hierarchical clustering of original clusters into metaclusters. FIG. 25C show density plot to identify stable states in the visualized UMAP. FIG. 25D show metacluster UMAP segregated by tissue of origin of the cells (spleen and tumor). FIG. 25E show bar graphs summarizes relative frequencies of myeloid metaclusters in spleen and tumor.

### DESCRIPTION OF CERTAIN EMBODIMENTS

### I. Definitions

Details of certain terms are given below. The terminology in this disclosure is understood to be useful for the purpose of providing a better description of particular embodiments and should not be considered limiting.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

About: In the context of the present disclosure, “about” when referring to a measurable value such as an amount, a temporal duration, and the like, is meant to encompass variations of 20%, +10%, +5%, ±1%, or ±0.1% from the specified value, as such variations are appropriate to perform the disclosed methods. For example, “about 10” refers to 9.5 to 10.5. A ratio of “about 5:1” refers to a ratio from 4.75:1 to 5.25:1.

Adjuvant: Any material added to vaccines to enhance or modify the immunogenicity of an antigen. Adjuvants can be delivery systems, such as particles based on inorganic salts (e.g., aluminum hydroxide or phosphate salts referred to as alum), water-in-oil or oil-in-water emulsions or polymer particles (e.g., PLGA) in which antigen is simply admixed with or adsorbed, incorporated within or linked indirectly or directly through covalent interactions. In some cases, adjuvants may also be amphiphilic compounds. Alternatively, adjuvants can be chemically defined molecules that bind to defined receptors and induce downstream signaling pathways, including immunostimulants such as pattern recognition receptor (PRR) agonists, such as synthetic or naturally occurring agonists of Toll-like receptors (TLRs), stimulator of interferon genes (STING), nucleotide-binding oligomerization domain-like receptors (NLRs), retinoic acid-inducible gene-I-like receptors (RLRs) or C-type lectin receptors (CLRs), as wells as biological molecules (a “biological adjuvant”), such as IL-2, RANTES, GM-CSF, TNF-α, IFN-7, G-CSF, LFA-3, CD72, B7-1, B7-2, OX-40L, 4-1BBL. Small molecule analogs of nucleotide bases, such as hydroxyadenine and imidazoquinolines, that bind to Toll-like receptors-7 (TLR-7) and TLR-7/8a, respectively, as well as agonists of TLR-2/6, TLR-4, STING and NOD are used as exemplary PRR agonists in the present disclosure. The person of ordinary skill in the art is familiar with adjuvants (see: Perrie et al., Int J Pharm 364:272-280, 2008 and Brito et al., Journal of controlled release, 190C:563-579, 2014). In general, a biological adjuvant listed herein can be joined to a peptide antigen conjugate of the present disclosure, for example, through any suitable means. In some examples, treatment with adjuvant (e.g., adjuvant delivery) refers to treatment with adjuvant alone without antigen.

Administration: To provide or give to a subject an agent, for example, a vaccine or immunostimulant. Exemplary routes of administration include, but are not limited to, oral, injection (such as subcutaneous, intramuscular, intradermal, intraperitoneal, and intravenous), transdermal, topical, intranasal, vaginal, and inhalation routes. “Administration of” and “administering a” compound should be understood to mean providing a compound, a prodrug of a compound, or a pharmaceutical composition as described herein. The compound or composition can be administered by another person to the subject or it can be self-administered by the subject.

Antigen: Any molecule that contains an epitope that binds to a T cell or B cell receptor and can stimulate an immune response, in particular, a B cell response and/or a T cell response in a subject. The epitopes may comprise peptides, glycopeptides, lipids or any suitable molecules that contain an epitope that can interact with components of specific B cell or T cell receptors. Such interactions may generate a response by the immune cell. “Epitope” refers to the region of a peptide antigen to which B and/or T cell proteins, i.e., B-cell receptors and T-cell receptors, interact. Antigens used in embodiments of the present disclosure may be selected from pathogens, cancerous cells, autoantigens, alloantigens or allergens. Many such antigens may be used according to embodiments of the inventions of the present disclosure and are discussed in greater detail throughout this specification. A “tumor antigen” or “tumor-associated antigen” as used herein refers to an antigen associated with a tumor or cancer. Examples of tumor antigens re discussed further below.

Antigen-presenting cell (APC): Any cell that presents antigen bound to MHC class I or class II molecules to T cells, including but not limited to monocytes, macrophages, dendritic cells, B cells, T cells and Langerhans cells.

Amphiphilic: The term “amphiphilic” is used herein to mean a substance containing both hydrophilic or polar and hydrophobic groups.

CD4: Cluster of differentiation 4, a surface glycoprotein that interacts with MHC Class II molecules present on the surface of other cells. A subset of T cells express CD4 and these cells are commonly referred to as helper T cells or CD4 T cells or CD4+ T cells.

CD8: Cluster of differentiation 8, a surface glycoprotein that interacts with MHC Class I molecules present on the surface of other cells. A subset of T cells express CD8 and these cells are commonly referred to as cytotoxic T cells (CTLs), killer T cells or CD8 T cells or CD8+ T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule. “Charged functional groups refer to functional groups that may be permanently charged or have charge depending on the pH. Charged functional groups may be partial or full integer values of charge, which may be positive or negative, are referred to as positively charged functional groups or negatively charged functional groups, respectively. The portion of a molecule that comprises one or more charged functional groups, which may be positive or negative, is referred to as a “charged group,” e.g., positively charged group or negatively charged group. Charged groups may comprise positive functional groups, negative functional groups or both positive and negative functional groups.

The net charge of the charged group may be positive, negative or neutral. Charged monomers refer to monomers that comprise charged groups. Charged amino acids are a type of charged monomer. Note: the net charge of a particle comprising amphiphiles and/or peptide antigen conjugates further comprising charged groups, e.g., charged monomers, such as charged amino acids, can be estimated by summing the charge of each functional group within the amphiphiles and/or peptide antigen conjugates.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group with an alkyne to form a triazole linker through strain-promoted [3+2]azide-alkyne cyclo-addition.

Copolymer: A polymer derived from two (or more) different monomers, as opposed to a homopolymer where only one monomer is used. Since a copolymer includes at least two types of constituent units (also structural units), copolymers may be classified based on how these units are arranged along the chain. A copolymer may be a statistical (or random) copolymer wherein the two or monomer units are distributed randomly; the copolymer may be an alternating copolymer wherein the two or more monomer units are distributed in an alternating sequence; or, e.g., the copolymer, e.g., a poly(amino acid) may be produced by solid-phase peptide synthesis (SPPS) and have a specific order of monomer units. The term “block copolymer” refers generically to a polymer composed of two or more contiguous blocks of different constituent monomers or comonomers (if a block comprises two or more different monomers). Block copolymer may be used herein to refer to a copolymer that comprises two or more homopolymer subunits, two or more copolymer subunits or one or more homopolymer subunits and one or more copolymer subunits, wherein the subunits may be linked directly by covalent bonds or the subunits may be linked indirectly via an intermediate non-repeating subunit, such as a junction block or linker. Blocks may be based on linear and/or brush architectures. Block copolymers with two or three distinct blocks are referred to herein as “diblock copolymers” and “triblock copolymers,” respectively. Copolymers may be referred to generically as polymers, e.g., a statistical copolymer may be referred to as a polymer or copolymer. Similarly, a block copolymer may be referred to generically as a polymer. While a copolymer used in herein means a polymer comprising two or more types of monomers, terpolymer is a copolymer with three monomer units.

Critical micelle concentration (CMC): Refers to the concentration of a material above which micelles spontaneously form to satisfy thermodynamic equilibrium.

Drug: refers to any pharmaceutically active molecule—including, without limitation, proteins, peptides, sugars, saccharides, nucleosides, inorganic compounds, lipids, nucleic acids, small synthetic chemical compounds, macrocycles, etc.—that has a physiological effect when ingested or otherwise introduced into the body. Pharmaceutically active compounds can be selected from a variety of known classes of compounds, including, for example, analgesics, anesthetics, anti-inflammatory agents, anthelmintics, anti-arrhythmic agents, antiasthma agents, antibiotics (including penicillins), anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin, biphosphonates, prostaglandins, radio-pharmaceuticals, hormones, sex hormones (including steroids), anti-allergic agents, stimulants and anoretics, steroids, sympathomimetics, thyroid agents, vaccines, vasodilators, and xanthines. Drugs may also be referred to as pharmaceutically active agents, pharmaceutically active substances or biologically active compounds or bioactive molecules. Any drug molecules in the formulae described herein are abbreviated “D.”

Drug delivery: A method or process of administering a pharmaceutical compound to achieve a therapeutic effect in humans or animals.

Effective amount: The amount of a compound, material, or composition effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art.

Graft copolymer: A polymer having a main polymer chain (e.g., polymer A) with one or more sidechains of a second polymer (e.g., polymer B). The first polymer A is linked through its monomers and sidechains to the second polymer B, which is bonded to individual monomers of polymer A thereby branching off from the chain of polymer A. A first polymer linked through an end group to a second polymer may be described as a block polymer (e.g., A-B type di-block) or an end-grafted polymer.

Hydropathy index/GRAVY value: Is a number representing the hydrophobic or hydrophilic characteristics of an amino acid or sequence of amino acids. There are a variety of scales that can be used to describe the relative hydrophobic and hydrophilic characteristics of amino acids comprising peptides. In the present disclosure, the Hydropathy scale of Kyte and Doolittle (Kyte J, Doolittle R F, J. Mol. Biol 157: 105-32, 1983) is used to calculate the grand average of hydropathy (GRAVY) value, sometimes referred to as the GRAVY score. The GRAVY value of a peptide is the sum of the Hydropathy values of all amino acids comprising the peptide divided by the length (i.e., number of amino acids) of the peptide. The GRAVY value is a relative value. The larger the GRAVY value, the more hydrophobic a peptide sequence is considered, whereas the lower the GRAVY value, the more hydrophilic a peptide sequence is considered.

Hydrophilic: Refers to the tendency of a material to disperse freely or be solubilized in aqueous solutions (sometimes referred to as aqueous media). A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble at concentrations of at least 1.0 mg/mL or more. Hydrophilic group refers to the portion of a molecule that is polar and/or charged and has good water solubility.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble in water, or are soluble in water only at concentrations of 1 mg/mL or less, typically 0.1 mg/mL or less or more preferably 0.01 mg/mL or less. Hydrophobic monomers are monomers, e.g., hydrophobic amino acids, that comprise hydrophobic groups and form polymers that are insoluble in water or insoluble in water at certain temperatures, pH and salt concentration. Hydrophobic group refers to a portion of a molecule that is hydrophobic. For example, a styrene monomer may be referred to as a hydrophobic monomer because poly(styrene) is a water insoluble polymer. Hydrophobic drugs refer to drug molecules that are insoluble or soluble only at concentrations of about 1.0 mg/mL or less in aqueous solutions at pH of about pH 7.4. Amphiphilic drugs are drug molecules that have the tendency to assemble into supramolecular structures, e.g., micelles, in aqueous solutions and/or have limited solubility in aqueous solutions at pH of about pH 7.4.

Hydrophobic molecule or hydrophobic block (H): In the present disclosure, the terms “hydrophobic molecule” and “hydrophobic block” (H) are used interchangeably herein, each as a general term to describe a molecule with limited water solubility, or amphiphilic characteristics, that can be linked to peptide antigens resulting in a peptide antigen conjugate that forms particles in aqueous conditions. The hydrophobic molecule (hydrophobic block) (H) in this context promotes particle assembly due to its poor solubility, or tendency to assemble into particles, in aqueous conditions over certain temperatures and pH ranges.

Hydrophobic molecules (H) as described herein are inclusive of amphiphilic molecules that may form supramolecular structures, such as micelles or bilayer-forming lamellar or multi-lamellar structures (e.g., liposomes or polymersomes), as well as compounds that are completely insoluble and form aggregates alone. The hydrophobic characteristics of the molecule may be temperature- and/or pH-responsive. In some embodiments, the hydrophobic molecule (H) is a polymer that is water soluble at low temperatures but is insoluble, or micelle-forming, at temperatures above, for example, 20° C., such as 20, 21, 22, 23, 24, 25, 26, 27, 28, 29, 30, 31, 32, 33, 34, 35, 36, 37, 38, 39 or 40° C. In other embodiments, the hydrophobic molecule (H) is a polymer that is water soluble at low pH, for example, at a pH below 6.5 but insoluble, for example, at a pH above 6.5. Examples of hydrophobic molecules (H) include but are not limited to fatty acids, cholesterol and its derivatives, long chain aliphatics, lipids and various polymers, such as polystyrene, poly(lactic-co-glycolic acid) (PLGA), as well as poly(amino acids) comprised of predominantly hydrophobic amino acids. In some embodiments, the hydrophobic molecule (H) is a hydrophilic polymer with multiple hydrophobic ligands attached. A variety of hydrophobic molecules useful for the practice of the present disclosure are disclosed herein.

Immune response: A change in the activity of a cell of the immune system, such as a B cell, T cell, or monocyte, as a result of a stimulus, either directly or indirectly, such as through a cellular or cytokine intermediary. An immune response may comprise a T cell response, such as a CD4 T cell response or a CD8 T cell response, and in some cases may be observable via an increase in CD4 and/or CD8 T cells in a subject. Such an immune response may result in the production of additional T cell progeny and/or in the movement of T cells. In other embodiments, the response is a B cell response, and results in the production of specific antibodies or the production of additional B cell progeny. In yet other embodiments, the response is an antigen-presenting cell response. An antigen may be used to stimulate an immune response leading to the activation of cytotoxic T cells that kills virally infected cells or cancerous cells. In other embodiments, an antigen may be used to induce tolerance or immune suppression. Thus, in certain embodiments, the immune response is specific for a particular antigen (an “antigen-specific response”). Accordingly, an “antigen-specific CD4+ and/or CD8+ T cell response” or “antigen-specific CD4 and/or CD8 T cell response” herein refers to a CD4+ and/or CD8+ T cell response against a particular antigen, such as a tumor antigen, such as an antigen provided in a vaccine or encoded by a vaccine. In such an antigen-specific CD4+ and/or CD8+ T cell response, may be characterized by an increase in the number of antigen-specific CD4 or CD8 T cells in the subject. The term “provides” in connection with an antigen-specific CD4 and/or CD8 T cell response means that the number of such antigen-specific CD4 and/or CD8 T cells increases. This may be because the treatment induces their production in the subject (i.e., by providing the antigen to the subject), and/or because the treatment provides such T cells to the subject (i.e., by an adoptive cell therapy).

Immunogenic composition: A formulation of materials comprising an antigen and optionally an immunomodulator that induces a measurable immune response against the antigen. For examples, vaccines are a type of immunogenic composition.

Immunomodulators: refers to a type of drug that modulates the activity of cells of the immune system, which includes immunostimulants and immunosuppressants.

Immunostimulants: refers to any synthetic or naturally occurring drugs that promote pro-inflammatory and/or cytotoxic activity by immune cells, and that, thus, may induce systemic inflammation and/or tumor specific inflammation (i.e. inflammation in the tumor microenvironment) when administered by IV. Exemplary immunostimulants include pattern recognition receptor (PRR) agonists, such as synthetic or naturally occurring agonists of Toll-like receptors (TLRs), stimulator of interferon gene agonists (STINGa), nucleotide-binding oligomerization domain-like receptor (NLR) agonists, retinoic acid-inducible gene-I-like receptors (RLR) agonists and certain C-type lectin receptor (CLR), as well as certain cytokines (e.g., certain interleukins), such as IL-2; certain chemokines or small molecules that bind chemokine receptors; certain antibodies, antibody fragments or synthetic peptides that activate immune cells, e.g., through binding to stimulatory receptors, e.g., anti-CD40, or, e.g., by blocking inhibitory receptors, e.g., anti-CTLA4, anti-PD1, etc. Various immunostimulants suitable for the practice of the present disclosure are described throughout the specification. For clarity, certain pharmaceutically active compounds that stimulate the immune system may be referred to as immunostimulants or more generally as drug molecules (abbreviated “D” in formulae).

In vivo delivery: Administration of a composition, such as a composition comprising amphiphilic block copolymers and drug(s), by topical, transdermal, suppository (rectal, vaginal), pessary (vaginal), intravenous, oral, subcutaneous, intraperitoneal, intrathecal, intramuscular, intracranial, inhalational, oral, or any other suitable route to a subject.

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker (sometimes referred to as linker molecule). The molecules may be linked indirectly by interposition of a component that binds non-covalently to both molecules independently. The term “Linker,” sometimes abbreviated “L” or “X,” used in chemical formulae herein means any suitable linker molecule. Specific linkers may be indicated by other symbols, such as L1, L2, X1, X2, X3, X4, X5, and U. Linker precursors may be indicated as U1, U2, and the like. Various linkers are described throughout the specification.

A “bilayer membrane” or “bilayer(s)” is a self-assembled membrane of amphiphiles or super-amphiphiles in aqueous solutions.

Micelles: Spherical receptacles having a single monolayer defining a closed compartment. Generally, amphiphilic molecules spontaneously form micellar structures in polar solvents. In contrast to bilayers, e.g., liposomal bilayers, micelles are “sided” in that they project a hydrophilic, polar outer surface and display a hydrophobic interior surface.

Mol %: Refers to the percentage of a particular type of monomeric unit (or “monomer”) that is present in a polymer. For example, a polymer having 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” or “monomer unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) comprise amino acid monomeric units. Monomeric units may also be referred to as monomers or monomer units or the like.

Net charge: The sum of electrostatic charges carried by a molecule or, if specified, a portion or section of a molecule.

Particle: A nano- or micro-sized supramolecular structure composed of an assembly of molecules. For example, amphiphiles and peptide antigen conjugates of the present disclosure form particles in aqueous solution. In some embodiments, particle formation by the amphiphiles and/or peptide antigen conjugates is dependent on pH or temperature. In some embodiments, the nanoparticles composed of amphiphiles and/or peptide antigen conjugates have an average diameter between 5 nanometers (nm) to 500 nm. In some embodiments, the nanoparticles composed of amphiphiles and/or peptide antigen conjugates form micelles and have an average diameter between 5 nanometers (nm) to 50 nm, such as between 10 and 30 nm. In some embodiments, the nanoparticles composed of amphiphiles and/or peptide antigen conjugates may be larger than 100 nm.

Pattern recognition receptors (PRRs): Receptors expressed by various cell populations, particularly innate immune cells that bind to a diverse group of synthetic and naturally occurring molecules. There are several classes of PRRs. Non-limiting examples of PRRs include Toll-like receptors (TLRs), RIG-I-like receptors (RLRs), NOD-like receptors (NLRs), Stimulator of Interferon Genes receptor (STING), and C-type lectin receptors (CLRs). Agonists of such PRRs are referred to as immunostimulant drugs and can be used to enhance and/or modify an immune response to an antigen. For more information on pattern recognition receptors, see Wales et al., Biochem Soc Trans., 35:1501-1503, 2007.

Peptide or polypeptide: Two or more natural or non-natural amino acid residues that are joined together in a series through one or more amide bonds. The amino acid residues may contain post-translational modification(s) (e.g., glycosylation, citrullination, homocitrullination, oxidation and/or phosphorylation). Such modifications may mimic post-translational modifications that occur naturally in vivo or may be non-natural. Any one or more of the components of the amphiphiles and/or peptide antigen conjugates may comprise peptides.

Peptide Modifications: Peptides may be altered or otherwise synthesized with one or more of several modifications as set forth below. In addition, analogs (non-peptide organic molecules), derivatives (chemically functionalized peptide molecules obtained starting from a peptide) and variants (homologs) of these peptides can be utilized in the methods described herein. The peptides described herein comprise a sequence of amino acids, analogs, derivatives, and variants, which may be either L- and/or D- versions. Unless otherwise specified, any peptide sequences referenced herein comprise L amino acids, preferably exclusively L amino acids. Such peptides may contain peptides, analogs, derivatives, and variants that are naturally occurring and otherwise.

Peptides can be modified through any of a variety of chemical techniques to produce derivatives having similar activity as the unmodified peptides, and optionally having other desirable properties. For example, carboxylic acid groups of the peptide, whether at the carboxyl terminus or at a side chain, can be provided in the form of a salt of a pharmaceutically-acceptable cation or esterified to form a CC₁-CC₁₆ ester, wherein CC refers to a carbon chain (and thus, CC1 refers to a single carbon and CC16 refers to 16 carbons), or converted to an amide. Amino groups of the peptide, whether at the amino terminus or at a side chain, can be in the form of a pharmaceutically-acceptable acid addition salt, such as the HCl, HBr, acetic, trifluoroacetic, formic, benzoic, toluene sulfonic, maleic, tartaric and other organic salts, or can be modified or converted to an amide, e.g., by acetylation.

Peptides may be modified to contain substituent groups that contain a positive or negative charge or both. The positive and/or negative charge may be affected by the pH at which the peptide is present.

Hydroxyl groups of the peptide side chains may be converted to C₁-C₁₆ alkoxy or to a C₁-C₁₆ ester using well-recognized techniques, or the hydroxyl groups may be converted (e.g., sulfated or phosphorylated) to introduce negative charge. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be used to form disulfide bonds or thioethers, for example through reaction with a maleimide. Thiols may be protected with any of a number of well-recognized protecting groups, such as acetamide groups. Those skilled in the art will also recognize methods for introducing cyclic structures into the peptides of this invention to select and provide conformational constraints to the structure that result in enhanced stability. Reference may be made to Greene et al., “Greene's Protective Groups in Organic Synthesis” Fourth Edition, John Wiley & Sons, Inc. 2006 for details of additional modifications that can be made to functional groups.

In some embodiments, cysteine residues of naturally occurring peptide antigens can be replaced with alpha aminobutyric acid or serine, and methionine residues can be replaced with norleucine, to yield nonnatural peptide antigens that induce immune responses that are cross-reactive with the naturally occurring peptide antigens. Preferred methods for preparing and using peptide antigens with nonnatural sequences are described throughout the specification.

Pharmaceutically acceptable vehicles: The pharmaceutically acceptable vehicles (or carriers) useful in this disclosure include conventional carriers, excipients, and diluents. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more therapeutic cancer vaccines, and additional pharmaceutical agents.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as an ointment or cream.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

Polar: A description of the properties of matter. Polar is a relative term and may describe a molecule or a portion of a molecule that has partial charge that arises from differences in electronegativity between atoms bonded together in a molecule, such as the bond between nitrogen and hydrogen. Polar molecules prefer interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may prefer interacting with a polar solvent such as water. In specific, non-limiting cases, introduction of additional polar groups may increase the solubility of a portion of a molecule.

Polymer: A molecule containing repeating structural units (monomers). As described in greater detail throughout the disclosure, polymers may be used for any number of components of amphiphiles, peptide antigens conjugates and drug molecule conjugates and may be natural or synthetic. Various compositions of polymers useful for the practice of the invention are discussed in greater detail elsewhere. Note: polymer is used throughout the specification to broadly encompass molecules with as few as three or more monomers, which may sometimes be referred to as oligomers.

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomer units resulting in the splitting out of a water molecule. In other forms of polymerization, a monomer is added one at a time to a growing chain through the staged introduction of activated monomers, such as during solid phase peptide synthesis (SPPS).

Polymersome: Vesicle, which is assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self-assembly,” a spontaneous, entropy-driven process of preparing a closed semi-permeable membrane.

Purified: A substance or composition that is relatively free of impurities or substances that adulterate or contaminate the substance or composition. The term purified is a relative term and does not require absolute purity. Substantial purification denotes purification from impurities. A substantially purified substance or composition is at typically at least 60%, 70%, 80%, 90%, 95%, 98%, or 99% pure.

Soluble: Capable of becoming molecularly or ionically dispersed in a solvent to form a homogeneous solution. When referring to an amphiphile, peptide antigen conjugate, drug molecule conjugate and/or drug molecule, soluble is understood to be a single molecule in solution that does not assemble into multimers or other supramolecular structures through hydrophobic or other non-covalent interactions. A soluble molecule is understood to be freely dispersed as single molecules in solution. Hydrophobic blocks (H) described herein are insoluble or soluble only to concentrations of about 0.1 mg/mL or less. Solubility can be determined by visual inspection, turbidity measurements or dynamic light scattering.

Solubilizing block: As used herein, a solubilizing block, denoted S, refers to a portion of a peptide antigen conjugate molecule that comprises functional groups that are aqueous soluble or water miscible, and/or that may be added to the peptide antigen conjugate to improve aqueous solubility of conjugate particles. In some cases, a solubilizing block has a positive or negative charge, and if charged may be referred to as a charged molecule, denoted C.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

The terms systemic inflammation, or systemic innate immune response or systemic innate immune activation or systemic innate activation, refer to systemic activation of the innate immune system, such as may be observable, for example, by increases in serum levels of pro-inflammatory cytokines (e.g., IL-1, IL-6, IL-12, TNF-alpha), Interferons (IFNs), e.g., IFN-alpha, or markers that track increases in IFN signaling (e.g., IFN type I or IFN-1 signaling), such as CXCL9 or CXCL10 (also referred to as IP-10) levels., as well as markers of immune cell activation, such as increased costimulatory molecule expression (e.g., CD80, CD86, PDL1, PD1) on immune cells in the blood, or increases in expression of proinflammatory genes or IFN signaling pathways. In some embodiments, changes in such expression may be determined, e.g., by technologies for assessing gene expression (e.g., RNA sequencing) or expression of gene products (e.g., Western Blot).

Targeting molecules: Are broadly defined as molecules that direct drug molecules to a specific tissue or cell population. Targeting molecules are defined by their intended use and therefore include structurally diverse molecules including without limitation antibodies, Fabs, peptides, aptamers, saccharides (e.g., saccharides that bind to lectin receptors and/or are recognized by cellular transporters), amino acids, neurotransmitters, etc. As targeting molecules are often selected from molecules that bind cellular receptors that can activate downstream signaling cascades and/or impact the activity of other linked molecules, targeting molecules are often classified as drug molecules (D) in the present disclosure. Additionally, targeting molecules can also have solubilizing effects, and may be considered either or both drug molecules (D) and/or solubilizing (SG) groups.

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells (e.g., regulatory T cells) can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Treating, preventing, or ameliorating a disease: “Treating” refers to an intervention that reduces at least one sign or symptom or marker of a disease or pathological condition after it has begun to develop, or that inhibits a sign or symptom or marker of a disease or pathological condition from developing or continuing to develop. For example, treating a disease may result in a reduction in tumor burden, meaning a decrease in the number or size of tumors and/or metastases, a limitation or inhibition of the development of new tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing at least one sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, lack of relapse or recurrence or metastasis, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplasm: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, for example via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply and may have induced the regulatory T cells (Tregs) and myeloid derived suppressor cells (MDSC).

Tumor Microenvironment (TME) refers to the tumor and the local environment surrounding the tumor, which includes, for example, blood cells including fibroblasts and various immune cells and their products such as cytokines, blood vessels forming the vasculature in and around the tumor, stromal cells, and the extracellular matrix surrounding tumor cells as well as the tumor cells themselves.

The terms tumor specific inflammation, or tumor specific innate immune response or tumor specific innate immune activation, refer to inflammation that occurs in the tumor microenvironment (TME), such as in the vasculature surrounding the tumor cells, tumor draining lymphatics, tumor draining lymph nodes and/or in the tumoral tissue. Tumor specific inflammation may be observable, for example, by increases in tumor levels of pro-inflammatory cytokines (e.g., IL-1, IL-6, IL-12, TNF-alpha), Interferons (IFNs), e.g., IFN-alpha, or markers that track increases in IFN signaling (e.g., IFN-1 signaling), such as CXCL9 or CXCL10 (also referred to as IP-10) levels., as well as markers of immune cell activation, such as increased costimulatory molecule expression (e.g., CD80, CD86, PDL1, PD1) on immune cells in the tumor or draining lymphatics, or increases in expression of proinflammatory genes or IFN signaling pathways as may be determined, e.g., by technologies for assessing gene expression (e.g., RNA sequencing) or expression of gene products (e.g., Western Blot).

Unit dose: A discrete amount of the pharmaceutical composition comprising a predetermined amount of the active ingredient.

Vesicle: A fluid filled sac. In some embodiments the vesicle is a sac comprising an amphiphilic substance. In some embodiments, the sac is a nanoparticle-based vesicle, which refers to a vesicle with a size or dimensions in the nanometer range. In some embodiments, a polymer vesicle is a vesicle that is formed from one or more polymers.

### II. Exemplary Treatment Regimes and Kits

The present disclosure relates to methods of treatment for cancer in a subject that comprise inducing an immune response in the subject, with a first treatment, such as a vaccine or adoptive cell therapy, and inducing systemic and/or tumor specific inflammation in the subject with a second treatment, wherein, in most cases, there is a time interval (T) between the administration of the first treatment and the administration of the second treatment, and wherein the second treatment is administered intravenously to the subject. Thus, in some embodiments, the disclosure relates to a method for treating a cancer in a subject comprising (a) administering a first treatment that induces an immune response by providing or inducing production of antigen-specific CD4+ and/or CD8+ T cells in the subject, such as CD4+ T cells and/or CD8+ T cells against a tumor antigen included in the first treatment, and (b) following a time interval (T), and administering a second treatment that induces systemic and/or tumor specific inflammation in the subject, such as may be observed via increases in certain cytokines or cytokine signaling systemically in the subject, such as markers of IFN-I signaling such as IL12 and IFN-alpha. In some embodiments, the second treatment is administered intravenously (IV). In some cases, the first treatment is administered intravenously (IV), intraperitoneally (IP), intramuscularly (IM), subcutaneously (SC) or intradermally (ID) and the second treatment is administered intravenously (IV). In some embodiments, the first treatment is administered intravenously or intramuscularly and the second treatment is administered intravenously. In some embodiments, both the first and second treatments are administered intravenously. In some embodiments, the first treatment is administered intramuscularly and the second treatment is administered intravenously. In some cases, neither treatment is administered subcutaneously.

In some embodiments, the first treatment provided is a vaccine composition that provides a cancer antigen to the subject. This may be a peptide antigen vaccine, such as a peptide antigen conjugate or a chimeric polypeptide construct (e.g, a construct that links a peptide antigen to a heterologous protein for delivery), or it may be a nucleic acid vaccine, such as a viral vector, DNA vector, or RNA vector that encodes an antigen. In some cases, a vaccine, such as a peptide antigen conjugate, is configured to improve the solubility of an antigen, such as to couple the peptide covalently or noncovalently to additional components to improve its delivery. Thus, in some cases, the peptide antigen conjugate is coupled to polar or charged molecules and hydrophobic molecules at the N- and C-termini, optionally with peptide extensions or linkers, so as to form an amphiphilic structure such as a micelle or similar structure in solution. Particular exemplary peptide antigen conjugate vaccines that may be used in methods herein are discussed below and are also disclosed in a U.S. provisional application filed Oct. 25, 2022, to inventors G. Lynn et al., entitled Self-Assembling Nanoparticles, which is filed concurrently herewith and which is incorporated herein by reference. In some cases, a peptide antigen conjugate vaccine may also incorporate a drug molecule, such as a chemotherapeutic or immunostimulant drug, which, for example, can be incorporated into a micelle or similar particle noncovalently or covalently linked to the antigen peptide. Similarly, in some cases, a nucleic acid vaccine used as a first treatment may encode not only a tumor antigen, but in some cases may further encode other molecules such as immunostimulant proteins, such as certain cytokines. Vaccines as first treatments may also be administered with other adjuvant molecules. In some embodiments, the first treatment vaccine is not administered subcutaneously (by SC). In some embodiments, it is administered by IV or intramuscularly (IM). In some embodiments, it is administered by IM.

In other embodiments, a first treatment may comprise an adoptive cell therapy, such as a CAR-T, or T cell, including autologous T cells (e.g., TILs, MILs, peripheral T cells) or transgenic T cells engineered with a tumor-antigen specific TCRs, as well as antigen-loaded antigen-presenting cells (APCs), such as dendritic cells (DCs), or other immune cell intended to induce activation and expansion of CD4+ T cells and/or CD8+ T cells against the subject's cancer.

In some embodiments, a second treatment may comprise an immunostimulant, for example, intended to induce systemic and or tumor specific inflammation in the subject. In some embodiments, the second treatment is administered by IV. In some embodiments, the second treatment is not administered by local routes of administration, e.g., subcutaneously (by SC) or intramuscular (IM). In some embodiments, the second treatment comprises an immunostimulant drug, such as a small molecule or protein drug. In some embodiments, the second treatment comprises both an immunostimulant drug and an delivery system (or vehicle), such as an amphiphilic molecule, examples of which are provided below. In some cases, the second treatment is a polynucleotide vector or vaccine, in some embodiments encoding a tumor antigen and/or other proteins, such as a ChAdOx vaccine. In other embodiments, the second treatment comprises an immunostimulant drug as well as a vaccine, and in some cases, the vaccine may comprise the same antigen as a vaccine given as the first treatment. In some cases, the vaccine of the first treatment is the same as that of the second treatment, or alternatively, the vaccine antigen is the same in both the first and second treatments (i.e., two different conjugates comprising the same peptide antigen are used, or alternatively, one treatment comprises a polypeptide antigen or adoptive cell therapy where the cell expresses the antigen, while the other comprises a nucleic acid vaccine encoding the antigen). In some cases, in the first treatment, the vaccine is administered IM, and in the second treatment, the vaccine is administered IV. In other cases, the vaccine is administered IV in both the first and second treatments.

More specific examples of first and second treatments are also provided further below and in the Examples.

The time interval (T) between the first treatment and the second treatment is generally greater than 1 day, and in some embodiments is at least 3 days. In some cases, the time interval (T) between the first and second treatments is at least 3 days, at least 5 days, 5 to 90 days, 5 to 60 days, 5 to 30 days, 5 days to three weeks, one week to three weeks, one week to two weeks, 3 to 28 days, 5 to 28 days, 5 to 14 days, 7 to 28 days, 3 to 21 days, 5 to 21 days, 7 to 21 days, 3 to 14 days, 5 to 14 days, 7 to 14 days, 14 to 28 days, or 14 to 21 days. In some cases, more than one dose of a first treatment is administered before the time interval (T) and administration of a second treatment. In some cases, the first and second treatments are provided following or in conjunction with chemotherapy, radiation, or other cancer therapies. More specific combinations of first and second treatments with particular time intervals between them, and in some cases, exemplary dosages, are provided below and in the Examples.

In some embodiments, the cancer to be treated in the subject may be a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the cancer to be treated in the subject is a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma).

In several examples, a tumor is melanoma, lung cancer, lymphoma breast cancer or colon cancer.

In some cases, the cancer is an HPV+ cancer, such as a head and neck, vulvar, or cervical cancer.

### III. Exemplary Vaccine Compositions

In some embodiments, methods herein may utilize vaccines that deliver polypeptide tumor antigens, such as various types of peptide antigen conjugates and chimeric proteins comprising antigens. In some embodiments, a vaccine may comprise a chimeric protein comprising one or more tumor antigens. For example, an antigen may be fused to one or more domains of a heterologous protein to provide the antigen in an appropriate form for inducing an immune response, such as providing the antigen on the surface of a cell or in secreted form. In some embodiments, peptide antigen conjugates are utilized, which include one or more tumor antigens (A). In some embodiments, the peptide antigen conjugates may include other components covalently or noncovalently linked to a tumor antigen, including components that improve solubility and/or allow the peptide antigen conjugate to form a micellar structure. In some embodiments, a peptide antigen conjugate can further comprise optional first or second amphiphiles to assist further with solubility and/or tolerability. In some cases, a conjugate also comprises a drug molecule, such as a chemotherapy or immunostimulant drug molecule. In some embodiments, the drug molecule may be covalently linked to the peptide antigen conjugate, while in other cases it may not be covalently linked but may be noncovalently associated with the peptide antigen conjugate, for example. In some embodiments, the peptide antigen conjugates are as described in International Patent Publication No. WO 2018/187515. In some embodiments, the peptide antigen conjugates are as described in International Patent Publication No. WO 2020/072681. In some embodiments, the peptide antigen conjugates and amphiphiles are as described in International Patent Publication No. WO 2022/177993. In some embodiments, the peptide antigen conjugates and amphiphiles are as described in International Patent Application No. PCT/US2022/033819 filed Jun. 16, 2022. Each of these disclosures is incorporated herein by reference in its entirety. A description of exemplary antigens that may be administered in methods herein and peptide antigen conjugates, as well as other types of vaccines and treatments useful in methods herein now follows.

### A. Exemplary Antigens and Antigen Sequences for Cancer Vaccines

In some embodiments, a first and/or a second treatment delivers an antigen (abbreviated (A) in certain peptide antigen conjugate vaccine embodiments herein) to the subject. The antigen may be any antigen that is useful for inducing an immune response in a cancer subject, e.g. providing an antigen-specific CD4 and/or CD8 T cell response, such as a tumor antigen. In some embodiments, the tumor antigen is a self-antigen, neoantigen or tumor-associated viral antigen (e.g., HPV E6/E7). An antigen may be a polypeptide (i.e., a peptide antigen), but may also be selected from small molecules (sometimes referred to as haptens). For a peptide antigen, it may be delivered in protein form, or as part of a nucleic acid construct that encodes the antigen, for example. In some embodiments, a peptide antigen comprises an amino acid or amino acids with a post-translational modification (e.g., glycosylation, oxidation, phosphorylation, citrullination and/or homocitrullination), non-natural amino acids or peptide-mimetics. A peptide antigen may be any sequence of natural, non-natural or post-translationally modified amino acids, peptide-mimetics, or any combination thereof, that have an antigen or predicted antigen, i.e., an antigen with a T cell and/or B cell epitope. Peptide antigens also include post-translationally modified peptide antigens, including glycopeptides.

In some embodiments, particularly for peptide antigen conjugates, cysteine and methionine amino acids, found in naturally occurring peptide antigen sequences, may be replaced with amino acids that are not naturally found in those sequences, e.g., alpha aminobutyric acid (aBut) and norleucine (nLeu), respectively. Such replacements led to unexpected improvements in vaccine manufacturing and in vivo immunogenicity when preparing conjugates of the C-A-H variety described herein. Therefore, in some embodiments of peptide antigen conjugates herein, naturally occurring cysteine amino acids are replaced with alpha aminobutyric acid and methionine amino acids are replaced with norleucine.

In some embodiments in which a peptide antigen conjugate is administered to induce a CD4 and/or CD8 T cell response, one or more than one different antigen conjugate may be administered. In some cases, antigen conjugate particles with up to 50 different peptide antigen conjugates each having a unique peptide antigen (A) composition may be administered. In some embodiments, the immunogenic compositions comprise mosaic particles that comprise two or more different peptide antigen conjugates, e.g., up to about 100 different peptide antigen conjugates, typically no more than about 40 peptide antigen conjugates, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 peptide antigen conjugates. In other embodiments, the immunogenic compositions comprise mosaic particles that comprise 5 different peptide antigen conjugates. In still other embodiments, the immunogenic compositions comprise a single particle composition comprising of a single (1) peptide antigen conjugate composition. Accordingly, the term “antigen” (A) herein, for example, in the context of a peptide antigen conjugate or vaccine, refers to both one specific antigen and to such a plurality of antigens.

The number of peptide antigen conjugates may be selected to ensure that an adequate immune response can be induced in each subject. In preferred embodiments, vaccines for cancer treatment typically include up to about 40, though typically no more than 100, peptide antigen conjugates each comprising a unique peptide antigen (A) that comprises one or more CD4, CD8 T cell and/or B cell epitopes or predicted epitopes.

The length of a peptide antigen (A) depends on the specific application and is typically between about 5 to about 100 amino acids. In some embodiments, a peptide antigen (A) is between about 7 to 35 amino acids, e.g., 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. In still other cases, the peptide antigen is a full-length polypeptide, such as a protein antigen that may be recombinantly expressed as part of a viral or other polynucleotide vector vaccine. Peptide antigens (A) based on full-length tumor-associated proteins may also be delivered as the full-length sequence, or as an overlapping peptide pool wherein each peptide antigen (A) of the overlapping peptide pool is no more than 100 amino acids in length, preferably no more than 35 amino acids in length.

In some embodiments in which a vaccine delivers an antigen in polypeptide form, the peptide antigen (A) is 7 to 35 amino acids, typically about 25. Thus, a longer antigen may be divided into 7 to 35 amino acids, e.g., 25 amino acid, peptide antigens (A) wherein each peptide antigen (A) contains a unique composition of amino acids; or, the peptide antigens (A) can be overlapping peptide pools wherein an antigen is divided into a set number of 7 to 35 amino acid, e.g., 25 amino acid, peptide antigens (A) that have overlapping sequences. For example, an overlapping peptide pool comprising a 100 amino acid antigen may be divided into eight 25 amino acid peptide antigens (A) that are each offset by 12 amino acids (i.e., each subsequent 25 amino acid peptide comprising a 100 amino acid peptide sequence starts at the 13th amino acid position from the prior peptide). Those skilled in the art understand that many permutations exist for generating a peptide pool from an antigen.

In some embodiments, the peptide antigen (A) is a minimal CD8 or CD4 T cell epitope that comprises the portions of a tumor antigen that are predicted in silico (or measured empirically) to bind MHC-I or MHC-II molecules. Algorithms for predicting MHC-I or MHC-II binding are widely available (see Lundegaard et al., Nucleic Acids Res., 36:W509-W512, 2008 and http://www.cbs.dtu.dk/services/NetNMC/). In some embodiments of a personalized therapy for a particular subject, the peptide antigen (A) comprising a peptide antigen conjugate may comprise a minimal CD8 T cell epitope from a tumor antigen that is typically a 7-13 amino acid peptide that is predicted to have <1,000 nM binding affinity for a particular MHC-I allele that is expressed by that subject. In some embodiments of a personalized therapy for a particular subject, the peptide antigen (A) may comprise a minimal CD4 T cell epitope from a tumor antigen that is an 8-20 amino acid peptide, or more preferably a 10-16 amino acid peptide, that is predicted to have <1,000 nM binding affinity for a particular MHC-II allele that is expressed by that subject. In certain embodiments, when a minimal CD8 or CD4 T cell epitope cannot be identified for a tumor antigen, an antigen contains multiple CD8 and CD4 T cell epitopes, and the peptide antigen (A) may be between 16-35 amino acids, e.g., up to 35 amino acids such that it may contain all possible CD8 or CD4 T cell epitopes.

In some embodiments, an antigen is a tumor antigen. Tumor antigens include self-antigens that are present on healthy cells but are preferentially expressed by tumor cells, or neoantigens, which are aberrant proteins that are specific to tumor cells and are unique to individual patients. Tumor antigens may also include viral antigens. Vaccines may be used to deliver antigens to the subject, for example, in a first treatment to provide antigen-specific CD4 and/or CD8 T cells in the subject. In such cases, vaccines may be used, either in their polypeptide form such as in a peptide antigen conjugate, or in nucleic acid form in a polynucleotide vaccine that encodes the antigen sequences. In some embodiments, a vaccine is administered as a first treatment but not as a second treatment, while in other embodiments, both a first treatment and a second treatment comprise a vaccine.

Exemplary self-antigens include antigens that are preferentially expressed by tumor cells, such as CLPP, Cyclin-A1, MAGE-A1, MAGE-C1, MAGE-C2, SSX2, XAgE1b/GAGED2a, Melan-A/MART-1, TRP-1, Tyrosinase, CD45, glypican-3, IGF2B3, Kallikrein 4, KIF20A, Lengsin, Meloe, MUC5AC, survivin, prostatic acid phosphatase, NY-ESO-1 and MAGE-A3.

Neoantigens arise from the inherent genetic instability of cancers, which can lead to mutations in DNA, RNA splice variants and changes in post-translational modification, all potentially leading to de novo protein products that are referred to collectively as neoantigens or sometimes predicted neoantigens. DNA mutations include changes to the DNA including nonsynonymous missense mutations, nonsense mutations, insertions, deletions, chromosomal inversions and chromosomal translocations, all potentially resulting in novel gene products and therefore neoantigens. RNA splice site changes can result in novel protein products and missense mutations can introduce amino acids permissive to post-translational modifications (e.g., phosphorylation) that may be antigenic. The instability of tumor cells can furthermore result in epigenetic changes and the activation of certain transcription factors that may result in selective expression of certain antigens by tumor cells that are not expressed by healthy, non-cancerous cells.

Peptide antigen conjugates used in personalized cancer vaccines may include peptide antigens (A) that comprise the portions of tumor antigens that are unique to tumor cells. Peptide antigens (A) comprising neoantigens arising from a missense mutation should encompass the amino acid change encoded by 1 or more nucleotide polymorphisms. Peptide antigens (A) comprising neoantigens that arise from frameshift mutations, splice site variants, insertions, inversions and deletions should encompass the novel peptide sequences and junctions of novel peptide sequences. Peptide antigens (A) comprising neoantigens with novel post-translational modifications should encompass the amino acids bearing the post-translational modification(s), such as a phosphate or glycan. In preferred embodiments, the peptide antigen (A) comprises the up to 25 amino acids on either side flanking the amino acid change or novel junction that arises due to a mutation. In certain embodiments, the peptide antigen (A) is a neoantigen sequence that comprises the 12 amino acids on either side flanking the amino acid change that arises from a single nucleotide polymorphism, for example, a 25 amino acid peptide, wherein the 13th amino acid is the amino acid residue resulting from the single nucleotide polymorphism. In some embodiments, the peptide antigen (A) is a neoantigen sequence that comprises the 12 amino acids on either side flanking an amino acid with a novel post-translational modification, for example, a 25 amino acid peptide, wherein the 13th amino acid is the amino acid residue resulting from the novel post-translational modification site. In other embodiments, the peptide antigen (A) is a neoantigen sequence that comprises 0-12 amino acids on either side flanking a novel junction created by an insertion, deletion or inversion. In some cases, the peptide antigen (A) comprising neoantigens resulting from novel sequences can encompass the entire novel sequence, including 0-25 amino acids on either side of novel junctions that may also arise.

Tumor antigens can be identified through various techniques that are familiar to one skilled in the art. Tumor antigens can be identified by assessing protein expression of tumor cells as compared with healthy cells, i.e., non-cancerous cells from a subject. Suitable methods for assessing protein expression include but are not limited to immunohistochemistry, immunofluorescence, western blot, chromatography (i.e., size-exclusion chromatography), ELISA, flow cytometry and mass spectrometry. Proteins preferentially expressed by tumor cells but not healthy cells or by a limited number of healthy cells (e.g., CD20) are suitable tumor antigens. DNA and RNA sequencing of patient tumor biopsies followed by bioinformatics to identify mutations in protein-coding DNA that are expressed as RNA and produce peptides predicted to bind to MHC-I or MHC-II alleles on patient antigen presenting cells (APCs), may also be used to identify tumor antigens that are suitable as peptide antigens (A) for immunogenic compositions of the present disclosure.

In some embodiments, tumor antigens are identified using mass spectrometry. Suitable peptide antigens (A) are peptides identified by mass spectrometry following elution from the MHC molecules from patient tumor biopsies but not from healthy tissues from the same subject (i.e., the peptide antigens are only present on tumor cells but not healthy cells from the same subject). Mass spectrometry may be used alone or in combination with other techniques to identify tumor antigens. Those skilled in the art recognize that there are many methods for identifying tumor antigens, such as neoantigens (see Yadav et al., Nature, 515:572-576, 2014) that are suitable as peptide antigens (A) for the practice of the disclosed invention. In some embodiments, the tumor antigens used as peptide antigens (A) are clonal or nearly clonal within the population of neoplastic cells, which may be considered heterogeneous in other respects.

Tumor antigens selected for use as peptide antigens (A) in personalized cancer vaccination schemes may be selected based on mass spectrometry confirmation of peptide-MHC binding and/or in silico predicted MHC binding affinity and RNA expression levels within tumors. These data provide information on whether or not a tumor antigen is expressed and presented by tumor cells and would therefore be a suitable target for T cells. Such criteria may be used to select the peptide antigens (A) used in a personalized cancer vaccine.

For patients with highly mutated tumors that have more than 50 tumor-associated neoantigens, a down-selection process may be used to select peptide antigens (A) for use in personalized cancer vaccines comprising peptide antigen conjugates. In some embodiments, a down-selection process is used to select peptide antigens (A) comprising epitopes predicted to have the highest MHC binding affinity and RNA expression levels within tumor cells. Additional criteria may be applied for the selection of tumor-associated self-antigens or neoantigens. For example, predicted immunogenicity or predicted capacity of the peptide antigen (A) to lead to T cells that react with other self-antigens, which may lead to autoimmunity, are additional criteria considered. For instance, peptide antigens (A) that comprise tumor antigens and have high predicted immunogenicity but also low potential to lead to autoimmunity are criteria used to select potential peptide antigens (A) for use in personalized cancer vaccines. In some embodiments, neoantigens that that would be expected to result in T cell or antibody responses that react with self-antigens found on healthy cells are not selected for use as peptide antigens (A). For patients with less than, for example, 20-50 predicted neoantigens, a down selection process may not be critical and so all 20-50 predicted neoantigens might be used as peptides antigens (A) in a personalized cancer vaccine.

Cancer vaccines may deliver antigens (A) that comprise tumor antigens that are patient-specific and/or tumor antigens that are shared between patients. For example, the tumor antigen can be a conserved self-antigen, such as NY-ESO-1 (testicular cancer) or gp100 (melanoma), or the antigen may be a cryptic epitope, such as Na17 (melanoma) that is not typically expressed by healthy cells but is conserved between certain cancer patients. Immunogenic compositions of the present disclosure may include peptide antigens (A) that arise from so-called hot-spot mutations that are frequent mutations in certain genes or gene regions that occur more frequently than would be predicted by chance. Non-limiting examples of hot spot mutations include the V600E mutation in BRAF protein, which is common to melanoma, papillary thyroid and colorectal carcinomas, or KRAS G12 mutations, which are among the most common mutations, such as KRAS G12C. A number of suitable self-antigens as well as neoantigens that arise from hotspot mutations are known and are incorporated herein by reference: see Chang et al., Nature Biotechnology, 34:155-163, 2016; Vigneron, N., et al, Cancer Immunology, 13:15-20, 2013.

In some embodiments, the peptide antigen (A) can be from a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the peptide antigen (A) can be from a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the peptide antigen (A) is a tumor antigen from a breast cancer, such as a ductal carcinoma or a lobular carcinoma. In some embodiments, the peptide antigen (A) is a tumor antigen from a prostate cancer. In some embodiments, peptide antigen (A) is a tumor antigen from a skin cancer, such as a basal cell carcinoma, a squamous cell carcinoma, a Kaposi's sarcoma, or a melanoma. In some embodiments, the peptide antigen (A) is a tumor antigen from a lung cancer, such as an adenocarcinoma, a bronchiolaveolar carcinoma, a large cell carcinoma, or a small cell carcinoma. In some embodiments, the peptide antigen (A) is a tumor antigen from a brain cancer, such as a glioblastoma or a meningioma. In some embodiments, the peptide antigen (A) is a tumor antigen from a colon cancer. In some embodiments, the peptide antigen (A) is a tumor antigen from a liver cancer, such as a hepatocellular carcinoma. In some embodiments, the peptide antigen (A) is a tumor antigen from a pancreatic cancer. In some embodiments, peptide antigen (A) is a tumor antigen from a kidney cancer, such as a renal cell carcinoma. In some embodiments, the peptide antigen (A) is a tumor antigen from a testicular cancer.

In some embodiments, the peptide antigen (A) is a tumor antigen derived from premalignant conditions, such as variants of carcinoma in situ, or vulvar intraepithelial neoplasia, cervical intraepithelial neoplasia, or vaginal intraepithelial neoplasia.

In some embodiments, the peptide antigen (A) is an antigen from an infectious agent, such as a virus, a bacterium, or a fungus. In some embodiments of vaccines against HPV, including HPV+ cancers, such as HPV+ head and neck, vulvar, or cervical cancer, the peptide antigens are selected from MHQKRTAMFQDPQERPRKLPQLCTELQTT (SEQ ID NO: 56), PRKLPQLCTELQTTIHDIILECVYCKQQL (SEQ ID NO: 57), HDIILECVYCKQQLLRREVYDFAFRDLCI (SEQ ID NO: 58), RREVYDFAFRDLCIVYRDGNPYAVCDKCL (SEQ ID NO: 59), YRDGNPYAVCDKCLKFYSKISEYRHYCYS (SEQ ID NO: 60), FYSKISEYRHYCYSLYGTTLEQQYNKPLC (SEQ ID NO: 61), YGTTLEQQYNKPLCDLLIRCINCQKPLCP (SEQ ID NO: 62), LLIRCINCQKPLCPEEKQRHLDKKQRFHN (SEQ ID NO: 63), EKQRHLDKKQRFHNIRGRWTGRCMSCCR (SEQ ID NO: 64), IRGRWTGRCMSCCRSSRTRRETQL (SEQ ID NO: 65), MHGDTPTLHEYMLDLQPETTDLYCYEQ (SEQ ID NO: 66), DLQPETTDLYCYEQLNDSSEEEDEI (SEQ ID NO: 67), YEQLNDSSEEEDEIDGPAGQAEPDR (SEQ ID NO: 68), DEIDGPAGQAEPDRAHYNIVTFCCKCD (SEQ ID NO: 69), RAHYNIVTFCCKCDSTLRLCVQSTHVDIRTLE (SEQ ID NO: 70), LCVQSTHVDIRTLEDLLMGTLGIVCPICSQKP (SEQ ID NO: 71) and QLYQTCKAAGTCPSDVIPKI (SEQ ID NO: 72). In preferred embodiments of vaccines against HPV, one or more cysteine and/or methionine residues of naturally occurring peptide antigens are replaced with alpha-aminobutyric acid (“B”) and/or norleucine (“n”), respectively. Non-limiting examples include nHQKRTAnFQDPQERPRKLPQLBTELQTT (SEQ ID NO: 73) nHQKRTAnFQDPQERPRKLPQLCTELQTT (SEQ ID NO: 74), MHQKRTAMFQDPQERPRKLPQLBTELQTT (SEQ ID NO: 75), PRKLPQLBTELQTTIH-DIILEBVYBKQQL (SEQ ID NO: 76), HDIILEBVYBKQQLLRREVYDFAFRDLBI (SEQ ID NO: 77), RREVYDFAFRDLBIVYRDGNPYAVBDKBL (SEQ ID NO: 78), YRDGNPYAVBDKBLKFYSKISEYRHYBYS (SEQ ID NO: 79), FYSKISEYRHYBYSLYGTTLEQQYNKPLB (SEQ ID NO: 80), YGTTLEQQYNKPLDLLIRBINBQKPLBP (SEQ ID NO: 81), LLIRBINBQKPLBPEEKQRHLDKKQRFHN (SEQ ID NO: 82), EKQRHLDKKQRFHNIRGRWTGRCnSCCR (SEQ ID NO: 83), EKQRHLDKKQRFHNIRGRWTGRBnSBBR (SEQ ID NO: 84), EKQRHLDKKQRFHNIRGRWTGRBMSBBR (SEQ ID NO: 85), IRGRWTGRCnSCCRSSRTRRETQL (SEQ ID NO: 86), IRGRWTGRBnSBBRSSRTRRETQL (SEQ ID NO: 87), IRGRWTGRBMSBBRSSRTRRETQL (SEQ ID NO: 88), nHGDTPTLHEYnLDLQPETTDLYCYEQ (SEQ ID NO: 89), nHGDTPTLHEYnLDLQPETTDLYBYEQ (SEQ ID NO: 90), nHGDTPTLHEYnLDLQPETTDLYMYEQ (SEQ ID NO: 91), DLQPETTDLYBYEQLNDSSEEEDEI (SEQ ID NO: 92), YEQLNDSSEEEDEIDGPAGQAEPDR (SEQ ID NO: 93), DEIDGPAGQAEPDRAHYNIVTFBBKBD (SEQ ID NO: 94), RAHYNIVTFBBKBDSTLRLBVQSTHVDIRTLE (SEQ ID NO: 95), LCVQSTHVDIRTLEDLLnGTLGIVCPICSQKP (SEQ ID NO: 96), LBVQSTHVDIRTLEDLLnGTLGIVBPIBSQKP (SEQ ID NO: 97), LBVQSTHVDIRTLEDLLMGTLGIVBPIBSQKP (SEQ ID NO: 98) and QLYQTBKAAGTBPSDVIPKI (SEQ ID NO: 99) or any fragments thereof having at least 6 amino acids in length, preferably at least 9 amino acids, or derivatives thereof.

In other embodiments of vaccines against HPV, including HPV+ cancers, such as HPV+ head and neck, vulvar, or cervical cancer, the peptide antigens are selected from ALQAIELQLTLETIYNSQYSNEKWTLQDV (SEQ ID NO: 100), NSQYSNEKWTLQDVSLEVYLTAPTGCIKK (SEQ ID NO: 101), SVTVVEGQVDYYGLYYVHEGIRTYFVQFK (SEQ ID NO: 102), LKGDANTLKCLRYRFKKHCTLYTAVSSTWHWT (SEQ ID NO: 103), KHKSAIVTLTYDSEWQRDQFLSQVKIPKT (SEQ ID NO: 104), MHQKRTAMFQDPQERPRKLPQLCTELQTT (SEQ ID NO: 105), PRKLPQLCTELQTTIHiDIILECVYCKQQL (SEQ ID NO: 106), HDIILECVYCKQQLLRREVYDFAFRDLCI (SEQ ID NO: 107), RREVYDFAFRDLCIVYRDGNPYAVCDKCL (SEQ ID NO: 108), YRDGNPYAVCDKCLKFYSKISEYRHYCYS (SEQ ID NO: 109), FYSKISEYRHYCYSLYGTTLEQQYNKPLC (SEQ ID NO: 110), YGTTLEQQYNKPLCDLLIRCINCQKPLCP (SEQ ID NO: 111), CPEEKQRHLDKKQRFHNIRGRWTGRCMSCCR (SEQ ID NO: 112), MHGDTPTLHEYMLDLQPETTDLYCYEQ (SEQ ID NO: 113), AGQAEPDRAHYNIVTFCCKCDSTLRLCVQ (SEQ ID NO: 114) and LCVQSTHVDIRTLEDLLMGTLGIVCPICSQKP (SEQ ID NO: 115), wherein in preferred embodiments one or more cysteine and/or methionine residues are replaced with alpha-aminobutyric acid and/or norleucine, respectively, for example,

(SEQ ID NO: 100)
ALQAIELQLTLETIYNSQYSNEKWTLQDV,
(SEQ ID NO: 116)
NSQYSNEKWTLQDVSLEVYLTAPTGBIKK,
(SEQ ID NO: 102)
SVTVVEGQVDYYGLYYVHEGIRTYFVQFK,
(SEQ ID NO: 117)
LKGDANTLKBLRYRFKKHBTLYTAVSSTWHWT,
(SEQ ID NO: 104)
KHKSAIVTLTYDSEWQRDQFLSQVKIPKT,
(SEQ ID NO: 118)
nHQKRTAnFQDPQERPRKLPQLBTELQTT,
(SEQ ID NO: 119)
PRKLPQLBTELQTTIHDIILEBVYBKQQL,
(SEQ ID NO: 120)
HDIILEBVYBKQQLLRREVYDFAFRDLBI,
(SEQ ID NO: 121)
RREVYDFAFRDLBIVYRDGNPYAVBDKBL,
(SEQ ID NO: 122)
YRDGNPYAVBDKBLKFYSKISEYRHYBYS,
(SEQ ID NO: 123)
FYSKISEYRHYBYSLYGTTLEQQYNKPLB,
(SEQ ID NO: 124)
YGTTLEQQYNKPLBDLLIRBINBQKPLBP,
(SEQ ID NO: 125)
BPEEKQRHLDKKQRFHNIRGRWTGRBnSBBR,
(SEQ ID NO: 126)
nHGDTPTLHEYnLDLQPETTDLYBYEQ,
(SEQ ID NO: 127)
AGQAEPDRAHYNIVTFBBKBDSTLRLBVQ
and
(SEQ ID NO: 128)
LBVQSTHVDIRTLEDLLnGTLGIVBPIBSQKP.

In some embodiments of cancer vaccines for prostate cancer, the cancer vaccine comprises peptide antigens selected from fragments of prostate specific antigen (PSA), APLILSRIVGGWECEKHSQPWQVLVASRGRAVCGGVLVHPQWVLTAAHCIRNKSVI LLGRHSLFHPEDTGQVFQVSHSFPHPLYDMSLLKNRFLRPGDDSSTHDLMLLRLSEPA ELTDAVKVMDLPTQEPALGTTCYASGWGSIEPEEFLTPKKLQCVDLHVISNDVCAQV HPQKVTKFMLCAGRWTGGKSTCSGDSGGPLVCNGVLQGITSWGSEPCALPERPSLY TKVVHYRKWIKDTIVANP (SEQ ID NO: 129). In certain preferred embodiments, the peptide antigens (A) selected from fragments of PSA are typically selected from 7 to 55 amino acid stretches of PSA that may optionally overlap. Non-limiting examples include but are not limited to: CGGVLVHPQWVLTAAHCIRNKSVILLGRHSLFHPE (SEQ ID NO: 130), SLFHPEDTGQVFQVSHSFPHPLYDMSLLKNRFLRP (SEQ ID NO: 131), PCALPERPSLYTKVVHYRKWIKDTIVANP (SEQ ID NO: 132)

In some embodiments of cancer vaccines for prostate cancer, the cancer vaccine comprises peptide antigens selected from fragments of prostatic acid phosphatase (PAP), FFWLDRSVLAKELKFVTLVFRHGDRSPIDTFPTDPIKESSWPQGFGQLTQLGMEQHY ELGEYIRKRYRKFLNESYKHEQVYIRSTDVDRTLMSAMTNLAALFPPEGVSIWNPILL WQPIPVHTVPLSEDQLLYLPFRNCPRFQELESETLKSEEFQKRLHPYKDFIATLGKLSG LHGQDLFGIWSKVYDPLYCESVHNFTLPSWATEDTMTKLRELSELSLLSLYGIHIKQK EKSRLQGGVLVNEILNHMKRATQIPSYKKLIMYSAHDTTVSGLQMALDVYNGLLPP YASCHLTELYFEKGEYFVEMYYRNETQHEPYPLMLPGCSPSCPLERFAELVGPVIPQ DWSTECMTTNSHQGTEDSTD (SEQ ID NO: 133). In certain preferred embodiments, the peptide antigens (A) selected from fragments of PAP are typically selected from 7 to 55 amino acid stretches of PAP that may optionally overlap. Non-limiting examples include but are not limited to: RTLMSAMTNLAALFPPEGVSIWNPILLWQPIPVHT (SEQ ID NO: 134), PILLWQPIPVHTVPLSEDQLLYLPFRNCPRFQELE (SEQ ID NO: 135), ATEDTMTKLRELSELSLLSLYGIHKQKEKSRLQGG (SEQ ID NO: 136), LQGGVLVNEILNHMKRATQIPSYKKLIMYSAHDTT (SEQ ID NO: 137), MALDVYNGLLPPYASCHLTELYFEKGEYFVEMYYR (SEQ ID NO: 138), YFEKGEYFVEMYYRNETQHEPYPLMLPGCSPSCPL (SEQ ID NO: 139)

In some embodiments of cancer vaccines for prostate cancer, the cancer vaccine comprises peptide antigens selected from fragments of STEAP1, MESRKDITNQEELWKMKPRRNLEEDDYLHKDTGETSMLKRPVLLHLHQTAHADEF DCPSELQHTQELFPQWHLPIKIAAIIASLTFLYTLLREVIHIPLATSHQQYFYKIPILVINK VLPMVSITLLALVYLPGVIAAIVQLHNGTKYKKFPHWLDKWMLTRKQFGLLSFFFA VLHAIYSLSYPMRRSYRYKLLNWAYQQVQQNKEDAWIEHDVWRMEIYVSLGIVGL AILALLAVTSIPSVSDSLTWREFHYIQSKLGIVSLLLGTIHALIFAWNKWIDIKQFVWY TPPTFMIAVFLPIVLIFKSILFLPCLRKKILKIRHGWEDVTKINKTEICSQL (SEQ ID NO: 140). In certain preferred embodiments, the peptide antigens (A) selected from fragments of STEAP1 are typically selected from 7 to 55 amino acid stretches of STEAP1 that may optionally overlap. Non-limiting examples include but are not limited to:

(SEQ ID NO: 141)
LFPQWHLPIKIAAIIASLTFLYTLLREVIHPLATS,
(SEQ ID NO: 142)
YTLLREVIHPLATSHQQYFYKIPILVINKVLPMVS,
(SEQ ID NO: 143)
RKQFGLLSFFFAVLHAIYSLSYPMRRSYRYKLLNWAYQ,
(SEQ ID NO: 144)
EDAWIEHDVWRMEIYVSLGIVGLAILALLAVTSIP,
(SEQ ID NO: 145)
LAVTSIPSVSDSLTWREFHYIQSKLGIVSLLLGTI,
(SEQ ID NO: 146)
DIKQFVWYTPPTFMIAVFLPIVLIFKSILFLPCLR

In some embodiments of cancer vaccines for prostate cancer, the cancer vaccine comprises peptide antigens selected from fragments of 5T4, SSPTSSASSFSSSAPFLASAVSAQPPLPDQCPALCECSEAARTVKCVNRNLTEVPTDLP AYVRNLFLTGNQLAVLPAGAFARRPPLAELAALNL SGSRLDEVRAGAFEHLPSLRQL DLSHNPLADLSPFAFSGSNASVSAPSPLVELILNHIVPPEDERQNRSFEGMVVAALLA GRALQGLRRLELASNHFLYLPRDVLAQLPSLRHLDL SNNSLVSLTYVSFRNLTHLESL HLEDNALKVLHNGTLAELQGLPHIRVFLDNNPWVCDCHMADMVTWLKETEVVQG KDRLTCAYPEKMRNRVLLELNSADLDCDPILPPSLQTSYVFLGIVLALIGAIFLLVLYL NRKGIKKWMHNIRDACRDHMEGYHYRYEINADPRLTNLSSNSDV (SEQ ID NO: 147). In certain preferred embodiments, the peptide antigens (A) selected from fragments of 5T4 are typically selected from 7 to 55 amino acid stretches of 5T4 that may optionally overlap. Non-limiting examples include but are not limited to:

(SEQ ID NO: 148)
SPTSSASSESSSAPFLASAVSAQPPLPDQCPALCE,
(SEQ ID NO: 149)
RNLTEVPTDLPAYVRNLFLTGNQLAVLPAGAFARR,
(SEQ ID NO: 150)
ALQGLRRLELASNHFLYLPRDVLAQLPSLRHLDLS,
(SEQ ID NO: 151)
LSNNSLVSLTYVSFRNLTHLESLHLEDNALKVLHN,
(SEQ ID NO: 152)
DCDPILPPSLQTSYVFLGIVLALIGAIFLLVLYLN.

In some embodiments, the peptide antigen (A) may be identified and selected on the basis of screening a subject's own T cells (e.g., tumor-infiltrating lymphocytes (TILS), marrow-infiltrating lymphocytes (MILS) or peripheral blood lymphocytes), for autoreactivity against tumor-derived antigens. Alternatively, the peptide antigens may be selected using in silico methods to predict potential autoantigens that (i) have a predicted high affinity for binding a subjects' own MHC-I and/or MHC-II molecules and (ii) are expressed. In some embodiments, the peptide antigen is selected from either protein coding DNA or non-protein coding DNA. In other embodiments the antigen (A) is selected from splice variants. In still other embodiments the protein antigen is selected from mass-spec based profiling of epitopes eluted from MHC-I and MHC-II derived from tumor cells.

In some cases, a cancer vaccine comprises peptide antigens (A) comprising T cell epitopes and B cell epitopes, and optionally haptens comprising tumor-specific glycans. In certain preferred embodiments, peptide antigens comprising B cell epitopes are selected from glycopeptides. Various tumor associated glycopeptides are known in the art. In certain preferred embodiments of cancer vaccines, at least one peptide antigen conjugate further comprises an antigen selected from a glycopeptide selected from Mucin 1 derived peptides with O-linked glycosylation at serine and threonine residues. Non-limiting examples include the peptide antigen sequences HGVT*S*APDT*RPAPGS*T*APPA (SEQ ID NO: 153), DT*RPAPGS*T*APPAHGVT*S*AP (SEQ ID NO: 154), GS*T*APPAHGVT*S*APDT*RPAPGS*T*APPA (SEQ ID NO: 155), GVT*S*APDT*RPAP (SEQ ID NO: 156), APDT*RPAPGS*T*A (SEQ ID NO: 157), GS*T*APPAHGVT*S*AP (SEQ ID NO: 158), VT*S*AP (SEQ ID NO: 168), DT*RPAP (SEQ ID NO: 159) and GS*T*AP (SEQ ID NO: 160), wherein * is an O-linked glycan and each occurrence is independently selected from sialyl lewis x, sialyl lewis a, lewis y, lewis x, Tn, sTn, TF and sTF glycans.

In embodiments in which a polynucleotide vaccine is administered, in some cases the vaccine may include a coding sequence for one of the above antigens such that it is expressed in the subject in its polypeptide form.

### B. Peptide Antigen Conjugates of Formula S-[E1]-A-[E2]-[U]-H [D] and Optional Amphiphiles

In some embodiments, an antigen included in a first and/or second treatment herein may be delivered as a polypeptide, for example in some embodiments as part of a chimeric protein or as part of a peptide antigen conjugate. In some embodiments, a peptide antigen conjugate has the formula S-[E1]-A-[E2]-[U]-H [D], in which A is the antigen, H is a hydrophobic molecule, S is a solubilizing block, E1 (alternatively denoted B1 herein, for example in certain chemical drawings) is an N-terminal extension, E2 (alternatively denoted B2 herein) is a C-terminal extension, U is a linker (alternatively denoted L herein, for example in certain chemical drawings), D is a drug molecule, [ ] denotes that the group is optional, and a dash (-) indicates a covalent linkage. Thus, groups E1, E2, U, and the drug D may or may not be present, as indicated by the brackets. In some embodiments, where drug D is present, it is covalently linked to the hydrophobic block (H), i.e., S-[E1]-A-[E2]-[U]-H-D, where the dash (-) indicates a covalent linkage. In other instances, the drug D can be noncovalently associated within a micellar particle, for example, of the S-[E1]-A-[E2]-[U]-H D formula, hence the absence of a dash (-). In some embodiments, the solubilizing block S is a charged molecule C that comprises one or more functional groups that are charged at physiological pH, such as a pH 7.4. Accordingly, in some cases, the peptide antigen conjugate has a net electrostatic charge greater than or equal to +3 or less than or equal to −3 in an aqueous buffer at a pH of 7.4. In some cases, the hydrophobic molecule (H) is water insoluble at pH 7.4. In some cases, the peptide antigen conjugate has a net electrostatic charge greater than or equal to +3 or less than or equal to −3 in an aqueous buffer at a pH of 7.4, and also the hydrophobic molecule (H) is water insoluble at pH 7.4. In other cases, the peptide antigen conjugate is uncharged at neutral pH, such as pH 7.4.

This general architecture leads to a variety of possible structures. And it should be appreciated that these formulas do not necessarily require that S and H are at the N- and C-termini of a peptide antigen. Instead, they can be at either terminal or both at the same termini (e.g., via a trifunctional linker). In some cases, a conjugate only includes the S-A-H (e.g., C-A-H) components, while in other cases, it may have the formula S-E1-A-H, S-A-E2-H, S-E1-A-E2-H, S-A-U-H, S-E1-A-U-H, S-A-E2-U-H, or S-E1-A-E2-U-H, in all cases optionally with or without an associated drug D. In some embodiments, the drug (D) is covalently linked to the hydrophobic block (H). More generally, non-limiting examples of peptide antigen conjugates of this overall architecture include S-A-H, S-E1-A-H, S-A-E2-H, S-E1-A-E2-H, A-H(S), A-E2-H(S), E1-A-H(S), E1-A-E2-H(S), S1-A-H(S2), S1-A-E2-H(S2), S1-E1-A-H(S2), S1-E1-A-E2-H(S2), H-A-S, H-E1-A-S, H-A-E2-S, H-E1-A-E2-S, H(S)-A, H(S)-E1-A, H(S)-A-E2, H(S)-E1-A-E2, H(S1)-A-S2, H(S1)-E1-A-S2, H(S1)-A-E2-S2, H(S1)-E1-A-E2-S2, S-A-U-H, S-E1-A-U-H, S-A-E2-U-H, S-E1-A-E2-U-H, A-U-H(S), A-E2-U-H(S), E1-A-U-H(S), E1-A-E2-U-H(S), S1-A-U-H(S2), S1-A-E2-U-H(S2), S1-E1-A-U-H(S2), S1-E1-A-E2-U-H(S2), H-U-A-S, H-U-E1-A-S, H-U-A-E2-S, H-U-E1-A-E2-S, H(S)-U-A, H(S)-U-E1-A, H(S)-U-A-E2, H(S)-U-E1-A-E2, H(S1)-U-A-S2, H(S1)-U-E1-A-S2, H(S1)-U-A-E2-S2, H(S1)-U-E1-A-E2-S2, S-A-P, S-E1-A-P, S-A-E2-P, S-E1-A-E2-P, A-P(S), A-E2-P(S), E1-A-P(S), E1-A-E2-P(S), S1-A-P(S2), S1-A-E2-P(S2), S1-E1-A-P(S2), S1-E1-A-E2-P(S2), P-A-S, P-E1-A-S, P-A-E2-S, P-E1-A-E2-S, P(S)-A, P(S)-E1-A, P(S)-A-E2, P(S)-E1-A-E2, P(S1)-A-S2, P(S1)-E1-A-S2, P(S1)-A-E2-S2, P(S1)-E1-A-E2-S2, S-A-U-P, S-E1-A-U-P, S-A-E2-U-P, S-E1-A-E2-U-P, A-U-P(S), A-E2-U-P(S), E1-A-U-P(S), E1-A-E2-U-P(S), S1-A-U-P(S2), S1-A-E2-U-P(S2), S1-E1-A-U-P(S2), S1-E1-A-E2-U-P(S2), P-U-A-S, P-U-E1-A-S, P-U-A-E2-S, P-U-E1-A-E2-S, P(S)-U-A, P(S)-U-E1-A, P(S)-U-A-E2, P(S)-U-E1-A-E2, P(S1)-U-A-S2, P(S1)-U-E1-A-S2, P(S1)-U-A-E2-S2 or P(S1)-U-E1-A-E2-S2. In these formulas above, P represents a particle. Non-limiting examples of particles (P) include, polymer particles, such as poly(lactic-co-glycolic acid) (PLGA), polymersomes or polaxmers; lipid-based micelles, liposomes, or multi-lamellar vesicles; oil in water emulsions, such as mineral oil-in-water and water-in-mineral oil emulsions; and inorganic salt particles, such as aluminum phosphate or aluminum hydroxide salt particles (i.e. Alum). In some embodiments, the Particle (P) is a liposomal nanoparticle. In other embodiments, the Particle (P) is an iron particle. In still other embodiments, the Particle (P) is a polymer particle and A is a peptide antigen, for instance, as described above. Such structures may optionally further include a drug molecule D, which may be noncovalently associated with the peptide antigen conjugate for example within a micellar particle, or which may be covalently linked to the peptide antigen conjugate, such as to the hydrophobic block H. Other components of peptide antigen conjugates, i.e., S, E1, E2, U, and H, as well as an optional drug D, are described in more detail below.

In some cases, the peptide antigen conjugate further includes an amphiphile to assist with formation of micelles in aqueous solution, for example. The amphiphile may be a separate molecule that is mixed with the peptide antigen conjugate to form micelles, for example. Exemplary amphiphiles are described below, and may comprise several of the same components as the peptide antigen conjugates, such as a solubilizing block S, an optional linker denoted by either U or L, and a hydrophobic block H, with optional extensions or spacers, denoted alternatively B or E, between these components. Thus, such amphiphiles may be amphiphilic, with a water-soluble solubilizing block and a non-water soluble hydrophobic block on each end. In some embodiments, the amphiphiles do not comprise an antigen A. In some embodiments, the amphiphiles comprise or do not comprise a drug D. In some embodiments, where the drug D is present, it is covalently linked to the hydrophobic block (H). In some embodiments, the peptide antigen conjugate and/or amphiphile further comprises a dendron amplifier, as described below.

Exemplary amphiphile structures are described below and in International Patent Publication No. WO 2022/177993 and International Patent Application No. PCT/US2022/033819 filed Jun. 16, 2022 (WO 2022/266340). In some embodiments, an amphiphile has the formula S-[B]-[U]-H [D], where S and H are solubilizing and hydrophobic blocks as in the peptide antigen conjugates, B is an extension and may be the same or similar to those found in the peptide antigen conjugates such as E1 and E2, and U is a linker which may be the same or similar to those found in the peptide antigen conjugates, [ ] indicates that a group is optional, and “-” indicates a covalent linkage. As in the peptide antigen conjugates above, the S and H groups in such formulae can be found on either the N-terminal or C-terminal end of a peptide spacer or linker.

Non-limiting examples of the components of peptide antigen conjugate vaccines and associated exemplary amphiphiles, such as solubilizing blocks, hydrophobic blocks, linkers, and extensions (i.e., S, H, L or U, and B, E1, E2) are as described in the following sections.

### Solubilizing Block (S)

In some embodiments, a peptide antigen conjugate or an associated amphiphile comprises a solubilizing block (S) on one end of the molecule, which functions to impart solubility in aqueous solutions at certain temperature, pH and salt concentration. In some cases, the solubilizing block (S) is a charged molecule (C). In other cases, it may be polar, but uncharged. In some cases, the peptide antigen conjugate has a structure S-A-H or C-A-H such that a solubilizing block is present on one side of the antigen peptide and a hydrophobic block (H) (discussed below) is present on the other side, thus encouraging formation of a micellar structure in aqueous environments. Similarly, an amphiphile may have the structure S-[B]-[U]-H [D], such that the solubilizing block and hydrophobic block are similarly on opposite ends of the molecule, with an optional extension B and linker U in between. In some embodiments, the amphiphile may have the structure S-[B]-[U]-H-[D], in which D, when present, is covalently linked to H.

In certain embodiments, the solubilizing block (S) is soluble in aqueous solutions up to about 1-1,000 mg/mL, e.g., up to about 1 mg/mL, about 10 mg/mL, about 100 mg/mL, about 200 mg/mL, or about 500 mg/mL, though, typically not more than 1,000 mg/mL. In some embodiments, the solubilizing block (S) is soluble in aqueous solutions at certain concentrations, temperatures and/or pH ranges but becomes insoluble or less soluble in response to a change in concentration, temperature and/or pH. Exemplary solubilizing blocks (S) are molecules that are soluble at concentrations up to at least 1 mg/mL or up to at least about 10 mg/mL or up to at least about 100 mg/mL at or near physiologic pH (˜pH 7.4), between about pH 6.5 to pH 8.5 or between about pH 6.0 and pH 9.0, and at or near physiologic temperature (˜37° C.), such as between about 32-40° C., and at physiologic salt concentrations (˜10 g/L) and salt composition.

The solubilizing block may be chosen from any molecule that is water soluble and/or has hydrophilic characteristics. In some embodiments the solubilizing block (S) is selected from a linear, branched or brush polymer (or oligomer). The solubilizing block (S) can be a homopolymer or copolymer. The solubilizing block (S) can comprise one or many different types of monomer units. The solubilizing block (S) can be a statistical copolymer or alternating copolymer. The solubilizing block (S) can be a block copolymer, such as the A-B type, or the polymer can comprise a grafted copolymer, whereby two or more polymers are linked through a polymerization-type reaction.

The solubilizing block (S) may comprise polymers comprising naturally occurring and/or non-natural monomers and combinations thereof.

In some embodiments, the solubilizing block (S) is selected from natural biopolymers. Natural biopolymers selected as solubilizing blocks (S) may include peptides (sometimes referred to as poly(amino acids)) comprising hydrophilic amino acids. Non-limiting examples of hydrophilic amino acids include serine, sulfo-serine, glutamic acid, aspartic acid, lysine, ornithine, arginine. Biopolymers can be selected from hydrophilic polysaccharides, which may include but are not limited to glycogen, cellulose, dextran, alginate and chitosan.

Monomers comprising the solubilizing block (S) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylate and (meth)acrylamide monomers include N-2-hydroxypropyl(methacrylamide) (HPMA) and hydroxyethyl(methacrylate) (HEMA). Various monomers suitable for the solubilizing block (S) are described below.

In certain embodiments, the solubilizing block (S) comprises hydrophilic polymers selected from synthetic or natural poly(saccharides), such as glycogen, cellulose, dextran, alginate and chitosan. Hydrophilic polymers used as the solubilizing block (S) should have sufficient length to provide adequate surface coverage to stabilize particles of an S-A-H type. In preferred embodiments of solubilizing blocks comprising hydrophilic polymers, the hydrophilic polymer comprises 50 or monomer units, such as between 50 to 300, though, preferably between 50 and 100.

Solubilizing blocks (S) comprising linear polymers may comprise homopolymers comprising a single monomer composition or copolymers having two or more distinct compositions of monomers. In some embodiments, the homopolymer comprises neutral, hydrophilic monomers or charged monomers, e.g., positive, negative or zwitterion monomers. In other embodiments, the copolymer comprises neutral, hydrophilic monomers, and positive, negative or zwitterion monomers, or any combination thereof. Solubilizing blocks comprising linear polymers may comprise monomers linked to any solubilizing groups (SG) (or “moieties”), which generally refers to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof.

In some embodiments, the solubilizing block (S) comprises neutral hydrophilic monomers, which may be described generically as hydrophilic monomers. In some embodiments, the hydrophilic monomers are selected from (meth)acrylates or (meth)acrylamides (inclusive of acrylates, methacrylates, acrylamides and methacrylamides) of the chemical formula CH₂═CR¹¹—C(O)—R¹⁰ (“Formula II”), wherein the acryl side group R¹⁰ may be selected from one or more of —OR², —NHR¹² or —N(CH₃)R¹², where R¹¹ can be H or CH₃, and R¹² is independently selected from any hydrophilic substituent. Non-limiting examples of R¹² include but are not limited to H (except for OR¹³), CH₃, CH₂CH₃, CH₂CH₂OH, CH₂(CH₂)₂OH, CH₂CH(OH)CH₃, CHCH₃CH₂OH or (CH₂CH₂O)_{y}H, where y is an integer number of repeating units, typically 1 to 6, such as 1, 2, 3, 4, 5 or 6.

A non-limiting example of a neutral hydrophilic monomer of Formula II wherein R¹⁰=NHR¹², R¹¹=CH₃, and R¹³=CH₂CH(OH)CH₃ is N-2-hydroxypropyl(methacrylamide) (HPMA):

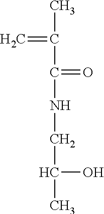

The above example, N-(2-hydroxpropyl(methacrylamide)) (HPMA), is an example of a neutral hydrophilic monomer of Formula II.

In some embodiments, the solubilizing block (S) comprises charged monomers that contain one or more functional groups (“charged functional group”) that either have a fixed charge or have net charge under certain physiological conditions. Thus, the solubilizing block comprises charged molecules (C). Non-limiting examples of charged monomers include any monomer that comprises amine, quaternary ammonium, sulfonic acid, sulfuric acid, sulfonium, phosphoric acid, phosphonic acid, phosphonium, carboxylic acid and/or boronic acid functional groups, as well as any combinations or salt forms thereof.

In some embodiments, charged monomers are selected from (meth)acrylates and (meth)acrylamides with chemical formula CH₂═CR¹⁴—C(O)—R¹³ (“Formula III”). The acryl side group R¹³ may be selected from one or more of the groups consisting of —OR¹⁵, —NHR¹⁵ or —N(CH₃)R¹⁵, where R¹⁴ can be H or CH₃ and R¹⁵ can be selected from, but is not limited to, H, linear alkyl structures such as (CH₂)_{y}NH₂, (CH₂)_{y}-imidazole, (CH₂)_{y}-pyridine amine, (CH₂)_{y}-(quinoline-amine), (CH₂)_{y}-pyridine amine, (CH₂)_{y}-naphthalene amine, (CH₂)_{y}CH(NH₂)COOH, (CH₂)_{y}COOH, (CH₂)_{y}CH(CH₃)COOH, (CH₂)_{y}C(CH₃)₂COOH, (CH₂)_{y}PO₃H₂, (CH₂)_{y}OPO₃H₂, (CH₂)_{y}SO₃H, (CH₂)_{y}OSO₃H, (CH₂)_{y}B(OH)₂, CH₂N(CH₃)₂, CH₂CH₂N(CH₃)₂, CH₂CH₂CH₂N(CH₃)₂, CH₂N(CH₂CH₃)₂, CH₂CH₂N(CH₂CH₃)₂, CH₂CH₂CH₂N(CH₂CH₃)₂, CH₂N(CH(CH₃)₂), CH₂CH₂N((CH(CH₃)₂), CH₂CH₂CH₂N(CH(CH₃)₂), CH[CH₂N(CH₃)₂]₂, CH(COOH)CHCH₂COOH, (CH₂)_{y}NH(CH₂)ⱼCOOH, (CH₂)_{y}N(CH₃)(CH₂)_{y}COOH, (CH₂)_{y}N⁺(CH₃)₂(CH₂)_{y}COOH, (CH₂)_{y}N⁺(CH₂—CH₃)₂(CH₂)_{y}COOH, [CH₂CH(CH₃)O]₅PO₃H₂, C(CH₃)₂CH₂SO₃H, and C₆H₄B(OH)₂ where y is an integer number of a repeating units, typically between 1 to 6, such as 1, 2, 3, 4, 5 or 6. In some embodiments of (meth)acrylates and (meth)acrylamides of Formula III, the acryl side group comprises tetraalkyl ammonium salts, nitrogen containing heterocycles, aminoaryl, or aminoheteroaryl, which may be linked to the monomer through any suitable means either directly or via a linker. Non-limiting examples of aryls, nitrogen containing heteroaryls and/or aminoheteroaryls include pyrrolyl, imidazolyl, pyridinyl, pyrimidinyl, pyrazinyl, diazepinyl, indolyl, quinolinyl, amino quinolinyl, amino pyridinyl, purinyl, pteridinyl, anilinyl, amino naphthyl or the like. In certain preferred embodiments of (meth)acrylates and (meth)acrylamides of Formula III, the acryl side group comprises carboxylic acid(s), which may be linked to the monomer through any suitable means either directly or via a linker. A non-limiting example of a charged monomer of Formula III wherein R¹³=—OR¹⁵, R⁴=CH₃ and R¹⁵=H is:

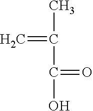

Dendron-based solubilizing blocks Certain embodiments of solubilizing blocks (S) comprise dendron amplifiers (“dendrons”), wherein the focal point of the solubilizing block (S) is linked either directly or indirectly via a spacer (e.g., E1 or E2) and/or Linker U to an antigen (A) and hydrophobic block (H), and the terminal groups (FGt) are either blind ended (unlinked) and function as solubilizing groups, or the terminal functional groups (FGt) are linked to solubilizing groups, wherein the solubilizing groups (SG) (or “moieties”) generally refer to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof. In some embodiments, the solubilizing block (S) comprises dendron architecture and the terminal functional groups (FGt) are unlinked and therefore FGt are the solubilizing groups (SG). In other embodiments, the solubilizing block (S) comprises dendron architecture and the terminal functional groups (FGt) are linked either directly or via a linker to a solubilizing group (SG).

In some cases, the dendron has 2 or more solubilizing groups (SG), e.g., between 2 and 32 solubilizing groups, or between 4 and 8 solubilizing groups. The solubilizing block (S) charge and composition can be tuned by varying the solubilizing groups (SG) to modulate biological activity. Examples of solubilizing groups are described below and throughout the specification.

In some embodiments, the solubilizing block (S) is a linear poly(amino acid) comprising charged amino acids, hydrophilic amino acids or a combination thereof. In some embodiments, a solubilizing block (S) comprising poly(amino acids) is linked to peptide antigen conjugates either directly or indirectly via an extension (E1 or E2) and/or Linker U.

Solubilizing blocks comprising poly(amino acids) may comprise amino acids linked to any solubilizing groups (SG) (or “moieties”), which generally refers to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof.

### Charged Molecules (C)

In certain embodiments, the solubilizing block (S) comprises a charged molecule (C). A charged molecule (C) refers to any molecule that has one or more functional groups that are positively or negatively charged in aqueous buffers at a pH of about 7.4. The functional groups comprising the charged molecule (C) may be partial or full integer values of charge. A charged molecule (C) may be a molecule with a single charged functional group or multiple charged functional groups. The net charge of the charged molecule (C) may be positive, negative or neutral. The charge of functional groups comprising the charged molecule (C) may be dependent or independent of the pH of the solution in which the charged molecule (C) is dispersed, such is the case, for example, for tertiary amines and quaternary ammonium compounds that are pH dependent and pH independent, respectively. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule.

The charged molecule (C) may either carry a net negative, net positive or neutral charge and depends on the net charge of the peptide antigen conjugate needed for the specific application of the invention disclosed herein. For example, most cell surfaces are known to carry a net negative charge. Thus, net positively charged particles may interact with all cell surfaces without a high degree of specificity. In contrast, net negatively charged particles will be electrostatically repulsed from most cell surfaces but have been shown to promote selective uptake by certain antigen-presenting cell populations. For example, positively charged particles delivered intravenously into the circulation have been found to accumulate in the liver and lungs as well as within antigen-presenting cells in the spleen, whereas negatively charged particles have been found to preferentially accumulate in antigen-presenting cells in the spleen following intravenous administration. Thus, the net charge of the charged molecule (C) can be adjusted to meet the specific demands of the application.

In some embodiments, the charged molecule (C) has a net negative charge and is comprised of functional groups that carry a negative charge at physiologic pH, at a pH of about 7.4. Suitable charged molecules (C) that carry a net negative charge include molecules bearing functional groups (e.g., functional groups with a pKa less than about 6.5) that occur as the conjugate base of an acid at physiologic pH, at a pH of about 7.4. These include but are not limited to molecules bearing carboxylates, sulfates, phosphates, phosphoramidates, and phosphonates. The charged molecule (C) bearing a carboxylate can be but is not limited to glutamic acid, aspartic acid, pyruvic acid, lactic acid, glycolic acid, glucuronic acid, citrate, isocitrate, alpha-keto-glutarate, succinate, fumarate, malate, and oxaloacetate and derivatives thereof. In preferred embodiments, the negatively charged molecule (C) is comprised of a molecule with between 1-20 negatively charged functional groups, such as 1, 2, 3, 4, 5, 6, 7, 8, 9, 10, 11, 12, 13, 14, 15, 16, 17, 18, 19 or 20 negatively charged functional groups, though, typically no more than 16 negatively charged functional groups. In some embodiments, the charged molecule (C) is a poly(glutamic acid) peptide of between 2-6 amino acids in length. A poly(glutamic acid) sequence comprised of 1, 2, 3, 4, 5 or 6 amino acids would be expected to carry a negative charge of −1, −2, −3, −4, −5 and −6 at pH 7.4, respectively. In additional embodiments, the charged molecule (C) is phosphoserine or sulfoserine.

In non-limiting examples, a poly(amino acid) comprising 12 aspartic acid monomers, e.g., Asp-Asp-Asp-Asp-Asp-Asp-Asp-Asp-Asp-Asp-Asp-Asp (SEQ ID NO:32), is used to prepare a solubilizing block (S) with a net negative charge of −12; a poly(amino acid) comprising 11 aspartic acid monomers, e.g., Asp-Asp-Asp-Asp-Asp-Asp-Asp-Asp-Asp-Asp-Asp (SEQ ID NO:33), is used to prepare a solubilizing block (S) with a net negative charge of −11; a poly(amino acid) comprising 10 aspartic acid monomers, e.g., Asp-Asp-Asp-Asp-Asp-Asp-Asp-Asp-Asp-Asp (SEQ ID NO:34), is used to prepare a solubilizing block (S) with a net negative charge of −10; a poly(amino acid) comprising 9 aspartic acid monomers, e.g., Asp-Asp-Asp-Asp-Asp-Asp-Asp-Asp-Asp (SEQ ID NO:35), is used to prepare a solubilizing block (S) with a net negative charge of −9; a poly(amino acid)) comprising 8 aspartic acid monomers, e.g., Asp-Asp-Asp-Asp-Asp-Asp-Asp-Asp (SEQ ID NO:36), is used to prepare a solubilizing block (S) with a net negative charge of −8; a poly(amino acid) comprising 7 aspartic acid monomers, e.g., Asp-Asp-Asp-Asp-Asp-Asp-Asp (SEQ ID NO:37), is used to prepare a solubilizing block (S) with a net negative charge of −7; a poly(amino acid) comprising 6 aspartic acid monomers, e.g., Asp-Asp-Asp-Asp-Asp-Asp (SEQ ID NO:38), is used to prepare a solubilizing block (S) with a net negative charge of −6; a poly(amino acid) comprising 5 aspartic acid monomers, e.g., Asp-Asp-Asp-Asp-Asp (SEQ ID NO:39), is used to prepare a solubilizing block (S) with a net negative charge of −5; a poly(amino acid) comprising 4 aspartic acid monomers, e.g., Asp-Asp-Asp-Asp (SEQ ID NO:40), is used to prepare a solubilizing block (S) with a net negative charge of −4; a poly(amino acid) comprising 3 aspartic acid monomers, e.g., Asp-Asp-Asp, is used to prepare a solubilizing block (S) with a net negative charge of −3; a poly(amino acid) comprising 2 aspartic acid monomers, e.g., Asp-Asp, is used to prepare a solubilizing block (S) with a net negative charge of −2. In the above examples, aspartic acid (Asp) may be replaced with any suitable negatively charged amino acid, including but not limited to glutamic acid, sulfo-serine, or phospho-serine, wherein the negatively charged amino acids may be the same or different.

In some embodiments the charged molecule (C) has a net positive charge and is comprised of positively charged functional groups. Suitable positively charged molecules (C) include those with functional groups that carry positive charge at physiologic pH, at a pH of about 7.4, such as the conjugate acid of weak bases, wherein the pKa of the conjugate acid of the base is greater than about 8.5. Suitable positively charged molecules (C) include but are not limited to molecules bearing primary, secondary and tertiary amines, as well as quaternary ammonium, guanidinium, phosphonium and sulfonium functional groups. Suitable molecules bearing ammonium functional groups include, for example, imidazolium, and tetra-alkyl ammonium compounds. In some embodiments, the charged molecule (C) is comprised of quaternary ammonium compounds that carry a permanent positive charge that is independent of pH.

Non-limiting examples of positively charged functional groups that have charge independent of pH include:

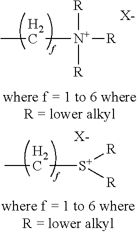

wherein X⁻ is any suitable counter anion.

In additional embodiments, the charged molecule (C) is comprised of functional groups that occur as the conjugate acid of a base at physiologic pH, such as, for example, primary, secondary and tertiary amines. In preferred embodiments, the positively charged molecule (C) is comprised of between 1-20 positively charged functional groups, such as 1, 2, 3, 4, 5, 6, 7, 8, 9, 10, 11, 12, 13, 14, 15, 16, 17, 18, 19 or 20 positively charged functional groups, though, typically no more than 16 charged functional groups. In some embodiments, the charged molecule (C) is a poly(lysine) peptide of between 1-6 amino acids in length. A poly(lysine) sequence comprised of 1, 2, 3, 4, 5 or 6 amino acids would be expected to carry a positive charge of +1, +2, +3, +4, +5 or +6 respectively, at pH 7.4. In additional embodiments, the charged molecule (C) is a poly(arginine) peptide of between 2-6 amino acids in length.

In non-limiting examples, a poly(amino acid) comprising 12 lysine monomers, e.g., Lys-Lys-Lys-Lys-Lys-Lys-Lys-Lys-Lys-Lys-Lys-Lys (SEQ ID NO:41), is used to prepare a solubilizing block (S) with a net positive charge of +12; a poly(amino acid) comprising 11 lysine monomers, e.g., Lys-Lys-Lys-Lys-Lys-Lys-Lys-Lys-Lys-Lys-Lys (SEQ ID NO:42), is used to prepare a solubilizing block (S) with a net positive charge of +11; a poly(amino acid) comprising 10 lysine monomers, e.g., Lys-Lys-Lys-Lys-Lys-Lys-Lys-Lys-Lys-Lys (SEQ ID NO:43), is used to prepare a solubilizing block (S) with a net positive charge of +10; a poly(amino acid) comprising 9 lysine monomers, e.g., Lys-Lys-Lys-Lys-Lys-Lys-Lys-Lys-Lys (SEQ ID NO:44), is used to prepare a solubilizing block (S) with a net positive charge of +9; a poly(amino acid) comprising 8 lysine monomers, e.g., Lys-Lys-Lys-Lys-Lys-Lys-Lys-Lys (SEQ ID NO:45), is used to prepare a solubilizing block (S) with a net positive charge of +8; a poly(amino acid) comprising 7 lysine monomers, e.g., Lys-Lys-Lys-Lys-Lys-Lys-Lys (SEQ ID NO:46), is used to prepare a solubilizing block (S) with a net positive charge of +7; a poly(amino acid) comprising 6 lysine monomers, e.g., Lys-Lys-Lys-Lys-Lys-Lys (SEQ ID NO:47), is used to prepare a solubilizing block (S) with a net positive charge of +6; a poly(amino acid) comprising 5 lysine monomers, e.g., Lys-Lys-Lys-Lys-Lys (SEQ ID NO:48), is used to prepare a solubilizing block (S) with a net positive charge of +5; a poly(amino acid) comprising 4 lysine monomers, e.g., Lys-Lys-Lys-Lys (SEQ ID NO:49), is used to prepare a solubilizing block (S) with a net positive charge of +4; a poly(amino acid) comprising 3 lysine monomers, e.g., Lys-Lys-Lys, is used to prepare a solubilizing block (S) with a net positive charge of +3; a poly(amino acid) comprising 2 lysine monomers, e.g., Lys-Lys, is used to prepare a solubilizing block (S) with a net positive charge of +2. In the above examples, Lysine (Lys) may be replaced with any suitable positively charged amino acid, including but not limited to trimethyl-lysine, ornithine or arginine, wherein the positively charged amino acids may be the same or different. In preferred embodiments of vaccines, wherein the at least one peptide antigen conjugate has net positive charge, the peptide antigen conjugate comprises a solubilizing block (S) that further comprises between 1 to 20 positively charged amino acids that comprise primary amines, including but not limited to lysine and ornithine.

Charged molecules (C) may additionally comprise small non-charged, hydrophilic amino acids, or hydrophilic linkers, e.g., ethylene oxide that function to i) improve water solubility and ii) increase the distance between charged functional groups to prevent incomplete ionization. For instance, ionization of one functional group on a polymer may impact the pKa of neighboring functional groups through local effects. For example, protonation of an amine in close proximity to a second amine may lower the pKa of the conjugate acid of the second amine. To reduce the impact of local effects on the ionization potential of neighbouring functional groups, a linker molecule may be used to increase the distance between charged functional groups comprising the charged molecule. The linker molecule may comprise between 1-5 small, non-charged hydrophilic amino acids, e.g., 1, 2, 3, 4, and 5 amino acids. Alternatively, the linker may comprise an ethylene oxide (i.e, PEG) linker between 1-4 monomers units, e.g., 1, 2, 3, or 4 ethylene oxide monomers in length. In preferred embodiments, 1 to 2 small, non-charged hydrophilic amino acids are placed between neighbouring charged amino acids comprising the charged molecule (C), wherein the amino acids are linked through amide bonds. In certain embodiments, a serine is placed between each charged amino acid comprising a charged molecule (C) with a net positive charge. In preferred embodiments, the charged molecule (C) is comprised of repeating dipeptides of lysine and serine, i.e. (Lys-Ser)ₙ, where n is typically any integer between 1-20, e.g., 1, 2, 3, 4, 5, 6, 7, 8, 9, 10, 11, 12, 13, 14, 15, 16, 17, 18, 19 or 20. As other examples, a serine is placed between each charged amino acid of a tripeptide charged molecule (C) with a net +2 charge, e.g., Lys-Ser-Lys; a serine is placed between each charged amino acid of a 5 amino acid charged molecule (C) with a net +3 charge, e.g., Lys-Ser-Lys-Ser-Lys (SEQ ID NO: 52); a serine is placed between each charged amino acid of a 7 amino acid charged molecule (C) with a net +4 charge, e.g., Lys-Ser-Lys-Ser-Lys-Ser-Lys (SEQ ID NO: 53). In the above examples, Lysine (Lys) may be replaced with any suitable positively charged amino acid, including but not limited to trimethyl-lysine or arginine, wherein the positively charged amino acids may be the same or different.

In certain embodiments, a serine is placed between each charged amino acid comprising a charged molecule (C) with a net negative charge. In preferred embodiments, the charged molecule is comprised of repeating dipeptides of aspartic acid and serine, i.e. (Asp-Ser)ₙ, where n is typically any integer between 1-20, e.g., 1, 2, 3, 4, 5, 6, 7, 8, 9, 10, 11, 12, 13, 14, 15, 16, 17, 18, 19 or 20. For example, a serine is placed between each charged amino acid of a tripeptide charged molecule (C) with a net −2 charge, e.g., Asp-Ser-Asp; a serine is placed between each charged amino acid of a 5 amino acid charged molecule (C) with a net −3 charge, e.g., Asp-Ser-Asp-Ser-Asp (SEQ ID NO: 54); a serine is placed between each charged amino acid of a 7 amino acid charged molecule (C) with a net −4 charge, e.g., Asp-Ser-Asp-Ser-Asp-Ser-Asp (SEQ ID NO: 55). In the above examples, aspartic acid (Asp) may be replaced with any suitable negatively charged amino acid, including but not limited to glutamic acid, sulfo-serine, or phospho-serine, wherein the negatively charged amino acids may be the same or different.

In additional embodiments, the charged molecule (C) is comprised of both negatively and positively charged amino acids. Di-peptides comprised of amino acids of opposite charge, e.g., Lys-Asp, are referred to as zwitterion dipeptides because they are predicted to have a net neutral, 0, charge at pH 7.4. One or more zwitterion dipeptides can be included in the charged molecule (C) as a means to i) improve water solubility and ii) provide a prevailing charge (e.g., net negative or net positive) over certain pH ranges. For instance, a zwitterion di-peptide can be used to increase the hydrophilic character of a peptide sequence without increasing or decreasing the charge of a peptide sequence at pH 7.4. However, the zwitterion can be used to impart a net charge at a particular pH. For instance, excluding the contribution of the N-terminal amine and the C-terminal carboxylic acid in this example, the zwitterion di-peptide, Lys-Asp, has a net charge of 0 at pH 7.4, but a net charge of +1 at pH<4 and a net charge of −1 at pH>10. One or more zwitterion di-peptides can be added to the sequence of charged molecules (C); for example, one di-peptide, Lys-Asp; two di-peptides Lys-Asp-Lys-Asp (SEQ ID NO:50); three di-peptides, Lys-Asp-Lys-Asp-Lys-Asp (SEQ ID NO:51) and so forth. In the above examples, Lysine (Lys) may be replaced with any suitable positively charged amino acid, including but not limited to trimethyl-lysine or arginine, and aspartic acid (Asp) may be replaced with any suitable negatively charged amino acid, including but not limited to glutamic acid, sulfo-serine, or phospho-serine, wherein the positively or negatively charged amino acids may be the same or different.

The composition of the charged molecule (C) may be selected to provide the net charge needed of a peptide antigen conjugate for the specific application. In several embodiments disclosed herein, the charged molecule (C) is a positively charged poly(amino acid) comprised of lysines or arginines, or lysines or arginines and non-charged amino acids. In some embodiments the charged moiety comprised sulfonium or quaternary ammonium functional groups that carry pH independent positive charge. In several embodiments disclosed herein, the charged molecule (C) is a negatively charged poly(amino acid) comprised of glutamic acid or aspartic acid, or glutamic acid or aspartic acid and non-charged amino acids. In some embodiments the charged moiety comprises phosphate or sulfate groups, such as sulfoserine or phosphoserine. In additional embodiments, the charged molecule is comprised of lysines or arginines and glutamic acid or aspartic acid, or lysines or arginines and glutamic acid or aspartic acid as well as non-charged amino acids. Both positive and negatively charged functional groups may be included on the same charged molecule (C). The charged molecule (C) may be positive, negative or neutral but the net charge of the peptide antigen conjugate should be non-zero, for example, greater than +3 or less than −3 net charges are preferred and depend on the specific application.

An additional consideration regarding charged molecules (C), is the counterion selected. Non-limiting examples of charged molecules (C) bearing functional groups with positive charge include but are not limited to halides, including chloride, bromide and iodide anions, and conjugate bases of acids, including, phosphate, sulfates, sulfites and carboxylate anions including formate, succinate, acetate and trifluoroacetate. Suitable counterions for charged molecules (C) bearing functional groups with negative charge include but are not limited to hydrogen and alkali and alkaline earth metals, including, for example, sodium, potassium, magnesium and calcium, or conjugate acids of weak bases, such as ammonium compounds.

### Formula V

In several embodiments, the charged molecule (C) is placed at the N-terminus of a peptide antigen conjugate of Formula V, wherein the charged molecule (C) is linked to an N-terminal extension (E1) comprised of a cathepsin cleavable tetrapeptide extension (E1=PN4-PN3-PN2-PN1) that is linked to the N-terminus of a peptide antigen (A) that is linked at the C-terminus to a C-terminal extension (E2) comprised of a combined immuno-proteasome and cathepsin cleavable hexapeptide extension (E2=PC1′-PC2′-PC3′-PC4′-PC5′-PC6′) that is linked to a Linker (L) that is linked to a hydrophobic molecule (H) or Particle (P). The peptide antigen (A) of a peptide antigen conjugate of Formula V is comprised of an integer number of amino acids, n, wherein n is typically between 7-35 amino acids and the hydrophobic molecule (H) is typically a poly(amino acid) of Formula I or II linked to an Adjuvant of Formula III.

A non-limiting example of a peptide antigen conjugate of Formula V comprising a charged molecule (C=Lys-Lys) linked to a cathepsin cleavable tetrapeptide extension (B₁=Lys-Pro-Leu-Arg (SEQ ID NO: 2) at the N-terminus of a peptide antigen (A) that is linked at the C-terminus to a cathepsin cleavable hexapeptide extension (B₂=Gly-Gly-Ser-Leu-Val-Arg (SEQ ID NO: 19) that is linked to a triazole Linker (L) that is linked to a hydrophobic molecule (H) comprised of a poly(amino acid) of Formula I that is linked to an Adjuvant of Formula III is provided here:

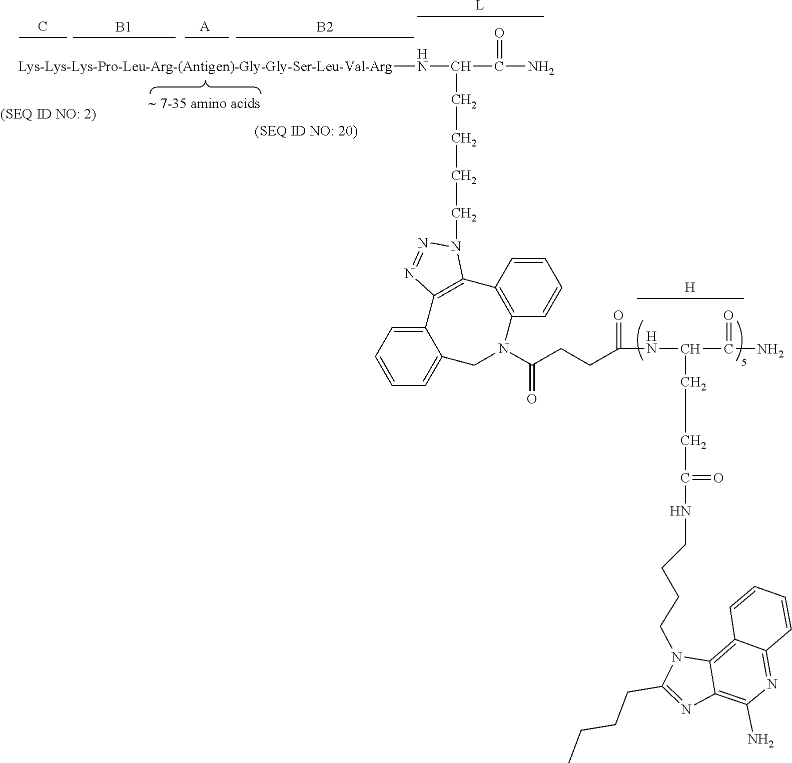

Note that the elements E1, E2, and U in this example are denoted above by their alternate names B1, B2, and L.

In additional embodiments, a charged molecule (C; or C₁ and C₂ when there are two charged molecules present) may be linked directly to the hydrophobic molecule (H) or to the Linker (L or U) that is linked to the C-terminal extension (E2 or B2) that is linked to the C-terminus of a peptide antigen (A) that is optionally linked at the N-terminus to an N-terminal extension (E1 or B1) that is optionally linked to an additional optional charged moiety (C₁); or the charged molecule (C; or C₁ and C₂ when there are two charged molecules present) may be linked directly to the hydrophobic molecule (H) or to the Linker (L or U) that is linked to the N-terminal extension (E1 or B1) that is linked to the N-terminus of a peptide antigen (A) that is optionally linked at the C-terminus to a C-terminal extension (E1 or B2) that is optionally linked to an additional optional charged moiety (C₂) to yield a peptide antigen conjugate of Formula VI, wherein [ ] denote that the group is optional:

[E1]-A-[E2]-U(C)-H, [E1]-A-[E2]-U-H(C), [C₁]-[E1]-A-[E2]-U(C₂)-H, [C₁]-[E1]-A-[E2]-U-H(C₂), H-U(C)-[E1]-A-[E2], H(C)-[E1]-A-[E2], H-U(C₁)-[E1]-A-[E2]-C₂ or H(C₁)-[E1]-A-[E2]-C₂ Formula VI

In several embodiments, the charged molecule (C) is placed at the C-terminus of a peptide antigen conjugate of Formula VI, wherein the charged molecule (C) is linked to a Linker (L) that is optionally linked to a C-terminal extension (E2 or B2) comprised of an immuno-proteasome, cathepsin or combined immuno-proteasome and cathepsin cleavable extension typically between 1 to 6 amino acids in length (B2=PC1′, PC1′-PC2′, PC1′-PC2′-PC3′, PC1′-PC2′-PC3′-PC4′, PC1′-PC2′-PC3′-PC4′-PC5′, or PC1′-PC2′-PC3′-PC4′-PC5′-PC6′) that is linked to the C-terminus of a peptide antigen (A) that is optionally linked at the N-terminus to a cathepsin cleavable extension typically between 1 to 4 amino acids in length (E1 or B1=PN1, PN2-PN1, PN3-PN2-PN1 or PN4-PN3-PN2-PN1), wherein the Linker (L or U) is additionally linked to a hydrophobic molecule (H), shown here:

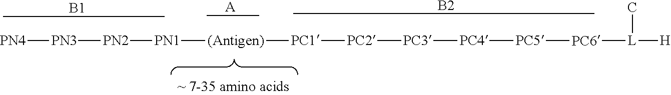

The peptide antigen (A) of the peptide antigen conjugate of Formula VI is comprised of an integer number of amino acids, n, wherein n is typically between 7-35 amino acids, or up to 50 amino acids, and the hydrophobic molecule is typically a poly(amino acid) of Formula I or II linked to an Adjuvant of Formula III.

A non-limiting example of a peptide antigen conjugate of Formula VI comprised of a charged molecule (e.g., C=Lys-Lys) linked via an amide bond to the C-terminus of a Linker (L) that is linked to a combined immuno-proteasome and cathepsin cleavable hexapeptide C-terminal extension (e.g., E2=Gly-Gly-Ser-Leu-Val-Arg (SEQ ID NO:19) that is linked to the C-terminus of a peptide antigen (A) that is linked at the N-terminus to a cathepsin cleavable tetrapeptide N-terminal extension (e.g., E1=Lys-Pro-Leu-Arg (SEQ ID NO:2)), wherein the Linker (L) is additionally linked to a hydrophobic molecule (H) that is comprised of a poly(amino acid) of Formula I that is linked to an Adjuvant of Formula III is provided:

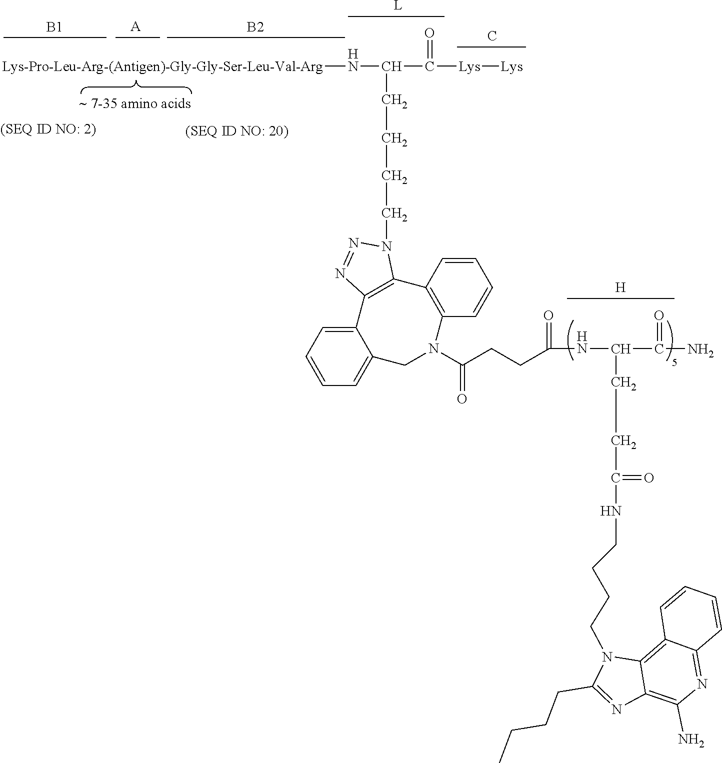

An additional non-limiting example of a peptide antigen conjugate of Formula VI, E1-A-E2-U-H(C), is a charged moiety (C=Lys-Lys-Lys-Lys-Lys) (SEQ ID NO: 48) linked via a linker to a hydrophobic molecule (H) that is comprised of a poly(amino acid) of Formula I that is linked to an Adjuvant of Formula III that is linked to a Linker (L) that is linked to a combined immuno-proteasome and cathepsin cleavable hexapeptide C-terminal extension (E2=Gly-Gly-Ser-Leu-Val-Arg (SEQ ID NO: 19)) that is linked to the C-terminus of a peptide antigen (A) and the N-terminus of the peptide antigen (A) is linked to a cathepsin cleavable tetrapeptide N-terminal extension (E1=Lys-Pro-Leu-Arg (SEQ ID No: 2)):

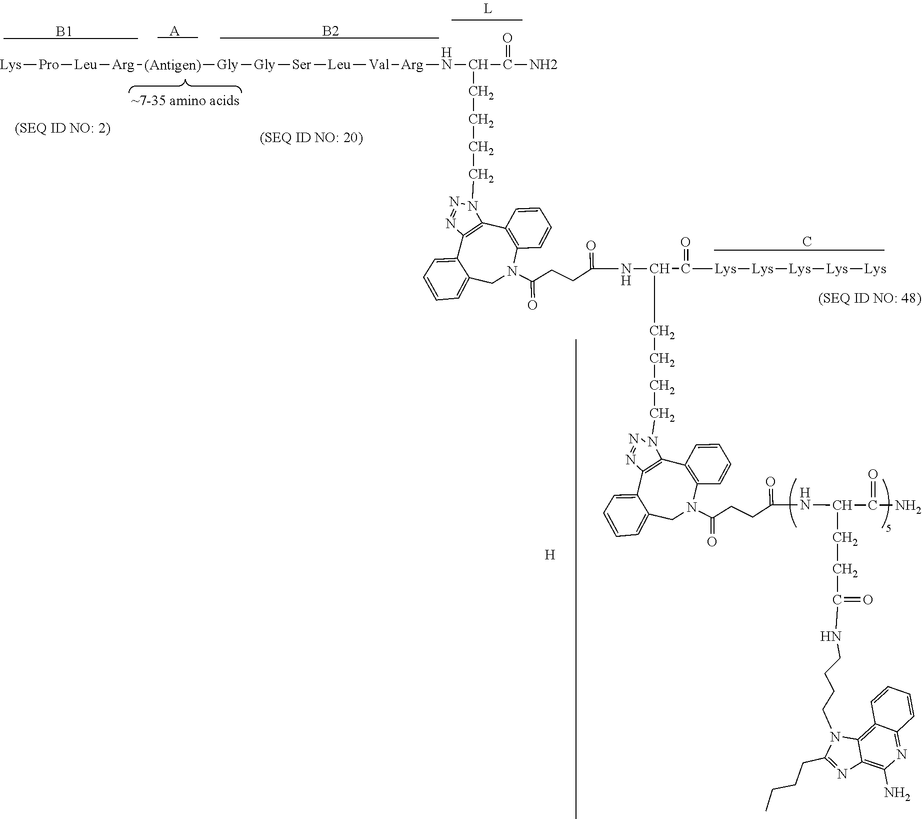

In a non-limiting example of a peptide antigen conjugate of Formula VI, E1-(A)₇₋₃₅-E2-L(-C)-H, a peptide antigen (A) with the sequence Ala-Lys-Phe-Val-Ala-Ala-Trp-Thr-Leu-Lys-Ala-Ala-Ala (SEQ ID NO: 166) is linked to an N-terminal extension (E1) with the sequence Ser-Leu-Val-Arg (SEQ ID NO: 11) and a C-terminal extension (E2) with the sequence Ser-Leu-Val-Arg (SEQ ID NO: 11) that is linked to a linker precursor X1, e.g., Lys(N3), that is linked to both a charged moiety (C) comprised of a dipeptide with the sequence Glu-Lys and a linker precursor X2, comprising a DBCO molecule that is linked to the hydrophobic molecule (H), for example: Ser-Leu-Val-Arg-Ala-Lys-Phe-Val-Ala-Ala-Trp-Thr-Leu-Lys-Ala-Ala-Ala-Ser-Leu-Val-Arg-Lys(N3-DBCO-H)-Glu-Lys (SEQ ID NO: 167), wherein the Glu-Lys sequence is linked to the C-terminus of the Linker (L or U) (Lys(N3-DBCO), resulting in a peptide antigen conjugate with a predicted net charge of +4 at pH 7.4. Here, the hydrophobic molecule (H) is assumed to have a negligible contribution to the charge of the peptide antigen conjugate. Note, that the composition of the charged moiety (C) and extension sequences (E1 and E2) can be selected to provide a particular number of charged residues that provide the desired net charge and hydropathy of the peptide sequence comprising the peptide antigen conjugate as described in greater detail below. In preferred embodiments, the number of charged functional groups comprising the charged moiety (C) is modulated such that the net charge of the peptide antigen conjugate comprising the charged moiety (C), peptide antigen (A), optional extensions (E1 and/or E2), Linker (L or U) and hydrophobic molecule (H) is between about −3 to −10 or between +3 to +10.

Peptide antigen conjugates of Formula VI, wherein the charged moiety (C) is linked to the hydrophobic molecule (H), may be advantageous for the rapid production of personalized therapies, such as personalized cancer vaccines. The hydrophobic molecule (H) that is linked to a charged molecule (C) and a linker precursor X2 (e.g., X2 comprising a cyclooctyne) can be prepared in bulk and then readily combined with any peptide antigen (A) bearing a linker precursor X1 (e.g., X1 comprising an azide) to form a peptide antigen conjugate of the Formula VI, [C₁]-[E1]-A-[E2]-U-H(C₂), or H(C)-U-[E1]-A-[E2]-[C₂], wherein [ ] denotes the group is optional.

The function of the charged moiety (C) is to stabilize nanoparticles formed by peptide antigen conjugates in aqueous conditions. While the hydrophobic molecule (H) induces particle formation of peptide antigen conjugates, the optional charged molecule (C) provides a countervailing force that prevents flocculation and, in some embodiments, drives the peptides antigen conjugates to assemble into nanoparticle micelles with a surface charge provided by the charged moiety (C).

In some embodiments, the peptide antigen conjugate does not comprise a charged molecules, such as [E1]-A-[E2]-[U]-H, where [ ] denotes that the group is optional. Non-limiting examples include, A-H, A-U-H, A-E2-H, A-E2-U-H, E1-A-E2-U-H. Peptide antigen conjugates that do not comprise a charged molecule (C) may undergo aggregation in aqueous conditions. To improve stability of particles formed by peptide antigen conjugates that do not comprise a charged moiety (C), a charged or amphiphilic molecule can be added. In some embodiments, a first peptide antigen conjugate that does not comprise a charged moiety (C) (i.e., [E1]-A-[E2]-[U]-H) is mixed with a second peptide antigen conjugate comprising a charged moiety (e.g., S-[E1]-A-[E2]-[U]-H) in a DMSO solution and then resuspended in aqueous conditions to form stable nanoparticles. In other embodiments, a peptide antigen conjugate that does not comprise a charged molecule (C) (i.e. [E1]-A-[E2]-[U]-H) is mixed with a hydrophobic molecule (H) linked to a charged molecule (C), such as C—H, in a DMSO solution and then resuspended in aqueous conditions to form stable nanoparticles.

In some embodiments, a peptide antigen conjugate that does not comprise a charged molecule (C), such as [E1]-A-[E2]-[U]-H, where [ ] denotes that the group is optional, is combined with an amphiphilic carrier, S-[E1]-[A′]-[E2]-[U]-H, wherein [ ] denotes the group is optional and optional A′ is a conserved antigen (i.e. not patient-specific). In some embodiments, a peptide antigen conjugate comprising a charged molecule (C) is combined with an amphiphilic carrier. The amphiphilic carrier serves to stabilize nanoparticles, such as nanoparticle micelles formed by peptide antigen conjugates.

For compositions of vaccines comprising at least one peptide antigen conjugate comprising a solubilizing block, wherein the vaccine further comprises an amphiphilic carrier molecule (“amphiphile,” e.g., of formula S-[B]-[U]-H), the number of charged functional groups present on the solubilizing block (S) of the peptide antigen conjugate may be selected to ensure net charge of the peptide antigen conjugate at physiologic pH 7.4 is greater than or equal to +2 or greater than equal to +3, though, typically no more than +10, and the solubilizing block is typically selected from poly(amino acids) comprising lysine or ornithine. For compositions of vaccines meant for intravenous administration, wherein the at least one peptide antigen conjugate comprises a solubilizing block (S) and wherein the vaccine further comprises an amphiphilic carrier molecule (“amphiphile,” e.g., of formula S-[B]-[U]-H), the number of charged functional groups present on the solubilizing block (S) of the peptide antigen conjugate is typically selected to ensure net charge of the peptide antigen conjugate at physiologic pH 7.4 is greater than or equal to +2 or greater than equal to +3, but typically no more than +6, more preferably between +3 and +5, and the solubilizing block is typically selected from poly(amino acids) comprising lysine or ornithine. The process for designing and manufacturing peptide antigen conjugates to achieve a specific net charge has been described by Lynn and colleagues (see: Lynn et al., Nature Biotechnology. 2020) and in patent application WO2018187515, which are incorporated by reference herein in their entirety.

### Counter-Ion Selection

An additional consideration when a charged molecule (C) is used as a solubilizing block is the counterion selected. Non-limiting examples of charged molecules (C) bearing functional groups with positive charge include but are not limited to halides, including chloride, bromide and iodide anions, and conjugate bases of acids, including, phosphate, sulfates, sulfites and carboxylate anions including formate, succinate, acetate and trifluoroacetate. Suitable counterions for charged molecules (C) bearing functional groups with negative charge include but are not limited to hydrogen and alkali and alkaline earth metals, including, for example, sodium, potassium, magnesium and calcium, or conjugate acids of weak bases, such as ammonium compounds. Suitable amines used to form the ammonium salt include but are not limited to ammonium, primary amines, such as tris(hydroxymethyl)aminomethane (“TRIS”), secondary amines based on di-alkyl amines, such as dimethyl amine and diethyl amine, tertiary amines based on tri-alkyl amines, such as trimethylamine, di-isopropryl ethylamine (DIPEA) and triethylamine (TEA), as well as quaternary ammonium compounds. Unexpectedly, tris(hydroxymethyl)aminomethane (or Tris) as the ammonium salt of acids as the counterion of amphiphilic block copolymers with negative charge has improved solubility in both water-miscible organic solvents, such as DMSO, DMF, acetone and ethanol, and aqueous solutions. For these reasons, the protonated form of tris(hydroxymethyl)aminomethane is a preferred counter-ion to use in the preparation of salts of conjugate bases of acids present on the amphiphilic block copolymers of the present disclosure.

### Zwitterion Peptides

In additional embodiments, the solubilizing block (S) comprises both negatively and positively charged amino acids, or amino acids with both positively and negatively charged functional groups. Dipeptides comprising amino acids of opposite charge, e.g., Lys-Asp, are referred to as zwitterion dipeptides because they are predicted to have a net neutral, 0, charge at pH 7.4. One or more zwitterion dipeptides can be included in the solubilizing block (S) as a means to i) improve water solubility and ii) provide a prevailing charge (e.g., net negative or net positive) over certain pH ranges. For instance, a zwitterion di-peptide can be used to increase the hydrophilic character of a peptide sequence without increasing or decreasing the charge of a peptide sequence at pH 7.4. However, the zwitterion can be used to impart a net charge at a particular pH. For instance, excluding the contribution of the N-terminal amine and the C-terminal carboxylic acid in this example, the zwitterion di-peptide, Lys-Asp, has a net charge of 0 at pH 7.4, but a net charge of +1 at pH<4 and a net charge of −1 at pH>10. One or more zwitterion di-peptides can be added to the sequence of poly(amino acid)-based solubilizing blocks; for example, one di-peptide, Lys-Asp; two di-peptides Lys-Asp-Lys-Asp; three di-peptides, Lys-Asp-Lys-Asp-Lys-Asp and so forth. In the above examples, Lysine (Lys) may be replaced with any suitable positively charged amino acid, including but not limited to trimethyl-lysine, ornithine or arginine, and aspartic acid (Asp) may be replaced with any suitable negatively charged amino acid, including but not limited to glutamic acid, sulfo-serine, or phospho-serine, wherein the positively or negatively charged amino acids may be the same or different.

The solubilizing block (S) comprising poly(amino acids) may additionally comprise small non-charged, hydrophilic amino acids, or hydrophilic linkers, e.g., ethylene oxide that function to i) improve water solubility and ii) increase the distance between charged functional groups to prevent incomplete ionization. For instance, ionization of one functional group on a polymer may impact the pKa of neighboring functional groups through local effects. For example, protonation of an amine in close proximity to a second amine may cause a reduction in the pKa of the conjugate acid of the second amine. To reduce the impact of local effects on the ionization potential of neighboring functional groups, a linker molecule may be used to increase the distance between charged functional groups. The linker molecule may comprise between 1 to 5 small, non-charged hydrophilic amino acids, e.g., 1, 2, 3, 4, and 5 amino acids. Alternatively, the linker may comprise an ethylene oxide (i.e., PEG) linker between 1 to 4, or more, monomer units, e.g., 1, 2, 3, or 4 ethylene oxide monomers in length. In certain embodiments of solubilizing blocks comprising poly(amino acids), 1 to 2 non-bulky, non-charged hydrophilic amino acids are placed between neighboring charged amino acids, wherein the amino acids are linked through amide bonds. In certain embodiments, a serine is placed between all or some of the charged amino acids comprising the poly(amino acid)-based solubilizing block (S).

### Solubilizing Groups (SG)

Solubilizing blocks (S) may comprise certain solubilizing groups (SG) (or “moieties”) that are defined broadly as any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof.

In certain embodiments, the solubilizing block (S) comprises solubilizing groups (SG) selected from sugar molecules comprising one or more sugar monomers, e.g., monosaccharides, disaccharides, trisaccharides, oligosaccharides and the like. Non-limiting examples of solubilizing groups selected from sugar molecules include but are not limited to glucose, glucosamine, N-acetyl glucosamine, galactose, galactosamine, N-acetyl galactosamine, mannose and sialyl lewis^{X} (sLeX), which may be linked to solubilizing blocks through any suitable linker at any suitable attachment point, e.g.:

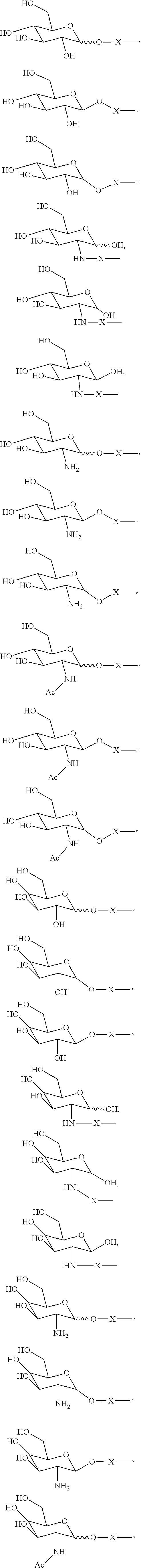

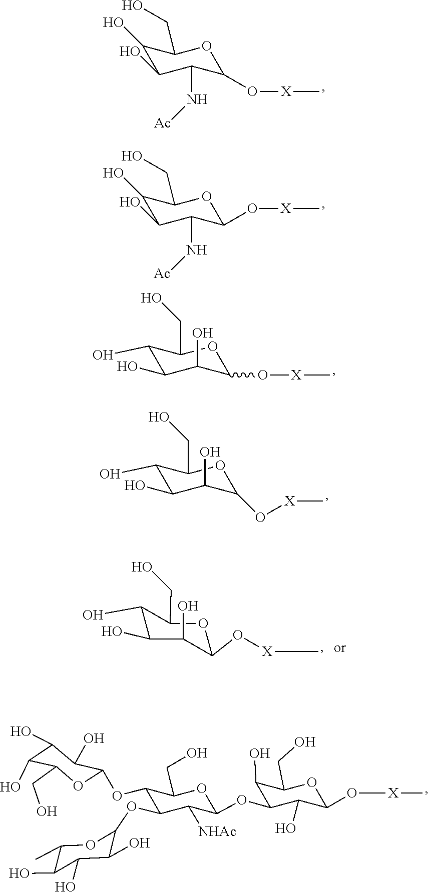

wherein X is any suitable linker molecule, which may be present or absent, and when present is typically selected from lower alkyl or PEG groups.

In some embodiments, the solubilizing block (S) comprises solubilizing groups (SG) that have net positive or net negative charge in aqueous buffers at a pH of about 7.4. The charge of the solubilizing groups (SG) may be dependent or independent of the pH of the solution in which the solubilizing block (S) is dispersed, such is the case, for example, for tertiary amines and quaternary ammonium compounds that are pH dependent and pH independent, respectively. Non-limiting examples of solubilizing groups that have net positive or net negative charge at certain pH in aqueous solutions or have pH independent charge are provided here for clarity:

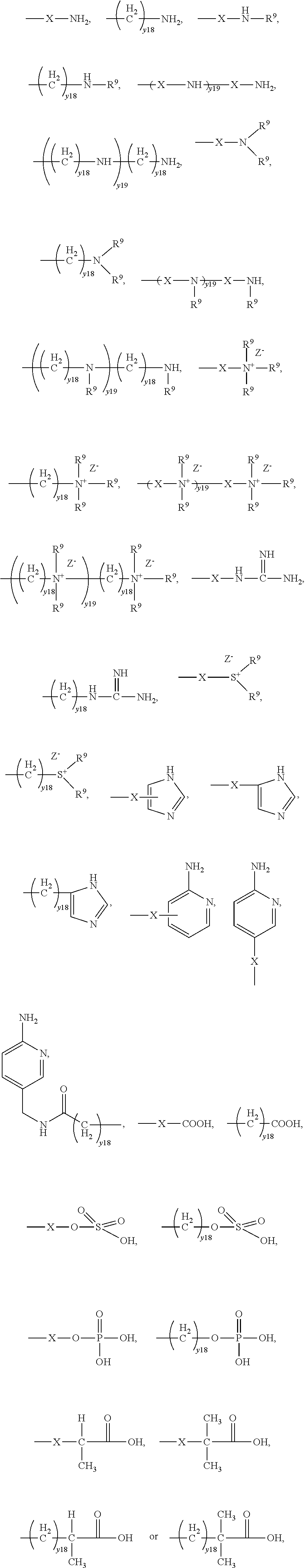

wherein X is any suitable linker molecule, which may be present or absent, and when present is typically selected from lower alkyl or PEG, y18 and y19 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z is any suitable counter anion, which is typically selected from conjugate bases of weak acids or halide ions, such as Cl⁻, I⁻, or Br⁻.

In certain embodiments, the solubilizing block (S) comprises solubilizing groups (SG) selected from zwitterions that have 0 net charge, or net 0 charge in aqueous conditions at certain pH. In some embodiments, the solubilizing block (S) comprises solubilizing groups (SG) selected from zwitterions that have 0 net charge at pH 7.4, but have net positive charge at reduced pH, e.g., tumor pH between about 5.5 to 7.0. Non-limiting examples of solubilizing groups comprising zwitterions are provided here for clarity:

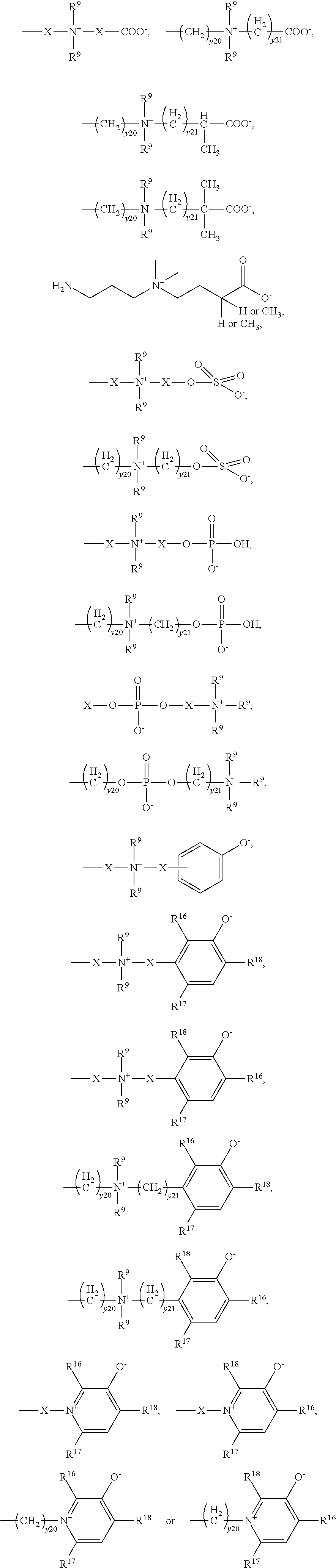

wherein X is any suitable linker, which may be present or absent, and when present is typically selected from lower alkyl or PEG groups, y20 and y21 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, R¹⁶, R¹⁷ and R¹⁸ are each independently selected from —H, CH₃, F and —NO₂.

In some embodiments, the solubilizing group (SG) may further comprise a targeting moiety and/or drug molecule. As a non-limiting example, certain sugar molecules may improve solubility and therefore function as a solubilizing group; additionally, the sugar molecule may bind to cell surface receptors and/or exert a physiological effect and therefore also function as a targeting moiety and/or drug molecule (D). Accordingly, solubilizing groups (SG) comprising mannose bind to mannose receptors and therefore target cells and tissues expressing such receptors; additionally, binding to the mannose receptor can promote phagocytosis and may therefore exert a physiological effect. Additional non-limiting examples of solubilizing groups (SG) that may perform two or more functions include targeting molecules comprising hydrophilic peptides, glycopeptides, antibodies, fragments of antibodies, nanobodies, nucleic acid aptamers and related molecules that are both hydrophilic and bind to specific cells or tissues.

### Linkage of Solubilizing Group (SG) to the Solubilizing Block (S)

Solubilizing groups (SG) may be linked to the solubilizing block (S) through any suitable means, including any suitable linker molecule. In certain preferred embodiments of dendron-based solubilizing blocks (S), the terminal functional group is a carboxylic acid, and the solubilizing group is linked via an ester or, more preferably, an amide bond. In certain other preferred embodiments of dendron-based solubilizing blocks (S), the terminal functional group is an amine, and the solubilizing group is linked to the terminal functional group via an amide or carbamate bond.

In preferred embodiments, solubilizing groups (SG) are linked to the solubilizing block (S) through a covalent bond via a suitable linker X, which is typically selected from lower alkyl or PEG groups. Particular suitable linkers X that are preferred for joining SG to S are referred to as X5. In non-limiting examples, solubilizing blocks (S) selected from either polymers comprising monomers comprising amines or dendrons comprising terminal functional groups (FGt) comprising amines, e.g., —NH₂, are covalently linked to solubilizing groups (SG) via a suitable linker, X5, through reaction with activated carboxylic acids (LG-C(O)—R¹⁹) to yield —NH—C(O)—R¹⁹; activated mixed carbonates (LG-C(O)—O—R¹⁹) or chloroformates (Cl—C(O)—O—R¹⁹) to yield NH—C(O)—O—R¹⁹; aldehydes or ketones (CR²²(O)—R¹⁹) to yield Schiff base of formula CR²²(—NH)—R¹⁹; alkenes (C(R²²)(R²³)═C(R²⁴)(R¹⁹) to yield Michael-addition products (e.g., NH—C(R²²)(R²³)—CH(R²⁴)(R¹⁹) or —N(C(R²²)(R²³)—CH(R²⁴)(R¹⁹))₂); or, alkyl or aryl halide (LG-R¹⁹, wherein LG=Cl, Br or I), to yield —NH—R¹⁹, —N(—R¹⁹)₂ and/or —N+(—R¹⁹)₃. In additional non-limiting examples, solubilizing blocks (S) selected from either polymers comprising monomers comprising carboxylic acids or dendrons comprising terminal functional groups (FGt) comprising carboxylic acids, e.g., —COOH (or —C(O)-LG), are covalently linked to solubilizing groups (SG) via a suitable linker, X5, through reaction with an amine (NH₂—R¹⁹) to yield —C(O)—NH—R¹⁹ or methylamine (R¹⁹—N(CH₃)(H) or R¹⁹—NHMe) to yield —C(O)—N(CH₃)(R¹⁹).

In the above non-limiting examples, LG is any suitable leaving group, and R¹⁹ may be selected from but is not limited to —(CH₂)ₜ—SG, —(CH₂CH₂O)ₜ—CH₂CH₂—SG, —(CH₂)ₜ—C(O)—NH—(CH₂)ᵤ—SG, —(CH₂CH₂O)ₜCH₂CH₂C(O)—NH—(CH₂)ᵤ—SG, —(CH₂)ₜ—NH—C(O)—NH—(CH₂)ᵤ—SG and (CH₂CH₂O)ₜCH₂CH₂NH—C(O)—(CH₂)ᵤ—SG where t and u are each independently an integer typically selected from between 1 to 6, such as 1, 2, 3, 4, 5 or 6. Exemplary X5 for linking S to SG (i.e., S—X5-SG) are typically selected from —NH—(CH₂)ₜ—, —NH—(CH₂CH₂O)ₜ—CH₂CH₂—, —NH—(CH₂)ₜ—C(O)—NH—(CH₂)ᵤ—, —NH—(CH₂CH₂O)ₜCH₂CH₂C(O)—NH—(CH₂)ᵤ—, NH—(CH₂)ₜ—NH—C(O)—NH—(CH₂)ᵤ—, —NH(CH₂CH₂O)ₜCH₂CH₂NH—C(O)—(CH₂)ᵤ—, —C(O)—(CH₂)ₜ—, —C(O)—(CH₂CH₂O)₁—CH₂CH₂—, —C(O)—(CH₂)ₜ—C(O)—NH—(CH₂)ᵤ—, C(O)—(CH₂CH₂O)ₜCH₂CH₂C(O)—NH—(CH₂)ᵤ—, C(O)—(CH₂)ₜ—NH—C(O)—NH—(CH₂)ᵤ— or —C(O)—(CH₂CH₂O)ₜCH₂CH₂NH—C(O)—(CH₂)ᵤ—, where t and u are each independently an integer typically selected from between 1 to 6, such as 1, 2, 3, 4, 5 or 6.

A non-limiting example of a solubilizing block (S) with dendron architecture, in the context of an amphiphile, comprises a dendron of second generation and comprises monomeric units selected from FG1-CH(R¹)₂, wherein FG1 (and the focal point) is NH₂, R¹ is (OCH₂CH₂)_{y}—FG2, y is 1 and FG2 (and FGt) is COOH, wherein the terminal functional group (FGt) carboxylic acids are linked to NH₂—R¹⁹ to yield —C(O)—NH₂—R¹⁹ wherein R¹⁹ is —(CH₂CH₂O)ₜ—CH₂CH₂—SG, t=1 and the solubilizing group is selected from a glucose is provided below for clarity:

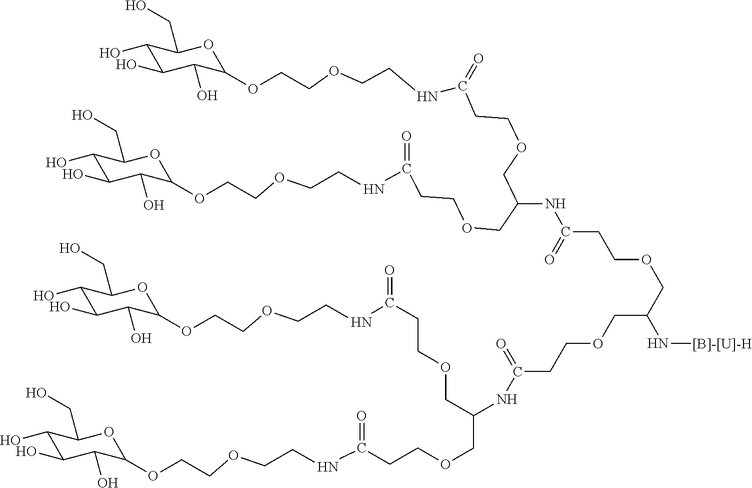

In the case of an amphiphile of S-[B]-[U]-H structure, the solubilizing block (S) is linked either directly or indirectly via a spacer (B) and/or Linker U to a hydrophobic block (H), which may further comprise a drug molecule D. In the above example, X5 is —NH—R¹⁹ and R¹⁹ is —(CH₂CH₂O)₁—CH₂CH₂—SG, which may be written as —NH—(CH₂CH₂O)ₜ—CH₂CH₂— (SG not shown), wherein t=1 and SG is α glucose.

### Dendron Amplifier

Dendron amplifiers are a specific type of linker moiety that functions to increase the valency (i.e., the number) of groups present on any components of amphiphiles, peptide antigen conjugates or drug molecule conjugates described herein. For instance, in preferred embodiments of solubilizing blocks (S), dendron amplifiers are used to increase the valency of solubilizing groups (referred to as “SG” in formulae) that are present on the surface of the solubilizing block (S). In other embodiments, dendron amplifiers are used to increase the valency of solubilizing blocks (S) and spacers (B) linked to a hydrophobic block (H).

Dendron amplifiers (also referred to as “dendrons”) are regularly branched molecules that are often symmetric and typically comprise repeating units of monomers that comprise three or more functional groups (FG) and a branch point. Dendron amplifiers may be expressed by the formula, (FG′)-T-(FGt)d, wherein FG′ and FGt are the focal point and terminal functional groups, respectively, which are selected from any suitable functional group; T is any suitable linker and “d” is any integer greater than 1, typically between 2 to 32, though, more preferably between 2 and 8, such as 2, 3, 4, 5, 6, 7, and 8. The multiple by which dendron amplifiers increase the terminal functional group (FGt) can be expressed as FGt=β^{γ}, wherein β is the number of branches that occur for each generation of the dendron and the symbol γ is the number of generations, wherein the number of branches is any integer, though, typically between 2 to 6, and the number of generations is any integer, though, typically between 1 to 10. Terminal functional groups present on solubilizing blocks that are free (i.e., unreacted), may also be referred to as solubilizing groups (SG).

Dendron amplifiers may comprise repeats of a monomer comprising a first functional group (FG1) and a second functional group (FG2), wherein the first functional group is reactive towards the second functional group. For instance, a non-limiting example of a 2^{nd} generation dendron amplifier with β=2 comprising repeats of a monomer comprising a first functional group (FG1) and a second functional group (FG2), wherein the first functional group is reactive towards the second functional group, is shown here for clarity.

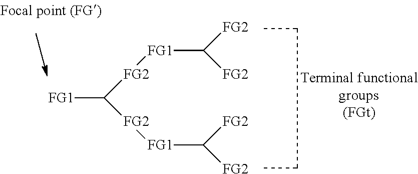

Wherein, the first functional group at the starting point is also referred to as the focal point functional group (FG′) and the terminal FG2 are referred to as the terminal functional groups or FGt.

A non-limiting example of a 3^{rd} generation dendron formed from monomers comprising a first and second functional group wherein β=2 is shown here for clarity:

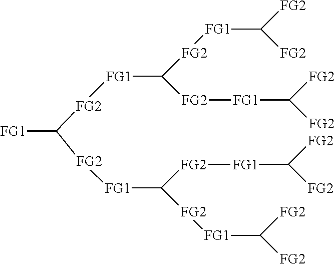

A non-limiting example of a 2^{nd} generation dendron amplifier with β=3 comprising repeats of a first monomer comprising a first functional group (FG1) and a second functional group (FG2), wherein the first functional group is reactive towards the second functional group, is shown here for clarity:

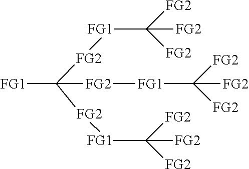

Monomers comprising a first functional group and a second functional group, wherein the first functional group is reactive towards the second functional group, and the monomer comprises at least one first functional group and two or more second functional groups may be selected from any suitable monomer. Non-limiting examples include FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, FG1-CH(R¹)₂, FG1-C(R¹)₃, wherein R¹ is independently selected from (CH₂)_{y3}-FG2, (OCH₂CH₂)_{y3}-FG2 or CH₂(OCH₂CH₂)_{y3}-FG2) and y2 and y3 are each an integer number of repeating units selected from between 1 to 6.

A non-limiting example of FG1-CH(R¹)₂, wherein FG1 is NH₂, R¹ is CH₂(OCH₂CH₂)_{y3}-FG2, y3 is 1 and FG2 is COOH is shown here for clarity:

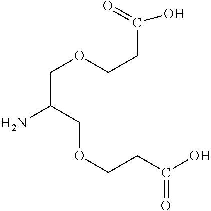 [SMILES: NC(COCCC(=O)O)COCCC(=O)O]

Wherein the above monomer is used to produce a 2^{nd} generation amplifying linker, the structure is:

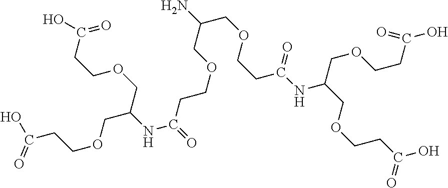 [SMILES: NC(COCCC(=O)NC(COCCC(=O)O)COCCC(=O)O)COCCC(=O)NC(COCCC(=O)O)COCCC(=O)O]

Additional non-limiting examples of monomers comprising a first functional group and a second functional group, wherein the first functional group is reactive towards the second functional group, and the monomer comprises at least one first functional group and two or more second functional groups include FG1-(CH₂)_{y2}N(R²)₂, FG1-(CH₂CH₂O)_{y2}CH₂CH₂N(R²)₂, wherein R² is independently selected from (CH₂)_{y3}-FG2, (CH₂CH₂O)_{y3}(CH₂)_{y4}-FG2, (CH₂OCH₂CH₂)_{y3}-FG2) and y2, y3 and y4 are each an integer of repeating units selected from between 1 to 6. Note: in the above example, FG′ is an amine and the 4 FGt are carboxylic acids.

A non-limiting example of FG1-(CH₂CH₂O)_{y1}CH₂CH₂N(R²)₂, wherein FG1 is NH₂, R² is (CH₂CH₂O)_{y3}(CH₂)_{y4}-FG2, y2 is 2, y3 is 1, y4 is 2 and FG2 is COOH is shown here for clarity:

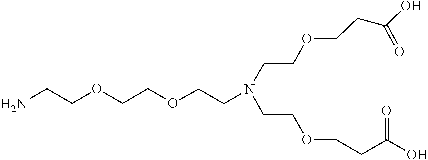 [SMILES: NCCOCCOCCN(CCOCCC(=O)O)CCOCCC(=O)O]

In still additional non-limiting examples of monomers comprising a first functional group and a second functional group, wherein the first functional group is reactive towards the second functional group, and the monomer comprises at least one first functional group and two or more second functional groups include certain amino acids, such as glutamic acid, aspartic acid, lysine or ornithine. A non-limiting example of a 3^{rd} generation lysine dendron is shown here for clarity:

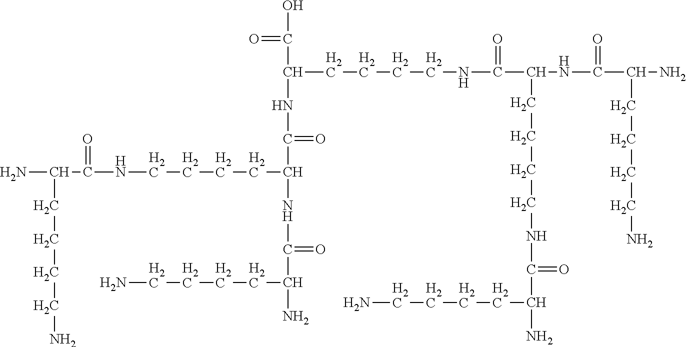 [SMILES: NCCCCC(N)C(=O)NCCCCC(NC(=O)C(N)CCCCN)C(=O)NCCCCC(NC(=O)C(CCCCNC(=O)C(N)CCCCN)NC(=O)C(N)CCCCN)C(=O)O]

Dendron amplifiers may comprise repeats of two monomers, wherein a first monomer comprises three or more first functional groups (FG1) and the second monomer comprises two or more second functional groups (FG2), wherein the first functional group is reactive towards the second functional group. For instance, a non-limiting example of a 2^{nd} generation dendron amplifier with β=2 comprising repeats of a first and second monomer, wherein the first monomer comprises three first functional groups (FG1) and the second monomer comprises two second functional groups (FG2), wherein the first functional group is reactive towards the second functional group, is shown here for clarity:

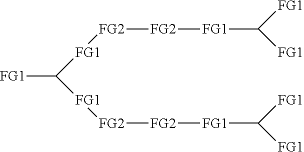

A non-limiting example of a 1^{st} generation dendron amplifier with β=2 comprising repeats of a first and second monomer, wherein the first monomer comprises three first functional groups (FG1) and the second monomer comprises three second functional groups (FG2), wherein the first functional group is reactive towards the second functional group, is shown here for clarity:

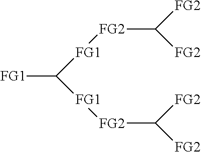

Dendron amplifiers may be used to join together any three or more components of amphiphiles, peptide antigen conjugates and drug molecule conjugates. The focal point functional group (FG′) and the terminal functional groups (FGt) may be further functionalized, i.e., reacted to fit a particular purpose.

### Extensions (E1 and E2)

The optional N- and C-terminal extensions (E1 and E2) denote moieties linked to the N- and C-terminus of the peptide antigen (A), respectively. The N- and C-terminal extensions E1 and E2 may comprise any one or more of the following: amino acids, including non-natural amino acids; hydrophilic ethylene oxide monomers (e.g., PEG); hydrophobic alkane chains; or the like; or combinations thereof. The N- and C-terminal extensions E1 and E2 are attached to the peptide antigen (A) through any suitable means, e.g., through amide bonds.

In some embodiments, the extensions (E1 and E2) function to control the rate of degradation of the peptide antigen (A) but may also perform any one or more additional functions. In some embodiments, the N- or C-terminal extension (E1 and E2) may be free (wherein one end of the N- or C-terminal extension is linked to the peptide antigen (A) and the other end is not linked to another molecule) and serve to slow degradation of the peptide antigen; for example, a E1 peptide-based extension may be linked to the N-terminus of the peptide antigen through an amide bond to slow degradation. In other embodiments, the N- and/or C-terminal extensions (E1 and/or E2) may be linked to a heterologous molecule and may function as a linker as well as to modulate peptide antigen (A) degradation. The N- and/or C-terminal extensions providing a linker function may link the peptide antigen either directly or indirectly through a Linker U to a hydrophobic block (H) and or solubilizing block (S). In some embodiments, the extensions (E1 and/or E2) function to provide distance, i.e., space, between any two heterologous molecules. In other embodiments, the extensions (E1 and/or E2) function to impart hydrophobic or hydrophilic properties to the peptide antigen conjugate. In still other embodiments, the composition of the extensions (E1 and/or E2) may be selected to impart rigidity or flexibility. In other embodiments, the N- and/or C-terminal extensions (E1 and/or E2) may help stabilize the particles formed by the peptide antigen conjugate.

In some embodiments, the extensions (E1 and/or E2) comprise charged functional groups, e.g., charged amino acid residues (e.g., arginine, ornithine, lysine, glutamic acid, aspartic acid, etc.), that impart charge at pH 7.4. The number of charged residues present in the extension can be used to modulate the net charge of the peptide antigen conjugate. Peptide-based extensions (E1 and/or E2) that are recognized by proteases and impart a particular electrostatic charge to stabilize particles formed by peptide antigen conjugates are described later.

Additionally, in some embodiments, C-terminal extensions (E2) added to peptide antigens (A) are selected to facilitate manufacturing of a peptides comprising the formula [S]-[E1]-A-E2-[U1], wherein [ ] denotes the group is optional. Accordingly, the amino acid sequence of peptide-based E2 can be selected to disrupt peptide 3-sheet formation and prevent sequence truncation during solid-phase peptide synthesis. In non-limiting examples, a C-terminal di-peptide linker (E2), Gly-Ser, is incorporated during solid-phase peptide synthesis as a pseudoproline dipeptide (e.g., Gly-Ser(Psi(Me,Me)pro)). In additional embodiments, a proline is included in E2, e.g., Ser-Pro-Leu-Arg (SEQ ID NO:4); whereby the proline is included to both facilitate manufacturing and promote processing of the extension by endosomal proteases.

In some embodiments, the peptide antigen (A) is linked at the C-terminus to an E2 extension that is linked either directly or indirectly through a Linker (U) to a hydrophobic block, e.g., wherein the peptide antigen conjugate has the structure A-E2-U-H or A-E2-H. In some embodiments, an E1 extension is linked to the N-terminus of the peptide antigen (A) and an E2 extension is linked at the C-terminus of the peptide antigen (A), wherein either E1 or E2 are linked either directly or via a Linker (U) to a hydrophobic block (H), e.g. wherein the peptide antigen conjugate has the structure E1-A-E2-U-H, H-U-E1-A-E2, E1-A-E2-H, or H-E1-A-E2. In other embodiments, a peptide antigen (A) is linked at the N-terminus to an E1 extension that is linked either directly or via a Linker (U) to a hydrophobic block (H), e.g., wherein the peptide antigen conjugate has the structure H-U-E1-A or H-E1-A. In some embodiments, a solubilizing block is linked to an extension, E1 or E2, that is linked to the N- or C-terminus of the peptide antigen (A), respectively, wherein the extension that is not linked to the solubilizing block (S) is linked either directly or via a Linker (U) to the hydrophobic block (H), e.g., wherein the peptide antigen conjugate has the structure S-E1-A-E2-U-H, H-U-E1-A-E2-S, E1-A-E2-H, H-E1-A-E2-S.

In additional embodiments, solubilizing blocks (S) are linked to both E1 and E2 extensions that are linked to both the N- and C-termini of the peptide antigen (A), respectively; or, solubilizing blocks (S) are linked to the E1 extension linked to the N-terminus of the peptide antigen (A) but not to the E2 extension attached to the C-terminus of the peptide antigen (A), which may be linked either directly or through a Linker (U) to a hydrophobic block (H). A linker precursor U1 or Linker (U) may be linked to either of the extensions (E1 or E2) through any suitable means, such as an amide bond.

In some embodiments, the extensions (Eland E2) are peptide sequences that are selected for recognition and hydrolysis by enzymes, such as proteases. The extensions (E1 and E2) are preferably cleavable peptides, including amino acids recognized by either or both endosomal proteases and/or the immunoproteasome.

In some embodiments, the N-terminal extension (E1) is a peptide sequence between about 1 to 8 amino acids in length, such as 1, 2, 3, 4, 5, 6, 7, or 8 amino acids, typically no more than 10 amino acids in length that is linked to the peptide antigen (A) through an amide bond formed between a carboxyl group of the E1 and the alpha amine of the N-terminal residue of the peptide antigen (A). The amide bond between E1 and the peptide antigen (A) may be cleaved by enzymes.

It is customary to number the amino acid positions in order of proximal to distal from the cleavage site, with amino acid positions C-terminal to the cleavage site indicated by the prime symbol (e.g., Pn′). For example, for a tetrapeptide extension (PN4-PN3-PN2-PN1) linked to the N-terminus of a peptide antigen (A) that is an octapeptide (PA1′-PA2′-PA3′-PA4′-PA5′-PA6′-PA7′-PA8′), e.g., PN4-PN3-PN2-PN1-PA1′-PA2′-PA3′-PA4′-PA5′-PA6′-PA7′-PA8′, the amide bond between PN1-PA1′ is recognized and hydrolyzed by an enzyme.

In some embodiments, the N-terminal extension (E1) is an enzyme degradable tetrapeptide that is recognized by endosomal proteases, wherein the PN1 position of a tetrapeptide extension (e.g., PN4-PN3-PN2-PN1) is preferably selected from arginine, lysine, citrulline, glutamine, threonine, leucine, norleucine, or methionine, for example, PN4-PN3-PN2-Arg; PN2 is selected from glycine, valine, leucine or isoleucine; PN3 is selected from glycine, serine, alanine, proline or leucine; and, PN4 is selected from glycine, serine, arginine, lysine, aspartic acid or glutamic acid. In some embodiments, the N-terminal extension (E1) is an enzyme degradable tripeptide that is recognized by endosomal proteases, wherein the PN1 position of a tripeptide extension (e.g., PN3-PN2-PN1) is preferably selected from arginine, lysine, citrulline, glutamine, threonine, leucine, norleucine, or methionine; PN2 is selected from glycine, valine, leucine or isoleucine; and PN3 is selected from glycine, serine, alanine, proline or leucine. In some embodiments, the N-terminal extension (E1) is an enzyme degradable di-peptide that is recognized by endosomal proteases, wherein the PN1 position of a dipeptide extension (e.g., PN2-PN1) is preferably selected from arginine, lysine, citrulline, glutamine, threonine, leucine, norleucine, or methionine; and PN2 is selected from glycine, valine, leucine or isoleucine. In still additional embodiments, the N-terminal extension (E1) is an amino acid that is recognized by endosomal proteases, wherein the PN1 position is preferably selected from arginine, lysine, citrulline, glutamine, threonine, leucine, norleucine, or methionine.

In other embodiments, the N-terminal extension (E1) is an enzyme degradable peptide that is recognized by the immunoproteasome, wherein the P1 position of a tetrapeptide extension (PN4-PN3-PN2-PN1) is preferably selected from isoleucine, leucine, norleucine or valine, for example, PN4-PN3-PN2-Leu.

In additional embodiments, the N-terminal extension (E1) is an enzyme degradable peptide that is recognized by both endosomal proteases and the immunoproteasome, wherein the PN5 and PN1 positions of an octapeptide extension (PN8-PN7-PN6-PN5-PN4-PN3-PN2-PN1) are selected from arginine, lysine, citrulline, glutamine, threonine, leucine, norleucine, or methionine for the PN5 position recognized by cathepsins, and isoleucine, leucine, norleucine or valine for the PN1 position recognized by the immuno-proteasome; for example, PN8-PN7-PN6-Arg-PN4-PN3-PN2-Leu. A non-limiting example of an N-terminal extension (E1) recognized by cathepsins and the immuno-proteasome is Lys-Pro-Leu-Arg-Tyr-Leu-Leu-Leu (SEQ ID NO:5).

Non-limiting examples of tetrapeptide N-terminal extensions (E1) that are recognized by the immunoproteasome include: Ser-Leu-Val-Cit (SEQ ID NO:6), Ser-Leu-Val-Leu (SEQ ID NO:7), Ser-Pro-Val-Cit (SEQ ID NO:8), Glu-Leu-Val-Arg (SEQ ID NO:9), Ser-Pro-Val-Arg (SEQ ID NO:10), Ser-Leu-Val-Arg (SEQ ID NO:11), Lys-Pro-Leu-Arg (SEQ ID NO:2), Lys-Pro-Val-Arg (SEQ ID NO:12), Glu-Leu-Val-Cit (SEQ ID NO:13), Glu-Leu-Val-Leu (SEQ ID NO:14), Glu-Pro-Val-Cit (SEQ ID NO:15), and Lys-Pro-Val-Cit (SEQ ID NO:16). Non-limiting examples of tripeptide N-terminal extensions (E1) include: Leu-Val-Cit, Leu-Val-Leu, Pro-Val-Cit, Leu-Val-Arg, Pro-Val-Arg, Pro-Leu-Arg, Gly-Val-Ser. Non-limiting examples of di-peptide N-terminal extensions (E1) include: Val-Cit, Val-Leu, Val-Arg, Leu-Arg. Non-limiting examples of single amino acid N-terminal extensions (E1) include Cit, Arg, Leu or Lys. In the above examples, Arg can be replaced with Lys; Lys can be replaced with Arg; Glu can be replaced with Asp; and Asp can be replaced with Glu. Note that Cit=citrulline.

In some embodiments, the E2 is a degradable peptide linked to the C-terminal residue of the peptide antigen (A) and comprises amino acid sequences that are recognized and hydrolyzed by certain proteases. In some embodiments, the C-terminal extension (E2) is a peptide sequence between about 1 to 8 amino acids in length, such as 1, 2, 3, 4, 5, 6, 7, or 8 amino acids, typically no more than 10 amino acids. In preferred embodiments, the C-terminal extension (E2) is linked to the peptide antigen (A) via an amide bond formed between the C-terminal carboxyl group of the peptide antigen (A) and the alpha amine of the N-terminal residue of the extension (E2). The amide bond between E2 and the peptide antigen (A) may be cleaved by enzymes. Note: that it is customary to number the amino acid positions in order of proximal to distal from the cleavage site, with amino acid positions C-terminal to the cleavage site indicated by the prime symbol (e.g., Pn′). For example, for a tetrapeptide extension (PC1′-PC2′-PC3′-PC4′) linked to the C-terminus of an octapeptide antigen (PA8-PA7-PA6-PA5-PA4-PA3-PA2-PA1), e.g., PA8-PA7-PA6-PA5-PA4-PA3-PA2-PA1-PC1′-PC2′-PC3′-PC4′, the amide bond between PA1-PC1′ is recognized and hydrolyzed by an enzyme.

In preferred embodiments of C-terminal extensions (E2), the C-terminal extension (E2) comprises amino acid sequences that are selected to promote immunoproteasome recognition and cleavage and optionally endosomal protease recognition. As peptide antigens (A) typically contain a C-terminal residue, for example, leucine, that promotes hydrolysis by the immunoproteasome, e.g., at the amide bond proximal to the C-terminal residue of the peptide antigen (A), extensions linked to the C-terminus of the peptide antigen (A) should be selected to promote immuno-proteasome recognition and cleavage at the amide bond proximal to the C-terminus of the peptide antigen (A). The immuno-proteasome favors small, non-charged amino acids at the PC1′ position adjacent to the C-terminal amino acid, PA1, of the peptide antigen (A), e.g., the amide bond between PA1-PC1′. However, endosomal proteases favor bulky hydrophobic amino acids (e.g., leucine, norleucine, methionine or glutamine) and basic amino acids (i.e., arginine and lysine). Therefore, C-terminal extensions may be selected to promote recognition by either or both classes of proteases.

In some embodiments, a peptide antigen (A) with the sequence PA8-PA7-PA6-PA5-PA4-PA3-PA2-PA1 is linked to a C-terminal peptide extension (E2) with the sequence PC1′ . . . PCn′, wherein n is an integer value from 1 to 8, for example, PA8-PA7-PA6-PA4-PA3-PA2-PA1-PC1′ . . . PCn′. The composition of the C-terminal extension (E2) depends on the length of the extension sequence used. In some embodiments, the C-terminal extension, E2, is a single amino acid PC1′ selected from Gly, Ala, Ser, Arg, Lys, Cit, Gln, Thr, Leu, Nle or Met. In additional embodiments, the C-terminal extension, E2, is a dipeptide, PC1′-PC2′, wherein PC1′ is selected from Gly, Ala or Ser; and PC2′ is selected from Gly, Ala, Ser, Pro, Arg, Lys, Cit, Gln, Thr, Leu, Nle, or Met. In additional embodiments, the C-terminal extension, E2, is a tripeptide, PC1′-PC2′-PC3′, wherein P1′ is selected from Gly, Ala, or Ser; PC2′ is selected from Gly, Ala, Ser, or Pro; and PC3′ is selected from Gly, Ser, Arg, Lys, Cit, Gln, Thr, Leu, Nle or Met.

In additional embodiments, the C-terminal extension, E2, is a tetrapeptide extension, PC1′-PC2′-PC3′-PC4′, wherein PC1′ is selected from glycine, alanine or serine; PC2′ is selected from glycine, alanine, serine, proline or leucine; PC3′ is selected from glycine, alanine, serine, valine, leucine or isoleucine; and PC4′ is selected from arginine, lysine, citrulline, glutamine, threonine, leucine, norleucine or methionine. In additional embodiments, the C-terminal extension, E2, is a pentapeptide, PC1′-PC2′-PC3′-PC4′-PC5′, wherein PC1′ is selected from glycine, alanine or serine; PC2′ is selected glycine, alanine, serine, proline, arginine, lysine, glutamic acid or aspartic acid; PC3′ is selected from glycine, alanine, serine, proline or leucine; PC4′ is selected from glycine, alanine, valine, leucine or isoleucine; and PC5′ is selected from arginine, lysine, citrulline, glutamine, threonine, leucine, norleucine or methionine. In additional embodiments, the C-terminal extension, E2, is a hexapeptide, PC1′-PC2′-PC3′-PC4′-PC5′-PC6′, wherein PC1′ is selected from glycine, alanine or serine; PC2′ is selected from glycine, alanine, serine or proline; PC3′ is selected from glycine, serine, proline, arginine, lysine, glutamic acid or aspartic acid; PC4′ is selected from proline or leucine; PC5′ is selected from glycine, alanine, valine, leucine or isoleucine; and PC6′ is selected from arginine, lysine, citrulline, glutamine, threonine, leucine, norleucine or methionine.

Non-limiting examples of hexapeptide C-terminal extensions (E2) include Gly-Gly-Lys-Leu-Val-Arg (SEQ ID NO:17), Gly-Gly-Lys-Pro-Leu-Arg (SEQ ID NO:18), Gly-Gly-Ser-Leu-Val-Arg (SEQ ID NO:19), Gly-Gly-Ser-Leu-Val-Cit (SEQ ID NO:20), Gly-Gly-Ser-Pro-Val-Cit (SEQ ID NO:21), Gly-Gly-Ser-Leu-Val-Leu (SEQ ID NO:22), Gly-Gly-Glu-Leu-Val-Arg (SEQ ID NO:23), Gly-Gly-Glu-Leu-Val-Leu (SEQ ID NO:24).

Non-limiting examples of pentapeptide C-terminal extensions (E2) include Gly-Ser-Leu-Val-Arg (SEQ ID NO:25), Gly-Ser-Leu-Val-Cit (SEQ ID NO:26), Gly-Lys-Pro-Val-Cit (SEQ ID NO:27), Gly-Lys-Pro-Val-Arg (SEQ ID NO:28), Gly-Ser-Leu-Val-Leu (SEQ ID NO:29), Gly-Glu-Leu-Val-Leu (SEQ ID NO:30).

Non-limiting examples of tetrapeptide C-terminal extensions (E2) include Ser-Leu-Val-Cit (SEQ ID NO:6), Ser-Leu-Val-Leu (SEQ ID NO:7), Ser-Pro-Val-Cit (SEQ ID NO:8), Glu-Leu-Val-Arg (SEQ ID NO:9), Ser-Pro-Val-Arg (SEQ ID NO:10), Ser-Leu-Val-Arg (SEQ ID NO:11), Lys-Pro-Leu-Arg (SEQ ID NO:2), Glu-Leu-Val-Cit (SEQ ID NO:13), Glu-Leu-Val-Leu (SEQ ID NO:14), Glu-Pro-Val-Cit (SEQ ID NO:15), Glu-Gly-Val-Cit (SEQ ID NO:31).

Non-limiting examples of tripeptide C-terminal extensions (E2) include Gly-Ser-Gly, Gly-Ser-Arg, Gly-Ser-Leu, Gly-Ser-Cit, Gly-Pro-Gly, Gly-Pro-Arg, Gly-Pro-Leu, Gly-Pro-Cit. Non-limiting examples of di-peptide C-terminal extensions (E2) include Gly-Ser, Gly-Pro, Val-Cit, Gly-Arg Gly-Cit. Non-limiting examples of single amino acid C-terminal extensions (E2) include Gly, Ser, Ala, Arg, Lys, Cit, Val, Leu, Met, Thr, Gln or Nle. In the above examples, Arg can be replaced with Lys; Lys can be replaced with Arg; Glu can be replaced with Asp; and Asp can be replaced with Glu.

The C-terminal extension (E2) linked to the C-terminus of the peptide antigen (A) may be selected for recognition (i.e., hydrolysis) by both the immunoproteasome and endosomal proteases. In non-limiting examples, a peptide antigen (A) with the sequence PA8-PA7-PA6-PA5-PA4-PA3-PA2-PA1 is linked at the C-terminus to a C-terminal tetrapeptide extension (E2) with the sequence PC1′-PC2′-PC3′-PC4′, wherein PC1′ is selected from glycine, alanine or serine and PC4′ is selected from arginine, lysine, citrulline, glutamine, threonine, leucine, norleucine, or methionine, for example, Ser-P3-P2-Arg. In some embodiments, an antigen with the sequence PA8-PA7-PA6-PA5-PA4-PA3-PA2-PA1 is linked at the C-terminus to a C-terminal hexapeptide extension (E2) with the sequence PC1′-PC2′-PC3′-PC4′-PC5′-PC6′, wherein PC1′ and PC2′ are selected from glycine, alanine, proline or serine and PC6′ is selected from arginine, lysine, citrulline, glutamine, threonine, leucine, norleucine, or methionine, for example, Gly-Gly-PC3′-PC4′-PC5′-Arg. A non-limiting example of a C-terminal extension (E2) that promotes processing by both the immuno-proteasome and cathepsins that is linked to the C-terminus of the peptide antigen (A) is Gly-Gly-Lys-Pro-Leu-Arg (SEQ ID NO:18). An additional non-limiting example of a C-terminal extension (E2) that is linked at the C-terminus of a peptide antigen (A) that favors processing by the immunoproteasome and cathepsins is Gly-Gly-Ser-Leu-Val-Cit (SEQ ID NO:20) or Gly-Gly-Ser-Pro-Val-Cit (SEQ ID NO:21).

### Linkers (L or U)

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof, which, for covalent linkers, further comprise two or more functional groups, which may be the same or different, that are used to link any two molecules, e.g., any two components of amphiphiles, peptide antigen conjugates and/or drug conjugates, though covalent bonds. Linkers herein may be denoted by the symbol U or L.

In some embodiments, a carbon linker can include a C1-C18 alkane linker, e.g., a lower alkyl linker, such as C1-C6 (i.e., from one to six methylene units), which can serve to increase the space between two or more molecules, i.e., different components, while longer chain alkane linkers can be used to impart hydrophobic characteristics. Alternatively, hydrophilic linkers, such as ethylene oxide linkers, may be used in place of alkane linkers to increase the space between any two or more heterologous molecules and increase water solubility. In other embodiments, the linker can be a cyclic and/or aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immunoproteasome).

For linking two components of amphiphiles, peptide antigen conjugates and drug conjugates, wherein at least one of the components comprises a peptide, it was found that linkers comprising between 2 and 7 methylene groups improved coupling of the two or components. In non-limiting examples, increasing the number of methylene units between the amide and the amine of the N-terminal amino acid of peptide-based hydrophobic blocks (H) led to improved coupling to other molecules, including U2, antigens (A), extension (E2), spacers (B) and solubilizing blocks (S). Therefore, in preferred embodiments, the N-terminal amino acid of poly(amino acid)-based hydrophobic blocks (H) comprises two or more, typically between 2 and 7, such as 1, 2, 3, 4, 5, 6, 7 methylene units. For clarity, an amino acid with 2 methylene units is beta-alanine and an amino acid with 5 methylene units is amino-hexanoic acid. In certain preferred embodiments, the N-terminal amino acid of peptide-based hydrophobic blocks (H) is amino-hexanoic acid (sometimes referred to as Ahx; CAS number 60-32-3). In other embodiments, the N-terminal amino acid of peptide-based hydrophobic blocks (H) is beta-alanine.

In some embodiments, the linker may comprise poly(ethylene oxide) (PEG). The length of the linker depends on the purpose of the linker. For example, the length of the linker, such as a PEG linker, can be increased to separate any two or more components, for example, to reduce steric hindrance, or in the case of a hydrophilic PEG linker can be used to improve water solubility. The linker, such as PEG, may be between about 1 and about 24 monomers in length, such as 1, 2, 3, 4, 5, 6, 7, 8, 9, 10, 11, 12, 13, 14, 15, 16, 17, 18, 19, 20, 21, 22, 23, 24 monomers in length or more. When used as a spacer (B), the PEG may be up to 45 monomers in length or more, though, typically between 4 and 36 monomers in length.

In some embodiments, wherein the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, wherein the linker comprises a carbon chain, the linker may comprise a chain of between about 12 and about 20 carbons. In some embodiments, wherein the linker comprises a carbon chain, the linker may comprise a chain of between no more than 18 carbons, typically between about 1 and 6 carbon atoms.

The linkage used to join any two or more molecules, e.g., any two or more components of amphiphiles, peptide antigen conjugates and/or drug conjugates may comprise any suitable functional group, including but not limited to amides, esters, ethers, thioethers, silyl ethers, disulfides, carbamates, carbamides, hydrazides, hydrazones, acetals and triazoles.

In non-limiting examples of a covalent linkage, a click chemistry reaction may result in a triazole that links, i.e., joins together, any two components of the amphiphile, peptide antigen conjugate, or drug molecule conjugate. In several embodiments, the click chemistry reaction is a strain-promoted [3+2]azide-alkyne cyclo-addition reaction. An alkyne group and an azide group may be provided on respective molecules to be linked by “click chemistry”. In some embodiments, an antigen (A) bearing an azide functional group is coupled to a hydrophobic block (H) having an appropriate reactive group, such as an alkyne, for example, a dibenzylcyclooctyne (DBCO).

In some embodiments, an amine is provided on one molecule and may be linked to another molecule by reacting the amine with any suitable electrophilic group such as carboxylic acids, acid chlorides, activated esters (for example, NHS ester), which results in an amide bond; the amine may be reacted with alkenes (via Michael addition); the amine may be reacted with aldehydes and ketones (via Schiff base); or, the amine may be reacted with activated carbonates or carbamates to yield a carbamate.

In some embodiments, the linker is cleavable under intracellular conditions, such that cleavage of the linker results in the release of any component linked to the linker, for example, a drug molecule (D).

For example, the linker can be cleavable by enzymes localized in intracellular vesicles (for example, within a lysosome or endosome or caveolae) or by enzymes, in the cytosol, such as the proteasome, or immunoproteasome. The linker can be, for example, a peptide linker that is cleaved by protease enzymes, including, but not limited to proteases that are localized in intracellular vesicles, such as cathepsins in the lysosomal or endosomal compartments of cells.

The peptide linker is typically between 1-10 amino acids, such as 1, 2, 3, 4, 5, 6, 7, 8, 9, 10, or more (such as up to 20) amino acids long, such as 11, 12, 13, 14, 15, 16, 17, 18, 19, 20 or more amino acids long. When used as a spacer (B), the peptide linker may be up to about 45 amino acids. Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly (SEQ ID NO:1) linker). Other examples of such linkers are described, for example, in U.S. Pat. No. 6,214,345, incorporated herein by reference. In certain such embodiments, the peptide linker cleavable by an intracellular protease is a Val-Cit linker or a Phe-Lys linker (see, for example, U.S. Pat. No. 6,214,345, which describes the synthesis of doxorubicin with the Val-Cit linker). Note: for examples of amino acids and peptides provided in throughout the specification (either within the text of figures), unless otherwise specified, it should be understood that the peptides and amino acids are L-amino acids.

The cleavable peptide linker can be selected to promote processing (i.e., hydrolysis) of the peptide linker following intracellular uptake by immune cells. The sequence of the cleavable peptide linker can be selected to promote processing by intracellular proteases, such as cathepsins in intracellular vesicles or the proteasome or immunoproteasome in the cytosolic space.

In several embodiments, linkers comprising peptide sequences of the formula Pn . . . P4-P3-P2-P1 are used to promote recognition by cathepsins, wherein P1 is selected from arginine, lysine, acetyl lysine (i.e., the epsilon amine is acetylated), boc protected lysine (i.e., the epsilon amine is boc protected), citrulline, glutamine, threonine, leucine, norleucine, alpha-aminobutyric acid (abbreviated as “a-But” herein) or methionine; P2 is selected from glycine, serine, leucine, valine or isoleucine; P3 is selected from glycine, serine, alanine, proline, or leucine; and P4 is selected from glycine, serine, arginine, lysine, acetyl lysine (i.e., the epsilon amine is acetylated), boc protected lysine, aspartic acid, glutamic acid or beta-alanine. In non-limiting examples a tetrapeptide linker of the formula P4-P3-P2-P1 linked through an amide bond to another molecule and has the sequence Lys-Pro-Leu-Arg (SEQ ID NO:2). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprising peptide sequences are selected to promote recognition by the proteasome or immunoproteasome. Peptide sequences of the formula Pn . . . P4-P3-P2-P1 are selected to promote recognition by proteasome or immunoproteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In non-limiting examples, a cleavable linker of the formula P4-P3-P2-P1 that is recognized by the proteasome is linked through an amide bond at P1 to another molecule and has the sequence Tyr-Leu-Leu-Leu (SEQ ID NO:3). Sequences that promote degradation by the proteasome or immunoproteasome may be used alone or in combination with cathepsin cleavable linkers. In some embodiments, amino acids that promote immunoproteasome processing are linked to linkers that promote processing by endosomal proteases. A number of suitable sequences to promote cleavage by the immunoproteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In certain embodiments, drug molecules (D) are linked to hydrophobic blocks (H) via linker X1 comprising an enzyme degradable peptide. A non-limiting example is shown here:

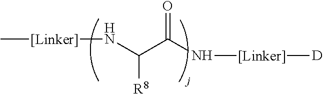

wherein D is a drug molecule; “Linker” is any suitable linker molecule; j denotes any integer, though, j is typically 1 to 6 amino acids, such as 1, 2, 3, 4, 5 or 6 amino acids; R⁸ is any suitable amino acid side group; the N-terminal amine of the peptide is linked either directly or via the ends, e.g., to the N- or C-termini of a hydrophobic block (H) comprising poly(amino acids), either directly or via U, or through reactive monomers comprising the hydrophobic block (H); and, brackets “[ ]” denote that the group is optional.

In certain preferred embodiments of drug molecules linked to hydrophobic blocks (H) via linker X1 comprising an enzyme degradable peptide, the drug molecule (D) is linked directly to the peptide through an amide bond as shown here:

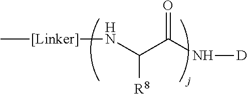

In non-limiting examples of the above structure, wherein the N-terminal Linker group is present and selected from beta alanine the structure is:

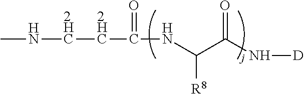

In some embodiments, the drug molecule (D) is linked to the peptide via a self-immolative carbamate linker. A non-limiting example is shown here:

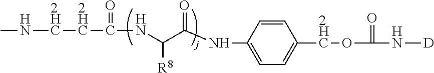

In the above example, wherein j is 4 and the amino acids are Serine-Lysine(Ac)-Valine-nor-Leucine, the structure is:

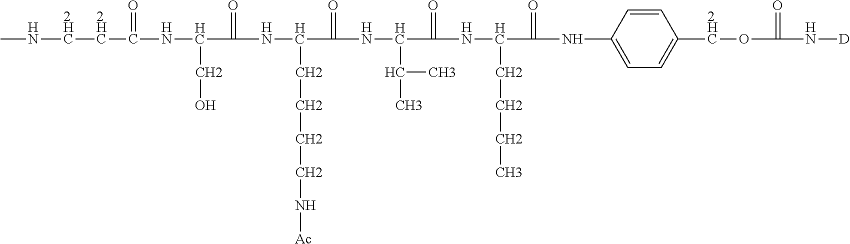

In some embodiments, drug molecules (D) are linked to hydrophobic blocks (H) through a sulfatase degradable linker X1, wherein hydrolysis of a sulfate by sulfatase results in release of the drug molecule from the linker. A number of arylsulfatase and alkysulfatase degradable linkers have recently been described (e.g., see: Bargh, et al., 2020, Chem. Sci. 11, 2375). In some embodiments of the present disclosure, drug molecules are linked to hydrophobic blocks (H) through sulfatase degradable linkers. Non-limiting examples are shown here for clarity:

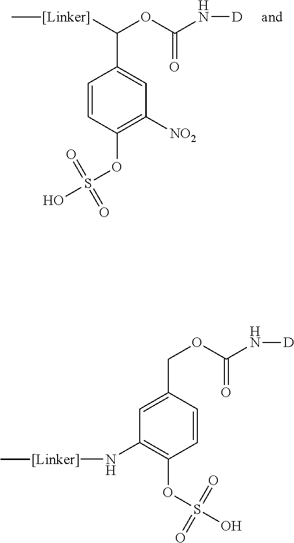

wherein D is a drug molecule; “Linker” is any suitable linker molecule linked either directly or via ends, e.g., to the N- or C-termini of a hydrophobic block (H) comprising poly(amino acids), either directly or via U, or through reactive monomers comprising the hydrophobic block (H); and, brackets “[ ]” denote that the group is optional.

Non-limiting examples of the above structures, wherein the “Linker” is present and selected from short alkyl linkers linked to the hydrophobic block through an amide are shown here for clarity:

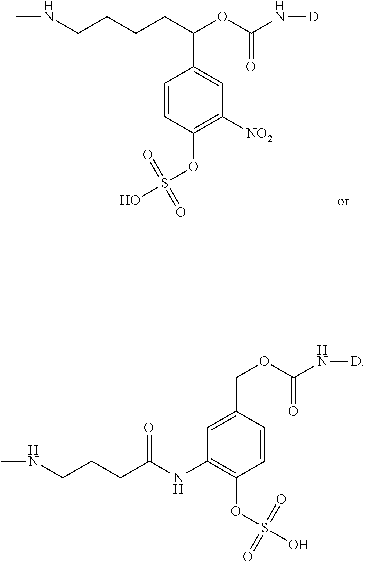

In other embodiments, any two or more components may be joined together through a pH-sensitive linker X that is sensitive to hydrolysis under acidic conditions. A number of pH-sensitive linkers are familiar to those skilled in the art and include for example, a hydrazone, carbohydrazone, semicarbazone, thiosemicarbazone, cis-aconitic amide, orthoester, acetal, ketal, silylether or the like (see, for example, U.S. Pat. Nos. 5,122,368; 5,824,805; 5,622,929; Dubowchik and Walker, 1999, Pharm. Therapeutics 83:67-123; Neville et al., 1989, Biol.

### Chem. 264:14653-14661).

In certain embodiments, different components (e.g., drug molecule and hydrophobic block (H)) are linked together through pH-sensitive linkers that are stable at blood pH, e.g., at a pH of about 7.4, but undergo more rapid hydrolysis at endosomal/lysosomal pH, ˜pH 5-6.5. In certain, preferred embodiments, drug molecules (D) are linked to hydrophobic blocks (H) through reactive monomers via a pH-sensitive bonds, such as hydrazone bonds that result from the reaction between a ketone and a hydrazine. The functional group hydrazine linked to a carbonyl is sometimes referred to as hydrazide, though, hydrazine is meant to broadly refer to —NH—NH₂ groups, including when linked to carbonyl, e.g., C(O)—NH—NH₂. pH-sensitive linkages, such as a hydrazone, provide the advantage that the bond is stable at physiologic pH, at about pH 7.4, but is hydrolyzed at lower pH values, such as the pH of intracellular vesicles.

In certain preferred embodiments, drug molecules are linked by a linker X1 comprising a ketone and may be represented by the formula:

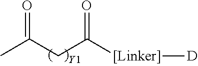

wherein D is any drug molecule; “Linker” is any suitable linker molecule; y1 denotes an integer between 1 to 6, preferably 4; brackets “[ ]” denote that the group is optional; and, wherein the ketone in the above example is used to link the linker linked drug molecule (D) to a reactive monomer through a hydrazone bond.

In the above example, wherein y1 is 4 and the drug molecule is linked directly (i.e., the “Linker” is absent) via an amide bond, the structure is:

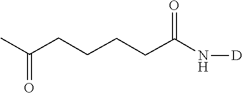

In preferred embodiments, drug molecules linked to ketones are linked to hydrophobic blocks (H) through hydrazone or carbohydrazone bonds. Non-limiting examples of drug molecules linked to a glutamic acid-based reactive monomer (N) through hydrazone and carbohydrazone bonds are shown here:

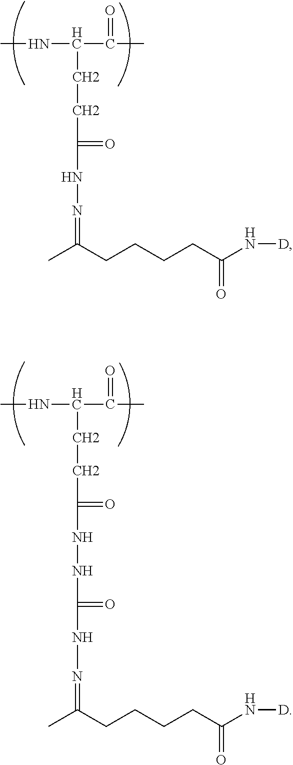

In some embodiments, the drug molecule comprises a ketone and may be linked directly to reactive monomers through hydrazone or carbohydrazone.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935).

In some embodiments, the linker X1 linking a hydrophobic block (H) and one or more drug molecules (D) is a short alkyl or PEG linker. In other preferred embodiments, the linker X1 linking a hydrophobic block (H) and one or more drug molecules (D) is an enzyme degradable linker, such as a cathepsin degradable peptide or sulfatase degradable linker. In other preferred embodiments, the linker X1 linking a hydrophobic block (H) and one or more drug molecules (D) comprises an enzyme degradable peptide and a self-immolative linker.

X can be any suitable linker, though, in preferred embodiments, the linker X linking any two or more groups, is a short alkyl (i.e., lower alkyl) or PEG linker, e.g., a PEG linker with between about 1 to about 24 monomeric units.

In some embodiments, Linker Precursors are used to form a Linker U and are selected for site-selectivity, i.e., a reaction only takes place between U1 and U2 and between no other groups. In some embodiments, Linker Precursor U1 comprises an activated carboxylic acid and is reacted with a Linker Precursor U2 that comprises an amine to form Linker U comprising an amide; or, U1 comprises an amine and is reacted with U2 that comprises an activated carboxylic acid to form Linker U comprising an amide. In some embodiments, Linker Precursor U1 comprises a maleimide and is reacted with Linker Precursor U2 that comprises a thiol to form a Linker U comprising a thioether bond; or, U1 comprises a thiol and is reacted with U2 that comprises a maleimide to form a Linker U comprising a thioether bond. In some embodiments, Linker Precursor U1 comprises an azide and is reacted with Linker Precursor U2 that comprises an alkyne to form a Linker U that comprises a triazole; or, U1 comprises an alkyne and is reacted with a U2 that comprises an azide to form a Linker US comprising a triazole.

In some embodiments, the Linker U preferably comprises an amide, thioether or triazole. In some embodiments, Linker Precursor U1 comprises a strained alkyne (e.g., dibenzocyclooctyne (DBCO), bicyclononyne (BCN) or the like) that is reacted with Linker Precursor U2 which comprises an azide to form the Linker U which comprises a triazole. In some embodiments, Linker Precursor U1 comprises an azide that is reacted with the Linker Precursor U2 that comprises a strained alkyne (e.g., dibenzocyclooctyne (DBCO), bicyclononyne (BCN) or the like) to form the Linker U which comprises a triazole. In non-limiting examples, the Linker Precursor U2 comprising DBCO is linked to the hydrophobic block (H) via a suitable linker X (e.g., DBCO-NHS, CAS number 1353016-71-3) and the Linker Precursor U1 (e.g. azido acid, such as azidopentanoic acid; azido amino acid, such as azido-lysine (abbreviated Lys(N3), CAS number 159610-92-1; or, azido amine, such as azido-butylamine) is linked to the solubilizing block fragment or peptide antigen fragment via a suitable linker X.

### Hydrophobic Molecule or Hydrophobic Block (H)

The hydrophobic molecule or hydrophobic block (sometimes designated “H” in formulae) is a molecule with substantially limited water solubility, or is amphiphilic in properties, and capable of assembling into supramolecular structures, e.g., micellar, nano- or micro-particles in aqueous solutions. In certain embodiments, the hydrophobic block (H) is insoluble, or forms micelles, in aqueous solutions at concentrations of about 1.0 mg/mL or less, e.g., about 0.1 mg/mL or about 0.01 mg/mL. In some embodiments, the hydrophobic block is soluble in aqueous solutions at certain concentrations, temperatures and/or pH ranges but becomes insoluble in response to a change in concentration, temperature and/or pH. For instance, in some embodiments, the hydrophobic block is a hydrophobic polymer that is temperature-responsive, i.e., the hydrophobic polymer is soluble in aqueous solutions at temperatures below a transition temperature (Tₜᵣ) but becomes insoluble at temperatures above the transition temperature. Preferred hydrophobic blocks (H) are molecules that have a solubility of at least less than about 1.0 mg/mL, such as less than about 0.1 mg/mL or less than about 0.01 mg/mL, at or near physiologic pH (˜pH 7.4), between about pH 6.5 to pH 8.5 or between about pH 6.0 and pH 9.0, and at or near physiologic temperature (˜37° C.) and physiologic salt concentrations (˜10 g/L) and salt composition.

The hydrophobic block (H) may be chosen from any molecule comprising higher alkanes, cyclic aromatics, fatty acids, compounds deriving from terpenes/isoprenes, or polymers or oligomers that have limited water solubility and/or amphiphilic characteristics.

Exemplary higher alkanes include but are not limited to octane, nonane, decane, undecane, dodecane, tridecane, tetradecane, pentadecane, hexadecane, heptadecane and octadecane. Exemplary cyclic aromatics include but are not limited to phenyl. Exemplary saturated and unsaturated fatty acids include but are not limited to myristic acid, palmitic acid, stearic acid or oleic acid. In some embodiments, the hydrophobic block (H) is a fatty acid, for example myristic acid. In other embodiments, the hydrophobic block (H) comprises a diacyl lipid, such as 1,2-dioleoyl-sn-glycero-3-phosphoethanolamine or 1,2-distearoyl-sn-glycero-3-phosphoethanolamine or a lipopeptide, e.g., Pam2Cys. In some embodiments, the fatty acid or lipid based hydrophobic block (H) may further comprise a PEG. Exemplary compounds deriving from terpenes/isoprene include sterol derivatives, such as cholesterol, and squalene. In some embodiments, the hydrophobic block (H) comprises cholesterol. In some embodiments, the hydrophobic block (H) comprises a saponin, e.g., QS-21.

In some embodiments the hydrophobic block (H) is a linear, branched or brush polymer (or oligomer). The hydrophobic block (H) can be a homopolymer or copolymer. The hydrophobic block (H) can comprise one or many different types of monomer units. The hydrophobic block (H) can be a statistical copolymer or alternating copolymer. The hydrophobic block (H) can be a block copolymer, such as the A-B type, or the polymer can comprise a grafted copolymer, whereby two or more polymers are linked through polymer analogous reaction.

The hydrophobic block (H) may comprise polymers comprising naturally occurring and/or non-natural monomers and combinations thereof.

In some embodiments, the hydrophobic block (H) is selected from natural biopolymers. Natural biopolymers may include peptides (sometimes referred to as poly(amino acids)) which comprise hydrophobic amino acids. Non-limiting examples of hydrophobic amino acids include leucine, isoleucine, norleucine, valine, tryptophan, phenylamine, tyrosine and methionine, as well as hydrophilic amino acids that have been modified, such as by acetylation or benzoylation to have hydrophobic characteristics. Natural biopolymers that are water soluble in their native form may be used but must be modified chemically to make such natural biopolymers water insoluble and suitable for use as hydrophobic block (H). For example, biopolymers which comprise of hydrophilic amino acids, such as glutamic acid or lysine residues may be modified at the gamma carboxyl or epsilon amine groups, respectively, for the attachment of a hydrophobic molecule, such as a hydrophobic drug molecule, to increase the hydrophobicity of the resulting modified biopolymer. Similarly, biopolymers can be selected from hydrophilic polysaccharides, which may include but are not limited to glycogen, cellulose, dextran, alginate and chitosan, but such polysaccharides should be modified chemically, for example via acetylation or benzoylation of hydrophilic functional groups to render the resulting modified polysaccharide water insoluble. In still further embodiments the hydrophobic block comprises monomers selected from lactic acid and/or glycolic acid.

Monomers comprising the hydrophobic block (H) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylates and (meth)acrylamides include benzyl methacrylamide (BnMAM) and benzyl methacrylate (BnMA), respectively.

Certain monomers described herein as hydrophobic monomers may be water soluble under certain conditions but are hydrophobic and water insoluble at certain conditions in aqueous solutions. Non-limiting examples include temperature-responsive monomers, such as N-isopropylmethacrylamide (NIPMAM); a homopolymer comprising entirely of NIPMAM may be water soluble at room temperature but may become insoluble and form particles at elevated temperatures. Such distinctions are made to facilitate description of certain embodiments. In some embodiments, the hydrophobic block comprises a majority of monomer units selected from hydrophobic monomers that are temperature-responsive (sometimes referred to as “temperature-responsive monomers”), such as NIPAM, NIPMAM, N,N′-diethylacrylamide (DEAAM), N-(L)-(1-hydroxymethyl)propyl methacrylamide (HMPMAM), N,N′-dimethylaminoethylmethacrylate (DMEMA), N—(N-ethylcarbamido)propylmethacrylamide, N-vinylisobutyramide (PNVIBA), N-vinyl-n-butyramide (PNVBA), N-acryloyl-N-propylpiperazine (PNANPP), N-vinylcaprolactam (PVCa), DEGMA, TEGMA, or poly(amino acids) or γ-(2-methoxyethoxy)esteryl-L-glutamate. In still other embodiments, the hydrophobic block (H) may comprise monomers of ethylene oxide, propylene oxide or combinations thereof

Hydrophobic blocks (H) comprising a polymer typically comprise hydrophobic monomers and one or more other types of monomers, such as reactive monomers optionally linked to a drug molecule, spacer monomers and/or charged monomers. In some embodiments of hydrophobic blocks (H) comprising a polymer (or oligomer), a majority of monomer units are selected from hydrophobic monomers. In other embodiments of hydrophobic blocks (H) comprising a polymer (or oligomer), a majority of monomer units are selected from reactive monomers linked to hydrophobic drug molecules. In still other embodiments of hydrophobic blocks (H) comprising a polymer (or oligomer), the polymer comprises hydrophobic monomers and reactive monomers linked to hydrophobic drug molecules. In still further embodiments of hydrophobic blocks (H) comprising a polymer (or oligomer), the polymer comprises hydrophobic monomers and charged monomers and optionally reactive monomers linked to hydrophobic drug molecules.

In preferred embodiments, the hydrophobic block (H) comprises a polymer (or oligomer) that comprises hydrophobic monomers that further comprise aryl groups. In certain embodiments, the hydrophobic block (H) comprises heteroaryl groups. In still other embodiments, the aryl or heteroaryl groups of the hydrophobic block (H) comprise an amino substituent. The present inventors found that hydrophobic blocks (H) comprising aminoaryl or aminoheteroaryl groups lead to improved manufacturability and solubility in water-miscible solvents. The present inventors also found that amphiphiles with hydrophobic blocks (H) comprising aromatic amines lead to formation of stable particles with low CMC.

In preferred embodiments, the hydrophobic block (H) comprises monomers that comprise aryl or heteroaryl groups. Exemplary aryl groups (sometimes referred to as “aromatics” or “aromatic rings”) include but are not limited to phenyl, naphthyl, and quinolinyl. Non-limiting examples include:

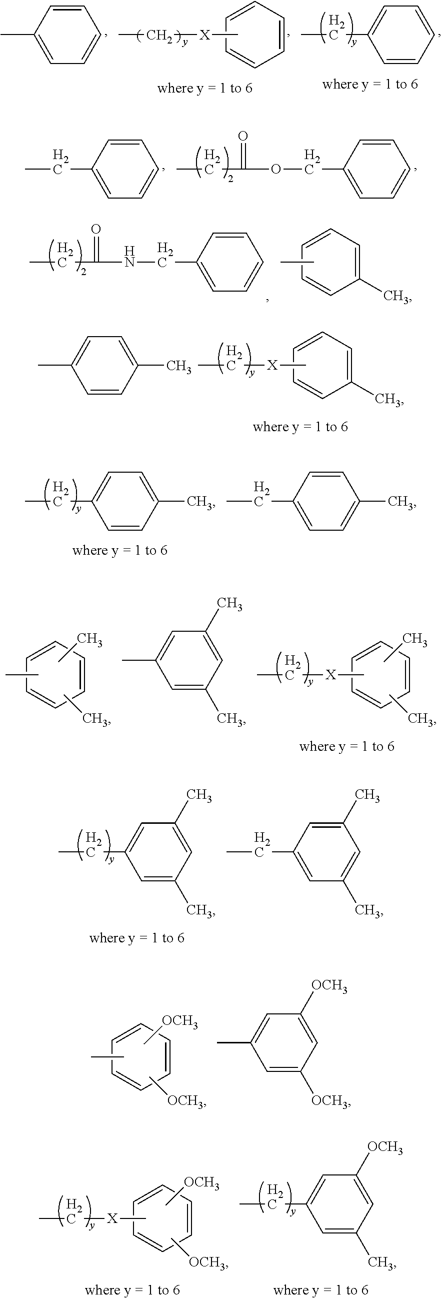

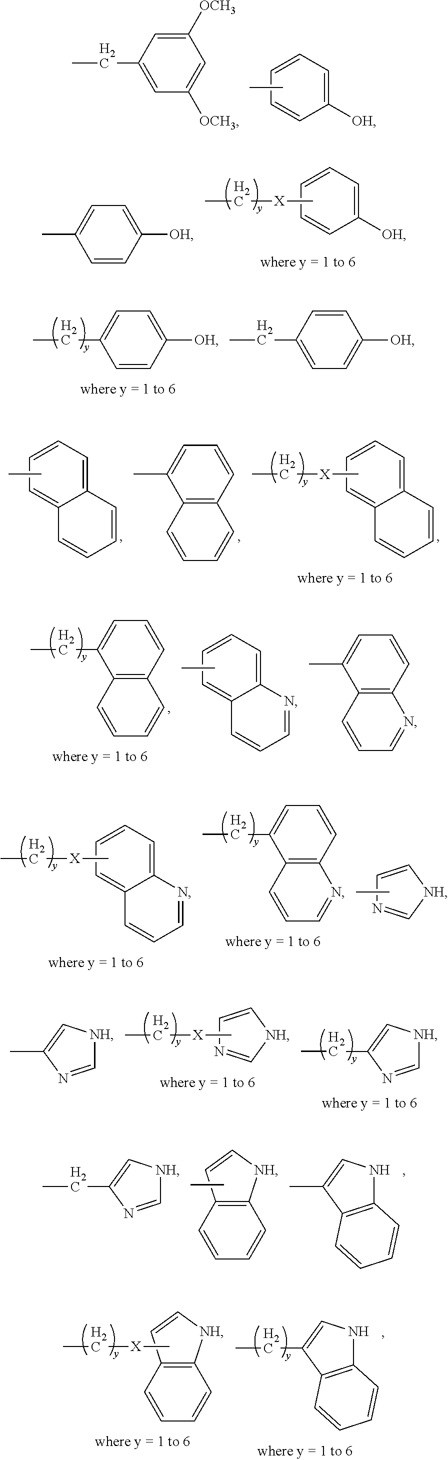

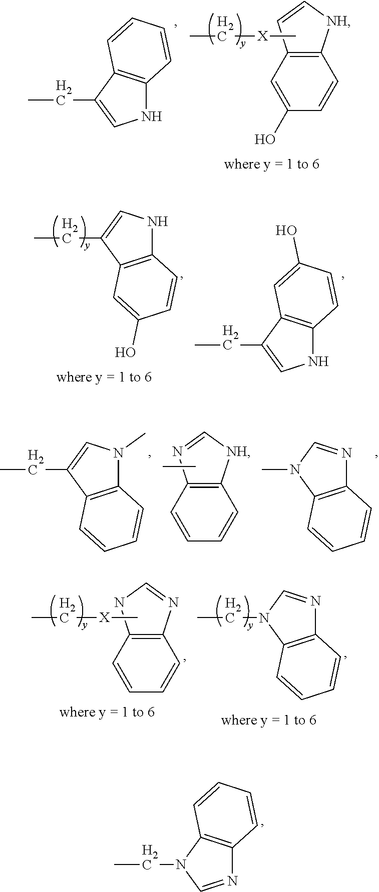

wherein X is any suitable linker molecule and y is an integer value, typically between 1 and 6.

In preferred embodiments, aryl or heteroaryl groups include but are not limited to

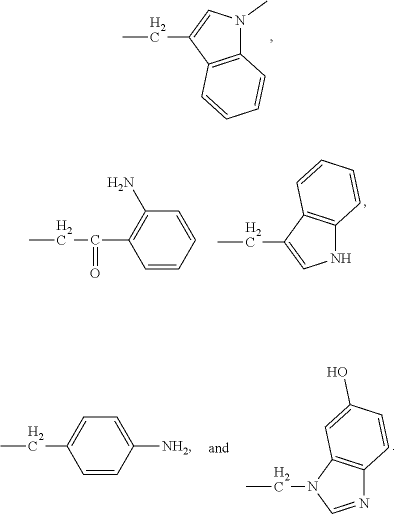

Furthermore, in the aforementioned aryl or heteroaryl groups one or more hydrogen atoms may be substituted for one or more fluorine atoms. In certain embodiments, the hydrophobic block comprises fluorinated aliphatic, aryl or heteroaryl groups, wherein one or more hydrogen atoms of the aforementioned groups comprising the hydrophobic monomer may be substituted for one or more fluorine atoms. The following non-limiting examples of fluorinated aryl groups may be present in hydrophobic monomers:

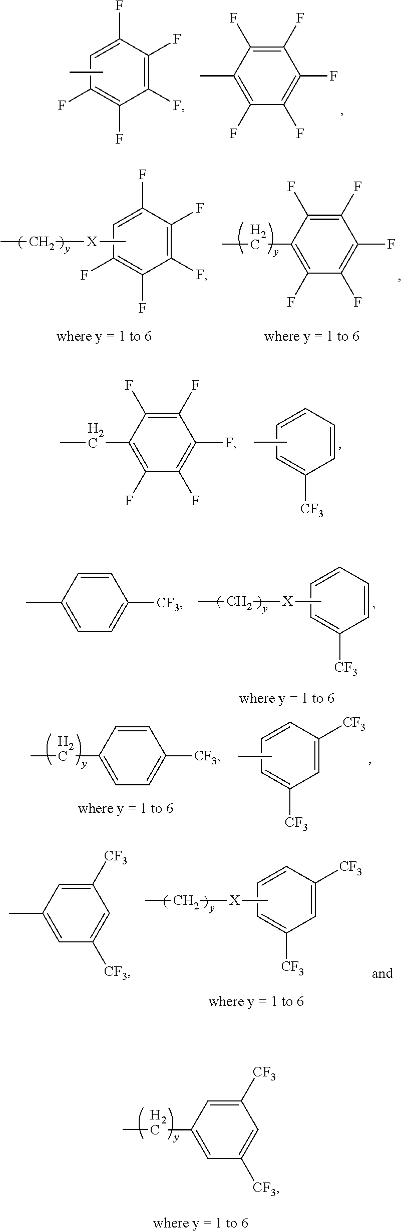

wherein X is any suitable linker molecule and y is an integer value, typically between 1 and 6.

The present inventors have unexpectedly found that hydrophobic blocks (H) comprising aminoaryl or aminoheteroaryl groups lead to improved manufacturing and solubility in polar aprotic solvents and alcohols. Therefore, in certain preferred embodiments, the hydrophobic block (H) comprises moieties of the formula —Ar—NHR, where Ar can be an aryl or heteroaryl, and R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Non-limiting examples of aminoaryl or aminoheteroaryl groups include but are not limited to:

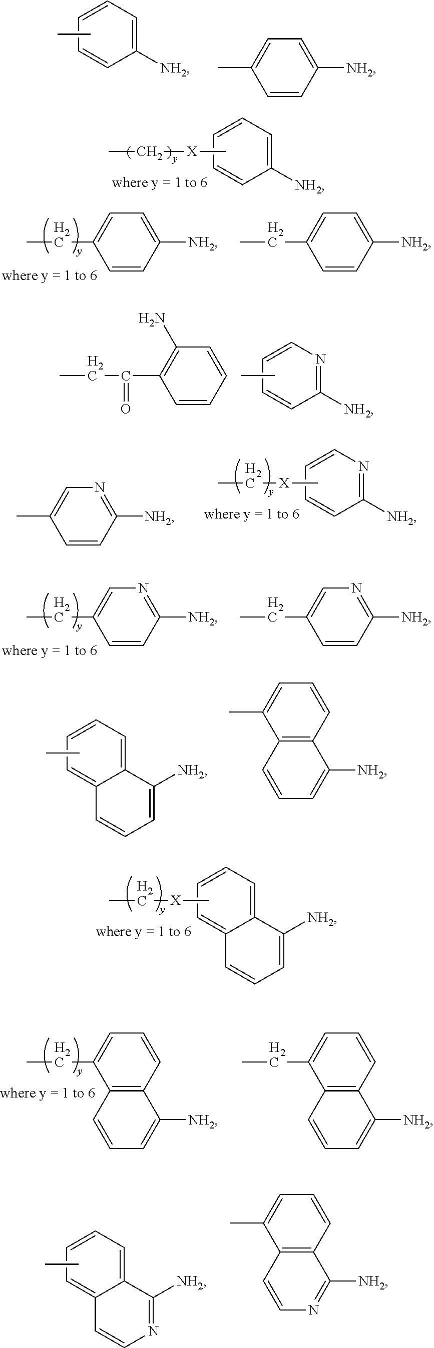

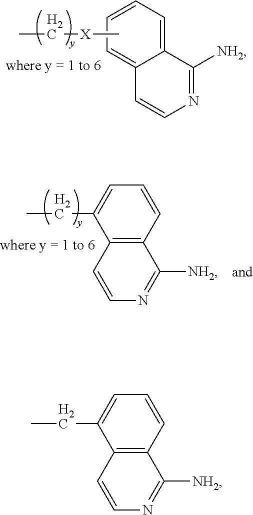

wherein X is any suitable linker molecule and y is an integer value, typically between 1 and 6.

In some embodiments, the hydrophobic block (H) comprises polymers (or oligomers) that further comprise hydrophobic monomers with fused aryl groups (e.g., naphthyl) or fused heteroaryl groups (e.g., xanthenyl or quinolinyl). In some embodiments, the hydrophobic block (H) comprises reactive monomers linked to hydrophobic drug molecules. In some embodiments, the hydrophobic drug molecules (e.g., imidazoquinolines) are aromatic and thus the reactive monomers linked to hydrophobic drug molecules comprising aromatic groups may also be described as hydrophobic monomers comprising aromatic groups or reactive monomers linked to drugs.

In some embodiments, the hydrophobic block (H) comprises a poly(amino acid) of Formula I.

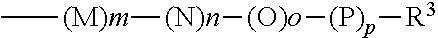

wherein the poly(amino acid) of Formula I comprises monomers selected from hydrophobic amino acids (M), reactive amino acids (N), spacer amino acids (o), charged amino acids (P) and combinations thereof provided that at least monomer M or N are present; m, n, o and p denote that there are an integer of repeat units of monomers M, N, o and P, respectively, which may be distributed along the polymer in a specific or random order; and R₃ is typically selected from hydrogen, NH₂, NH₂—CH₃, NH₂—(CH₂)₅CH₃, OH, or drug molecules (D) either linked directly or through X1.

In some embodiments, P is absent. In other embodiments, N, O, and P are each absent.

In some embodiments, P is

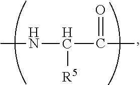

wherein each R⁵, independently, is a group that comprises 1 to 2 charged functional groups.

In some embodiments, O is

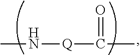

wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4.

In some embodiments, N is

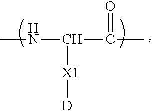

wherein each X1, independently, is a suitable linker; and each D, independently, is a drug molecule.

In some embodiments, M is

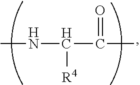

wherein each R⁴ is, independently, a hydrophobic group.

In some embodiments, the hydrophobic block (H) comprises a poly(amino acid) of Formula I.

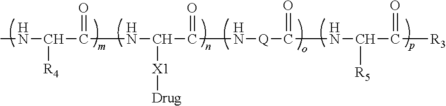

wherein the poly(amino acid) of Formula I comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; R³ is typically selected from hydrogen, NH₂, NH₂—CH₃, NH₂—(CH₂)_{y5}CH₃, OH, or drug molecules (D) either linked directly or through X1; R⁴ is any hydrophobic group typically selected from aryl or heteroaryl groups; R⁵ is any group that comprises one or more functional groups that are charged in aqueous solutions or are pH-responsive and charged in aqueous solutions at certain pH ranges; Q is typically selected from any lower alkyl or heteroalkyl including but not limited to (CH₂)_{y6} and (CH₂CH₂O)_{y7}CH₂CH₂, where y6 is any integer from 1 to 6 and y7 is an integer typically selected from 1 to 4; and, the N-terminus is linked to either (i) a solubilizing block (S) directly or indirectly via a spacer (B) and/or a Linker U; (ii) a peptide antigen (A) either directly or indirectly via an extension (E1 or E2) and/or Linker U; or (iii) a drug molecule either directly or via X1. Note: hydrophobic amino acids, reactive amino acids, spacer amino acids and charged amino acids are sometimes described more generally as hydrophobic monomers, reactive monomers, spacer monomers and charged monomers, respectively.

In some embodiments of poly(amino acids) of Formula I, R⁴ is

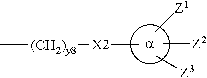

wherein,
α is aryl or heteroaryl;
X2 is present or absent and when present is a suitable linker;
y8 is selected from an integer from 0 and 6; and
Z¹, Z², and Z³ are each independently selected from H, F, hydroxy, amino, alkyl, and fluoroalkyl.

In preferred embodiments of poly(amino acids) of Formula I, a is aryl, e.g., phenyl or naphthyl. In other embodiments, a is heteroaryl, e.g., imidazolyl, pyridinyl, quinolinyl, isoquinolinyl, indolyl, and benzimidazolyl.

In preferred embodiments of poly(amino acids) of Formula I, X2 is absent. In other embodiments, X2 is present and is selected from C(O), CO₂(CH₂)_{y9}, and C(O)NH(CH₂)_{y9}, NHC(O) and NHC(O)(CH₂)_{y9}, wherein y9 is an integer typically selected from 1 to 6. In other embodiments, X2 is present and is selected from lower alkyl and PEG groups.

In preferred embodiments of poly(amino acids) of Formula I, the poly(amino acid) of Formula I comprises hydrophobic amino acids, M, selected from any natural or non-natural amino acid that comprises a hydrophobic group, R⁴. In preferred embodiments, R⁴ is selected from hydrophobic groups comprising aryl groups, heteroaryl groups, aminoaryl, and/or aminoheteroaryl. Non-limiting examples of R⁴ include but are not limited to:

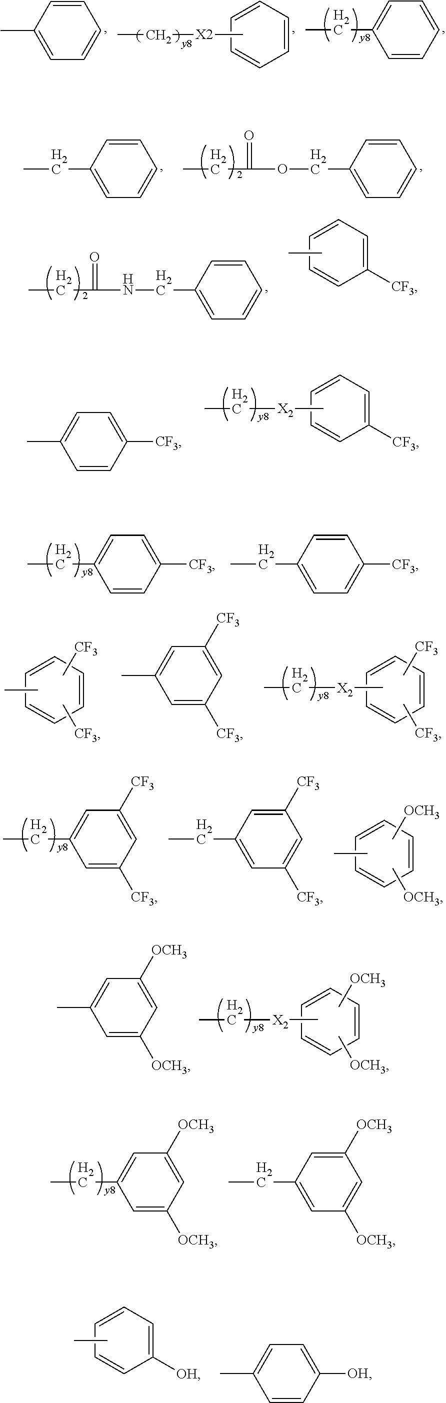

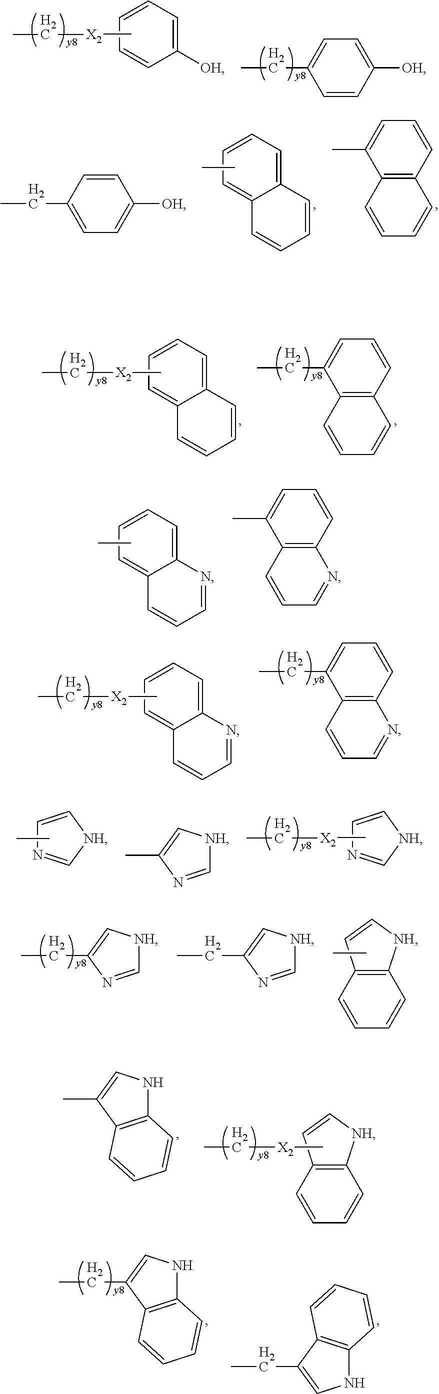

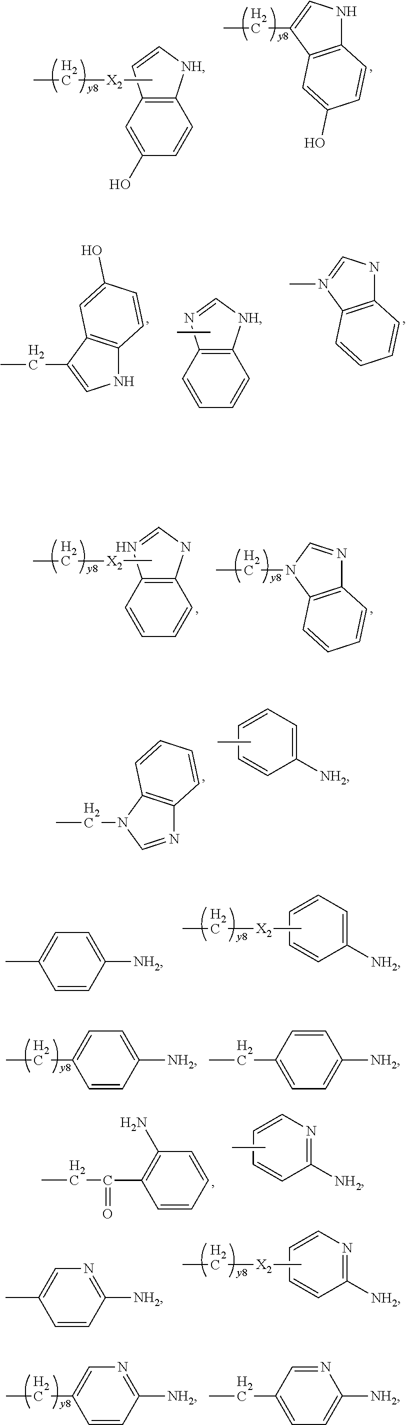

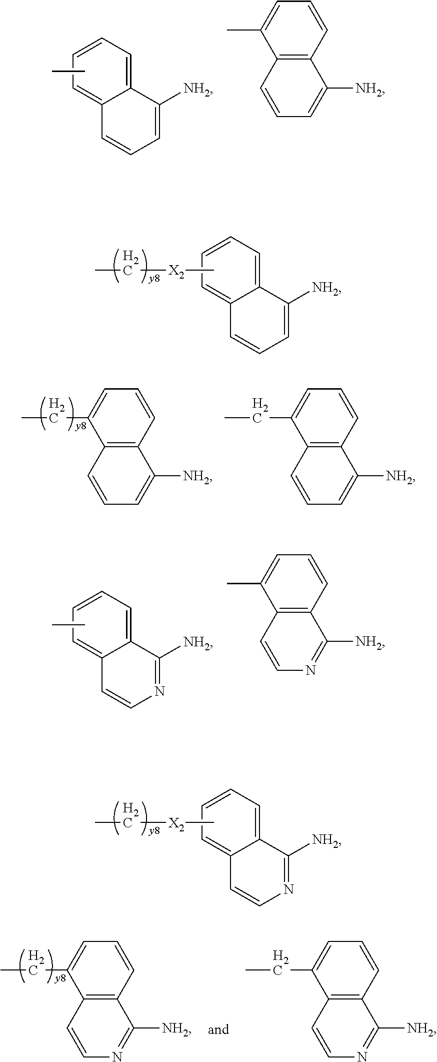

wherein X2 is any suitable linker molecule and y8 is an integer value, typically between 0 and 6. In preferred embodiments y8 is 1.

In non-limiting examples, wherein R⁴ is

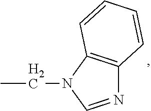

monomer M is:

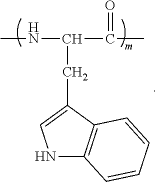

In some embodiments, the poly(amino acid)-based hydrophobic block (H) of Formula I comprises reactive amino acids, N, that are selected from any natural or non-natural amino acid, wherein a drug molecule (D) is linked directly or through X1 to the monomer. Suitable reactive amino acids include but are not limited to any amino acids bearing a group suitable for attachment of drug molecules, include amino acids with azide, alkyne, tetrazine, transcyclooctyne (TCO), protected hydrazine, ketone, aldehyde, certain hydroxyl groups, isocyanate, isothiocyanate, carboxylic acids, activated carboxylic acids, activated carbamates, activated carbamates, protected maleimide, thiol and/or amine groups.

X1 is any suitable linker for linking drug molecules, D, to the hydrophobic block (H), including to the reactive amino acid, N, of poly(amino acids) and is typically selected from —(CH₂)_{y10}-FG3 and —(CH₂)_{y10}—R⁶ (or —C(O)—(CH₂)_{y10}-FG3 and —C(O)—(CH₂)_{y10}—R⁶ when drugs are linked at the N-terminus or off of amine groups, or —NH—(CH₂)_{y10}-FG3 and —NH—(CH₂)_{y10}—R⁶ when drugs are linked at the C-terminus or off of carbonyl groups), wherein y10 is any integer, typically selected from 1 to 6, and R⁶ is typically selected from any one or more of —C(O)—NH—R⁷, —NH—C(O)—R⁷, —NH—C(O)—O—R⁷, —O—C(O)—NH—R⁷, —O—C(O)—R⁷, —C(O)—O—R⁷, —O—R⁷, O—C(O)—W, or —C(O)—W, wherein R⁷ is typically selected from any one or more of —(CH₂)_{y11}—W, —(CH₂)_{y11}—(OCH₂CH₂)_{y12}—W, —(CH₂)_{y11}—(OCH₂CH₂)_{y12}—(CH₂)_{y13}—W, —CHR⁸—C(O)—W, —CHR⁸—C(O)—(NH—CHR⁸—C(O))ⱼ—W, —(CH₂)_{y11}—C(O)—NH—CHR⁸—C(O)—W, —(CH₂)_{y11}—C(O)—NH—CHR⁸—C(O)—(NH—CHR⁸—C(O))ⱼ—W, —(CH₂)_{y11}—(OCH₂CH₂)_{y12}—C(O)—NH—CHR⁸—C(O)—W, —(CH₂)_{y11}—(OCH₂CH₂)_{y12}—(CH₂)_{y13}C(O)—NH—CHR⁸—C(O)—W, —(CH₂)_{y11}—(OCH₂CH₂)_{y12}—C(O)—NH—CHR⁸—C(O)—(NH—CHR⁸—C(O))ⱼ—W, —(CH₂)_{y11}—(OCH₂CH₂)_{y12}—(CH₂)_{y13}—C(O)—NH—CHR⁸—C(O)—(NH—CHR⁸—C(O))ⱼ—W, —CHR⁸—C(O)—NH—C₆H₄—CH₂—O—C(O)—W, —CHR⁸—C(O)—NH(CH₃)(CH₂)₂—O—C(O)—W, —CHR⁸—C(O)—(NH—CHR⁸—C(O))ⱼ—NH—C₆H₄—CH₂—O—C(O)—W, —CHR⁸—C(O)—(NH—CHR⁸—C(O))ⱼ—NH(CH₃)(CH₂)₂—O—C(O)—W, —(CH₂)_{y11}—C(O)—(NH—CHR⁸—C(O))ⱼ—NH—C₆H₄—CH₂—O—C(O)—W, —(CH₂)_{y11}—C(O)—(NH—CHR⁸—C(O))ⱼ—NH(CH₃)(CH₂)₂—O—C(O)—W, —(CH₂)_{y11}—(OCH₂CH₂)_{y12}—C(O)—(NH—CHR⁸—C(O))ⱼ—NH—C₆H₄—CH₂—O—C(O)—W, —(CH₂)_{y11}—(OCH₂CH₂)_{y12}—C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}—C(O)—NH—(CH₂)_{y14}—C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—NH—(CH₂)_{y14}—C(O)—(NH—CHR⁸—C(O))ⱼ—NH(CH₃)(CH₂)₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH—C₆H₄—CH₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH(CH₃)(CH₂)₂—O—C(O)—W, —CHR⁸—C(O)—NH—(CH₂)_{y15}—W, —CHR⁸—NH—C(O)—(CH₂)_{y15}—W, —CHR⁸—C(O)—(NH—CHR⁸—C(O))ⱼ—NH—(CH₂)_{y15}—W, —CHR⁸—NH—(C(O)—CHR⁸—NH)ⱼ—C(O)—(CH₂)_{y15}—W, where y11, y12, y13, y14, y15 and j are each independently selected from any integer typically selected from 1 to 6, R⁸ is any amino acid side group, and W can be independently selected from H (hydrogen), FG3, LG and w; wherein FG3 is any suitable functional group for attachment to the drug molecule, which may be selected from, but not limited to, carboxylic acid, activated carboxylic acids (e.g., carbonylthiazolidine-2-thione (“TT”), NHS or nitrophenol esters), carboxylic acid anhydrides, amine and protected amines (e.g., tert-butyloxycarbonyl protected amine), OSi(CH₃), alkene, azide, alkyne, stained-alkyne, halogen (e.g., fluoride, chloride), olefins and endo cyclic olefins (e.g., allyl), CN, OH, and epoxy, hydrazines (including hydrazides), carbohydrazides, aldehydes, ketones, carbamates and activated carbamates, LG is any suitable leaving group, which may be selected from, but not limited to any suitable leaving group (e.g., NHS, TT, nitrophenol, etc.), and, w is a group that results from either the reaction of FG4 with FG3 or the displacement of LG with FG4, and is typically selected from NH—, C(O)—, NH—C(O)—, C(O)—NH—, O—C(O)—NH—, C(O)—NH—N═C(CH₃)—, NH—N═C(CH₃)— or —C(CH₃)═N—NH—C(O)—, wherein w is always linked to D, either directly (i.e., w-D) or indirectly via X3 (i.e., w-X3-D).

Drug molecules (D) may be attached to the reactive amino acid, N, directly or via X1 through reaction of FG4 with FG3, wherein FG4 is any suitable functional group on the drug (D) that is reactive with FG3. Alternatively, drug molecules (D) may be linked to the reactive amino acid, N, via X1 through displacement of LG with any suitable FG4 comprising a nucleophile, e.g., a primary amine, or drug molecules (D) may be linked to the reactive amino acid, N, via X1 through displacement of an LG present on the drug molecule with any suitable FG3 comprising a nucleophile.

In preferred embodiments, FG3 is a carboxylic acid and FG4 is an amine, which react to form an amide. In non-limiting examples, X1 is selected from —(CH₂)_{y10}-FG3, y10 is 2, FG3 is a carboxylic acid, and FG4 present on the drug is an amine (i.e., NH₂-D), which react to form an amide, which may be represented as —(CH₂)₂—C(O)-D (amine not shown) or —(CH₂)₂—C(O)—NH-D (amine shown), indicating that the drug is linked via an amide bond at the carbonyl of X1, which (after amide bond formation) may be described as —(CH₂)_{y10}—R⁶ wherein y10 is 2, R⁶=C(O)—W, and W is the group w, which is NH— and is linked to D to give —(CH₂)₂—C(O)—NH-D.

The drug may additionally comprise a linker, X3, between the reactive functional group FG4 and the pharmacophore, e.g., FG4-X3-D. Specific, preferred compositions of X3 are described elsewhere.

In other embodiments, FG3 is an amine and FG4 is a carboxylic acid, which react to form an amide. In non-limiting examples, X1 is —(CH₂)_{y10}-FG3, y10 is 4, FG3 is an amine, and FG4 present on the drug is a carboxylic acid (i.e., COOH-D), which react to form an amide, which may be represented as —(CH₂)₄—NH-D (carbonyl not shown) or —(CH₂)₄—NH—C(O)-D (carbonyl shown), indicating that the drug is linked via an amide bond at the amine of X1.

In still other embodiments, FG3 is a ketone or aldehyde and FG4 is a hydrazide or carbohydrazide, which react to form a hydrazone. In non-limiting examples, X1 is —(CH₂)_{y10}—R⁶, y10 is 4, R⁶ is —NH—C(O)—R⁷, R⁷ is (CH₂)_{y11}—W, y11 is 2 and W is C(O)—CH₃, and FG4 present on the drug molecule is a hydrazide (NH₂—NH₂—C(O)-D), which reacts with X1, i.e., —(CH₂)₄—NH—C(O)—(CH₂)₂—C(O)—CH₃ to form a hydrazone bond, i.e., —(CH₂)₄—NH—C(O)—(CH₂)₂—C(CH₃)═N—NH—C(O)-D. In still other embodiments, FG3 is a hydrazide or carbohydrazide and FG4 is a ketone or aldehyde that reacts to form a hydrazone. In non-limiting examples, X1 is —(CH₂)_{y10}—R⁶, y10 is 2, R⁶ is —C(O)—W, W is FG3 and FG3 is —NH—NH₂ and FG4 present on the drug molecule is a ketone CH₃C(O)-D (or optionally CH₃C(O)—X3-D), which reacts with X1 to form —(CH₂)₄—C(O)—NH—NH₂ to form a hydrazone bind, i.e., form —(CH₂)₄—C(O)—NH—N═C(CH₃)-D.

In certain preferred compositions, drug molecules (D) are linked directly to the reactive amino acid, N. A non-limiting example of a reactive amino acid comprising a linker selected from —(CH₂)_{y10}-FG3, wherein y10=2, FG3 is carboxylic acid (i.e., the reactive amino acid is glutamic acid) linked to a drug molecule is shown below for clarity:

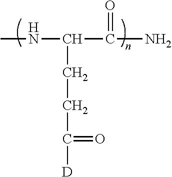

In certain other preferred embodiments, drug molecules (D) are linked to the reactive amino acid (N) via an enzyme degradable peptide and/or self-immolative linker, wherein the self-immolative linker is typically selected from —NH—C₆H₄—CH₂—O—C(O)— or —NH(CH₃)(CH₂)₂—O—C(O)— and FG4 present on the drug is an amine, e.g., NH₂-D or NH₂—X3-D, which results in a carbamate bond between the linker and the drug. In non-limiting examples, the reactive monomer comprises a linker selected from (CH₂)_{y10}—R⁶, wherein y10=2, R⁶ is —C(O)—NH—R⁷ and R⁷ is (CH₂)_{y11}—C(O)—(NH—CHR⁸—C(O))ⱼ—NH—C₆H₄—CH₂—O—C(O)—W, wherein y11 is 2, R⁸ is any amino acid group, j is an integer typically selected from 1 to 6, W is selected from the group w, which is NH— linked to the drug (D), as shown here:

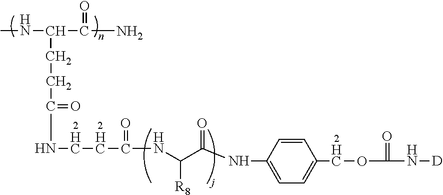

In preferred compositions of X1 comprising enzyme degradable linkers, the enzyme degradable linker typically comprises between 1 and 6 amino acids, such as 1, 2, 3, 4, 5 or 6 amino acids selected from single amino acids, dipeptides, tripeptides, tetrapeptides, pentapeptides and hexapeptides recognized and cleaved by enzymes, such as cathepsins and/or the immunoproteasome.

Reactive amino acids (N) may comprise functional groups that can impart charge; however, the classification of an amino acid as a reactive amino acid monomer is context-dependent and based on its intended use. For example, monomers comprising carboxylic acids may be referred to as charged monomers if the carboxylic acid is not used for drug attachment, whereas the same monomers linked to an amine bearing drug molecule, e.g., via an amide bind, would be considered a reactive monomer.

In some embodiments, the poly(amino acid)-based polymer of Formula I comprises spacer amino acids, O, that are selected from any natural or non-natural amino acid that are non-bulky and near neutral, such as a PEG amino acid spacer, e.g., Q of monomer O is a lower alkyl or PEG, e.g., —(CH₂)_{y6}—, —CH₂—CH₂—O— or —(CH₂—CH₂—O)_{y7}CH₂—CH₂—, wherein y6 and y7 are each independently an integer typically between 1 and 6. Alternatively, monomer 0, is selected from amino acids with a small, i.e., non-bulky, substituent selected from hydrogen, lower alkyl or a lower alkyl comprising a hydroxyl and is provided to increase the spacing or flexibility of the polymer backbone.

Non-limiting examples include:

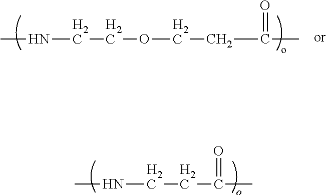

In some embodiments, the poly(amino acid)-based polymer of Formula I comprises optional co-monomer(s), P, that are selected from any natural or non-natural amino acid, wherein R₅ is selected from any group comprising a functional group that carries charge either permanently or at a specific pH in aqueous solutions. Non-limiting examples of charged amino acids include any natural or non-natural amino acid that comprise amine, quaternary ammonium, sulfonic acid, sulfuric acid, sulfonium, phosphoric acid, phosphonic acid, phosphonium, carboxylic acid, boronic acid functional groups and/or combination thereof, including zwitterions, which may be linked either directly or via a suitable linker molecule, as well as any composition of salts thereof. Non-limiting examples of salts include, e.g., positively charged functional groups, e.g., ammonium ions paired with halide (e.g., chloride) ions. Other non-limiting examples of suitable salts of charged amino acids include conjugate bases of carboxylic, sulfonic and phosphonic acids, paired with group 1 metals, such as sodium, or ammonium or guanidinium ions.

In some preferred embodiments of amphiphiles for nucleic acid delivery, the amphiphile comprises a hydrophobic block (H) further comprising a poly(amino acid)-based polymer of Formula I that includes R⁵ selected from groups that have net positive charge, which include but are not limited to:

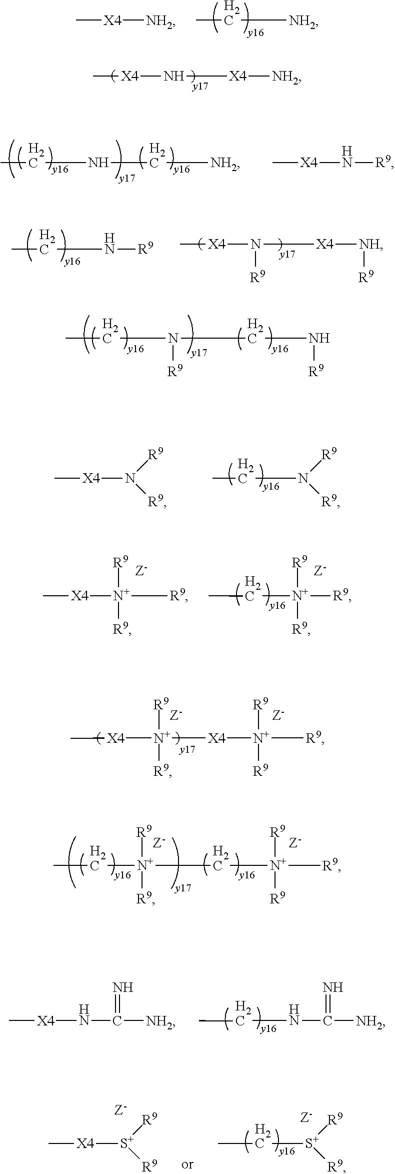

wherein X4 is any suitable linker, y16 and y17 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z⁻ is any suitable counter anion, which is typically selected from conjugate bases of weak acids or halide ions, such as Cl⁻, I⁻, or Br⁻.

The hydrophobic block (H) functions to drive particle assembly in aqueous solutions and therefore, in preferred embodiments of amphiphiles, peptide antigen conjugate or drug molecule conjugates, the hydrophobic block (H) comprises hydrophobic amino acids and/or reactive amino acids linked to hydrophobic drug molecules. In preferred embodiments of poly(amino acid)-based polymers of Formula I, the poly(amino acid)-based polymer (or oligomer) of Formula I comprises hydrophobic amino acids (M) and/or reactive amino acids (N) linked to hydrophobic drug molecules, and optionally spacer amino acids (O) and/or charged amino acids (P). In preferred embodiments of amphiphiles, peptide antigen conjugate or drug molecule conjugates used for peptide antigen delivery and/or for the delivery of neutral drug molecules, the hydrophobic block (H) is typically selected from poly(amino acid)-based polymers of Formula I comprising hydrophobic amino acids (M) and/or reactive amino acids (N) linked to hydrophobic drug molecules, and optionally spacer amino acids (O), but not charged amino acids (P). In contrast, wherein the amphiphiles, peptide antigen conjugate or drug molecule conjugates are used for nucleic acid delivery or for the delivery of charged drug molecules, the hydrophobic block (H) is typically selected from poly(amino acid)-based polymers of Formula I comprising hydrophobic amino acids (M) and/or charged amino acids (P), wherein the charge of the charge amino acid is opposite that of the nucleic acid or charged drug molecule, and optionally reactive amino acids (N) linked to hydrophobic drug molecules and spacer amino acids (O). Particular compositions of hydrophobic blocks (H) based on poly(amino acid)-based polymers or oligomers of Formula I that led to unexpected improvements in biological activity are described throughout the specification.

In some embodiments, the hydrophobic block (H) is a poly(amino acid) of Formula I comprising entirely hydrophobic monomers (m):

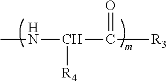

Non-limiting examples include:

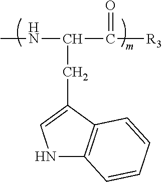

A non-limiting example of a poly(amino acid) of Formula I composed entirely of hydrophobic monomers (M) selected from tryptophan, wherein m is equal to 5 (i.e., 5 monomeric units), R³ is an amine and the N-terminal amine is linked to a solubilizing block (S) either directly or indirectly through a spacer (B) and/or linker U, is shown here for clarity:

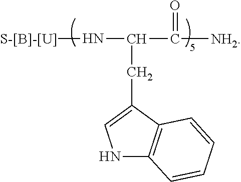

In some embodiments drug molecules (D) are linked via the N-terminus or C-terminus of hydrophobic blocks (H) comprising poly(amino acids) of Formula I. A non-limiting example is shown here for clarity:

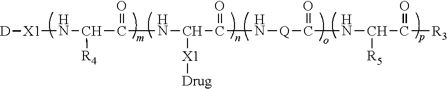

Wherein the poly(amino acid) comprises hydrophobic amino acids selected from tryptophan and R³ is NH₂ the structure is:

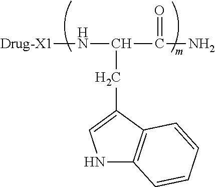

Wherein when X1 comprises a PAB-Cit-Val linked to the poly(amino acid) via a succinate linker the structure is:

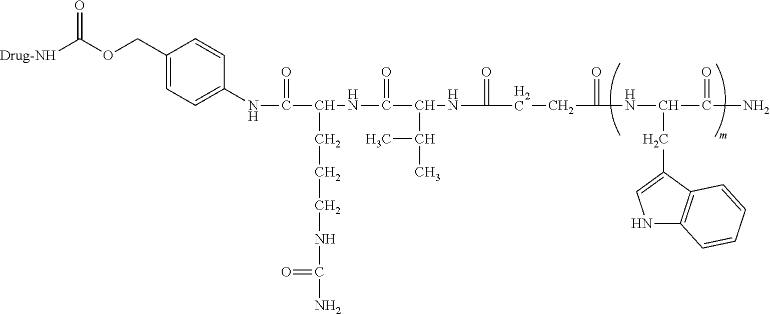

Alternatively, wherein X1, comprises a PAB-Cit-Val linked to the poly(amino acid) via Linker U resulting from the reaction between azide and DBCO, an exemplary strained alkyne, wherein the DBCO moiety is linked to poly(amino acid) via Ahx, the structure is:

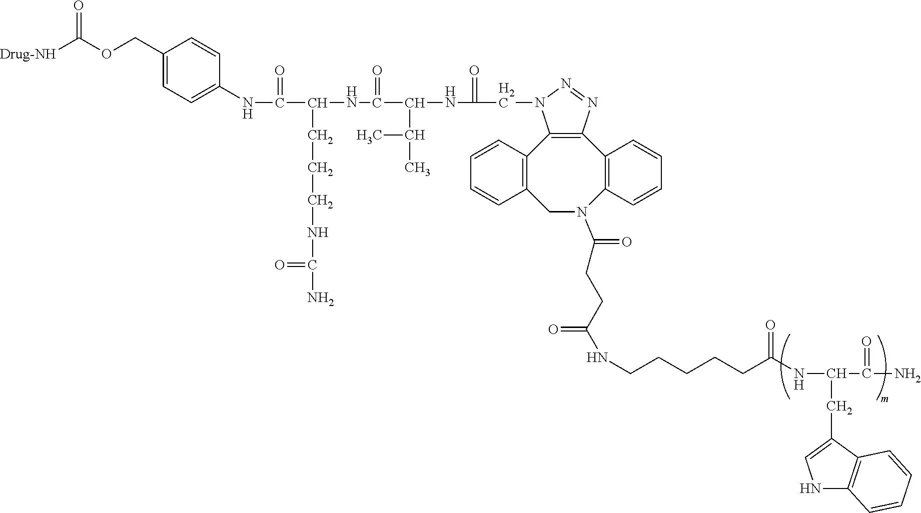

In some embodiments of poly(amino acid)-based hydrophobic blocks (H), the hydrophobic block (H) comprises 3 or more, preferably about 3 to about 100 hydrophobic amino acids (M) and/or reactive amino acids linked to drug molecules (D), though, more preferably between about 3 to 30 hydrophobic amino acids (M) and/or reactive amino acids linked to drug molecules (D), more preferably wherein the hydrophobic amino acids and/or reactive amino acids linked to drug molecules (D) further comprise aryl groups, heteroaryl, aminoaryl and/or aminoheteroaryl.

Hydrophobic Blocks (H) with Branched Architecture

In some embodiments, a hydrophobic block (H) is branched. In certain cases, the hydrophobic block (H) comprises a dendron, wherein the focal point is linked to either (i) a solubilizing block (S) either directly or indirectly via a spacer (B) and/or Linker U, (ii) an antigen (A) either directly or indirectly via an extension (E1 or E2) and/or Linker U; (iii) a drug molecule either directly or via a Linker U; or, (iv) a capping group, and the terminal functional groups (FGt) are linked to hydrophobic molecules, e.g., hydrophobic drug molecules, more preferably hydrophobic molecules comprising aromatic groups, e.g., hydrophobic drug molecules comprising aromatic groups.

Non-limiting examples of amphiphiles, peptide antigen conjugates or drug molecule conjugates comprising hydrophobic blocks (H) with dendron architecture, wherein the terminal functional groups (FGt) are linked to hydrophobic drug molecules are provided below for clarity:

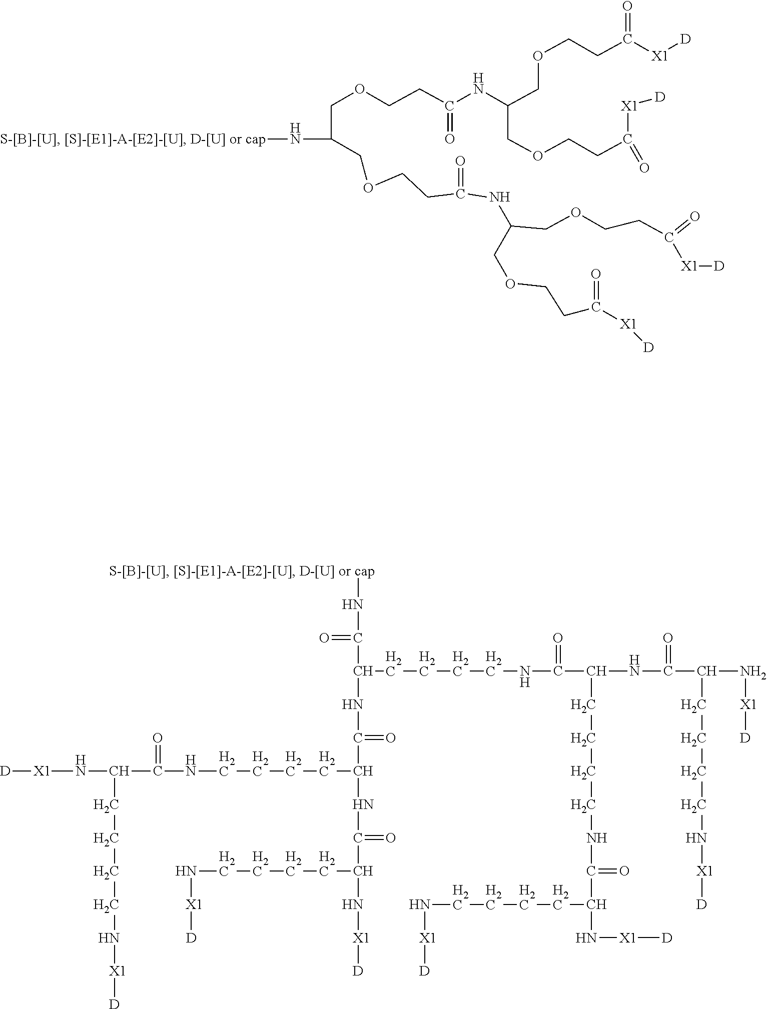

Wherein X1 is either present or absent and when present is any suitable linker and D is any suitable drug molecule, preferably selected from hydrophobic drug molecules comprising aromatic groups, and the focal point is attached to either (i) a solubilizing block (S) either directly or indirectly via a spacer (B) and/or Linker U, (ii) an antigen (A) either directly or indirectly via an extension (E1 or E2) and/or Linker U; (iii) a drug molecule either directly or via a Linker U; or, (iv) a capping group.

Additional examples of hydrophobic blocks (H) with dendron architecture that have particular utility for certain applications and/or lead to unexpected improvements in manufacturing and/or biological activity are provided throughout the specification.

Density (Mol %) of Hydrophobic Groups and/or Drug Molecules

The density (i.e., mol %) of the hydrophobic monomers (e.g., hydrophobic amino acids or reactive monomers linked to hydrophobic drug molecules) incorporated into polymer-based hydrophobic blocks (H), e.g., poly(amino acids) of Formula I, were found to have a major impact on particle stability and biological activity. Thus, the density (i.e., mol %) of hydrophobic monomers (e.g., hydrophobic amino acids or reactive monomers linked to hydrophobic drug molecules) incorporated into polymer-based hydrophobic blocks should be carefully selected. In general, the density (mol %) of hydrophobic monomers (e.g., hydrophobic amino acids or reactive monomers linked to hydrophobic drug molecules) required is inversely proportional to the length (i.e. degree of polymerization) of the polymer.

For instance, the preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) is typically 100 mol % for polymers (or “oligomers”) with 3 monomers; 75-100 mol % for polymers (or “oligomers”) with 4 monomers, such as 75 mol % or 100 mol % for polymers with 4 monomers; 60-100 mol % for polymers (or “oligomers”) with 5 monomers, such as 60 mol %, 80 mol % or 100 mol %; 50-100 mol % for polymers (or “oligomers”) with 6 monomers, such as 50 mol %, 66.6 mol %, 83.3 mol % and 100 mol %; 42-100 mol % for polymers (or “oligomers”) with 7 monomers, such as 42 mol %, 57 mol %, 71 mol %, 85.7 mol % and 100 mol %; 37.5-100 mol % for polymers (or “oligomers”) with 8 monomers, such as 37.5 mol %, 50 mol %, 75 mol %, 87.5 mol % and 100 mol %; 33.3-100 mol % for polymers (or “oligomers”) with 9 monomers, such as 33.3 mol %, 44.4 mol %, 55.6 mol %, 66.6 mol %, 77.9 mol %, 88.9 mol % and 100 mol %; 30-100 mol % for polymers (or “oligomers”) with 10 monomers, such as 30 mol %, 40 mol %, 50 mol %, 60 mol %, 70 mol %, 80 mol %, 90 mol % and 100 mol %. The preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) for polymers with between 11 and 20 monomers is typically between 20 mol % to 100 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol %, provided that at least 3 hydrophobic monomers (M) or reactive monomers (N) linked to hydrophobic drugs are present; 10-100 mol %, more preferably 20-80 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol % or 80 mol % for polymers with between 21 and 30 monomers, provided that at least 3 hydrophobic monomers (M) or reactive monomers (N) linked to hydrophobic drugs are present; and, 5-60 mol %, more preferably, 10-40 mol % for polymers with >30 monomers, such as 10 mol %, 11 mol %, 12, mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol % and 40 mol % for polymers with >30 monomers.

In the above examples, in preferred embodiments, the polymer is a poly(amino acid) and the monomer is selected from hydrophobic monomers (e.g., hydrophobic amino acid and/or reactive monomers linked to hydrophobic drug molecules) that comprise an aryl group, and, more preferably, a heteroaryl, aminoaryl, and/or aminoheteroaryl. Additionally, in the above examples, the hydrophobic monomer may be selected from two or more monomers, e.g., two or more distinct hydrophobic monomers (e.g., hydrophobic amino acids), or one or more hydrophobic monomers and one or more reactive monomers (e.g., reactive amino acids) linked to hydrophobic drugs, such that the total mol % of hydrophobic monomers falls within the preferred ranges.

### General Properties of Polymer-Based Hydrophobic Blocks (H)

The average molecular weight of polymer-based hydrophobic blocks (H) can be readily estimated based on the number and composition of monomers (e.g., amino acids for poly(amino acids) and is typically between about 500 g/mol to about 20,000 g/mol. In some embodiments, the polymer molecular weight is between about 1,000 and 5,000, or between about 5,000 and 10,000, or between about 10,000 and 20,000 g/mol.

The polydispersity, Mw/Mn, of the hydrophobic polymer or oligomer (H) typically ranges from about 1.0 to 2.0 and depends on the polymerization technique used. For instance, poly(amino acid)-based hydrophobic polymers or oligomers (H) are typically prepared by solid phase peptide synthesis and will have polydispersity of 1.0 as the polymers are molecularly defined. Polymers formed by chain growth polymerization will have polydispersities>1.0. The hydrophobic polymer or oligomer (H) may also comprise polymers based on cyclic monomers, such as poly(amino acid)-based hydrophobic polymers or oligomers (H) based on amino acid N-carboxyanhydrides (NCAs).

The size of the polymer-based hydrophobic block (H) may either be expressed by the molecular weight or degree of polymerization. For molecularly defined, monodisperse polymers, the length (or degree or degree polymerization) of the polymer can be calculated by dividing the molecular weight (e.g., theoretical or experimentally determined by mass spectrometry) by the average molecular weight of the monomer unit(s) comprising the polymer. For polydisperse polymers, the number-average molecular weight, abbreviated Mn, is preferred for estimating the degree of polymerization. As a non-limiting example, a polydisperse polymer with a Mn of 25 kDa and an average monomer molecular weight of 250 g/mol would have a degree of polymerization of 100. The molecular weight of a polymer can also be calculated by multiplying the degree of polymerization by the average monomer molecular weight.

In preferred embodiments of hydrophobic blocks (H), the molecular weight or Mn, is preferably between about 0.5 kDa and 60 kDa, such as about 0.5 kDa, 1 kDa, 1.5 kDa, 2 kDa, 2.5 kDa, 3 kDa, 3.5 kDa, 4 kDa, 4.5 kDa, 5 kDa, 6 kDa, 7 kDa, 8 kDa, 9 kDa, 10 kDa, 11 kDa, 12 kDa, 13, kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa or 60 kDa. More preferably, the molecular weight of the hydrophobic block is between about 0.5 kDa to about 20 kDa. In certain embodiments, the hydrophobic block (H) is a poly(amino acid) and has a molecular weight of between about 0.5 kDa and about 10 kDa or about 1.5 kDa to about 5 kDa.

Polymers described herein can be synthesized by any suitable means and should preferably have low or no polydispersity. For instance, poly(amino acids) described herein are typically produced by solid-phase peptide synthesis and are molecularly defined with no polydispersity. Similarly, PEG based spacers and dendrons described herein are produced by controlled processed and have little to no polydispersity. In contrast, polymers produced by radical polymerization will have some degree of polydispersity, which may be calculated by dividing the weight-average molecular weight Mw by Mn, i.e., polydispersity index (PDI)=Mw/Mn. Though, the polydispersity of polymers produced by radical polymerization may be controlled by the polymerization technique utilized. Therefore, in preferred embodiments, living polymerization, e.g., RAFT polymerization, is used to synthesize polymers with PDI less than 2.0, typically between about 1.01 and 1.2.

### C. Exemplary Peptide Antigen Conjugates of Formula S-[E1]-A-[E2]-[U]-H-[D]

Non-limiting examples of peptide antigen conjugates include those with a formula S-[E1]-A-[E2]-[U]-H-[D] or [D]-H-[U]-[E1]-A-[E2]-S or H-[D]-U-[E1]-A-[E2]-[S]. Nonlimiting examples include peptide antigen conjugates with tumor antigens such as viral antigens (e.g., HPV E6 derived antigen: KHKSAIVTLTYDSEWQRDQFLSQVKIPKT (SEQ ID NO: 104)), self-antigens (e.g., PSA derived: CGGVLVHPQWVLTAAHCIRNKSVILLGRHSLFHPE (SEQ ID NO: 130)) or tumor neoantigen (e.g., mutant Kras G12C mutant: MTEYKLVVVGACGVGKSALTIQLIQ (SEQ ID NO: 161)), combined with S, E1, E2, U, H, and D as described above, in which D is covalently linked to H. In some cases, the drug D is an immunostimulant, such as (a) one or more of a TLR-3, TLR-7, TLR-8, TLR-7/8, TLR-9, MDA5, RIG1, or STING agonist, or (b) a molecule that induces Flt3, IL-12, and/or type-I IFN signaling. In some cases, the drug D is a TLR-7/8 agonist.

Examples with particular antigen sequences A, include:

(SEQ ID NO: 104)
S-[E1]-KHKSAIVTLTYDSEWQRDQFLSQVKIPKT-
[E2]-[U]-H[D]
(SEQ ID NO: 130)
S-[E1]-CGGVLVHPQWVLTAAHCIRNKSVILLGRH
SLFHPE-[E2]-[U]-H [D],
(SEQ ID NO: 161)
S-[E1]-MTEYKLVVVGACGVGKSALTIQLIQ-[E2]-
[U]-H [D],
(SEQ ID NO: 104)
[D]H-[U]-[E1]-KHKSAIVTLTYDSEWQRDQFLSQ
VKIPKT-[E2]-S,
(SEQ ID NO: 130)
[D]H-[U]-[E1]-CGGVLVHPQWVLTAAHCIRNKSVI
LLGRHSLFHPE-[E2]-S,
and
(SEQ ID NO: 161)
[D]H-[U]-[E1]-MTEYKLVVVGACGVGKSALTIQLIQ-[E2]-S.

In some embodiments, the hydrophobic block H is Ahx-Glu(2B)-Trp-Glu(2B)-Trp-Glu(2B)-NH₂ (SEQ ID NO: 165), wherein 2B is a TLR-7/8 agonist or other immunostimulant drug. In some embodiments above, the hydrophobic block H is Ahx-Glu(2B)-Trp-Glu(2B)-Trp-Glu(2B)-NH₂ (SEQ ID NO: 165), wherein 2B is a TLR-7/8 agonist drug D. In some embodiments, U is presented and is selected from Lys(N3-DBCO). In some embodiments, E1 is included and is valine-citrulline (abbreviated Val-Cit or VZ, where Z=citrulline). In some embodiments, E2 is included and is SPVZ. Thus, in some nonlimiting examples, the structures are as follows, where the N-terminal S adjacent to [E1] or [E2] represents the solubilizing block, which can in some cases be a charged molecule C. Further examples include:

(SEQ ID NO: 162)
S-VZ-KHKSAIVTLTYDSEWQRDQFLSQVKIPKT-SPVZ-
Lys(N3-DBCO)-Ahx-Glu(2B)-Trp-Glu(2B)-Trp-
Glu(2B)-NH2,
(SEQ ID NO: 163)
S-VZ-CGGVLVHPQWVLTAAHCIRNKSVILLGRHSLFHPE-
SPVZ- Lys(N3-DBCO)- Ahx-Glu(2B)-Trp-Glu
(2B)-Trp-Glu(2B)-NH2,
and
(SEQ ID NO: 164)
S-VZ-MTEYKLVVVGACGVGKSALTIQLIQ-SPVZ-
Lys(N3-DBCO)-Ahx-Glu(2B)-Trp-Glu(2B)-
Trp-Glu(2B)-NH2.

In those examples, the N-terminal S is the solubilizing block, which may be a charged molecule C. 2B is a TLR-7/8 agonist drug D, and Z represents citrulline.

In certain embodiments of vaccines, the vaccine comprises one or more, typically between 1 to 40, peptide antigen conjugates of formula [S]-[E1]-A-[E2]-[U]-H-[D] or [D]-H-[U]-[E1]-A-[E2]-S or H-[D]-U-[E1]-A-[E2]-[S], and optionally further includes an amphiphile of formula S-B-[U]-H-[D], in some cases with cone architecture, wherein the amphiphile with optional cone architecture further comprises a solubilizing block comprising a PEG-based dendron with between 4 to 16 solubilizing groups and a PEG-based spacer with between 4 and 48 monomer units, more preferably 4 to 36 monomer units, most preferably 24 monomers units, additionally wherein the solubilizing groups comprise sugar molecules, carboxylic acids, amines and/or hydroxyls, and the hydrophobic block comprises a poly(amino acid) of Formula I. A non-limiting example is provided here for clarity:

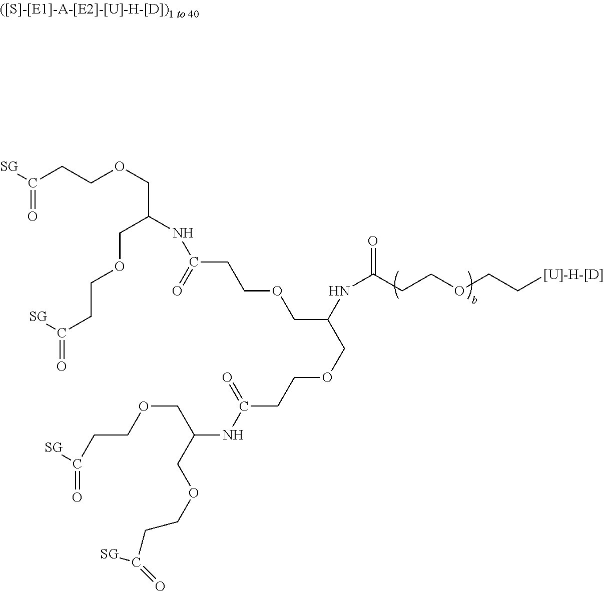

wherein b is an integer number of monomeric units comprising the spacer and is typically between 4 and 48, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 22, 43, 44, 45, 46, 47 or 48 monomeric units, preferably between about 4 and 36 monomer units, most preferably 24 monomeric units; SG is selected from sugar molecules, carboxylic acids, amines and/or hydroxyls that are linked to S either directly or via a suitable linker X, or, more preferably, X5; the hydrophobic block (H) is typically selected from poly(amino acids) of Formula I; S is a solubilizing block; E1 is a N-terminal extension; A is an antigen; E2 is a C-terminal extension; U is a linker; D is drug molecule; and [ ] denotes that the groups are optional. In some alternative embodiments the peptide antigen conjugates have the formula H-[U]-[E1]-A-[E2]-[S]. For clarity, each occurrence of any of the components of vaccines described herein, e.g., H, S, A, E1, E2, B, D and any linkers (e.g., U) are independently selected.

A non-limiting example of a vaccine comprising one or more, typically between 1 to 40, peptide antigen conjugates of formula [S]-[E1]-A-[E2]-[U]-H-[D] and an amphiphile of formula S-B-[U]-H-[D] with cone architecture, wherein the amphiphile with cone architecture further comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose and the hydrophobic block comprises a poly(amino acid) of Formula I, is provided here for clarity:

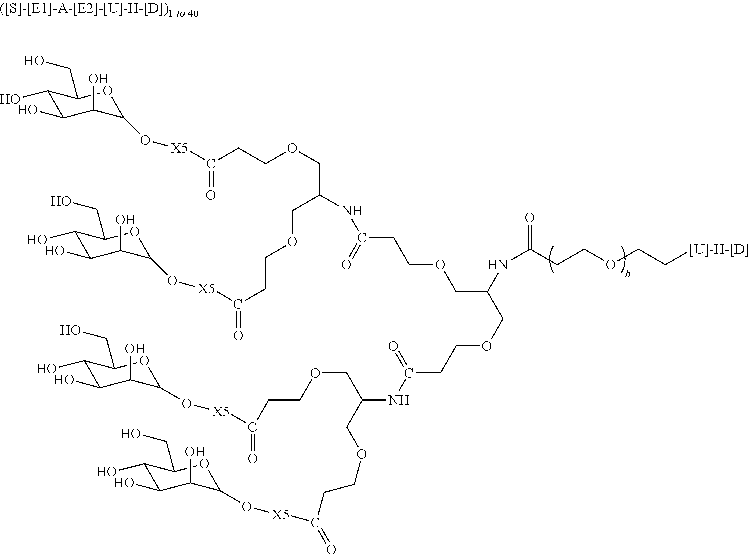

X5 is a suitable linker; b is an integer number of monomeric units comprising the spacer and is preferably between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I; S is a solubilizing block, E1 is a N-terminal extension, A is an antigen, E2 is a C-terminal extension, U is a linker, D is a drug molecule and [ ] denotes that the groups are optional. In some alternative embodiments, the peptide antigen conjugates have the formula H-[U]-[E1]-A-[E2]-[S].

In some embodiments of vaccines, the vaccine comprises one or more, typically between 1 to 40, peptide antigen conjugates of formula [S]-[E1]-A-[E2]-[U]-H-[D] or [D]-U-[E1]-A-[E2]-[S], and an amphiphile of formula S-B-[U]-H-[D] with cone architecture, wherein the amphiphile with cone architecture further comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose and the hydrophobic block of both the peptide antigen conjugate and the amphiphile comprises a poly(amino acid) of Formula I comprising hydrophobic monomers, M, with aryl, heteroaryl, aminoaryl and/or aminoheteroaryl groups:

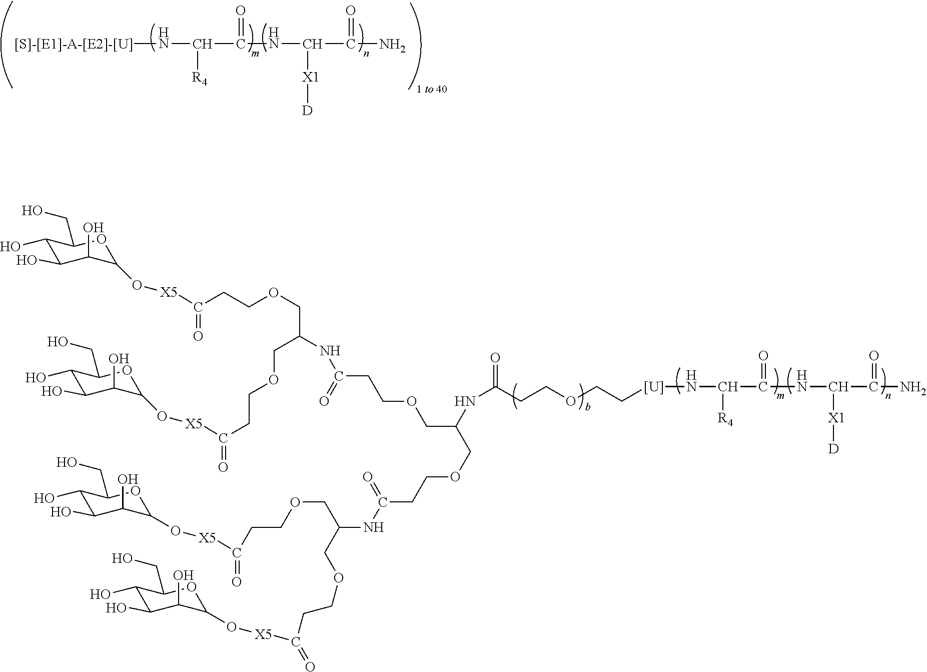

wherein X5 is a suitable linker; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I, wherein R⁴ is selected from aryl, heteroaryl, aminoaryl and/or aminoheteroaryl groups and m is typically between 3 and 30; A is an antigen, S is a solubilizing block, E1 is a N-terminal extension, E2 is a C-terminal extension, U is a linker and [ ] denotes that the groups are optional. In some alternative embodiments the peptide antigen conjugates have the formula H-[U]-[E1]-A-[E2]-[S].

In the above example, wherein the hydrophobic monomer is para-aminophenylalanine (sometimes abbreviated “F’) the structures of the peptide antigen conjugate and amphiphile are:

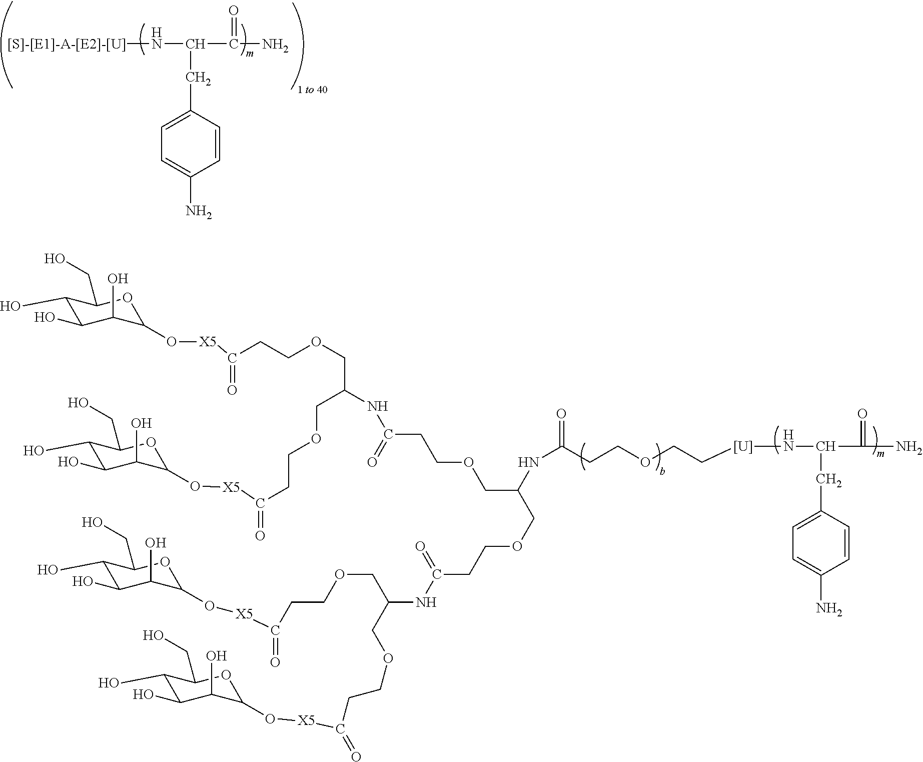

In certain embodiments of vaccines, a drug molecule is included in the hydrophobic block of the peptide antigen conjugate and/or amphiphile. A non-limiting example is provided here for clarity:

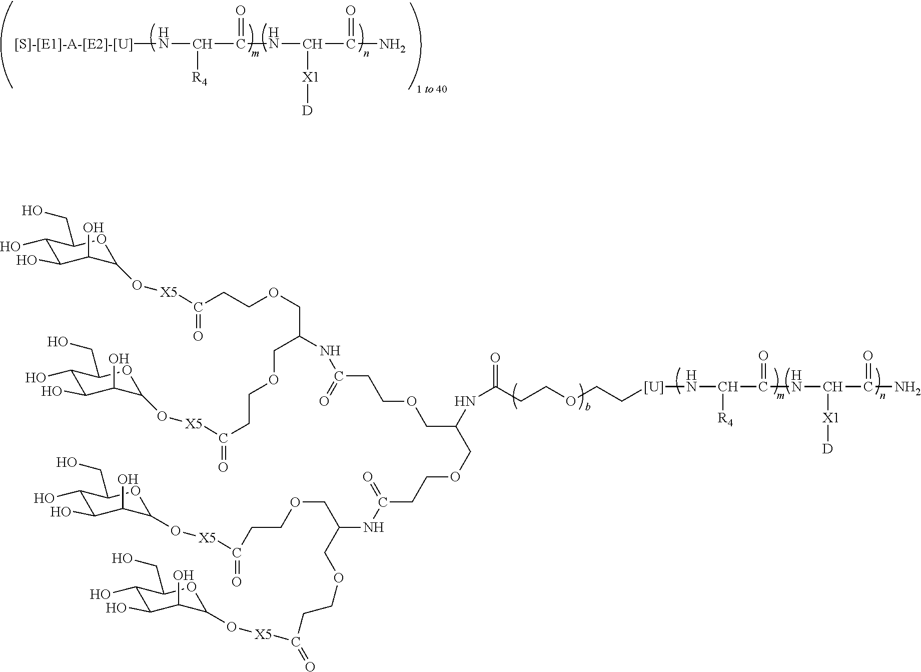

wherein X1 and X5 are suitable linkers; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block (H) comprises a poly(amino acid) of Formula I, wherein R₄ is selected from aryl, heteroaryl, aminoaryl and/or aminoheteroaryl groups; the drug (D) is any suitable immunomodulatory drug; m and n are an integer number of repeating units of monomers M and N, wherein the sum of m and n is typically between 3 and 30; A is an antigen, S is a solubilizing block, E1 is a N-terminal extension, E2 is a C-terminal extension, U is a linker and [ ] denotes that the groups are optional. In some alternative embodiments the peptide antigen conjugates have the formula H-[U]-[E1]-A-[E2]-[S].

A further non-limiting example is shown here for clarity:

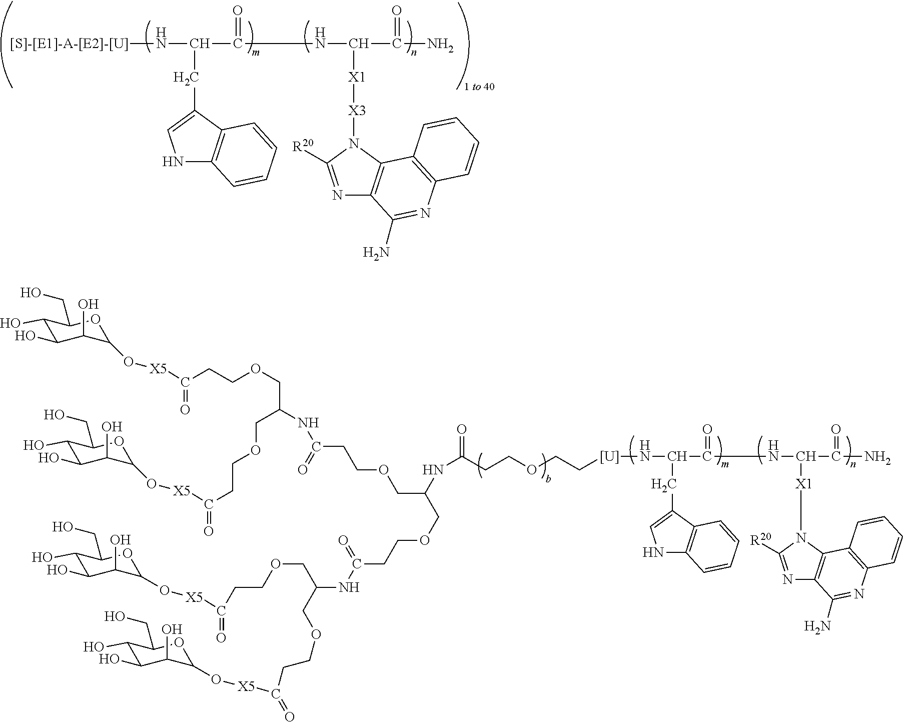

In some embodiments of vaccines wherein the hydrophobic block of the peptide antigen conjugate and/or amphiphile comprises a drug molecule, the hydrophobic block is linked to the antigen and amphiphile through a Linker U comprising a triazole. A non-limiting example is provided here for clarity:

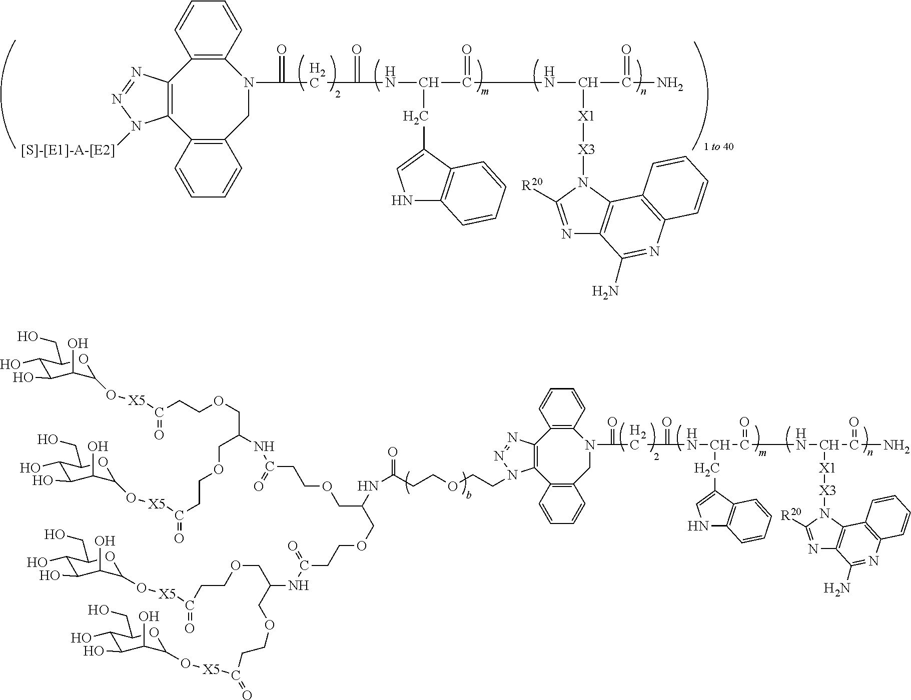

For vaccines wherein a unique set of peptide antigen conjugates is provided to each patient, one may use conjugates with hydrophobic blocks comprising poly(amino acids) of Formula I further comprising hydrophobic monomers, M, with aryl, heteroaryl, aminoaryl and/or aminoheteroaryl groups, and optionally charged amino acids (P) comprising amines, wherein the number of amino acids comprising the hydrophobic block is typically between 3 to 30; and to use amphiphiles with hydrophobic blocks comprising drug molecules. A non-limiting example is provided here for clarity:

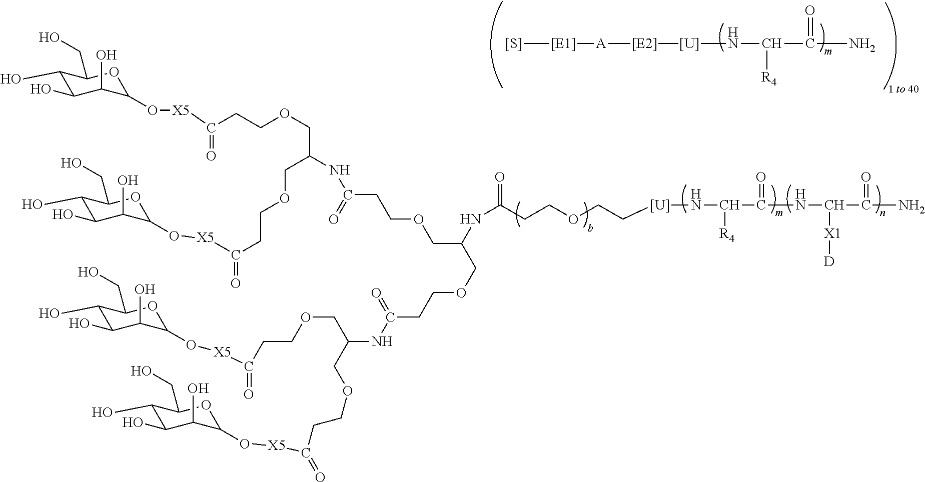

wherein X1 and X5 are each independently any suitable linker; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; the hydrophobic block of the peptide antigen conjugate comprises a poly(amino acid) of Formula I, wherein R⁴ is selected from aryl, heteroaryl, aminoaryl and/or aminoheteroaryl groups and m is typically between 3 and 30; the hydrophobic block of the amphiphile comprises a poly(amino acid) of Formula I, wherein R₄ is selected from aryl, heteroaryl, aminoaryl and/or aminoheteroaryl groups; the drug (D) is any suitable immunomodulatory drug; m and n are an integer number of repeating units of monomers M and N, wherein the sum of m and n is typically between 3 and 30; A is an antigen, S is a solubilizing block, E1 is a N-terminal extension, E2 is a C-terminal extension; U is a linker and [ ] denotes that the groups are optional. In some alternative embodiments the peptide antigen conjugates have the formula H-[U]-[E1]-A-[E2]-[S].

In still other embodiments, a vaccine comprises drug molecules selected from imidazoquinolines that are covalently linked to the hydrophobic block of the peptide antigen conjugate but not the amphiphile. A non-limiting example is shown here for clarity:

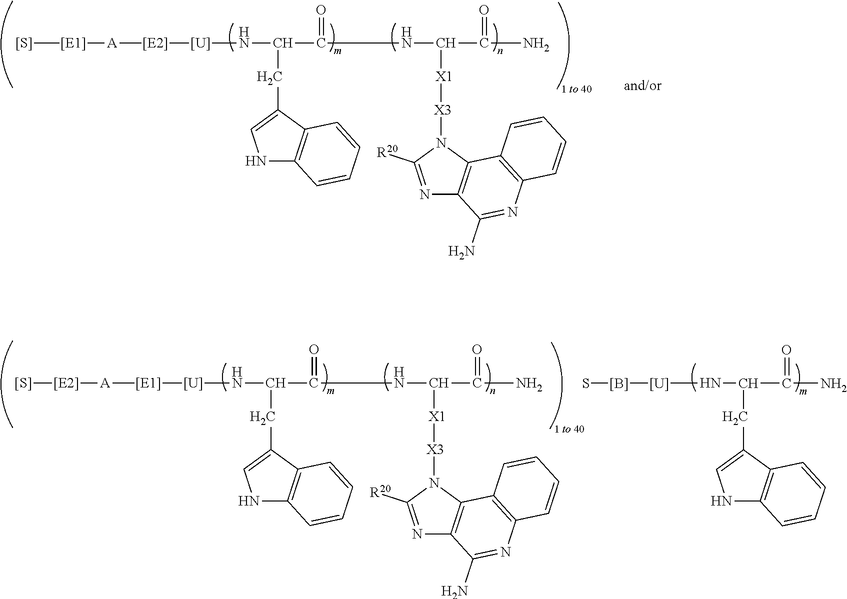

For example, wherein the amphiphile has dendron architecture and comprises a solubilizing block comprising a PEG-based dendron with 4 solubilizing groups (SG) and a PEG-based spacer with between 4 and 36 monomer units, additionally wherein the solubilizing groups comprise sugar molecules selected from mannose:

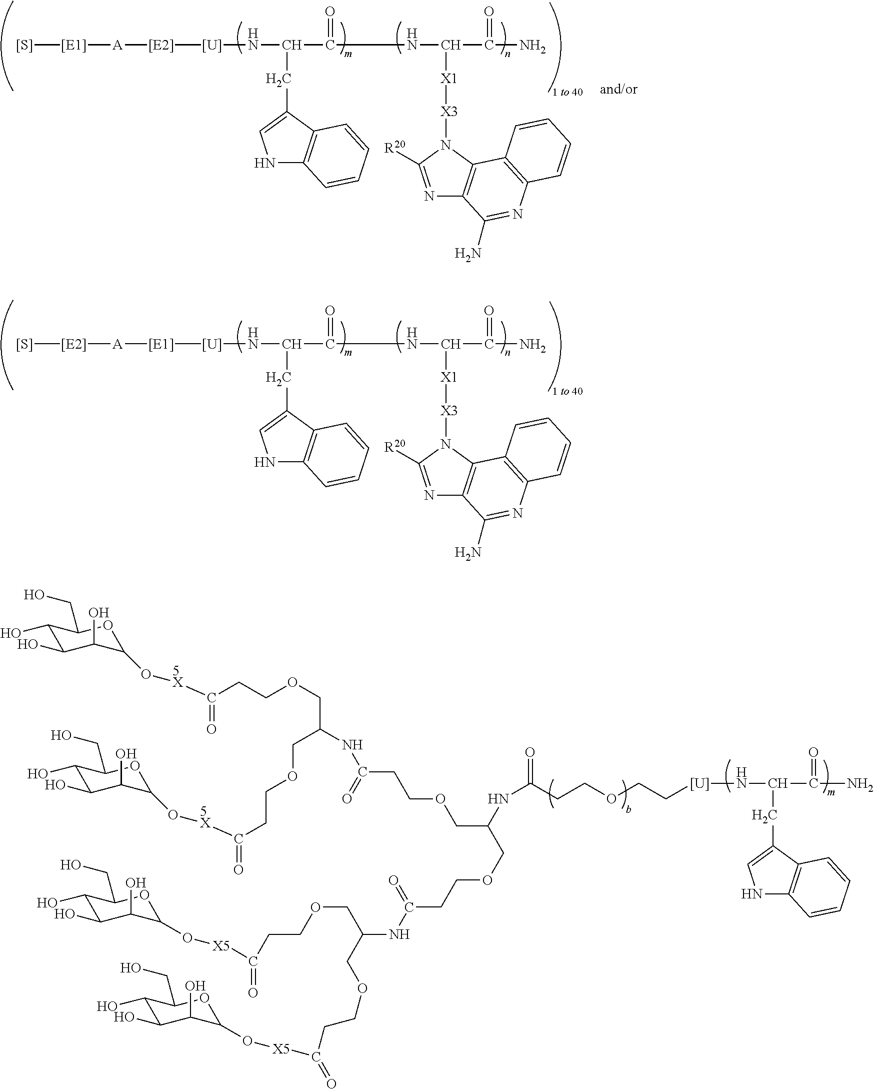

In some embodiments of cancer vaccines, the vaccine comprises one or more, typically between 1 to 40, peptide antigen conjugates of formula [S]-[E1]-A-[E2]-[U]-H-[D] or H-[D]-U-[E1]-A-[E2]-[S] and an amphiphile of formula S-B-[U]-H-[D] with cone architecture, wherein the amphiphile with cone architecture further comprises a solubilizing block comprising a PEG-based dendron with between 4 to 16 solubilizing groups and a PEG-based spacer with between 4 and 36 monomer units, additionally wherein the solubilizing groups comprise sugar molecules, carboxylic acids, amines and/or hydroxyls, and the hydrophobic block comprises a poly(amino acid) of Formula I. A non-limiting example is provided here for clarity:

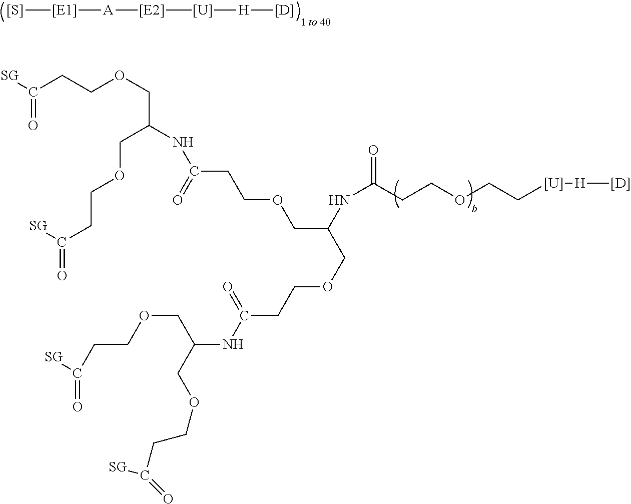

wherein b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; SG is selected from sugar molecules, carboxylic acids, amines and/or hydroxyls that are linked to S either directly or via a suitable linker X, or, more preferably, X5; the hydrophobic block (H) is typically selected from poly(amino acids) of Formula I; S is a solubilizing block, E1 is a N-terminal extension, A is an antigen selected from tumor antigens, E2 is a C-terminal extension, U is a linker, D is drug molecule and [ ] denotes that the groups are optional. In some alternative embodiments, the peptide antigen conjugates have the formula H-[U]-[E1]-A-[E2]-[S]. In preferred embodiments, the peptide antigen conjugate has net positive charge greater than or equal to +2, preferably between +3 and +5, and the solubilizing block of the peptide antigen conjugate is present and comprises a poly(amino acid) (or “peptide”) further comprising lysine and/or ornithine residues; the molar ratio of the peptide antigen conjugate to amphiphile is between 4:1 and 1:4, more preferably between 2:1 and 1:2 or about 1:1; and b comprises between 24 to 36 monomeric units.

In certain other embodiments of cancer vaccines, the vaccine comprises one or more, typically between 1 to 40, peptide antigen conjugates of formula [S]-[E1]-A-[E2]-[U]-H-[D] or H-[D]-U-[E1]-A-[E2]-[S] and an amphiphile of formula S-B-[U]-H-[D] with linear architecture, wherein the amphiphile with linear architecture further comprises a solubilizing block comprising a peptide with between 3 to 12 charged amino acids and a PEG-based spacer with between 4 and 36 monomer units, and the hydrophobic block comprises a poly(amino acid) of Formula I.

In some embodiments of cancer vaccines, the vaccine comprises an immunostimulatory drug molecule and one or more, typically between 1 to 40, peptide antigen conjugates of formula [S]-[E1]-A-[E2]-[U]-H-D or H-[D]-U-[E1]-A-[E2]-[S] and an amphiphile of formula S-B-[U]-H-D with cone architecture, wherein the amphiphile with cone architecture further comprises a solubilizing block comprising a PEG-based dendron with between 4 to 16 solubilizing groups and a PEG-based spacer with between 4 and 36 monomer units, additionally wherein the solubilizing groups comprise sugar molecules selected from mannose or Sialyl Lewis^{X} (sLeX), and the hydrophobic block comprises a poly(amino acid) of Formula I further comprising an imidazoquinoline of Formula IV. A non-limiting example is provided here for clarity:

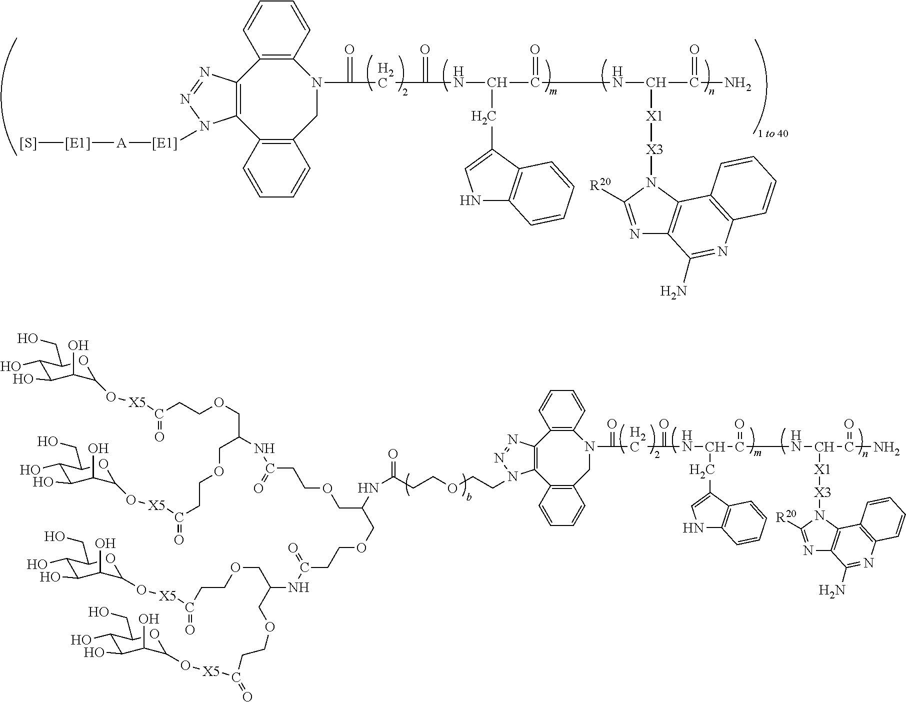

wherein X1, X3 and X5 are each independently any suitable linker molecule; b is an integer number of monomeric units comprising the spacer and is typically between 4 and 36, such as 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35 or 36 monomeric units; S is a solubilizing block, E1 is a N-terminal extension, E2 is a C-terminal extension, A is an antigen selected from tumor antigens, and [ ]denotes that the groups are optional. In some alternative embodiments the peptide antigen conjugates have the formula H-D-U-[E1]-A-[E2]-[S]. In preferred embodiments, the average net charge of the one or more peptide antigen conjugates is greater than or equal to +2, preferably between +2 and +6, more preferably between +3 and +5, and the solubilizing block of the peptide antigen conjugate is present and comprises a poly(amino acid) (or “peptide”) further comprising lysine and/or ornithine residues; the molar ratio of the peptide antigen conjugate to amphiphile is between 4:1 and 1:4, more preferably between 2:1 and 1:2 or about 1:1; and b comprises between 12 to 36 monomeric units, preferably 24 monomeric units.

For certain cases, drug molecules may be incorporated into the hydrophobic block of the amphiphile but not the hydrophobic block of the peptide antigen conjugate, such as:

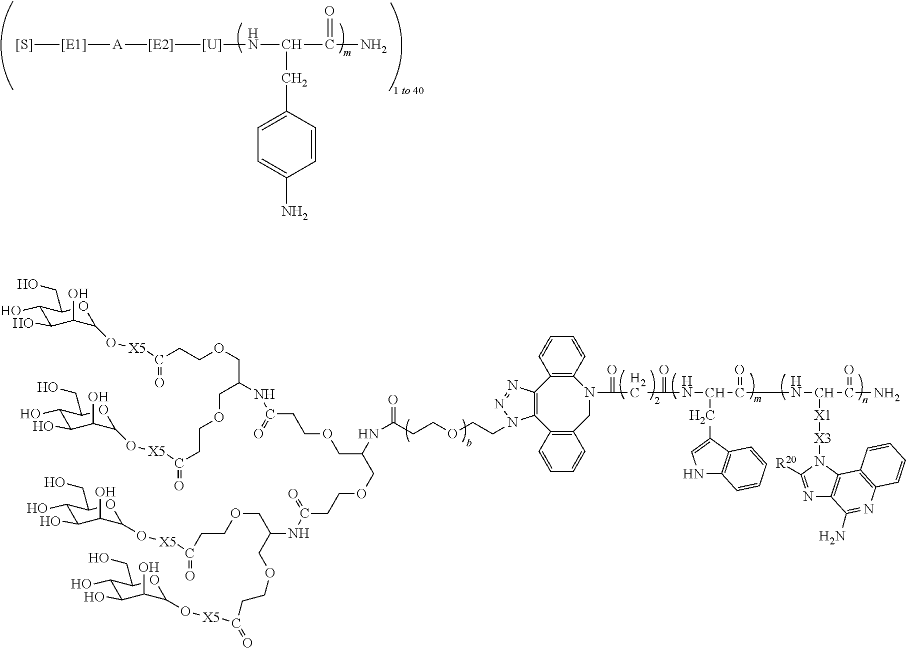

In some embodiments, a peptide antigen conjugate may, for example, comprise a structure as follows:

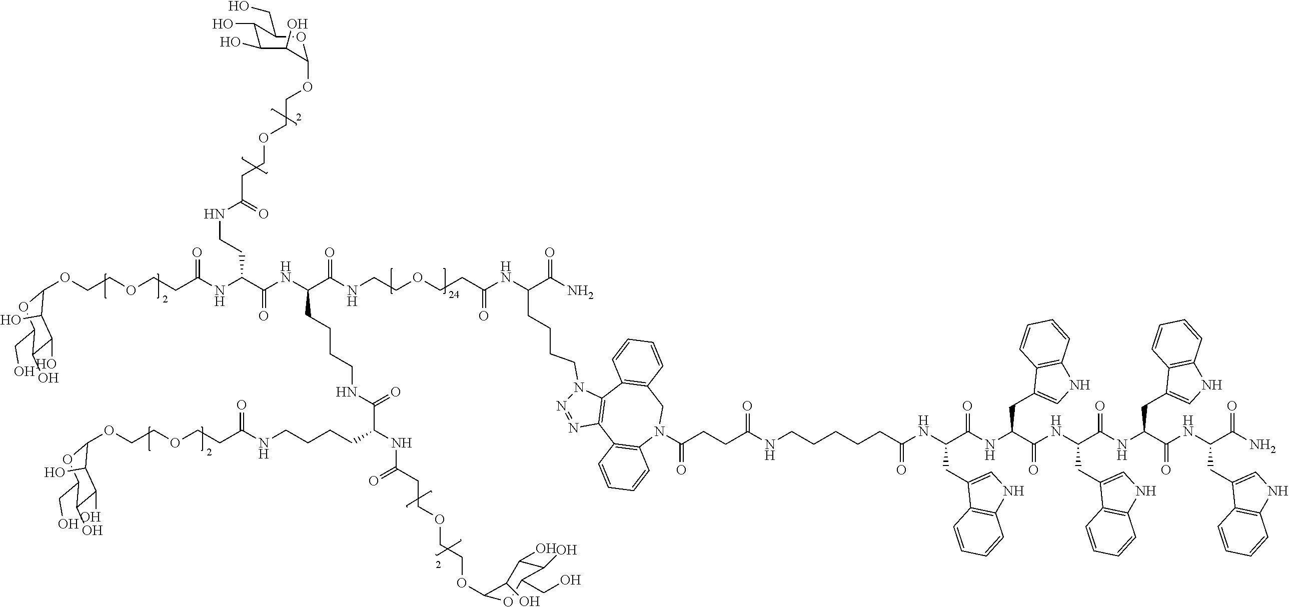

referred to as (Mannose-PEG₃)₄K2K-PEG₂₄-(X-DBCO)-Ahx-W₅ or Tetra(Mannose-PEG₃)₄K2K-PEG₂₄-(X-DBCO)-Ahx-W₅. (See WO 2022/177993, compound 299.)

### D. Peptide Antigen Conjugates of Formula PEG-[E1]-A-[E2]-[U]-H [D]

In some embodiments, a peptide antigen (A) included in a first and/or second treatment herein may be delivered as a polypeptide, for example in some embodiments as part of a peptide antigen conjugate with the formula PEG-[E1]-A-[E2]-[U]-H [D], in which A is the peptide antigen, H is a hydrophobic molecule, PEG is polyethylene glycol, E1 is an N-terminal extension, E2 is a C-terminal extension, U is a linker, D is a drug molecule, [ ] denotes that the group is optional, and a dash (-) indicates a covalent linkage. Thus, in some cases, a conjugate only includes the PEG-A-H components, while in other cases, it may have the formula PEG-E1-A-H, C-A-E2-H, PEG-E1-A-E2-H, PEG-A-U-H, PEG-E1-A-U-H, C-A-E2-U-H, or PEG-E1-A-E2-U-H, in all cases optionally with or without an associated drug D. In such conjugates, the E1, E2, U, and H entities may be as described above for the S-[E1]-A-[E2]-[U]-H [D] conjugates. Thus, in some cases, PEG replaces the solubilizing block S in such conjugates. In some cases, where the drug D is present, it is covalently linked to the hydrophobic molecule H, i.e., providing a formula such as PEG-[E1]-A-[E2]-[U]-H-[D].

In these peptide antigen conjugates, the E1, A, E2, U, H, and D components may be as described herein for the S-[E1]-A-[E2]-[U]-H [D] conjugates, for example. In some cases, the PEG contains from 4 to 45 monomer units, or from 4 to 36 monomer units, such as from 12-36, 12-24, or 24-36. In some embodiments, it was unexpectedly found that shorter PEG lengths were preferred to reduce PEG chain entanglement. Therefore, in some embodiments, the PEG contains from 4 to 24 monomers units, such as 4-20, 4-16, 4-12, 6-24, 6-20, 6-16, 6-12, 8-24, 8-20, 8-16 and 8-12. In some embodiments the PEG is terminated with a methoxy or ethoxy. In other embodiments, the PEG is terminated with an amine or hydroxyl group.

In some embodiments of peptide antigen conjugate of formula PEG-[E1]-A-[E2]-[U]-H [D] conjugates the antigen is a tumor-specific antigen (including viral antigens expressed by tumors). Exemplary antigens (A) are provided above.

Non-limiting examples include peptide antigen conjugates with tumor antigens such as viral antigens (e.g., HPV E6 derived antigen:

(e.g., HPV E6 derived antigen:
(SEQ ID NO: 104)
KHKSAIVTLTYDSEWQRDQFLSQVKIPKT),
self-antigens (e.g., PSA derived:
(SEQ ID NO: 130)
CGGVLVHPQWVLTAAHCIRNKSVILLGRHSLFHPE)
or
tumor neoantigen (e.g., mutant Kras G12C mutant:
(SEQ ID NO: 161)
MTEYKLVVVGACGVGKSALTIQLIQ),
which have the structure
PEG-[E1]-A-[E2]-[U]-H [D], such as:
(SEQ ID NO: 104)
PEG-[E1]-KHKSAIVTLTYDSEWQRDQFLSQVKIPKT-
[E2]-[U]-H [D],
(SEQ ID NO: 130)
PEG-[E1]-CGGVLVHPQWVLTAAHCIRNKSVILLGRHS
LFHPE-[E2]-[U]-H [D],
and
(SEQ ID NO: 161)
PEG-[E1]-MTEYKLVVVGACGVGKSALTIQLIQ-[E2]-
[U]-H [D].

The E1, E2, U, H, and D may be as described in the sections above. In some cases, if a drug D is present, it is covalently linked to the conjugate, such as to the hydrophobic block H, providing a structure PEG-[E1]-A-[E2]-[U]-H-[D]. In some cases, the drug D is an immunostimulant, such as (a) one or more of a TLR-3, TLR-7, TLR-8, TLR-7/8, TLR-9, MDA5, RIG1, or STING agonist, or (b) a molecule that induces Flt3, IL-12, and/or type-I IFN signaling. In some cases, the drug D is a TLR-7/8 agonist.
In the above examples, wherein the PEG is linked at the C-terminus the structure may comprise the formula [D]H-[U]-[E1]-A[E2]-PEG. The E1, E2, U, H, and D may be as described in the sections above. In some cases, if a drug D is present, it is covalently linked to the conjugate, such as to the hydrophobic block H, providing a structure PEG-[E1]-A-[E2]-[U]-H-[D]. In some cases, the drug D is an immunostimulant, such as (a) one or more of a TLR-3, TLR-7, TLR-8, TLR-7/8, TLR-9, MDA5, RIG1, or STING agonist, or (b) a molecule that induces Flt3, IL-12, and/or type-I IFN signaling. In some cases, the drug D is a TLR-7/8 agonist.

Examples with particular antigen sequences A, include:

(SEQ ID NO: 104)
[D]H-[U]-[E1]-KHKSAIVTLTYDSEWQRDQFLSQVK
IPKT-[E2]-PEG,
(SEQ ID NO: 130)
[D]H-[U]-[E1]-CGGVL VHPQWVLTAAHCIRNKSVI
LLGRHSLFHPE-[E2]-PEG,
and
(SEQ ID NO: 161)
[D]H-[U]-[E1]-MTEYKLVVVGACGVGKSALTIQLI
Q-[E2]-PEG

In some embodiments, the PEG is selected from hydroxy-terminated PEG between 4-24 monomer unites in length, and the hydrophobic block H is Ahx-Glu(2B)-Trp-Glu(2B)-Trp-Glu(2B)-NH₂ (SEQ ID NO: 165), wherein 2B is a TLR-7/8 agonist drug, and the above examples become:

(SEQ ID NO: 104)
OH-PEG₄₋₂₄-[E1]-KHKSAIVTLTYDSEWQRDQFLSQ
VKIPKT-[E2]-[U]-H [D]
(SEQ ID NO: 130)
OH-PEG₄₋₂₄-[E1]-CGGVLVHPQWVLTAAHCIRNKSVIL
LGRHSLFHPE-[E2]-[U]-H [D]
(SEQ ID NO: 161)
OH-PEG₄₋₂₄-[E1]-MTEYKLVVVGACGVGKSALTIQLI
Q-[E2]-[U]-H [D]
(SEQ ID NO: 104)
[D]H-[U]-[E1]-KHKSAIVTLTYDSEWQRDQFLSQVK
IPKT-[E2]-PEG₄₋₂₄-OH
(SEQ ID NO: 130)
[D]H-[U]-[E1]-CGGVL VHPQWVLTAAHCIRNKSVI
LLGRHSLFHPE-[E2]-PEG₄₋₂₄-OH
(SEQ ID NO: 161)
[D]H-[U]-[E1]-MTEYKLVVVGACGVGKSALTIQLIQ-
[E2]-PEG₄₋₂₄-OH

In some embodiments above, the hydrophobic block H is Ahx-Glu(2B)-Trp-Glu(2B)-Trp-Glu(2B)-NH₂ (SEQ ID NO: 165), wherein 2B is a TLR-7/8 agonist drug D. In some embodiments, U is presented and is selected from Lys(N3-DBCO). In some embodiments, E1 is included and is VZ. In some embodiments, E2 is included and is SPVZ. Thus, in some nonlimiting examples, the structures are as follows:

(SEQ ID NO: 162)
OH-PEG₄₋₂₄-VZ-KHKSAIVTLTYDSEWORDQFLSQVKIPKT-
SPVZ- Lys(N3-DBCO)-Ahx-Glu(2B)-Trp-Glu(2B)-
Trp-Glu(2B)-NH2,
(SEQ ID NO: 163)
OH-PEG₄₋₂₄-VZ-CGGVLVHPQWVLTAAHCIRNKSVILLGRHS
LFHPE-SPVZ-Lys(N3-DBCO)-Ahx-Glu(2B)-Trp-Glu
(2B)-Trp-Glu(2B)-NH2,
and
(SEQ ID NO: 164)
OH-PEG₄₋₂₄-VZ-MTEYKLVVVGACGVGKSALTIQLIQ-SPVZ-
Lys(N3-DBCO)-Ahx-Glu(2B)-Trp-Glu(2B)-Trp-Glu
(2B)-NH2

An unexpected finding reported herein is that peptide antigen conjugates of formula PEG-[E1]-A-[E2]-[U]-H [D] are better tolerated by the intravenous (IV) route than peptide antigen conjugates of formula C-[E1]-A-[E2]-[U]-H [D]. Therefore, in some embodiments of either the first or second treatment comprising peptide antigen conjugates delivered by the IV route, the peptide antigen conjugate is selected from peptide antigen conjugates of formula PEG-[E1]-A-[E2]-[U]-H [D] or [D]H-[E1]-A-[E2]-[U]-PEG.

### E. Exemplary Amphiphiles

In some embodiments, a peptide antigen conjugate is associated with one or more amphiphiles, for example, to help formation of micelles or to provide further molecules to which drug molecules may associate. In some cases, an amphiphile may have a formula S-[B]-[U]-H [D], where the S, H, and U components may be as described above. In some embodiments, B is a spacer comprising from 4 to 36 PEG units. In some embodiments, a drug D is included, such as an immunostimulant drug, and in some cases it may be covalently attached to the hydrophobic block H, i.e., providing formula S-[B]-[U]-H-[D].

In some embodiments, S of the amphiphile comprises a second or third generation dendrimer; H comprises a poly(amino acid) comprising hydrophobic amino acids (M). In some particular cases, S of an S-[B]-[U]-H formula amphiphile comprises a second or third generation dendrimer; B comprises from 4 to 36 PEG monomeric units; and H comprises a poly(amino acid) comprising hydrophobic amino acids (M). In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a polymer of para amino-phenylalanine. In some embodiments, B comprises from 4 to 36 PEG monomeric units; H of the amphiphile comprises a polymer of para amino-phenylalanine. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a poly(amino acid) comprising hydrophobic amino acids (M) and reactive amino acids (N), that comprise an imidazoquinoline. In some embodiments, B comprises from 4 to 36 PEG monomeric units; H of the amphiphile comprises a poly(amino acid) comprising hydrophobic amino acids (M) and reactive amino acids (N), that comprise an imidazoquinoline. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a poly(amino acid) of tryptophan and reactive amino acids (N) that comprise an imidazoquinoline. In some embodiments, B comprises from 4 to 36 PEG monomeric units; H of the amphiphile comprises a poly(amino acid) of tryptophan and reactive amino acids (N) that comprise an imidazoquinoline. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; B comprises from 4 to 36 PEG monomeric units; SG is present and comprises mannose. In some embodiments, S of the amphiphile comprises a second or third generation dendrimer; H of the amphiphile comprises a poly(amino acid) comprising hydrophobic amino acids (M); SG comprises mannose; and H of the peptide antigen conjugate comprises a poly(amino acid) comprising hydrophobic amino acids (M). In some embodiments, B comprises from 4 to 36 PEG monomeric units; H of the amphiphile comprises a poly(amino acid) comprising hydrophobic amino acids (M); SG comprises mannose. In some embodiments, S of the amphiphile comprises a second or third generation dendrimer; B comprises from 4 to 36 PEG monomeric units; H of the amphiphile comprises a poly(amino acid) comprising hydrophobic amino acids (M); SG comprises mannose. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a polymer of para amino-phenylalanine; SG comprises mannose. In some embodiments, B comprises from 4 to 36 PEG monomeric units; H of the amphiphile comprises a polymer of para amino-phenylalanine; SG comprises mannose. In some embodiments, S of the amphiphile comprises a second or third generation dendrimer; B comprises from 4 to 36 PEG monomeric units; H of the amphiphile comprises a polymer of para amino-phenylalanine; SG comprises mannose. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; and H of the amphiphile comprises a poly(amino acid) comprising hydrophobic amino acids (M) and reactive amino acids (N), that comprise an imidazoquinoline; SG comprises mannose. In some embodiments, B comprises from 4 to 36 PEG monomeric units; and H of the amphiphile comprises a poly(amino acid) comprising hydrophobic amino acids (M) and reactive amino acids (N) that comprise an imidazoquinoline; SG comprises mannose. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; SG comprises mannose. In some embodiments, S of the amphiphile comprises a second or third generation dendrimer; and H of the amphiphile comprises a poly(amino acid) of tryptophan and reactive amino acids (N) that comprise an imidazoquinoline; SG comprises mannose. In some embodiments, B comprises from 4 to 36 PEG monomeric units; H of the amphiphile comprises a poly(amino acid) of tryptophan and reactive amino acids (N) that comprise an imidazoquinoline; SG comprises mannose. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; B comprises from 4 to 36 PEG monomeric units; the amphiphile comprises amino-hexanoic acid. In some embodiments, S of the amphiphile comprises a second or third generation dendrimer; H of the amphiphile comprises a poly(amino acid) comprising hydrophobic amino acids (M); the amphiphile comprises amino-hexanoic acid. In some embodiments, B comprises from 4 to 36 PEG monomeric units; H of the amphiphile comprises a poly(amino acid) comprising hydrophobic amino acids (M); the amphiphile comprises amino-hexanoic acid. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a polymer of para amino-phenylalanine; the amphiphile comprises amino-hexanoic acid. In some embodiments, B comprises from 4 to 36 PEG monomeric units; H of the amphiphile comprises a polymer of para amino-phenylalanine; the amphiphile comprises amino-hexanoic acid. In some embodiments, S of the amphiphile comprises a second or third generation dendrimer; B comprises from 4 to 36 PEG monomeric units; H of the amphiphile comprises a polymer of para amino-phenylalanine; the amphiphile comprises amino-hexanoic acid. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; and H of the amphiphile comprises a poly(amino acid) comprising hydrophobic amino acids (M) and reactive amino acids (N), that comprise an imidazoquinoline; the amphiphile comprises amino-hexanoic acid. In some embodiments, B comprises from 4 to 36 PEG monomeric units; and H of the amphiphile comprises a poly(amino acid) comprising hydrophobic amino acids (M) and reactive amino acids (N) that comprise an imidazoquinoline; the amphiphile comprises amino-hexanoic acid. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; and H of the amphiphile comprises a poly(amino acid) of tryptophan and reactive amino acids (N) that comprise an imidazoquinoline. In some embodiments, B comprises from 4 to 36 PEG monomeric units; H of the amphiphile comprises a poly(amino acid) of tryptophan and reactive amino acids (N) that comprise an imidazoquinoline. In some embodiments, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the amphiphile comprises amino-hexanoic acid. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; B comprises from 4 to 36 PEG monomeric units; the dendrimer monomers comprise hydroxy acids and amino alcohols. In some embodiments, S of the amphiphile comprises a second or third generation dendrimer; H of the amphiphile comprises a poly(amino acid) comprising hydrophobic amino acids (M); the dendrimer monomers comprise hydroxy acids and amino alcohols. In some embodiments, B comprises from 4 to 36 PEG monomeric units; H of the amphiphile comprises a poly(amino acid) comprising hydrophobic amino acids (M); the dendrimer monomers comprise hydroxy acids and amino alcohols. In some cases, such an amphiphile is paired with a peptide antigen conjugate in which H of the peptide antigen conjugate comprises a poly(amino acid) comprising hydrophobic amino acids (M).

In some embodiments, S of the amphiphile comprises a second or third generation dendrimer; H of the amphiphile comprises a polymer of para amino-phenylalanine; the dendrimer monomers comprise hydroxy acids and amino alcohols. In some embodiments, B comprises from 4 to 36 PEG monomeric units; H of the amphiphile comprises a polymer of para amino-phenylalanine; the dendrimer monomers comprise hydroxy acids and amino alcohols. In some embodiments, S of the amphiphile comprises a second or third generation dendrimer; B comprises from 4 to 36 PEG monomeric units; H of the amphiphile comprises a polymer of para amino-phenylalanine; the dendrimer monomers comprise hydroxy acids and amino alcohols. In some cases, such an amphiphile is paired with a peptide antigen conjugate in which H of the peptide antigen conjugate comprises a poly(amino acid) comprising hydrophobic amino acids (M).

In some embodiments, B comprises from 4 to 36 PEG monomeric units; and H of the amphiphile comprises a poly(amino acid) comprising hydrophobic amino acids (M) and reactive amino acids (N) that comprise an imidazoquinoline; the dendrimer monomers comprise hydroxy acids and amino alcohols. In some embodiments, S of the amphiphile comprises a second or third generation dendrimer; B comprises from 4 to 36 PEG monomeric units; H of the amphiphile comprises a poly(amino acid) comprising hydrophobic amino acids (M) and reactive amino acids (N) that comprise an imidazoquinoline; the dendrimer monomers comprise hydroxy acids and amino alcohols. In some embodiments, S of the amphiphile comprises a second or third generation dendrimer; and H of the amphiphile comprises a poly(amino acid) of tryptophan and reactive amino acids (N) that comprise an imidazoquinoline; the dendrimer monomers comprise hydroxy acids and amino alcohols. In some cases, such an amphiphile is paired with a peptide antigen conjugate in which H of the peptide antigen conjugate comprises a poly(amino acid) comprising hydrophobic amino acids (M).

In some embodiments, B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols. In some embodiments, S of the amphiphile comprises a second or third generation dendrimer; B comprises from 4 to 36 PEG monomeric units; H of the amphiphile comprises a poly(amino acid) of tryptophan and reactive amino acids (N) that comprise an imidazoquinoline; the dendrimer monomers comprise hydroxy acids and amino alcohols.

In some embodiments of amphiphiles of formula S-[B]-[U]-H, the solubilizing block (S) comprises a dendron amplifier wherein the focal point is linked to the hydrophobic block (H) either directly or indirectly via a spacer (B) and/or Linker U and the terminal functional groups (FGt) either are unlinked and serve as the solubilizing groups or are linked to a solubilizing group (SG). Solubilizing groups (SG) are any molecules that are hydrophilic and/or charged; preferred solubilizing groups (SG) are described throughout the specification.

In some embodiments of amphiphiles of formula S-[B]-[U]-H-D, peptide antigen conjugates of formula [S]-[E1]-A-[E2]-[U]-H-D or H-D-[U]-[E1]-A-[E2]-[S]) and drug molecule conjugates of formula H-D, the hydrophobic block (H) comprises a dendron amplifier wherein the focal point is linked to either (i) a solubilizing block (S) either directly or indirectly via a spacer (B) and/or Linker U, (ii) an antigen (A) either directly or indirectly via an extension (E1 or E2) and/or Linker U; or (iii) a drug molecule either directly or via a Linker X1.

In some embodiments, the hydrophobic block (H) comprises a dendron amplifier and the terminal functional groups (FGt) are linked to hydrophobic drug molecules. In such embodiments, the focal point is linked to either (i) a solubilizing block (S) either directly or indirectly via a spacer (B) and/or Linker U, (ii) an antigen (A) either directly or indirectly via an extension (E1 or E2) and/or Linker U; or (iii) is unreacted or capped with a terminal group, such as an acetyl group. Capped or capping refers to the modification of a functional group, such as FGt, to make it less reactive and/or have neutral charge at pH 7.4. For example, an amine may be capped with an activated carboxylic acid (e.g., acetyl chloride) to result in a relatively less reactive amide; or, e.g., a strained alkyne may be capped with an alkyl-azide to result in a relatively less reactive triazole.

### F. Exemplary Nucleic Acid Vaccines

In some embodiments, a vaccine administered in a first or second treatment herein is a nucleic acid vaccine that encodes an tumor antigen, such as those described above, and optionally further encodes at least one additional polypeptide, such as an immunostimulant or the like.

Viral vectors have the advantage of being recognized as foreign by the immune system, inducing innate and adaptive immune responses. Example viral vectors for nucleic acid vaccines include adenovirus, adeno-associated virus (AAV), rhabdovirus, primate adenovirus vectors such as ChAdOx, vaccinia virus vectors such as modified vaccinia Ankara (MVA) virus vectors, and pox virus vectors. Further examples of viral vectors used in nucleic acid cancer vaccines include pox viral vectors such as vaccinia, rV-CEA, rA-CEA, Alvac, Alvac-CEA-B7.1, Alvac-CEA, rV-CEA-TRICOM, rF-CEA-TRICOM, Panvac, and Panvac-V/F. In some cases, a polynucleotide vaccine may be delivered by a non-viral DNA or RNA vector. Polynucleotide vectors may comprise a region encoding one or more tumor antigen polypeptides, for example. In some cases, such vectors may also encode molecules that either induce production of antigen-specific CD4 and/or CD8 T cells or that induce an inflammatory response, e.g., systemic or tumor-specific inflammation, such as certain cytokines or immune checkpoint molecules. Examples include CXCL8, Flt3, GM-CSF, IL1α, IL1β, IL1ra, IL2, IL3, IL4, IL5, IL6, IL7, IL8, IL9, IL10, IL11, IL12, IL13, IL15, IL16, IL17, IL18, IL19, IL20, IL21, IL22, IL23, IL24, IL25, IFNα, IFNβ, IFNγ, TGFβ, TNFα, TNFβ, 4-1BB, B7-1, B7-2, CD27, CD28, CD40, CD80, CD86, CTLA-4, ICOS, PD-1, CD27, CD30, CD137, CD40L, OX40, GITR, TIM-1, TIM-2, and TIM-3.

In some embodiments, a nucleic acid vaccine is a Chimpanzee adenovirus (ChAdOx) vaccine. ChAdOx vectors comprise a chimpanzee adenovirus construct that has been modified to render it replication-deficient. In some embodiments, the vector is a ChAdOx1, ChAd2, or ChAd63 vector. See, for example, Van Doremalen et al., Nature 586L578-582 (2020); Hitchings et al., Nat Commun. 12(1):6220 (2021); International Patent Publication no. WO2012/172277.

### G. Exemplary Adoptive Cell Therapies

In some embodiments, a first treatment comprises adoptive cell therapy (ACT). Adoptive cell therapy (ACT) is a strategy to modify the immune system to recognize tumor cells and thus carry out an anti-tumor effector function. ACT may comprise, in some embodiments, tumor-infiltrating lymphocytes (TILs), or gene-modified T cells expressing novel T cell receptors (TCRs) or chimeric antigen receptors (CARs).

For example, in TIL therapy, tumor-resident T cells are isolated and expanded after surgical resection of a tumor. Thereafter, the TILs are further expanded in a rapid expansion protocol. Before intravenous (IV) adoptive transfer into a patient, the patient is treated with a lymphodepleting preparative regimen and subsequent support of IL2.

TCRs and CARs are therapies with genetically modified peripheral blood T cells. Peripheral blood T cells can be isolated and genetically modified in vitro with viral vectors comprising an antigen to express a specific TCR or a specific CAR that targets the antigen, for example. With TCRs and CARs, large pools of tumor specific T cells can be generated. Antigen recognition by the modified TCRs requires antigen presentation via the major histocompatibility complex (MHC). However, cancer cells can escape T-cell mediated immune responses by downregulation or loss of the MHC expression. To circumvent the need for the presence of MHC on cancer cells for recognition by cancer-specific T cells, CARs were developed. CAR-modified T cells (CAR-T cells) may have the same effector function as TCR-modified T cells, but function independently of MHC expression.

Examples of CAR-T cell therapies include, for example, treatments for blood cancers such as B-cell acute lymphoblastic leukemia (ALL), B-cell non-Hodgkin lymphoma (NHL), follicular lymphoma, mantle cell lymphoma (MCL), and multiple myeloma. Examples of CAR-T cell therapies include tisangenlecleucel (Kymriah), axicabtagene ciloleucel (Yescarta), brexucabtangene autoleucel (Tecartus), lisocabtagene maraleucel (Breyanzi), idecabtagene vicleucel (Abecma), and ciltacabtagene autoleucel (Carvykti). In some examples, the CAR T cell therapies target one or more antigens on B cells, such as CD19 and BCMA.

### H. Exemplary Immunostimulants

In methods herein, a second treatment may comprise an immunostimulant. In some embodiments, an immunostimulant comprises one or more immunostimulants that induce Type-I IFNs, including agonists of TLR-3, TLR-7, TLR-8, TLR-9, RLR and STING. In some embodiments, an immunostimulant may comprise one or more immunomodulatory drug molecules are typically selected from immunostimulants that induce proinflammatory cytokines and/or Type-I IFNs, including agonists of TLR-1, TLR-2, TLR-3, TLR-4, TLR-5, TLR-6, TLR-7, TLR-8, TLR-9, CLRs, NLRs or combinations thereof. In some embodiments, an immunostimulant comprises one or more of a TLR-3, TLR-7, TLR-8, TLR-7/8, TLR-9, MDA5, RIG1, or STING agonist.

In some cases, immunostimulants are selected from pattern recognition receptor (PRR) agonists. Non-limiting examples of pattern recognition receptor (PRR) agonists include TLR-1/2/6 agonists (e.g., lipopeptides and glycolipids, such as Pam2cys or Pam3cys lipopeptides); TLR-3 agonists (e.g., dsRNA, such as PolyI:C, and nucleotide base analogs); TLR-4 agonists (e.g., lipopolysaccharide (LPS) derivatives, for example, monophosphoryl lipid A (MPL) and small molecule derivatives or analogs of pyrimidoindole); TLR-5 agonists (e.g., Flagellin); TLR-7 & -8 agonists (e.g., ssRNA and nucleotide base analogs, including derivatives of imidazoquinolines, hydroxy-adenine, benzonahpthyridine and loxoribine); and TLR-9 agonists (e.g., unmethylated CpG); Stimulator of Interferon Genes (STING) agonists (e.g., cyclic dinucleotides, such as cyclic diadenylate monophosphate and diABZI or derivatives thereof); C-type lectin receptor (CLR) agonists (such as various mono, di, tri and polymeric sugars that can be linear or branched, e.g., mannose, Lewis-X tri-saccharides, etc.); RIG-I-like receptor (RLR) agonists; NOD-like receptor (NLR) agonists (such as peptidogylcans and structural motifs from bacteria, e.g., meso-diaminopimelic acid and muramyl dipeptide); and combinations thereof. In some embodiments, the immunostimulant selected for use in a vaccine is selected from inorganic salts, including aluminum salts and or oils, such as squalene and its derivatives (e.g., MF59 and the like).

In some embodiments, an immunostimulant herein comprises a Toll-like receptor (TLR) agonist, such as an agonist of TLR-3, TLR-7, TLR-8, TLR-7/8, or TLR-9. One example is a TLR-7/8 agonist, such as an imidazoquinoline-based TLR-7/8 agonist. For example, the immunostimulant can be Imiquimod (R2137) or Resiquimod (R2148), which are approved by the FDA for human use for certain indications and uses. In some cases, the immunostimulant comprises a TLR-7 agonist, a TLR-8 agonist and/or a TLR-7/8 agonist. Numerous such agonists are known, including many different imidazoquinoline compounds.

For example, imidazoquinolines are synthetic immunomodulatory drugs that act by binding Toll-like receptors-7 and/or -8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod or resiquimod.

In some embodiments, an immunostimulant comprises a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the immunostimulant is an imidazoquinoline with the formula:

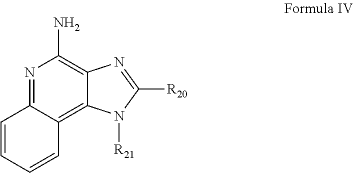

In Formula IV, R²⁰ is selected from one of hydrogen, optionally-substituted lower alkyl, or optionally-substituted lower ether; and R²¹ is selected from one of optionally substituted arylamine, or optionally substituted lower alkylamine. R²¹ may be optionally substituted to a linker that links to a polymer. In some embodiments, such as where the immunostimulant of Formula IV is used as a drug (D) in a peptide antigen conjugate, in some compounds wherein R²¹ was selected from a lower alkylamine, while the compound was less potent than R²¹ selected from an arylamine, the quality of response was improved. Thus, moderate potency compounds of Formula IV led to better quality responses.

In some embodiments, the R²⁰ included in Formula IV can be selected from hydrogen,

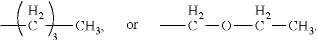

In some embodiments, R²¹ can be selected from,

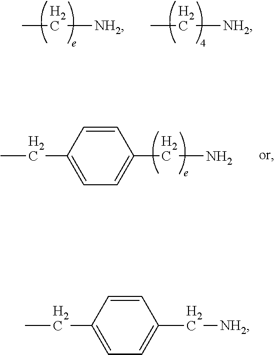

wherein e denotes the number of methylene unites is an integer from 1 to 4.

In some embodiments, R²¹ can be

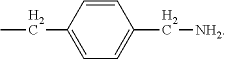

In some embodiments, R²¹ can be

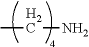

In some embodiments, R²⁰ can be

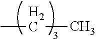

### and R²¹ can be

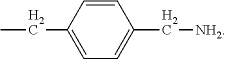

In some embodiments, at least one D is

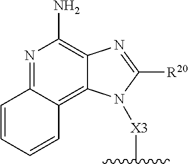

wherein R²⁰ is selected from H, alkyl, alkoxyalkyl, aryl, heteroaryl, aminoalkyl, amide and ester; and X3 is selected from alkyl, alkoxyalkyl, aralkyl, heteroaralkyl, aryl, heteroaryl and carboxy.

In some embodiments, wherein, R²⁰ is selected from H, alkyl and alkoxyalkyl; and X3 is selected from alkyl and aralkyl. In other embodiments, R²⁰ is butyl.

In some embodiments, X3 is alkyl.

In some embodiments, immunostimulatory drug molecules that are hydrophobic and/or amphiphilic are administered. Non-limiting examples include, squalene-based immunostimulants; lipid-based PRR agonists, such as mincle receptor agonists (e.g., trehalose dimycolate and trehalose dibehenate) lipopolysaccharide-based agonists of TLR-4, and lipopeptide-based agonists of TLR-1/2 and TLR-2/6; heteroaryl-based agonists of TLR-4 (e.g., pyrimidoindole); agonists of TLR-7/8 (e.g., imidazoquinolines and benzonaphthyridines) and STING (e.g., diABZI); and various hydrophobic immunosuppressants, including but not limited to certain inhibitors of mTOR/PI3K/AKT (e.g., KU-0062794, Torin 1, Torin 2, etc.), CDK8/19 (e.g., Cortistatin), retinoic acid-related orphan gamma t (RORγt) (e.g., SR1555) and histone deacetylase (HDACs) (e.g., TMP269), as well as certain agonists of aryl hydrocarbon receptors (AHR) (e.g., indole, indolo[3,2-b]carbazole (ICZ) and 3,3 diindolomethane), retinoic acid receptors (RAR) (e.g., all-trans retinoic acid, TTNPB (cas: 71441-28-6), AM580, BMS753, BMS961 and the like) and adenosine receptor (e.g., UK-432,097).

In some embodiments, immunostimulants are administered alone, for example, as second treatments herein. In other cases, an immunostimulant is administered with other compounds, such as an amphiphile, such as an amphiphile of formula S-[B]-[U]-H [D], in which the immunostimulant may comprise the drug molecule D. In some cases, the immunostimulant, represented as drug molecule D, is covalently attached to the hydrophobic block H of the amphiphile, e.g., S-[B]-[U]-H-[D], where “-” indicates a covalent bond. In other cases, the immunostimulant is not bound to the amphiphile covalently. In some embodiments, an immunostimulant, such as for a second treatment herein, may be administered in conjunction with or as part of a vaccine composition. For instance, it could be administered along with a nucleic acid vaccine. Or it could be administered along with a peptide antigen conjugate vaccine, and in some cases as a drug molecule D that is incorporated into the vaccine, as described herein.

Accordingly, in some cases, immunostimulants are linked to hydrophobic blocks to form drug molecule conjugates that are incorporated into peptide antigen conjugates, optionally further comprising amphiphiles, for example, through non-covalent interactions. In still other embodiments, immunostimulants may be incorporated into nanoparticles comprising peptide antigen conjugates through covalent attachment to an amphiphile (if present) and/or to peptide antigen conjugates. In some embodiments, a vaccine comprises nanoparticles that comprise amphiphiles, one or more peptide antigen conjugates and immunostimulants selected from imidazoquinolines, wherein the imidazoquinolines are linked to the hydrophobic blocks of the amphiphiles and/or peptide antigen conjugates. Exemplary compositions of vaccines are described in greater detail elsewhere.

In some embodiments, cytokine or chemokine levels are assessed in a sample from the subject, such as a blood sample, in order to determine the effect of the immunostimulant treatment. Cytokines and chemokines are secreted proteins with growth, differentiation, and activation functions that regulate and determine the nature of immune responses. They also control immune cell trafficking and the cellular arrangement of immune organs. In some embodiments, one or more chemokines are elevated. Examples of cytokines include CXCL8, Flt3, GM-CSF, IL1α, IL1β, IL1ra, IL2, IL3, IL4, IL5, IL6, IL7, IL8, IL9, IL10, IL11, IL12, IL13, IL15, IL16, IL17, IL18, IL19, IL20, IL21, IL22, IL23, IL24, IL25, IFNα, IFNβ, IFNγ, TGFβ, TNFα, and TNFβ. Examples of chemokines include CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CX3CL1. Accordingly, in some embodiments, methods herein further comprise determining the level of one or more cytokines, such as those listed above, following the first and/or second treatment. In some cases, the level may be determined in a blood sample from the subject, or in a tumor biopsy from the subject. In some cases, the treatment may be modified, for example, if cytokine levels indicate that systemic and/or tumor specific inflammation has not been induced by the immunostimulant.

In certain embodiments, the immunostimulant is intended to induce signaling by type-I interferons (IFN). In some embodiments, such signaling may be assessed by determining the level of IL12 or IFN-alpha in a blood sample from a subject. For example, increased IFN-I signaling leads to an increase in IL12 and/or IFN-alpha. Accordingly, in some embodiments, methods herein further comprise determining the level of IL12 and/or IFN-alpha following the first and/or second treatment. In some cases, the level may be determined in a blood sample from the subject, or in a tumor biopsy from the subject. In some cases, the treatment may be modified, for example, if cytokine levels indicate that systemic and/or tumor specific inflammation has not been induced by the immunostimulant.

In some embodiments, the levels of costimulatory molecules are assessed in a sample from the subject, such as a blood sample. Costimulatory molecules are cell surface molecules that act to amplify or counteract the initial activating signals provided to T cells from the T cell receptor (TCR) following its interaction with an antigen/major histocompatibility complex (MHC), thereby influencing T cell differentiation and fate. In some embodiments, one or more costimulatory molecules are elevated. Examples of costimulatory molecules include 4-1BB, B7-1, B7-2, CD27, CD28, CD40, CD80, CD86, CTLA-4, ICOS, PD-1, CD27, CD30, CD137, CD40L, OX40, GITR, TIM-1, TIM-2, and TIM-3. In some embodiments, the levels of ligands of the costimulatory molecules are assessed. Examples of ligands of costimulatory molecules include 4-1BBL, B7-1, B7-2, CTLA-4, ICOS-L, PDL-1, PDL-2, CD70, CD30L, CD40, CD80, CD86, OX40L, GITRL, TIM-1, TIM-4, H-ferritin, Semaphorin 4A (Sem4A), and Galectin-9. Accordingly, in some embodiments, methods herein further comprise determining the level of one or more co-stimulatory molecules, such as those listed above, following the first and/or second treatment. In some cases, the level may be determined in a blood sample from the subject, or in a tumor biopsy from the subject.

In some cases, the treatment may be modified, for example, if the levels indicate that systemic and/or tumor specific inflammation has not been induced by the immunostimulant.

### IV. Additional Exemplary First and Second Treatment Combinations

### A. First and Second Treatments Comprising Peptide Antigen Conjugates

Further, more particular combinations of first and second treatment herein are as follows. In certain cases herein, both the first treatment and the second treatment comprise peptide antigen conjugate vaccines, wherein the peptide antigen conjugate administered as the second treatment comprises or is administered in conjunction with an immunostimulant, wherein the first treatment is administered by IM or IV and the second treatment is administered by IV. In some cases, the antigen administered in the first and second treatments is the same. In some cases, the immunostimulant is also administered with both the first and with the second treatments.

In some cases, the first treatment comprises a peptide antigen conjugate administered by IM and a second treatment comprises a peptide antigen conjugate administered by IV, in some cases each comprising the same antigen and/or the same immunostimulant. In some cases, one or both peptide antigen conjugates further comprises an amphiphile. In some cases, the chemistry of the peptide antigen conjugates is generally also the same, however, there may be some differences to better accommodate IV and IM administration methods. For example, it was unexpectedly found herein that a peptide antigen conjugate formula of PEG-[E1]-A-[E2]-[U]-H [D] along with an amphiphile, such as of formula S-[B]-[U]-H [D], may be safer for IV administration than a conjugate of formula S-[E1]-A-[E2]-[U]-H [D]. Thus, in some cases a peptide antigen conjugate of formula S-[E1]-A-[E2]-[U]-H [D] may be used as a first IM treatment and a peptide antigen conjugate of PEG-[E1]-A-[E2]-[U]-H [D] along with an amphiphile may be used as a second IV treatment. Or alternatively the peptide antigen conjugate of PEG-[E1]-A-[E2]-[U]-H [D] optionally further comprising an amphiphile, may be administered both as a first treatment by IM and as a second treatment by IV. Alternatively, a peptide antigen conjugate of PEG-[E1]-A-[E2]-[U]-H [D] along with an amphiphile may be used as both a first IV treatment and a second IV treatment, with at least the second treatment conjugate further comprising an immunostimulant. In some cases, where drug D is present, it is covalently linked to the hydrophobic block (H), i.e., PEG-[E1]-A-[E2]-[U]-H-D, S-[B]-[U]-H-D, or S-[E1]-A-[E2]-[U]-H-D, where the dash (-) indicates a covalent linkage.

Accordingly, in some such cases, the method of treating a cancer subject may comprise (a) administering a first treatment to the subject by IV or IM and (b) following a time interval, administering a second treatment by IV, wherein the first treatment comprises a first peptide antigen conjugate of formula S-[E1]-A-[E2]-[U]-H [D] or of formula PEG-[E1]-A-[E2]-[U]-H [D] as described herein, i.e., wherein:
A is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage; and wherein the second treatment comprises a second antigen conjugate or antigen drug conjugate of formula PEG-[E1]-A-[E2]-[U]-H [D], and wherein the second antigen conjugate further comprises or is administered together with an immunostimulant as described herein. The S, E1, E2, U, H, and D components of the peptide antigen conjugate may be as described in the sections above, and the antigen A may also comprise those described in the sections above. In some cases, S is a charged molecule C that comprises one or more functional groups that are charged at physiological pH. In some cases, for example, with a conjugate comprises a charged molecule C, the peptide antigen conjugate has a net electrostatic charge greater than or equal to +3 or less than or equal to −3 in an aqueous buffer at a pH of 7.4. In some cases, the hydrophobic molecule (H) is water insoluble at pH 7.4. An immunostimulant may be administered in conjunction with a conjugate herein, or it may be incorporated in the peptide antigen conjugate, for example as a drug (D). In some cases, where the immunostimulant is present as a drug D, it is covalently linked to the hydrophobic block (H), i.e., S-[E1]-A-[E2]-[U]-H-D or PEG-[E1]-A-[E2]-[U]-H-D, where the dash (-) indicates a covalent linkage. In some cases an immunostimulant may comprise (a) one or more of a TLR-3, TLR-7, TLR-8, TLR-7/8, TLR-9, MDA5, RIG1, or STING agonist, and/or (b) a molecule that induces Flt3, IL-12, and/or type-I IFN signaling. In some cases, both the first and second conjugates comprise the immunostimulant.

In some cases, the immunostimulant is a TLR-7/8 agonist as described herein. In some cases, the first treatment comprises a conjugate of formula S-[E1]-A-[E2]-[U]-H [D] administered IM and the second treatment comprises a conjugate of formula PEG-[E1]-A-[E2]-[U]-H [D] administered IV. In some cases, the antigen is the same in both the S- and the PEG-conjugates. In some cases, the -[E1]-A-[E2]-[U]-H [D] components of the S- and PEG-conjugates are the same. In some cases, where drug D is present, it is covalently linked to the hydrophobic block (H), i.e., S-[E1]-A-[E2]-[U]-H-D or PEG-[E1]-A-[E2]-[U]-H-D, where the dash (-) indicates a covalent linkage. In some cases, the peptide antigen conjugate further comprises an amphiphile, such as an amphiphile described herein of the formula S-[B]-[U]-H [D], i.e., wherein S is a solubilizing block, H is a hydrophobic block, B is an extension, U is a linker, D is a drug molecule, [ ] denotes that the group is optional, and a dash (-) indicates a covalent linkage. In some cases, where drug D is present in the amphiphile, it is covalently linked to the hydrophobic block (H), i.e., S-[B]-[U]-H-D, where the dash (-) indicates a covalent linkage. In some cases, the first treatment provides antigen-specific CD4+ and/or CD8+ T cells in the subject and the second treatment induces systemic and/or tumor specific inflammation in the subject. In some cases, the time interval (T) between the first and second treatments is at least 5 days. In some cases, it is 5 to 90 days, 5 to 60 days, 5 to 30 days, 5 days to three weeks, one week to three weeks, one week to two weeks, 3 to 28 days, 5 to 28 days, 5 to 14 days, 7 to 28 days, 3 to 21 days, 5 to 21 days, 7 to 21 days, 3 to 14 days, 5 to 14 days, 7 to 14 days, 14 to 28 days, or 14 to 21 days. In some cases, it is 7-28 days. In some cases, it is 7-21 days. In some cases, the dose of the S-[E1]-A-[E2]-[U]-H [D] or PEG-[E1]-A-[E2]-[U]-H [D] conjugate is 250 nmol-40,000 nmol total conjugate, 500-20,000 nmol total conjugate, or 1000-10,000 nmol total conjugate. In some cases, the first treatment may be administered more than once prior to the time interval (T) and administration of the second treatment.

In some cases, the method further comprises determining one or more of the level of CD8+ T cells, IFN-alpha, and IL12 or other cytokines or co-stimulatory molecules in a sample from the subject following the first and/or second treatment. A sample may be a blood sample or a tumor biopsy, for example.

B. Peptide Antigen Conjugates Followed by Immunostimulants and/or Amphiphiles

In some embodiments, the method of treating a cancer subject may comprise (a) administering a first treatment to the subject by IV or IM and (b) following a time interval, administering a second treatment by IV, wherein the first treatment comprises a first peptide antigen conjugate of formula S-[E1]-A-[E2]-[U]-H [D] or of formula PEG-[E1]-A-[E2]-[U]-H [D] as described herein, i.e., wherein:
A Is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage; and wherein the second treatment comprises an immunostimulant as described herein, which in some cases may comprise (a) one or more of a TLR-3, TLR-7, TLR-8, TLR-7/8, TLR-9, MDA5, RIG1, or STING agonist, and/or (b) a molecule that induces Flt3, IL-12, and/or type-I IFN signaling. The S, E1, E2, U, H, and D components of the peptide antigen conjugate may be as described in the sections above, and the antigen A may also comprise those described in the sections above. In some cases, S is a charged molecule C that comprises one or more functional groups that are charged at physiological pH. In some cases, such as with a charged molecule solubilizing block, the peptide antigen conjugate has a net electrostatic charge greater than or equal to +3 or less than or equal to −3 in an aqueous buffer at a pH of 7.4. In some cases, the hydrophobic molecule (H) is water insoluble at pH 7.4. In some cases, both the first and second peptide antigen conjugates comprise the immunostimulant. In some cases, the immunostimulant is a TLR-7/8 agonist as described herein. The immunostimulant may be administered separately or may be incorporated within the conjugate as a drug molecule (D). In some cases, where the immunostimulant is present as a drug D, it is covalently linked to the hydrophobic block (H), i.e., S-[E1]-A-[E2]-[U]-H-D or PEG-[E1]-A-[E2]-[U]-H-D, where the dash (-) indicates a covalent linkage.

In some cases, the first treatment comprises a peptide antigen conjugate of formula S-[E1]-A-[E2]-[U]-H [D] administered IM and the second treatment comprises a peptide antigen conjugate of formula PEG-[E1]-A-[E2]-[U]-H [D] administered IV. In some cases, where drug D is present, it is covalently linked to the hydrophobic block (H), i.e., S-[E1]-A-[E2]-[U]-H-D or PEG-[E1]-A-[E2]-[U]-H-D, where the dash (-) indicates a covalent linkage. In some cases, the second treatment further comprises an amphiphile or an amphiphile plus drug (D), such as of the formula S-[B]-[U]-H [D], i.e., wherein S is a solubilizing block, H is a hydrophobic block, B is an extension, U is a linker, D is a drug molecule, [ ] denotes that the group is optional, and a dash (-) indicates a covalent linkage. In some cases, where drug D is present in the amphiphile, it is covalently linked to the hydrophobic block (H), i.e., S-[B]-[U]-H-D, where the dash (-) indicates a covalent linkage. The drug D in the amphiphile may be an immunostimulant or may be a further drug. Accordingly, in some cases, an amphiphile-drug may also be used as the second treatment immunostimulant.

In some cases, the first treatment provides antigen-specific CD4+ and/or CD8+ T cells in the subject and the second treatment induces systemic and/or tumor specific inflammation in the subject. In some cases, the time interval (T) between the first and second treatments is at least 3 days. In some cases, the time interval (T) between the first and second treatments is at least 5 days. In some cases, it is 5 to 90 days, 5 to 60 days, 5 to 30 days, 5 days to three weeks, one week to three weeks, one week to two weeks, 3 to 28 days, 5 to 28 days, 5 to 14 days, 7 to 28 days, 3 to 21 days, 5 to 21 days, 7 to 21 days, 3 to 14 days, 5 to 14 days, 7 to 14 days, 14 to 28 days, or 14 to 21 days. In some cases, it is 3-28 days. In some cases, it is 7-21 days. In some cases, the dose of the S-[E1]-A-[E2]-[U]-H [D] conjugate of the first treatment is 250 nmol-40,000 nmol total conjugate, 500-20,000 nmol total conjugate, or 1000-10,000 nmol total conjugate. In some cases the immunostimulant is a TLR-7/8 agonist, which is administered at a dose of 750 to 120,000 nmol, or 3000 to 30,000 nmol. In some cases, the first treatment may be administered more than once prior to the time interval (T) and administration of the second treatment. In some cases, where drug D is present, it is covalently linked to the hydrophobic block (H), i.e., S-[E1]-A-[E2]-[U]-H-D, where the dash (-) indicates a covalent linkage.

In some cases, the method further comprises determining one or more of the level of CD8+ T cells, IFN-alpha, and IL12 or other cytokines or co-stimulatory molecules in a sample from the subject following the first and/or second treatment. A sample may be a blood sample or a tumor biopsy, for example.

### C. Combinations of Peptide Antigen Conjugates and Polynucleotide Vectors

In some embodiments, the method of treating a cancer subject may comprise (a) administering a first treatment to the subject by IV or IM and (b) following a time interval, administering a second treatment by IV, wherein the first treatment comprises a peptide antigen conjugate of formula S-[E1]-A-[E2]-[U]-H [D] or of formula PEG-[E1]-A-[E2]-[U]-H [D] as described herein, i.e., wherein:
A is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage; and wherein the second treatment comprises a polynucleotide vaccine vector optionally encoding a tumor antigen. The S, E1, E2, U, H, and D components of the peptide antigen conjugate may be as described in the sections above, and the antigen A may also comprise those described in the sections above. In some cases, S is a charged molecule C that comprises one or more functional groups that are charged at physiological pH. In some cases, the peptide antigen conjugate has a net electrostatic charge greater than or equal to +3 or less than or equal to −3 in an aqueous buffer at a pH of 7.4. In some cases, the hydrophobic molecule (H) is water insoluble at pH 7.4. In some cases, the first and/or second treatment also comprises administering an immunostimulant comprising (a) one or more of a TLR-3, TLR-7, TLR-8, TLR-7/8, TLR-9, MDA5, RIG1, or STING agonist, and/or (b) a molecule that induces Flt3, IL-12, and/or type-I IFN signaling. Such a molecule may be administered in conjunction with a vaccine or may be comprised within a vaccine composition, such as a drug (D) in a peptide antigen conjugate. In some cases, where drug D is present, it is covalently linked to the hydrophobic block (H), i.e., S-[E1]-A-[E2]-[U]-H-D or PEG-[E1]-A-[E2]-[U]-H-D, where the dash (-) indicates a covalent linkage. In some cases, both the first and second treatments comprise the immunostimulant. In some cases, the immunostimulant is a TLR-7/8 agonist as described herein. In some cases, the polynucleotide vector may further encode an immunostimulant or immunomodulatory protein.

In some cases, the first treatment comprises a peptide antigen conjugate of formula S-[E1]-A-[E2]-[U]-H [D] administered IM. In some cases, the first treatment comprises a peptide antigen conjugate of formula PEG-[E1]-A-[E2]-[U]-H [D] administered IV. In some cases, where drug D is present, it is covalently linked to the hydrophobic block (H), i.e., S-[E1]-A-[E2]-U-H-D, where the dash (-) indicates a covalent linkage. In some cases, the first treatment further comprises an amphiphile or an amphiphile plus further drug (D), such as of the formula S-[B]-[U]-H [D], i.e., wherein S is a solubilizing block, H is a hydrophobic block, B is an extension, U is a linker, D is a drug molecule, [ ] denotes that the group is optional, and a dash (-) indicates a covalent linkage. In some cases, where drug D is present in the amphiphile, it is covalently linked to the hydrophobic block (H), i.e., S-[B]-[U]-H-D, where the dash (-) indicates a covalent linkage. In some cases, the vaccines of the first and second treatments deliver the same antigen to the subject, i.e., the first treatment comprises a peptide antigen, while the second treatment encodes the same peptide antigen. In some cases, the polynucleotide vaccine of the second treatment is an adenovirus, adeno-associated virus, rhabdovirus, ChAdOx, MVA virus, DNA or RNA vector encoding a tumor antigen. In some cases, it is a ChAdOx vector, such as ChAdOx1.

For example, an unexpected finding herein is that peptide antigen conjugates administered by IM as a first treatment, followed by a ChAdOx vector administered IV as a second treatment led to superior safety and a higher level of T cells in vivo compared with administration of peptide antigen conjugates by IV as a first treatment followed by ChAdOx IV as a second treatment and also compared to administration of ChAdOx vectors by IV as both first and second treatment.

In some cases, the first treatment provides antigen-specific CD4+ and/or CD8+ T cells in the subject and the second treatment induces systemic and/or tumor specific inflammation in the subject. In some cases the time interval (T) between the first and second treatments is at least 3 days. In some cases the time interval (T) between the first and second treatments is at least 5 days. In some cases it is 5 to 90 days, 5 to 60 days, 5 to 30 days, 5 days to three weeks, one week to three weeks, one week to two weeks, 3 to 28 days, 5 to 28 days, 5 to 14 days, 7 to 28 days, 3 to 21 days, 5 to 21 days, 7 to 21 days, 3 to 14 days, 5 to 14 days, 7 to 14 days, 14 to 28 days, or 14 to 21 days. In some cases, it is 3-28 days. In some cases, it is 7-21 days. In some cases, the dose of the S-[E1]-A-[E2]-[U]-H [D] conjugate of the first treatment is 250 nmol-40,000 nmol total conjugate, 500-20,000 nmol total conjugate, or 1000-10,000 nmol total conjugate. In some cases the first treatment or second treatment includes an immunostimulant such as a TLR-7/8 agonist. A TLR-7/8 agonist may be administered at a dose of 750 to 120,000 nmol, or 3000 to 30,000 nmol. In some cases, the first treatment may be administered more than once prior to the time interval (T) and administration of the second treatment. In some cases, where drug D is present, it is covalently linked to the hydrophobic block (H), i.e., S-[E1]-A-[E2]-[U]-H-D, where the dash (−) indicates a covalent linkage.

In some cases, the method further comprises determining one or more of the level of CD8+ T cells, IFN-alpha, and IL12 or other cytokines or co-stimulatory molecules in a sample from the subject following the first and/or second treatment. A sample may be a blood sample or a tumor biopsy, for example.

### D. Adoptive Cell Therapy Combinations

Methods herein also comprise administering an adoptive cell therapy (ACT) as a first treatment and intravenously administering a vaccine in conjunction with or comprising an immunostimulant as a second treatment. In some cases, the ACT and the vaccine are administered sequentially such that the vaccine is administered from 3 days prior to administration of the ACT to 14 days following administration of the ACT, such as from one day prior to 7 days following administration of the ACT.

In some cases, the ACT is as described previously herein. For example, ACT may comprise a T cell isolated from a subject such as TIL, MIL or peripheral T cell, or the ACT may be a transgenic T cell (e.g., encoding a specific TCR), or CAR T therapy as described above. In some cases, the vaccine is any of a S-[E1]-A-[E2]-[U]-H [D] peptide antigen conjugate, including or not including an amphiphile such as an S-[B]-[U]-H [D] amphiphile, a PEG-[E1]-A-[E2]-[U]-H [D] peptide antigen conjugate, including or not including an amphiphile such as an S-[B]-[U]-H [D] amphiphile, or a polynucleotide vaccine, such as an adenovirus, adeno-associated virus, rhabdovirus, ChAdOx, MVA virus, or RNA vector encoding an tumor antigen. In some cases, the vaccine to be administered IV is either a PEG-[E1]-A-[E2]-[U]-H [D] peptide antigen conjugate including an amphiphile of formula S-[B]-[U]-H [D], or a ChAdOx vaccine. For example, as noted above, the PEG-[E1]-A-[E2]-[U]-H [D] conjugate formula or a S-[E1]-A-[E2]-[U]-H [D] formula with an amphiphile of formula S-[B]-[U]-H [D] was found to be safer for IV administration than a S-[E1]-A-[E2]-[U]-H [D] conjugate formula without amphiphile. In some cases, where drug D is present, it is covalently linked to the hydrophobic block (H), i.e., S-[E1]-A-[E2]-[U]-H-D, S-[B]-[U]-H-D, or PEG-[E1]-A-[E2]-[U]-H-D, where the dash (-) indicates a covalent linkage.

Methods herein also comprise administering an adoptive cell therapy (ACT) as a first treatment and intravenously administering an immunostimulant without a vaccine. In some embodiments, the immunostimulant may comprise (a) at least one TLR-3, TLR-7, TLR-8, TLR-7/8, TLR-9, MDA5, RIG1, or STING agonist, (b) a molecule that induces Flt3, IL-12, and/or type-I IFN signaling, and/or (c) an amphiphile, such as such as an amphiphile of formula S-[B]-[U]-H [D] described above. In some cases, the amphiphile may also incorporate an immunostimulant drug as the drug (D), for example.

In some cases, the method further comprises determining one or more of the level of CD8+ T cells, IFN-alpha, and IL12 or other cytokines or co-stimulatory molecules in a sample from the subject following the first and/or second treatment. A sample may be a blood sample or a tumor biopsy, for example.

### V. Kits Comprising Treatment Combinations

The disclosure herein also encompasses kits comprising first and/or second treatments as described herein. For example, a kit herein may comprise one or more components described herein to be administered as a first treatment, such as a composition delivering an tumor antigen, including a polypeptide or nucleotide vaccine composition, such as a peptide antigen conjugate or a polynucleotide vector encoding an antigen, or a composition intended to be provided to a subject's immune cells or blood cells for an adoptive cell therapy. A kit may further comprise an immunostimulant to be administered as a second treatment, in some cases in conjunction with a vaccine composition and/or an amphiphile, and the kit may also comprise the amphiphile and/or vaccine composition of the second treatment. Thus, for example, a kit herein may be used to provide most or all of the components for administration to the subject. In some cases, a kit may further include instructions for use. A kit may also include instruments for administration of the components, such as needles and syringes for injection (e.g., by IM) or equipment for administration by IV. In some cases, a kit may further include elements for determining the impact of the first and second treatments, such as, in some cases, for measuring the level of antigen-specific CD4 and/or CD8 T cells in a subject following administration of the first treatment, and assays for determining whether the second treatment induces systemic and/or tumor specific inflammation, such as to determine increases in IFN-I signaling in the subject.

Further nonlimiting exemplary aspects of the methods herein are discussed in the Examples section that now follows.

### EXAMPLES

### Example 1. Introduction and Methods for Examples 1-8

### A. Introduction

A key tenet of cancer immunotherapy is to harness the patient's own immune system to mediate tumor regression. Central to the anti-tumor immune response are T cells, which can kill tumor cells in an antigen-specific manner. Advances in the understanding of T cell biology has led to several important therapeutic strategies including checkpoint blockade, adoptive cell therapy and cancer vaccines (Waldman et al., 2020). A primary goal of therapeutic cancer vaccines is to promote tumor regression by inducing antigen-specific T cells in vivo (Saxena et al., 2021). Research efforts have focused on uncovering tumor antigens, in particular targeting tumor-specific mutations termed neoantigens (neoAg). The selective expression of neoAgs on tumor cells would potentially minimize autoimmune-related toxicities. Although promising, the clinical outcomes and immune responses measured in cancer vaccine trials have been limited in late-stage patients thus far (Melero et al., 2014; Romero et al., 2016). Hence, several challenges need to be addressed to improve the efficacy of personalized cancer vaccines (Hegde and Chen, 2020). A better understanding of the tumor immune contexture is critical: knowing the key molecular and cellular drivers dampening the T cell response at the tumor site can inform the rational design of vaccine formulations and influence delivery strategies.

Previously, a self-assembling nanoparticle vaccine platform was developed; the platform is capable of co-delivery of long peptides containing neoAgs with a Toll-like receptor 7/8 agonist (SNP-7/8a) (Lynn et al., 2020). Specifically, the platform comprises a self-assembling nanoparticle comprising a peptide antigen conjugate vaccine called “SNP” in these Examples. This peptide antigen conjugate is of a formula C-E1-A-E2-U-H-D, in which C is a solubilizing block (S) that is charged at physiological pH (e.g. 7.4), A is a peptide tumor antigen, specifically a neoantigen (neoAg), E1 and E2 are N-terminal and C-terminal extensions, U is a linker, H is a hydrophobic block, and D is the TLR 7/8 agonist drug, and wherein the dash “-” indicates a covalent linkage. By modifying the route of vaccination, the quality of antigen-specific CD8+ T cells can be altered: subcutaneous (SNP-SC) or intravenous delivery (SNP-IV) generated more terminally-differentiated or stem-like CD8+ T cells respectively (Baharom et al., 2021). Following SNP-SC vaccination, despite high magnitude CD8+ T cell responses, there was limited control of tumor growth in therapeutic murine tumor models. In contrast, SNP-IV was able to control the growth of established tumors; this was associated with the generation of stem-like CD8+ T cells capable of replenishing effector cells upon treatment with checkpoint inhibitors such as anti-PD-L1. In addition to providing high quality CD8+ T cells, it was hypothesized that SNP-IV could also be potentially beneficial in altering the tumor microenvironment (TME) through systemic innate activation by TLR7/8a.

The TME is a highly complex and dynamic milieu populated by numerous different cell types that may play unique roles in promoting or suppressing the anti-tumor activity of T cells. An immune infiltrate continuum ranging from “inflamed” to “immune desert” is often used to describe the immune microenvironment of human tumors. Preclinical studies in mice face challenges in modeling human tumors in terms of reflecting the true immune landscape (Mosely et al., 2017). MC38 tumors, often used as a murine model of human colorectal cancer (CRC), is largely composed of immunosuppressive cells (Mariathasan et al., 2018). Using single cell RNA-sequencing (scRNA-seq), recent studies carefully characterized the immune infiltrate in tumors of CRC patients and murine models, and identified highly conserved myeloid cells, including macrophages, monocytes and conventional dendritic cells (cDCs) that are present in both human and mouse tumors (Zhang et al., 2020). cDCs can be further subdivided into type 1 (cDC1) or type 2 (cDC2) lineages: cDC1s excel at cross-presentation for priming of CD8⁺ T cells whereas cDC2s are specialized at priming CD4+ T cells (Guilliams et al., 2014).

The heterogeneity of myeloid cells with T cell immune-suppressive functions have been reported in various murine and human cancer types. Recent data using high dimensional single cell technologies have led to refined understanding of the developmental relationships and phenotypic markers used to identify and classify these cells (Cheng et al., 2021). Myeloid-derived suppressor cells (MDSCs), often described in the context of late-stage cancers as having regulatory functions, can be thought of as a cellular state rather than a cellular identity: different myeloid cells can upregulate suppressive genes involved in inhibitory pathways such as arginine metabolism depending on the environmental stimuli (Hegde et al., 2021). Tumor-associated macrophages (TAMs) is another broad term used to refer to a heterogenous population of myeloid cells of embryonic or monocytic origin that evolved with or infiltrate into the tumors respectively (Bleriot et al., 2020; Hourani et al., 2021). An area of therapeutic interest is in modulating TAMs to polarize them towards more pro-inflammatory or anti-tumoral capacity, often referred to as an “M1 phenotype” as opposed to a more pro-tumoral “M2 phenotype” characterized by anti-inflammatory signaling (Yang et al., 2020). An important cytokine involved in modulating macrophages are type I interferons (IFNα and IFNβ), known to regulate the induction of more than 100 downstream interferon-stimulated genes (Borden, 2019; Dunn et al., 2006; U'Ren et al., 2010; Zitvogel et al., 2015). Importantly, the polarization of macrophages within the TME has been described in the context of successful or improved response to checkpoint blockade (Gubin et al., 2018; Lam et al., 2021).

The examples below relate to how SNP-IV mediates tumor regression through two distinct innate and adaptive immune mechanisms. As disclosed herein, neither vaccine-induced tumor-specific CD8⁺ T cells alone nor systemic innate immune activation alone were sufficient for controlling tumor growth. Rather, the optimal cancer vaccine regimen may require priming of tumor-specific CD8⁺ T cells followed by systemic IFN-I to alter the TME.

These findings have implications in the understanding of cancer vaccines and immunotherapies, highlighting the need for innate immune activation to modulate the TME for optimal T cell function.

### B. Materials and Methods

### C. Reagents and Resources

TABLE 1
Reagents and resources.

| | | |
|---|---|---|
| REAGENT or RESOURCE | SOURCE | IDENTIFIER |

Antibodies

| | | |
|---|---|---|
| Anti-mouse B220 (PE-Cy7); clone RA3- | BD Biosciences | Cat# 552772 |
| 6B2 | | |
| Anti-mouse CCR7 (BV421); clone 4B12 | Biolegend | Cat# 120120 |
| Anti-mouse CD3 (BUV395); clone145- | BD Biosciences | Cat# 563565 |
| 2C11 | | |
| Anti-mouse CD3 (Alexa700); clone 17A2 | Biolegend | Cat# 100216 |
| Anti-mouse CD4 (BUV395); clone RM4-4 | BD Biosciences | Cat# 740209 |
| Anti-mouse CD8 (APCeFluor780); clone | eBioscience | Cat# 47-0081-82 |
| 53-6.7 | | |
| Anti-mouse CD11b (AF700); clone MI/70 | BD Biosciences | Cat# 557960 |
| Anti-mouse CD11c (PE); clone HL3 | BD Biosciences | Cat# 553802 |
| Anti-mouse CD16/32; clone 2.4G2 | BD Biosciences | Cat# 553142 |
| Anti-mouse CD19 (BUV395); clone 1D3 | BD Biosciences | Cat# 563557 |
| Anti-mouse CD39 (PE-Dazzle594); clone | Biolegend | Cat# 143812 |
| Duha59 | | |
| Anti-mouse CD40 (AF647); clone 3/23 | Biolegend | Cat# 123613 |
| Anti-mouse CD44 (BUV737); clone IM7 | BD Biosciences | Cat# 564392 |
| Anti-mouse CD45 (BUV661); clone 30-F11 | BD Biosciences | Cat# 565079 |
| Anti-mouse CD64 (BV785); clone X54- | BD Biosciences | Cat# 741024 |
| 5/7.1 | | |
| Anti-mouse CD66a (BV605); clone CC1 | BD Biosciences | Cat# 750882 |
| Anti-mouse CD80 (PE-Dazzle594); clone | BD Biosciences | Cat# 562504 |
| 16-10A1 | | |
| Anti-mouse CD86 (BV711); clone GL1 | BD Biosciences | Cat# 740688 |
| Anti-mouse CD172α (PerCP-eF710); clone | Life Technologies | Cat# 46-1721-82 |
| P84 | | |
| Anti-mouse Eomes (PerCP-eF710); clone | Invitrogen | Cat# 46-4875-82 |
| Dan11mag | | |
| Anti-mouse F4/80 (PE-Cy5); clone BM8 | eBioscience | Cat# 15-4801-82 |
| Anti-mouse IA/IE (AF488); clone | Biolegend | Cat# 107616 |
| M5/114.15.2 | | |
| Anti-mouse LY6A/E (PE-CF594); clone D7 | BD Biosciences | Cat# 562730 |
| Anti-mouse Ly6C (APC-eF780); clone AL- | BD Biosciences | Cat# 560596 |
| 21 | | |
| Anti-mouse Ly6G (BUV563); clone 1A8 | BD Biosciences | Cat# 565707 |
| Anti-mouse NK1.1 (BUV395); clone PK136 | BD Biosciences | Cat# 564144 |
| Anti-mouse NKG2A (PE-Cy7); clone | Biolegend | Cat# 142810 |
| 16A11 | | |
| Anti-mouse PD-1 (BV421); clone 29F.A12 | Biolegend | Cat# 135218 |
| Anti-mouse SiglecH (BUV805); clone 440C | BD Biosciences | Cat# 748291 |
| Anti-mouse Tim-3 (BV605); clone RMT3- | Biolegend | Cat# 119721 |
| 23 | | |
| Anti-mouse TNFα (BV650); clone MP6- | Biolegend | Cat# 506333 |
| XT22 | | |
| Anti-mouse XCR1 (BV650); clone ZET | Biolegend | Cat# 148220 |
| Anti-mouse IFNα/β receptor-1; clone | BioXCell | Cat# BP0241 |
| MAR1-5A3 | | |
| Anti-mouse PD-L1; clone 10F.9G2 | BioXCell | Cat# BP0101 |
| TotalSeq-C0301 anti-mouse Hashtag 1; | Biolegend | Cat# 155861 |
| clones M1/42, 30-F11 | | |
| TotalSeq-C0302 anti-mouse Hashtag 2; | Biolegend | Cat# 155863 |
| clones M1/42, 30-F11 | | |
| TotalSeq-C0303 anti-mouse Hashtag 3; | Biolegend | Cat# 155865 |
| clones M1/42, 30-F11 | | |
| TotalSeq-C0304 anti-mouse Hashtag 4; | Biolegend | Cat# 155867 |
| clones M1/42, 30-F11 | | |
| TotalSeq-C0305 anti-mouse Hashtag 5; | Biolegend | Cat# 155869 |
| clones M1/42, 30-F11 | | |
| TotalSeq-C0306 anti-mouse Hashtag 6; | Biolegend | Cat# 155871 |
| clones M1/42, 30-F11 | | |
| TotalSeq-C0307 anti-mouse Hashtag 7; | Biolegend | Cat# 155873 |
| clones M1/42, 30-F11 | | |
| TotalSeq-C0308 anti-mouse Hashtag 8; | Biolegend | Cat# 155875 |
| clones M1/42, 30-F11 | | |
| TotalSeq-C0309 anti-mouse Hashtag 9; | Biolegend | Cat# 155877 |
| clones M1/42, 30-F11 | | |
| TotalSeq-C0310 anti-mouse Hashtag 10; | Biolegend | Cat# 155879 |
| clones M1/42, 30-F11 | | |

Chemicals, peptides, and recombinant proteins

| | | |
|---|---|---|
| Dasatinib | Selleckchem | S1021 |
| Isoflurane, USP | Baxter Healthcare | NDC 10019-360-60 |
| | Corp. | |
| Paraformaldehyde (PFA, 16%) | Electron | 15710 |
| | Microscopy | |
| | Sciences | |
| Tween-20 | Sigma | P-7949 |
| Heparin | Fresenius Kabi | NDC 63323-540-05 |
| Dimethylsulfoxide (DMSO) | Sigma-Aldrich | 276855-100 mL |
| Reps1 SNP vaccine | Vaccitech North | N/A |
| | America | |
| Reps1 SNP vaccine labelled with AF647 | Vaccitech North | N/A |
| | America | |
| Trp1 SNP vaccine | Vaccitech North | N/A |
| | America | |
| ABTS (2,2′-Azino-bis(3- | Sigma | A3219 |
| ethylbenzothiazoline-6-sulfonic acid)) | | |
| Murine IL-12 ABTS ELISA Kit | Peprotech | 900-K97 |
| Reps1 Tetramer (H-2D^{b}, AQLANDVVL) | Gift from J. | N/A |
| | Finnigan | |
| Trp1 Tetramer (H-2D^{b}, TAPDNLGYM) | Gift from J. | N/A |
| | Finnigan | |

Critical commercial assays

| | | |
|---|---|---|
| Mouse IFN Alpha All Subtype ELISA Kit, | PBL Assay Science | Cat# 42115-1 |
| High Sensitivity | | |
| eBioscience ™ Foxp3/Transcription Factor | Invitrogen | Cat# 00-5523-00 |
| Staining Set | | |
| LIVE/DEAD ™ Fixable Blue Dead Cell | ThermoFisher | Cat# L34962 |
| Stain Kit | Scientific | |
| ArC ™ Amine Reactive Compensation Bead | ThermoFisher | Cat# A10346 |
| Kit | Scientific | |
| BD Horizon Brilliant Stain Buffer | BD Biosciences | 566385 |
| Streptavidin PE (SaPE) | BD Biosciences | S866 |
| Trypsin-EDTA (0.25%) | Gibco, Life Tech | 25200-056 |
| ViaStain AOPI Staining Solution | Nexcelom | CS2-0106 |
| | Bioscience | |
| Milliplex MAP mouse cytokine/chemokine | Millipore Sigma | MCYTMAG-70K- |
| Magnetic Kit | | PX32 |
| Phosphate buffered saline (PBS) | Gibco | 10010-023 |
| RPMI 1640 | Cytiva, HyClone | SH30027.02 |
| | Labs | |
| FBS | Gibco | 10438-026 |
| Penicillin/Streptomycin/Glutamine (100X) | Gibco, Life Tech | 10378-016 |
| Non-essential Amino Acids (100X) | Cytiva, HyClone | SH30238.01 |
| | Labs | |
| Sodium Pyruvate (100 mM) | Cytiva, HyClone | SH30239.01 |
| | Labs | |
| Collagenase D | Roche | 11088882001 |
| DNase I | Roche | 04536282001 |
| ACK (Ammonium-Chloride-Potassium) | Quality Biological | 118-156-101 |
| lysing buffer | | |
| Chromium Single Cell 50 Reagent Kit | 10X Genomics | |
| Dynabead MyOne Silane | Thermo Fisher | Cat#37002D |
| | Scientific | |
| SPRIselect for Size Selection | Beckman Coulter | Cat#B23319 |

Experimental models: Cell lines

| | | |
|---|---|---|
| MC38 Murine colorectal cancer cell line | Genentech, L. | N/A |
| | Delamarre | |

Experimental models: Organisms/strains

| | | |
|---|---|---|
| Mouse: wild-type C57BL/6J mice | Jackson Labs | 000664 |
| Mouse: B6.SJL-Ptprca Pepcb/BoyJ | Jackson Labs | 002014 |
| Mouse: B6(Cg)-Zbtb46tm1(HBEGF)Mnz/J | Jackson Labs | 019506 |

Software and algorithms

| | | |
|---|---|---|
| Flowjo v10 | Tree Star | N/A |
| GraphPad Prism v8 | GraphPad software | N/A |
| xPONENT software | Luminex | N/A |
| Living Image v4.5 | PerkinElmer | N/A |
| R, v4.1.2 | R Project | https://www.r-project.org/ |
| Cellranger v6.0.0 | 10 X Genomics | https:// |
| | | support.10xgenomics.com |
| Seurat v4.1.0 | Seurat | https://cran.r- |
| | | project.org/web/packages/ |
| | | Seurat/index.html |
| Monocle 3 v1.0.0 | Monocle | https://github.com/cole- |
| | | trapnell-lab/monocle3/ |
| | | releases/tag/1.0.0 |
| Slingshot v2.2.0 | Slingshot | https://bioconductor.org/ |
| | | packages/release/bioc/ |
| | | html/slingshot.html |
| DoubletFinder v 2.0.3 | DoubletFinder | https://github.com/chris- |
| | | mcginnis-ucsf/DoubletFinder |
| pheatmap v1.0.12 | Pheatmap | https://cran.r- |
| | | project.org/web/packages/ |
| | | pheatmap/index.html |
| dittoSeq v1.6.0 | DittoSeq | https://bioconductor.org/ |
| | | packages/release/bioc/ |
| | | html/dittoSeq.html |
| biomaRt v2.50.3 | BiomaRt | https://bioconductor.org/ |
| | | packages/release/bioc/ |
| | | html/biomaRt.html |
| EnhancedVolcano v1.12.0 | Enhanced Volcano | https://bioconductor.org/ |
| | | packages/release/bioc/ |
| | | html/EnhancedVolcano.html |
| MetaScape v20220101 | MetaScape | https://metascape.org/ |
| | | gp/index.html#/main/ |
| QIAGEN IPA vDec. 2021 | QIAGEN | https:// |
| | | digitalinsights.qiagen.com/ |
| | | products-overview/ |
| | | discovery-insights- |
| | | portfolio/analysis-and- |
| | | visualization/qiagen-ipa/ |

Deposited data

| | | |
|---|---|---|
| scRNA-seq | Gene Expression | GSE |
| | Omnibus | |

### D. Experimental Models and Subject Details

### a) Mice

Wild-type (WT) C₅₇BL/6J, B6.SJL-Ptprca Pepcb/BoyJ, and B6(Cg)-Zbtb46tm1(HBEGF)Mnz/J (zDC-DTR) mice were purchased from The Jackson Laboratory and housed in specific-pathogen-free conditions. Upon arrival, mice were given 1 week to adjust to the new animal facility prior to being used. Mice used in studies were between 8-10 weeks old. All mice used were females. All animal experiments were performed at the Vaccine Research Center at the National Institutes of Health (NIH) with the approval of the Institutional Animal Care and Use Committee at the NIH. Experiments complied with the ethical guidelines set by the Institutional Animal Care and Use Committee and animals were humanely killed at defined end points.

### b) Tumor Cell Lines

The MC38 cell line was a kind gift from L. Delamarre (Genentech). The MC38 cells were grown in media comprised of DMEM+10% FBS+1% penicillin/streptomycin/glutamine+1% non-essential amino acids+1 mM sodium pyruvate. Stocks of MC38 were generated upon receipt of the cells and used for tumor experiments. Cells were tested regularly for Mycoplasma contamination; none tested positive throughout the studies.

### E. Methods

### a) Vaccines

SNP vaccines were produced as described previously (Lynn et al., 2020). Peptide antigens modified to form nanoparticles as part of a SNP vaccine were produced by GenScript. These peptides were linked to hydrophobic blocks containing an imidazoquinoline-based TLR-7/8 agonist (Vaccitech North America, USA) using a click chemistry reaction. For the pharmacokinetics studies, SNP vaccines were produced by linking Alexa Fluor 647 to hydrophobic blocks.

### b) Immunizations and Treatments

SNP vaccines were prepared in sterile PBS (Gibco) and administered subcutaneously to each footpad (50 μl per site) or intravenously via tail vein injection (100 μl) at a dose of 8 nmol and 32 nmol respectively. 50 μg of polyIC:LC (Hiltonol) was administered intravenously via tail vein injection (100 μL). Animals were treated with 200 μg per mouse of anti-PD-L1 (1° F.9G2; Bio X Cell) in 100 μl of PBS via intraperitoneal injection. For IFNα receptor (IFNAR) blockade, mice were treated 500 μg of anti-IFNAR1 antibody (MAR1-5A3; Bio X Cell) in 100 μl of PBS via intraperitoneal injection.

### c) Tumor Implantation

For each tumor implantation, a frozen cell aliquot was thawed and cultured in MC38 media at 37° C. and 5% CO2, passaged once and collected using trypsin EDTA (Gibco). Then, 105 cells in sterile PBS per mouse were implanted subcutaneously on the right flank. Tumors were measured twice a week using digital calipers. Tumor volume was estimated using the formula: (tumor volume=short×short×long/2). Animals were killed when tumors surpassed 1,000 mm³.

d) Generation of zDC-DTR Bone Marrow Chimeras

Eight-week-old recipient CD45.1 mice received 13 Gy of γ-irradiation (2 doses of 6.5 Gy each) before IV reconstitution with bone marrow from zDC-DTR mice. Eight weeks after reconstitution, successful chimerism was assessed by flow cytometry. Mice were used in studies eight weeks after reconstitution. To deplete DCs, mice were treated with 20 ng/g of diptheria toxin (DT) either intraperitoneally or intratumorally on day 13 (1 day before boosting) followed by 4 ng/g of DT on day 16.

### e) Blood and Tissue Processing

Heparin-treated blood was collected and lysed with ACK lysis buffer (Quality Biological) to isolate PBMCs. Lungs, liver, kidneys and tumors were collected in digestion media containing Roswell Park Memorial Institute (RPMI) 1640, 10% FCS, 50 U ml-1 DNase I (Sigma-Aldrich) and 0.2 mg/ml collagenase D (Sigma-Aldrich). Tissues were mechanically disrupted using the respective programs on the gentleMACS dissociator (Miltenyi Biotec) and incubated at 37° C. for 30-45 min in a shaking incubator. Spleens were mechanically disrupted and lysed with ACK lysis buffer. Lymph nodes were mechanically disrupted in BioMasher tubes (Nippi). All single-cell suspensions were filtered through a 70-μm cell strainer and resuspended in PBS for flow cytometry staining.

### f) Flow Cytometry

For T cell tetramer analysis, cells were assessed for viability with LIVE/DEAD Fixable Blue Dead Cell Stain Kit (Invitrogen) in PBS containing 50 nM dasatinib (STEMCELL Technologies) for 30 min at room temperature. Samples were then washed and blocked with anti-CD16/CD32 (BD Biosciences). Cells were then stained with fluorescently conjugated tetramer in cell staining buffer (PBS and 2% FCS) containing 50 nM dasatinib to enhance staining. Cells were simultaneously stained with the following surface antibodies to: CD8 (clone 53-6.7), PD-1 (clone 29F.A12), Tim-3 (clone RMT3-23), CD44 (clone IM7), CD39 (clone Duha59), and NKG2A (clone 16A11) purchased from BioLegend and CD4 (clone RM4-4) purchased from BD Biosciences. After a 1-h incubation at 4° C., cells were washed twice in cell staining buffer, fixed and permeabilized using the FoxP3 transcription factor staining buffer set (eBioscience). Cells were stained overnight at 4° C. with CD3 (clone 17A2) from BD Biosciences. Stain was washed off the following morning and samples resuspended in eBioscience FoxP3 transcription factor permeabilization wash buffer after which samples were acquired on an LSRFortessa X50 (BD Biosciences) using the FACSDiva software v8.0.1 (BD Biosciences).

For the mononuclear phagocyte uptake analysis, cells were assessed for viability with the LIVE/DEAD Fixable Blue Dead Cell Stain Kit for 10 min at room temperature. After FcR blocking, cells were stained for 20 minutes at room temperature with the following surface antibodies: NK1.1 (clone PK136), CD19 (clone 1D3), CD3 (clone 145-2C11), Ly6G (clone 1A8), CD45 (clone 30-F11), Siglec-H (clone 440c), CD86 (clone GL1), CD11c (clone L3), CD80 (clone 16-10A1), B220 (clone RA3-6B2), CD64 (clone X54-5/7.1), CD11b (clone MI/70) and Ly6C (clone AL-21) purchased from BD Biosciences, CCR7 (clone 4B12), MHC class II (I-A/I-E, clone M5/114.15.2), CD169 (clone 3D6.112) and XCR1 (clone ZET) purchased from BioLegend, and CD172a (clone P84) from Thermo Fisher Scientific. The stain was washed off and cells were then fixed in 0.5% PFA in PBS until they were acquired on an LSRFortessa X50 (BD Biosciences) using the FACSDiva software v8.0.1 (BD Biosciences).

### g) In Vivo Imaging

Whole-body imaging of mice after immunization with Alexa Fluor 647-labeled vaccines was performed using the IVIS Spectrum In Vivo Imaging System (Perkin Elmer) and analyzed using the manufacturer's software (Living Image 4.5, PerkinElmer).

### h) ELISA and Luminex

Serum from whole blood was collected at specified time points after vaccination. Commercially available ELISA kits were used to measure IL-12 subunit p40 (PeproTech) and all subtypes of IFNα (PBL Assay Science) according to the manufacturer's protocols. A commercially available Luminex kit (Millipore Sigma) was used according to the manufacturer's protocols to assess multiple analytes from serum samples.

i) Cell Sorting for scRNA-Seq

Spleens and tumors from mice that had been boosted one day prior were collected and processed into single cell suspensions by mechanical dissociation. Samples were stained with LIVE/DEAD Fixable Blue Dead Cell Stain Kit for 10 min at room temperature. Then samples were washed with FACS buffer (2% FBS in PBS) and stained with Fc block (Anti-mouse CD16/32, BD Biosciences) prior to addition of a surface stain. The surface stain antibody master mix contained: CD3 BUV395, CD19 BUV395, CD45 BUV661, CD11c PE, and CD11b AF700. Each sample was also stained with a unique hashtag antibody. Samples were incubated in surface stain for 20 minutes at room temperature after which all surface stain antibodies were washed off Samples were resuspended in FACS buffer and sorted by fluorescence activated cell sorting to isolate the live CD45+CD11b+CD11c+ cells. Sorted samples were pooled together by tissue prior to loading in duplicate into a Chromium single cell sorting system (10× Genomics). Expression and hashtag library construction was performed following the Chromium Single Cell VDJ Library protocol with a loading target of 1×104 per lane. At the conclusion, there were 4 expression and hash tag libraries from spleen samples and another 4 from tumor samples. The libraries were sequenced on a NovaSeq 6000 S2 chip.

j) Pre-Processing of scRNA-Seq Data

The raw scRNA-seq data (FASTQ files, 10× Genomics) were aligned to mm10 mouse reference genome using the Cell Ranger Single Cell software v6.0.0 (10× Genomics). The output count matrices were imported into Seurat R package v4.1.0 (Hao et al., 2021), and Seurat objects were created using CreateSeuratObject function (min.cells=3). The hashtagged data were demultiplexed using the HTODemux function, and singlet cells were predicted based on the Hashtag oligo classification for downstream analysis. Following dimension reduction and unsupervised clustering, further doublet cells were predicted and removed using DoubletFinder R package v2.0.3 (McGinnis et al., 2019), by Artificial Nearest Neighbours and assuming 12% doublet formation rate. Finally, all Seurat objects were merged as a single Seurat object and used for integration.

k) Integration of scRNA-Seq Datasets by Seurat

The scRNA-seq expression count data were log-normalized using NormalizeData function, and the variable genes were identified using FindVariableFeatures (selection.method=“vst”, nfeatures=2000) of Seurat package. Next, the cross-dataset anchors were identified using FindIntegrationAnchors function (dims=1:20, k.anchor=5, k.filter=30, reduction=“cca”) of Seurat. These anchors were then used for the integration step in which a cell-to-cell distance (batch-corrected) matrix was imputed using IntegrateData function (dims=1:20, k.weight=100) of Seurat.

### l) Dimension Reduction and Clustering

Two rounds of dimension reduction and clustering was performed on the imputed cell-to-cell distance matrix using specific functions embedded in Seurat R package. In the first round, the linear-dimensionality reduction (PCA) was performed on the whole scaled distance matrix using RunPCA function (npcs=50), and the data passed through FindNeighbors function and a non-linear dimensionality reduction by means of Uniform Manifold Approximation and Projection (UMAP) using RunUMAP function and umap-learn method (dims=1:12). Next, a graph-based unsupervised clustering of the single cells was performed using FindClusters function (resolution=0.8). The generated clusters were annotated based on their expression of canonical markers, and the clusters aligning with Mono/Mac/DC identity were subjected to the second round of the PCA and UMAP (dims=1:10) and unsupervised clustering (resolution=1.5). The second-round generated clusters, termed as original clusters were combined into meta-clusters following their hierarchical ordering based on their Euclidian distance calculated by mean expression of their top 50 DEGs and using Pheatmap R package v1.0.12.

### m) Comparing Clusters Distribution

The dittoSeq R package v1.6.0 (Bunis et al., 2020) was used to calculate and plot the distribution of each Mon/Mac/DC meta-cluster within each experimental group (condition). A meta-cluster distribution was defined as:

$$Distribution ( \% ) = \frac{Number of cell s in the metacluster}{Total number of Mon / Mac / DC} \times 100$$

The statistical cross-condition comparison of the meta-cluster distribution (carried out in triplicates) was done in GraphPad Prism software v8.0 using parametric one-way ANOVA followed by Dunnett's multiple comparison test comparing SC-IV (Reps1) and SC-IV (irr) groups versus SC-SC (Reps1). A P value<0.05 was considered as statistically significant.

### n) Predicting Cell Cycle Phase

Human cell cycle gene lists (embedded in Seurat package) were converted to their mouse equivalents using biomaRt R package v2.50.3 (Durinck et al., 2009), and then were implemented by Seurat pipeline for calculating cell-cycle scores and predicting cell-cycle phase of each single cell (G1, S and G2M).

### o) Differential Expression Gene (DEG) Analysis

DEG analysis was performed on the merged log-normalized count matrix using Wilcoxon Rank Sum test embedded in FindMarkers function of Seurat (min.pct=0.1, logfc.threshold=0.25). A Bonferroni-corrected P value<0.05 was used to describe significant DEGs among Mon/Mac meta-clusters. Cross-condition up-regulated and down-regulated genes were characterized following intra-condition combining of whole Mono/Mac meta-clusters and preforming DEG testing in SC-SC (Reps1), SC-IV (Reps1), SC-IV (irr) groups versus untreated group (min.pct=0.1, logfc.threshold=0.25); P value<0.05 was considered as statistically significant.

### p) Biological Pathways Analysis

To infer the biological pathways enriched in each Mono/Mac meta-cluster, their top-50 DEGs was imported into the web-based MetaScape portal (Zhou et al., 2019). In addition, to explore the cross-condition changes of the biological pathways, ingenuity pathways analysis (IPA) was performed on all statistically significant up-regulated and down-regulated genes [SC-SC (Reps1), SC-IV (Reps1), SC-IV (irr) groups versus untreated group] using QIAGEN IPA software.

### q) Human Bulk RNA-Seq Data Analysis

Human orthologues (“huChil3”) of mouse Chil3+ monocytes were identified using the nicheNet R package (Browaeys et al., 2020). Normalized count data of sorted bulk RNA-seq populations from 12 tumor types was obtained from GEO under accession GSE184398 and log 2 transformed. Subsequently, the average expression of huChil3 genes was calculated in each sample after scaling (z-score transformation) the expression of each gene across samples.

r) Human scRNA-Seq Data Analysis

Human single-cell RNA-seq data: The MoMac-VERSE dataset (“2021_MoMac_VERSE.rds”) was obtained online (https://gustaveroussy.github.io/FG-Lab/) and subsequently analyzed with the Seurat package in R (Hao et al., 2021). First, the atlas were filtered to contain only datasets including cancer patients that were sequenced with 10×/Droplet sequencing technology. Next, the huChil3 geneset was restricted to only those genes robustly expressed in the dataset (average expression across clusters>5) and calculated an enrichment score for the resulting geneset in each cell (“AddModuleScore”). To compare the score across datasets and clusters, the median huChil3 geneset score in each cluster for each dataset was calculated and compared these median scores per dataset across clusters.

For individual dataset analyses the MoMac-VERSE was further filtered to only contain cells from a particular study and Variable gene selection, scaling, PCA and UMAP calculation were carried out with standard parameters (custom: Number of PCs used for nearest neighbor and UMAP calculation: 30 PCs, resolution for clustering: 0.3).

For survival analyses using TCGA datasets, Batch corrected normalized TCGA Pan-Cancer mRNA data was obtained from UCSC Xenabrowser (https://xenabrowser.net/) (n=11,060). Samples containing NA expression values were removed. The data was additionally filtered to only contain samples from primary solid tumors (sample code 01; n=9,702). Survival data was obtained from Table S1 in Thorsson et al. (Thorsson et al., 2018) and linked to the Pan-Cancer dataset via the unique TCGA Participant Barcode. Inferred general monocyte levels were obtained from Table S6 of Aran et al. (Aran et al., 2017) and similarly linked to the expression data via the unique TCGA Participant Barcode. Survival information and estimated monocyte levels were available for n=8,963 samples analyzed. HuChil3 geneset scores were calculated in each of these TCGA sample as the average of the human orthologs found expressed in the MoMac-VERSE after gene-wise scaling (z-score) across samples. High and low expression groups were defined using a median split of scores and Kaplan Meier curves were calculated in R.

### F. Quantification and Statistical Analysis

### a) Statistical Analysis of Biological Data

All results are presented as the median with s.d. Statistics were assessed using a Kruskal-Wallis test with Dunn's correction for multiple comparisons [immunogenicity], two-way analysis of variance (ANOVA) with Bonferroni correction [tumor growth curves], log-rank test [survival curves], Mann-Whitney U-test [cytokines] or one-way ANOVA with Dunnett's test for multiple comparisons [scRNA-seq [populations](Prism; GraphPad Software v8.4.2).

### Example 2. Antigen-Specific CD8⁺ T Cells Generated by SNP-SC Controlled Tumor Growth when Followed by SNP-IV

Subcutaneous administration of the SNP-7/8a (SNP-SC), a self-assembling nanoparticle vaccine, can generate neoAg-specific CD8⁺ T cells that are terminally differentiated based on transcriptional profiling (Baharom et al., 2021). Although SNP-SC-generated CD8⁺ T cells could control tumor growth in a prophylactic setting, they were ineffective in a therapeutic setting. In contrast, SNP-7/8a administered intravenously (SNP-IV) can generate stem-like CD8⁺ T cells that were effective in mediating tumor regression in established tumors when delivered in combination with anti-PD-L1 treatment (Baharom et al., 2021). Of note, SNP-IV also can induce systemic innate activation marked by high levels of pro-inflammatory mediators such as IFNα and IL-12. The Examples herein delineate the role of CD8⁺ T cell magnitude and quality from the effects of systemic innate immune activation by SNP-IV.

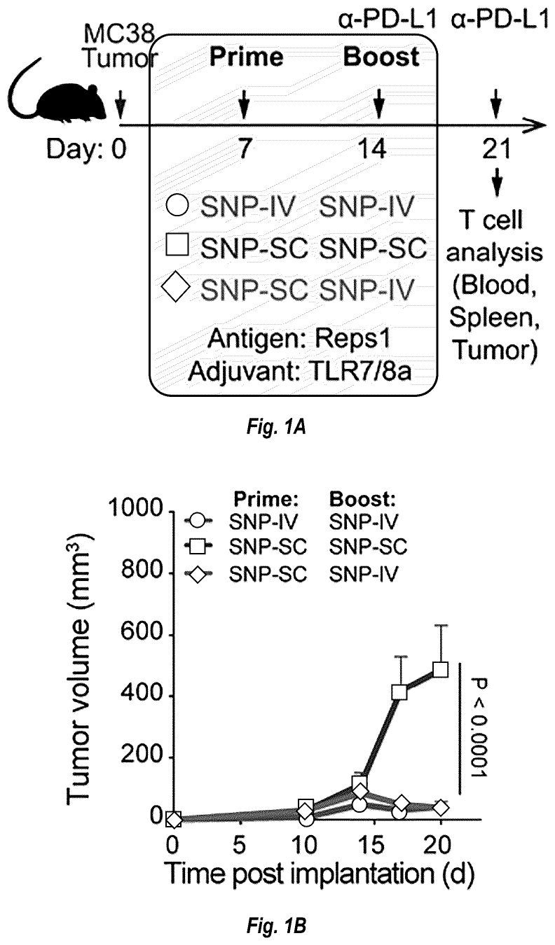

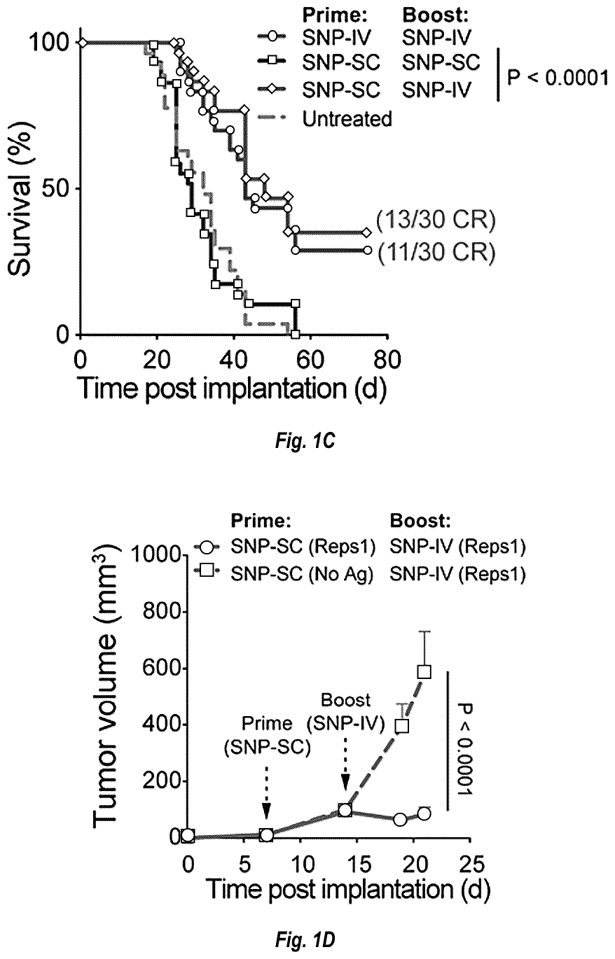

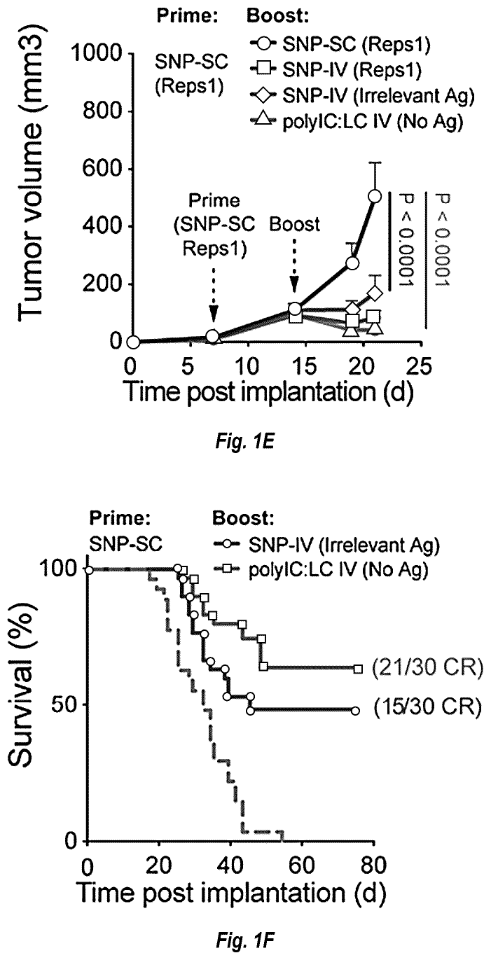

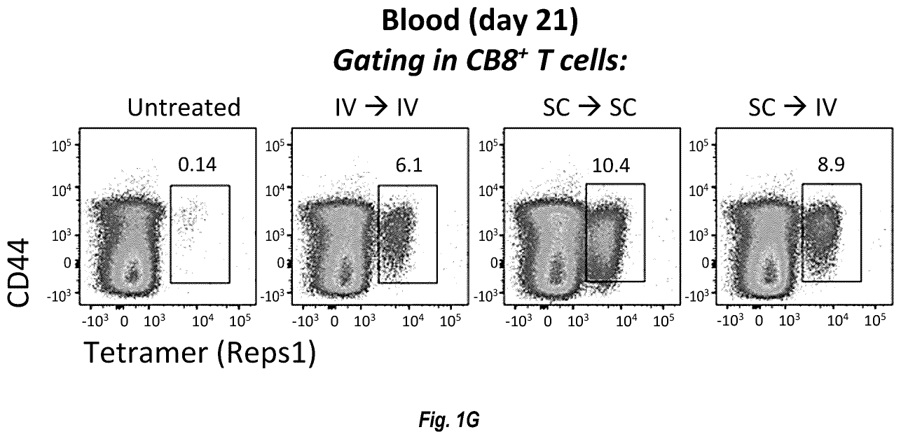

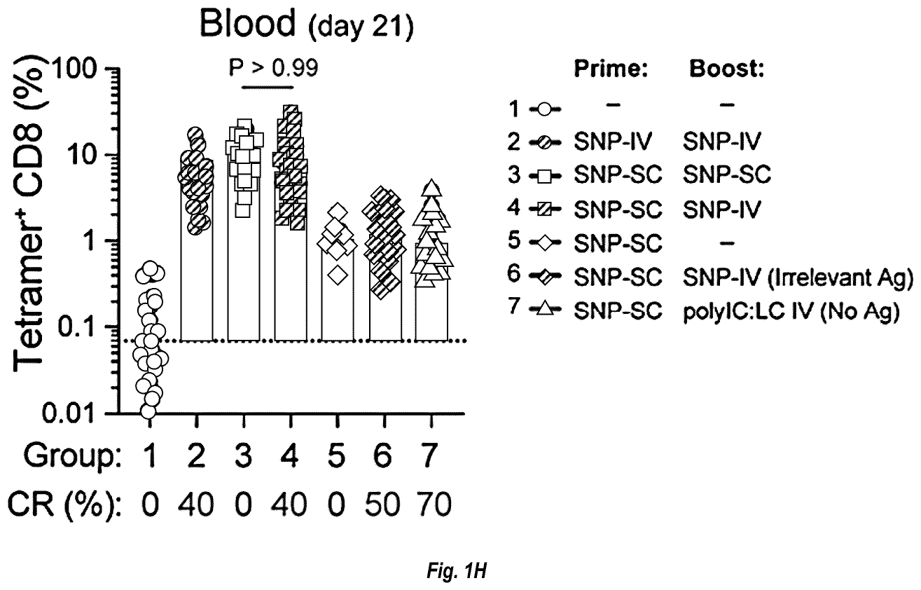

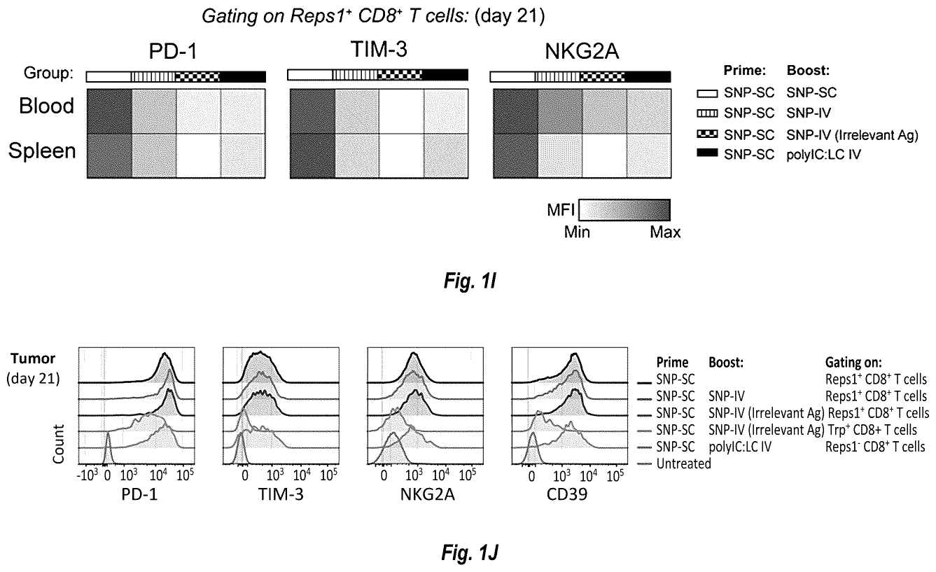

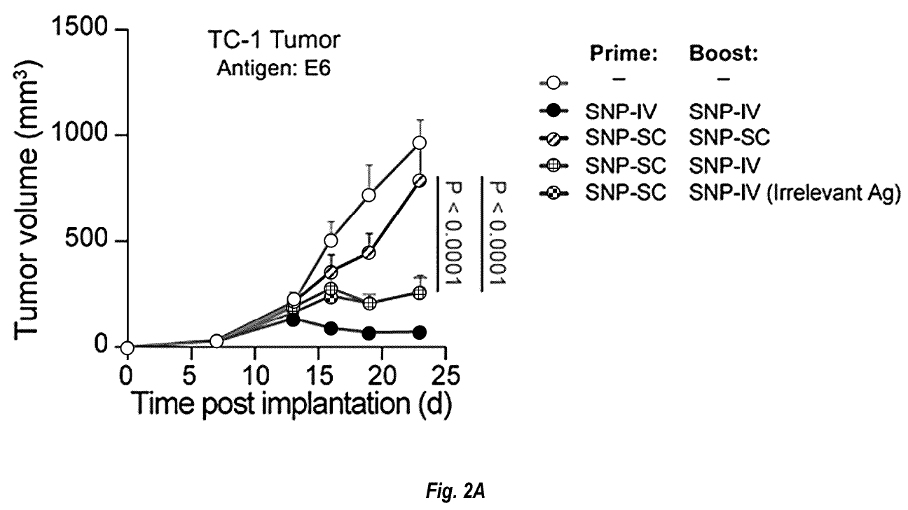

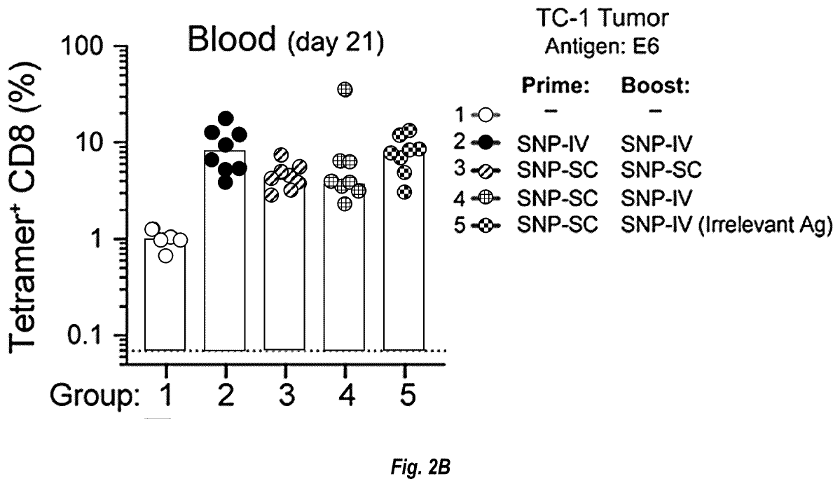

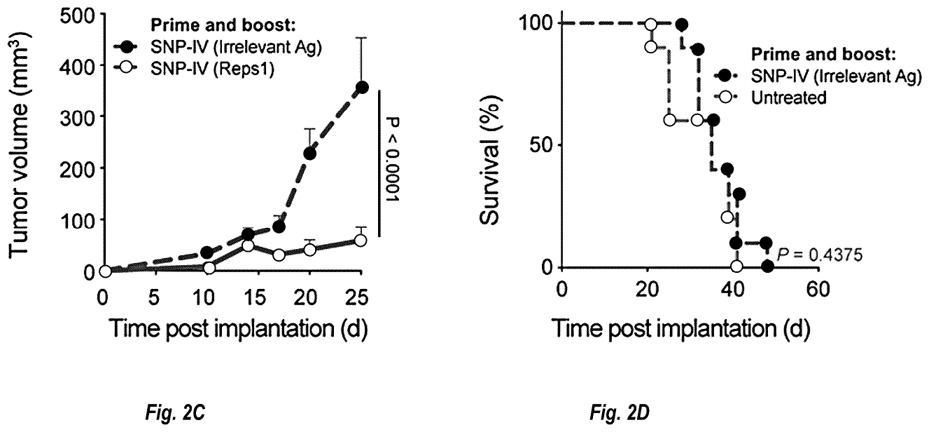

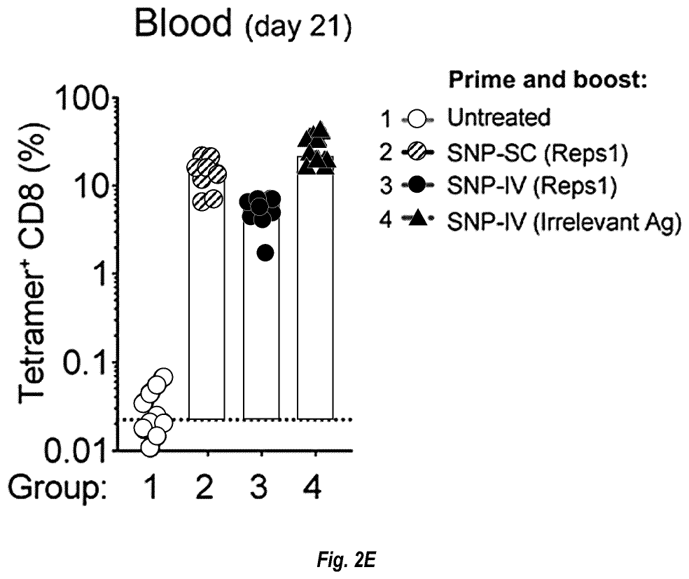

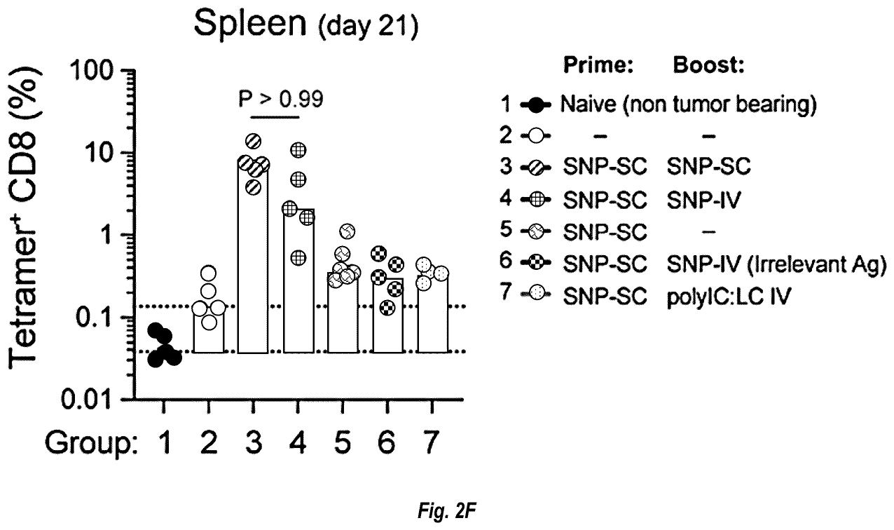

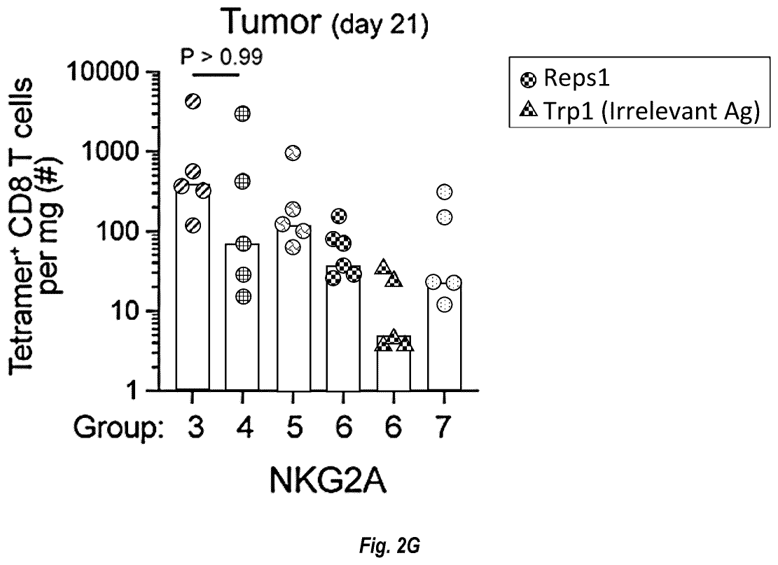

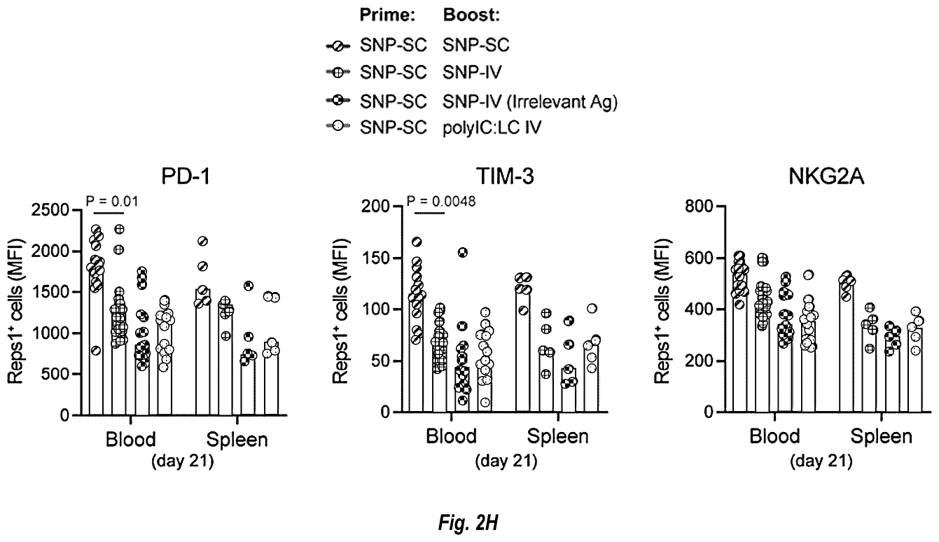

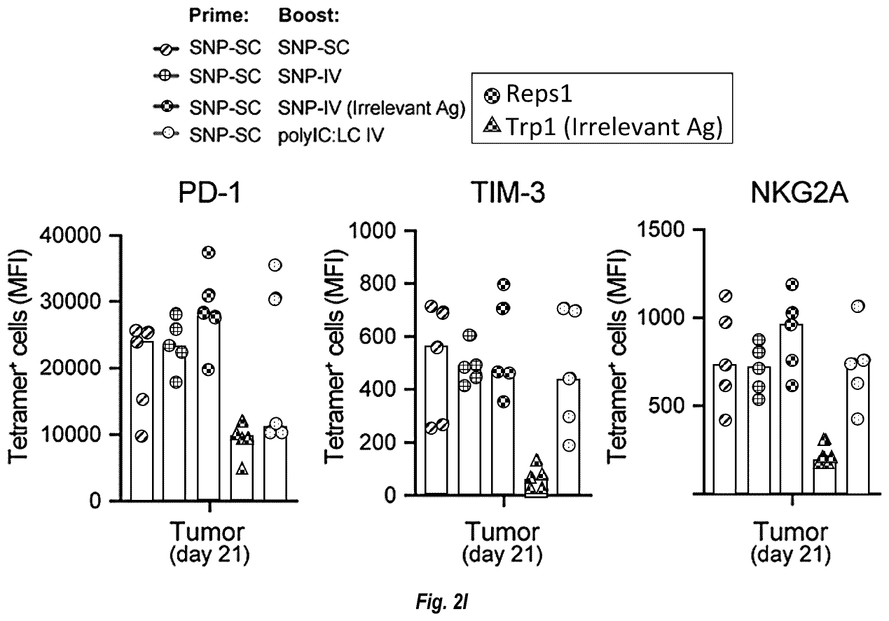

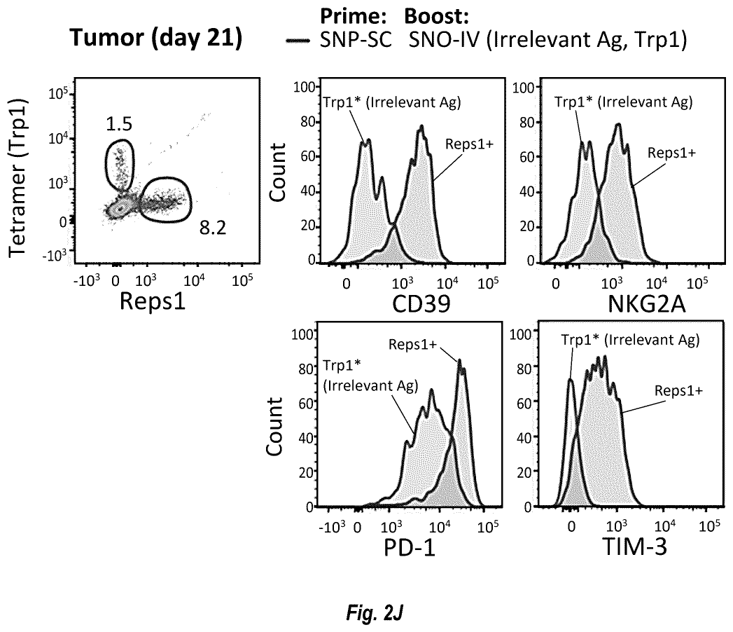

Mice were implanted with MC38 tumors on the subcutaneous flank and were treated on day 7 (prime) and day 14 (boost) with SNP-7/8a containing Reps1, an MC38 neoAg, together with anti-PD-L1 (FIG. 1A). Consistent with prior data, the group of mice that received SNP-IV (as a first treatment and as a second treatment, i.e., as a prime and boost) demonstrated mediated tumor regression whereas the group of mice that received SNP-SC (prime and boost) did not demonstrate control of tumor growth (FIG. 1B). However, mice that were first primed with SNP-SC followed by an SNP-IV boost also demonstrated control of tumor growth and improved survival similar to mice treated with prime and boost doses (i.e., two doses) of SNP-IV (FIG. 1C). To confirm and extend these findings in a different tumor model, mice were implanted with TC-1 tumors and treated with SNP-7/8a containing HPV E6 antigen. Consistent with observations in mice implanted with MC38 tumors, TC-1 tumor-bearing mice that were treated with SNP-SC followed by SNP-IV had significantly smaller tumors than mice boosted with SNP-SC (FIG. 2A) despite the generation of similar magnitudes of antigen-specific CD8⁺ T cells (FIG. 2B). These data suggest that SNP-IV is not required at the time of priming, and terminally-differentiated CD8⁺ T cells generated by SNP-SC can also mediate tumor regression when boosted with SNP-IV.

To first show that tumor regression required neoAg-specific CD8⁺ T cells, mice were vaccinated with SNP-IV containing an irrelevant antigen (prime and boost). SNP-IV was ineffective in controlling tumors (FIG. 2C) or extending survival (FIG. 2D) in the absence of antigen-specific CD8⁺ T cells (FIG. 2E). Mice that were primed with SNP-SC containing no tumor-specific antigen followed by SNP-IV containing the MC38 antigen was also not effective at controlling tumor growth (FIG. 1D). To assess the requirement for antigen at the time of boost, mice were primed with SNP-SC containing Reps1, followed by SNP-IV containing an irrelevant antigen (FIG. 1E). Remarkably, SNP-SC prime followed by SNP-IV boost with an irrelevant antigen resulted in improved control of tumor growth and a 50% survival rate (FIG. 1F). These data show that neoAg⁺ CD8⁺ T cells generated by SNP-SC given once was sufficient to mediate tumor regression when followed by systemic innate stimulation, even in the absence of antigen at the time of boost. Furthermore, the anti-tumor effect by the boost is not unique to TLR7/8a signaling as IV administration of polyIC:LC, another adjuvant that induces IFN-I signaling, after SNP-SC prime could also significantly mediate tumor regression and prolong survival when given 7 days after priming with SNP-SC (FIGS. 1E and F).

### Example 3. Tumor Regression Induced by SNP-SC Prime Followed by Adjuvant Administered IV Did not Require Expansion of Tumor-Specific CD8⁺ T Cell Responses

To assess whether there are differences in the magnitude of neoAg-specific CD8⁺ T cells after SNP-SC or SNP-IV boost, whole blood was sampled on day 21, a week after the boost and measured the tetramer⁺ population by flow cytometry (FIG. 1G). SNP-SC given twice, or SNP-SC followed by SNP-IV generated similar frequencies of tetramer⁺ CD8⁺ T cells at approximately 10% of total CD8+ T cells in circulation. The magnitude of neoAg⁺ CD8⁺ T cells is also comparable in the spleen and tumor (FIGS. 2F and 2G). Groups that received SNP-IV containing an irrelevant antigen or polyIC:LC IV at the time of boost showed no increase in the magnitude of neoAg-specific CD8⁺ T cells compared to mice that only received one shot of SNP-SC containing Reps1 at approximately 1% of total CD8⁺ T cells in circulation (FIG. 1H). In summary, SNP-SC primed neoAg-specific CD8⁺ T cells may promote anti-tumor efficacy if followed by IV administration of SNP-7/8a or polyIC:LC even with ten-fold lower CD8⁺ T cell responses compared to the other vaccinated groups.

As markers of exhaustion and antigen experience on CD8⁺ T cells are important for improving the therapeutic effects of immune checkpoint blockade, the expression of PD-1, TIM-3 and NKG2A post vaccination were assessed. SNP-SC boosted cells in circulation showed a higher expression of exhaustion markers PD-1, TIM-3 and NKG2A compared to the other groups that received SNP-IV (Reps1 or irrelevant antigen) or polyIC:LC IV (FIGS. 1I and 2H). However, the tumor-infiltrating Reps1⁺ CD8⁺ T cells showed similar expression levels of PD-1, TIM-3, NKG2A and CD39 regardless of treatment received (FIGS. 1J and 2I). Given the protective effect of boosting with SNP-IV containing an irrelevant antigen (Trp1), the quality of tumor-infiltrating CD8⁺ T cells were compared to assess whether they may be playing a bystander effect. Compared to intratumoral Reps1⁺ CD8⁺ T cells, Trp1⁺ CD8⁺ T cells expressed lower levels of exhaustion markers including CD39, reflecting a lack of tumor antigen experience (FIG. 2J). Overall, there were no striking differences in the expression of exhaustion markers on CD8⁺ T cells, especially in the tumor, suggesting that the systemic innate stimulation provided by SNP-IV or polyIC:LC given IV may be affecting immune cells other than T cells.

### Example 4. SNP-IV but not SNP-SC Resulted in Intratumoral Vaccine Distribution and DC Maturation

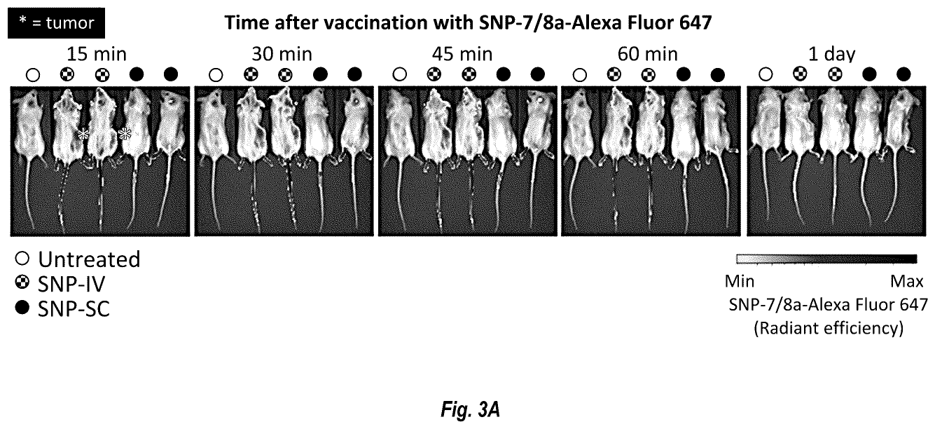

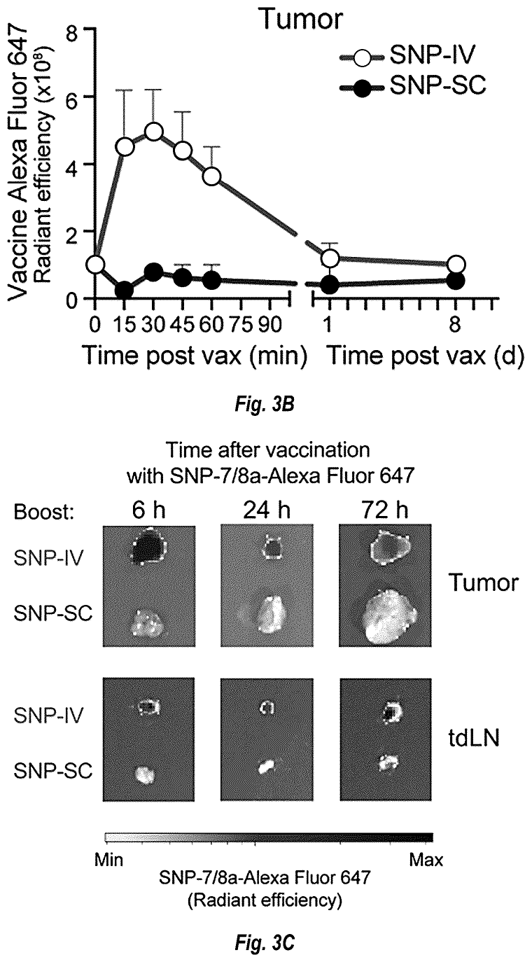

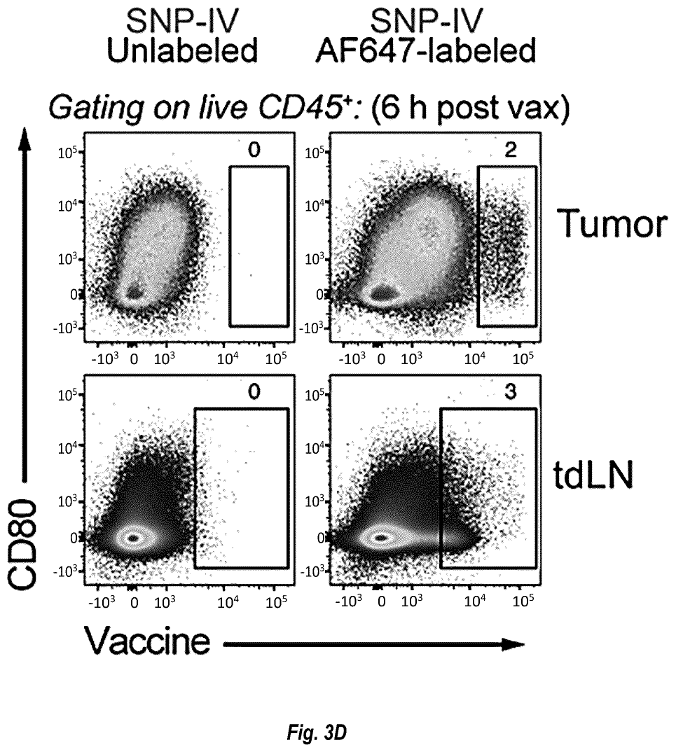

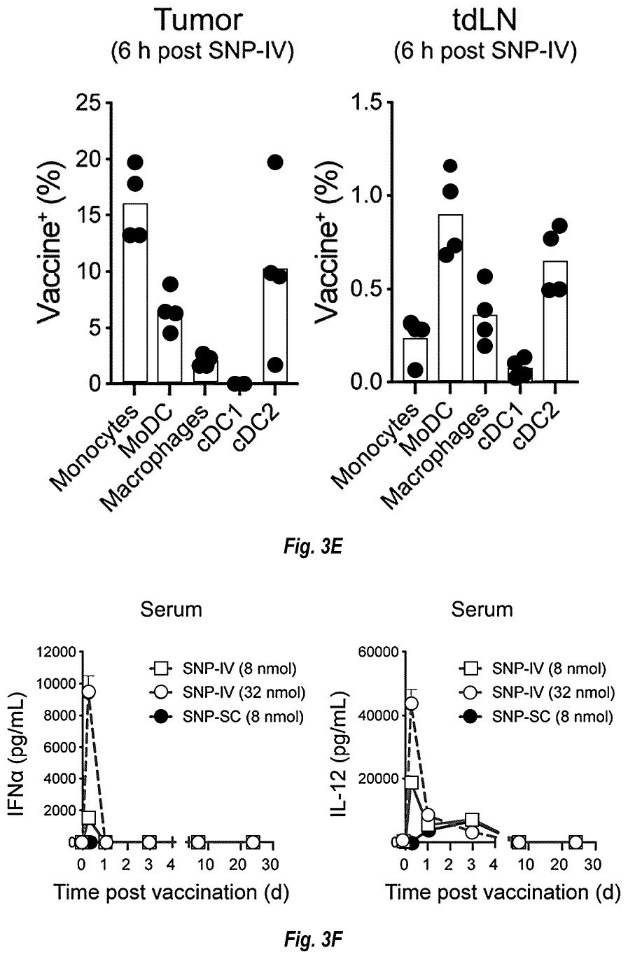

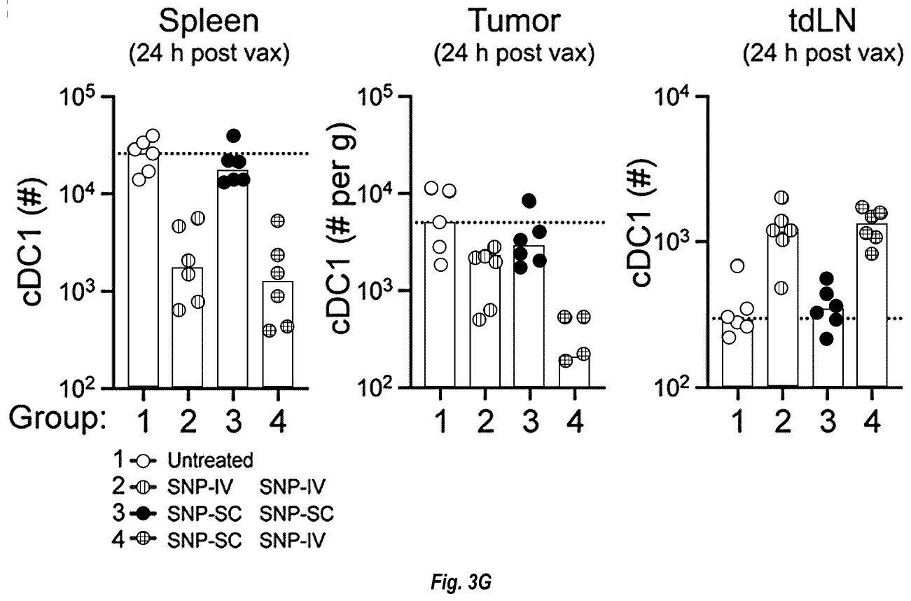

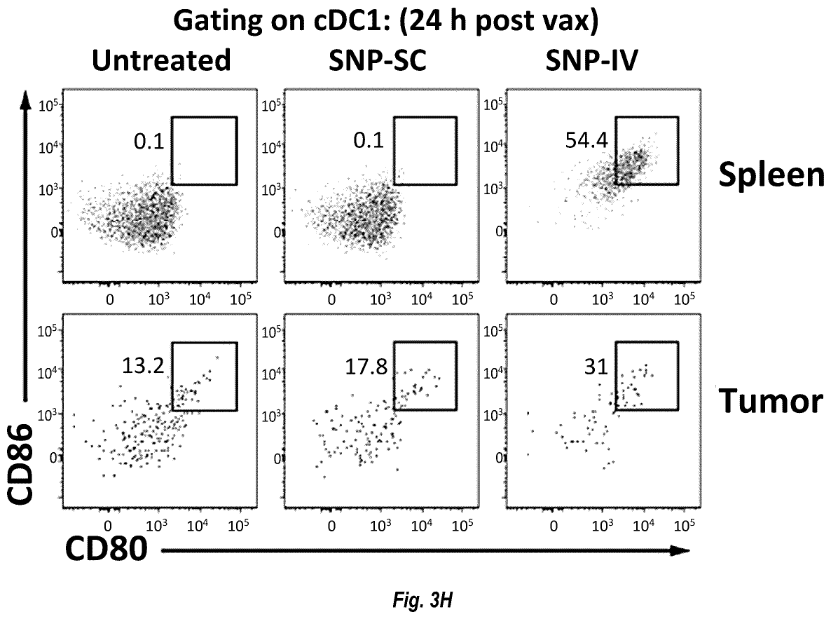

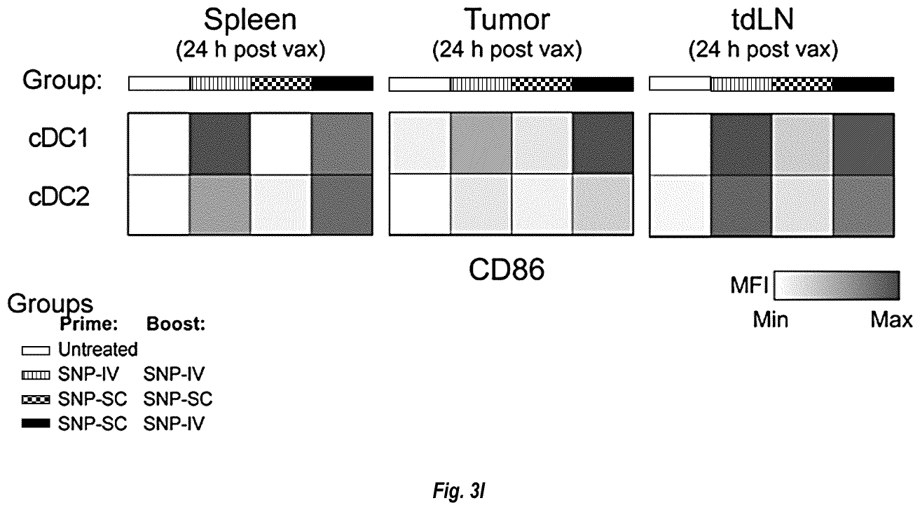

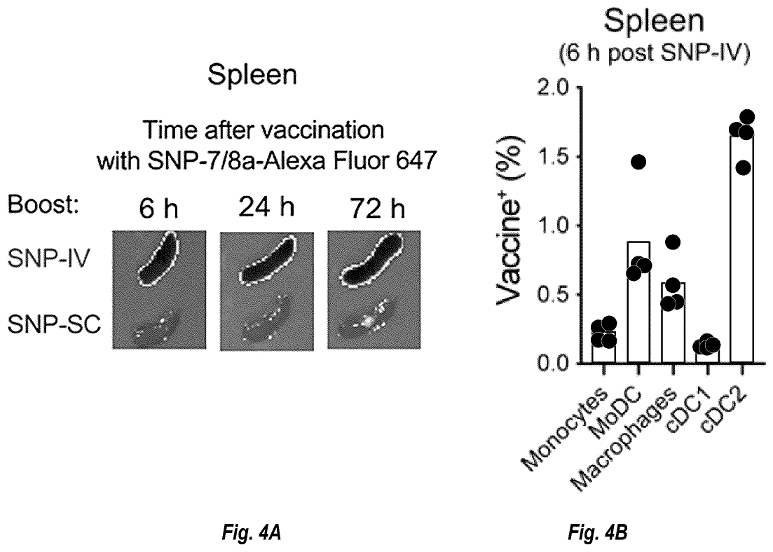

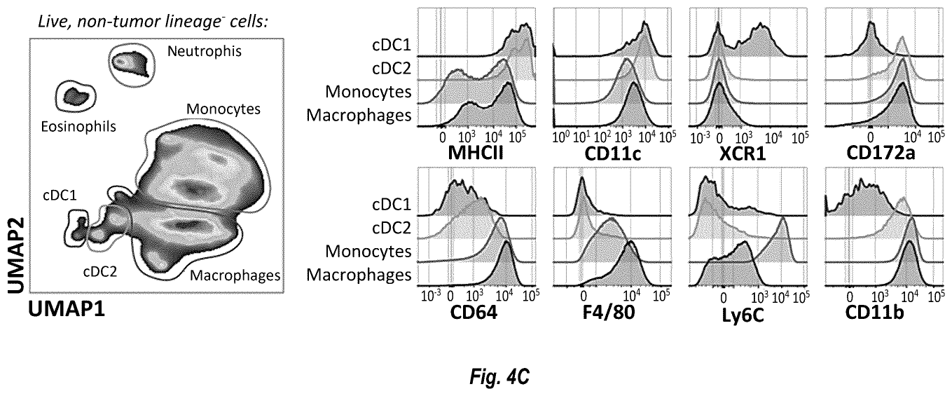

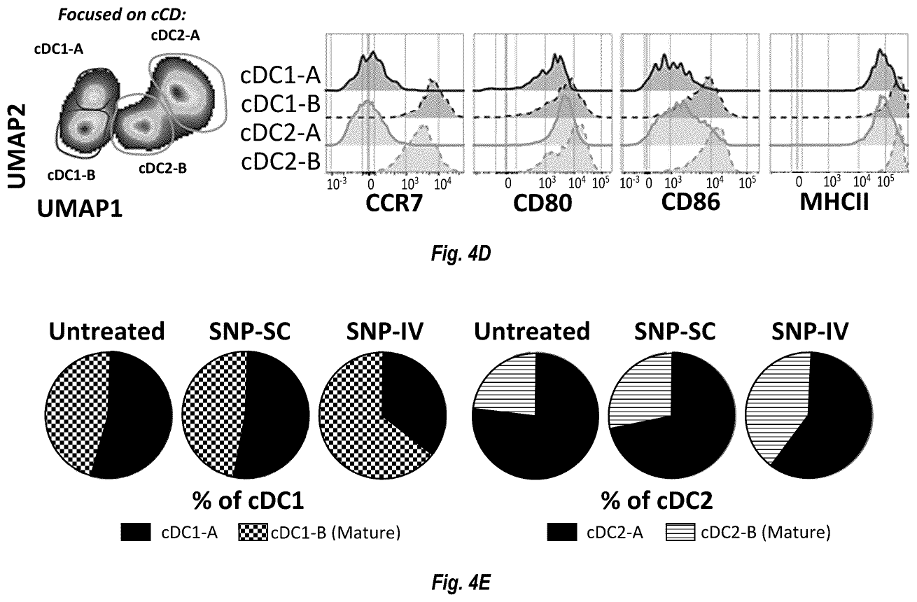

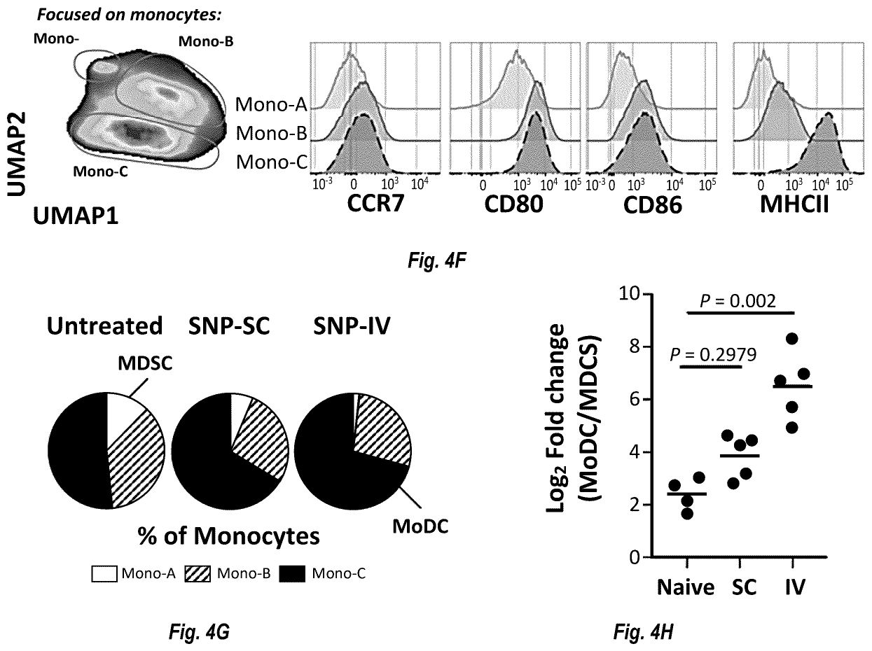

To further demonstrate differences between boosting with SNP-SC or SNP-IV that may influence anti-tumor immunity, vaccine pharmacokinetics were tracked by performing live imaging of animals using an Alexa Fluor 647-labeled SNP-7/8a. Systemic distribution of the vaccine was detected by in vivo imaging after SNP-IV but not SNP-SC (FIG. 3A). Moreover, fluorescently labeled vaccine could be localized in the tumor primarily in the first hour and remained detectable at low levels after 24 hours (FIG. 3B). Assessment of explanted tumors and tumor-draining lymph nodes collected at 6 hours, 24 hours and 72 hours post vaccination confirmed the detection of fluorescently labeled vaccine after SNP-IV but not SNP-SC vaccination (FIG. 3C). Additionally, SNP-IV also led to detectable vaccine in the spleens, suggesting systemic vaccine distribution (FIG. 4A). Following tissue digestion, single cell suspensions were stained for flow cytometry. Consistent with the live imaging data, a population of vaccine⁺ cells could be detected at 6 hours post vaccination within the CD45⁺ leukocyte compartment (FIG. 3D). In the tumor, tumor-draining LN and the spleen, multiple different myeloid cell populations including monocytes, monocyte-derived DCs (moDCs), cDC1s and cDC2s had taken up the vaccine (FIG. 3E and FIG. 4B).

Given the systemic distribution of vaccine after SNP-IV but not SNP-SC, the levels of pro-inflammatory cytokines in the sera of mice were assessed at 6 hours after vaccination (FIG. 3F). High levels of systemic IFNα and IL-12 were measured by ELISAs after SNP-IV, especially at the higher dose of 32 nmol that is used in the therapeutic studies, compared to SNP-SC (FIG. 3F). As IFNα is known to promote cross-presentation of antigens by cDC1s for CD8⁺ T cell priming (Le Bon et al., 2003), cDC1s in the spleen, tumor and tumor-draining LNs were quantified at 24 hours after vaccination. Unsupervised uniform manifold approximation and projection (UMAP) analysis of myeloid cells revealed 6 clusters including eosinophils, neutrophils, macrophages, monocytes and cDCs (FIG. 4C). Consistent with earlier observations (Baharom et al., 2021), cDC1 numbers were reduced in the spleen and tumor after SNP-IV (FIG. 3G). Compared to untreated animals, a three-fold increase in cDC1s was detected in the tumor-draining LNs after SNP-IV but not SNP-SC. To assess DC maturation, a combination of markers were used, including the co-stimulatory molecules CD80 and CD86, the chemokine receptor CCR7, as well as MHCII (FIG. 4D). A higher proportion of mature cDC1s were observed after SNP-IV compared to SNP-SC in the spleen and tumor (FIG. 3H and FIG. 4E) as well as the tumor-draining LNs (FIG. 3I). Monocyte-derived cells can also be identified with varying expression levels of MHCII (FIG. 4F). Interestingly, a larger proportion of moDC expressing high MHCII can be detected in the tumors after SNP-IV compared to SNP-SC or untreated mice (FIG. 4G and FIG. 4H). The adjuvant polyIC:LC administered by IV route was also able to induce DC maturation in the spleen and tumor-draining LN, comparable to SNP-IV (FIG. 4I). Altogether, the data suggest that IV administration of SNP-7/8a results in systemic vaccine distribution including the spleen, tumor and tdLN where DCs can undergo maturation to promote licensing of neoAg-specific CD8⁺ T cells.

### Example 5. ScRNA-Seq of Tumors Revealed that Intratumoral Chil3⁺ Monocytes were Reduced after SNP-IV

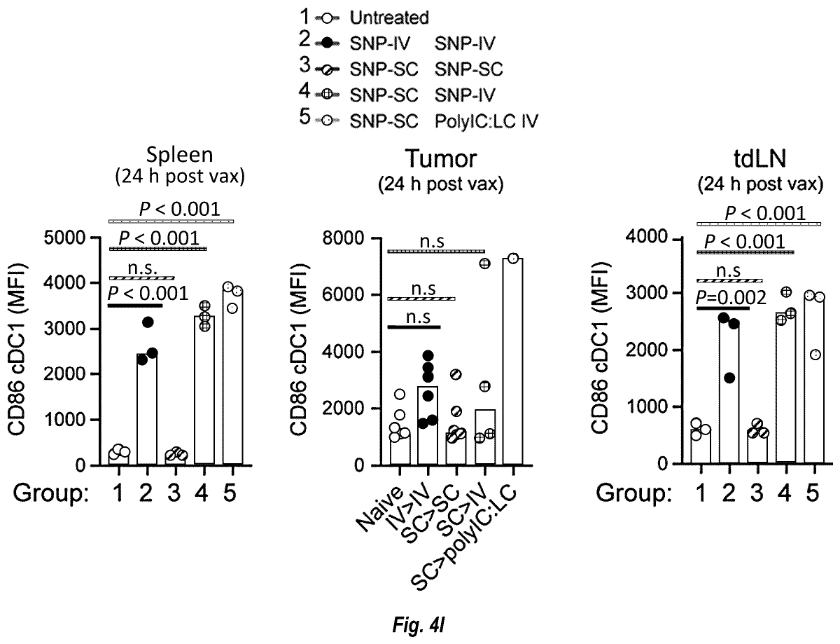

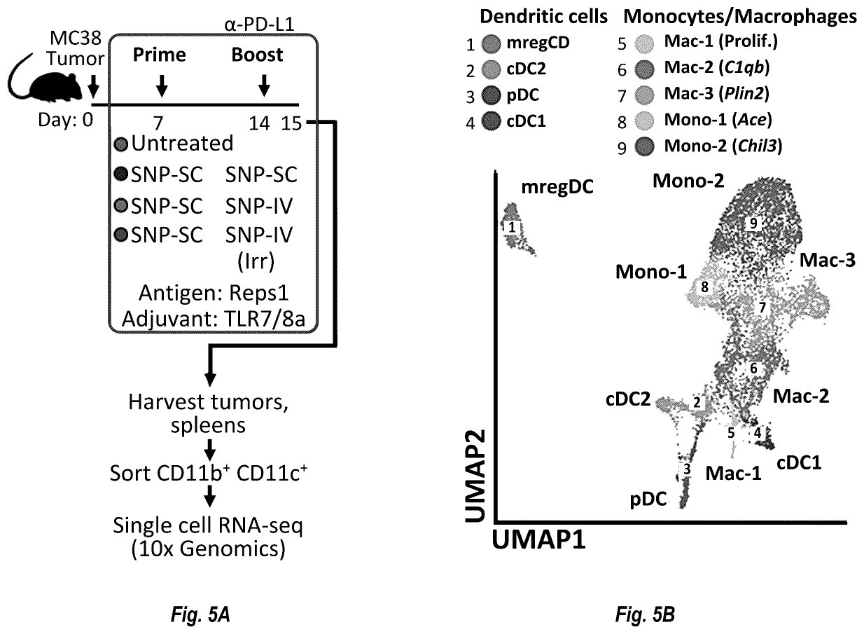

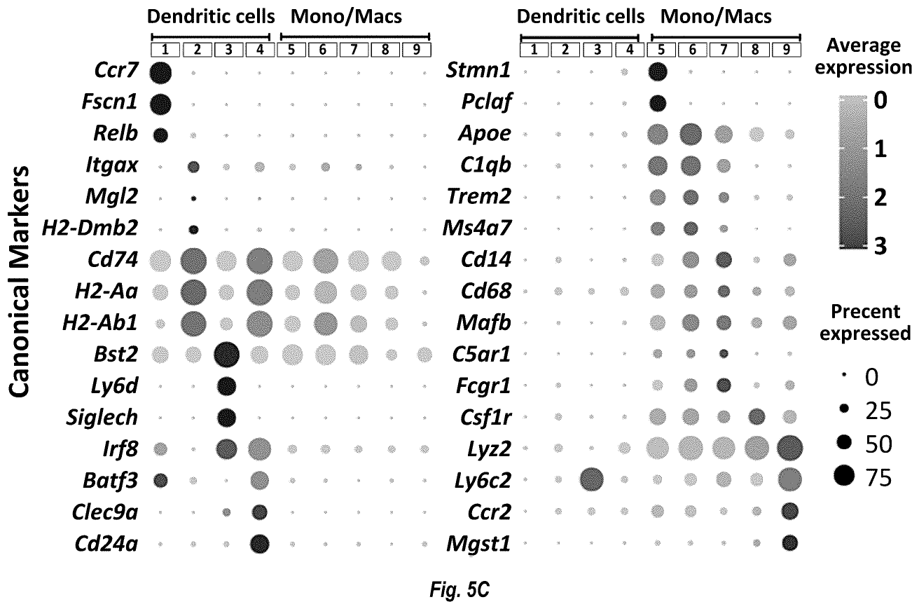

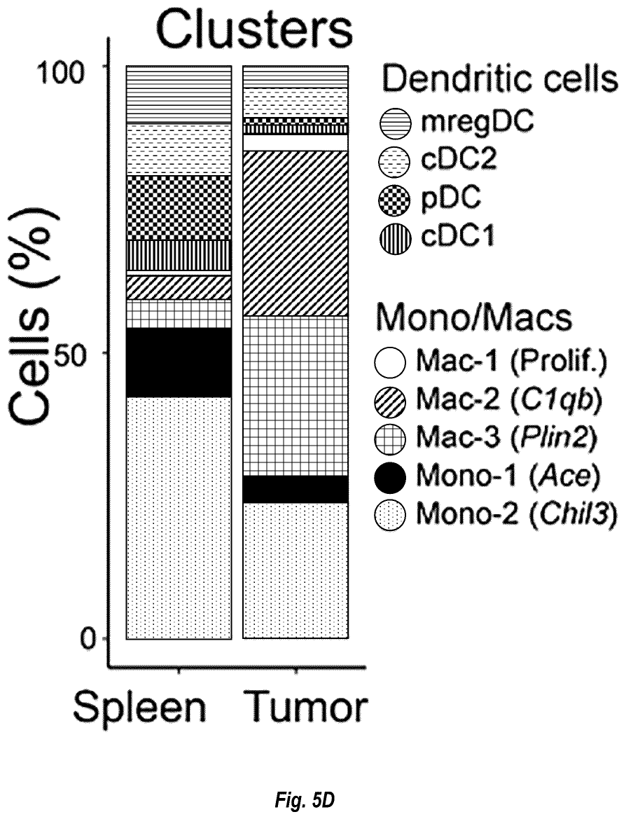

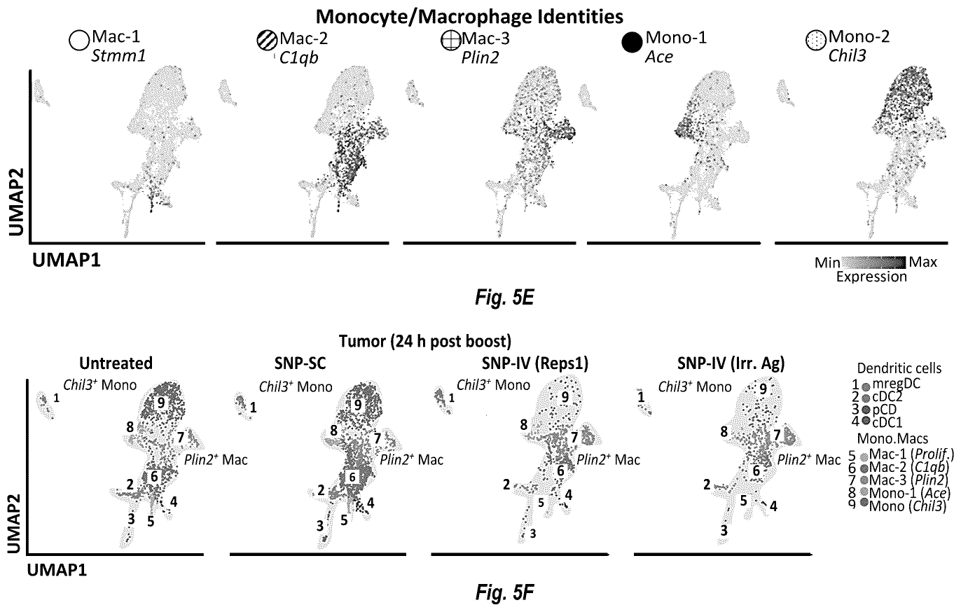

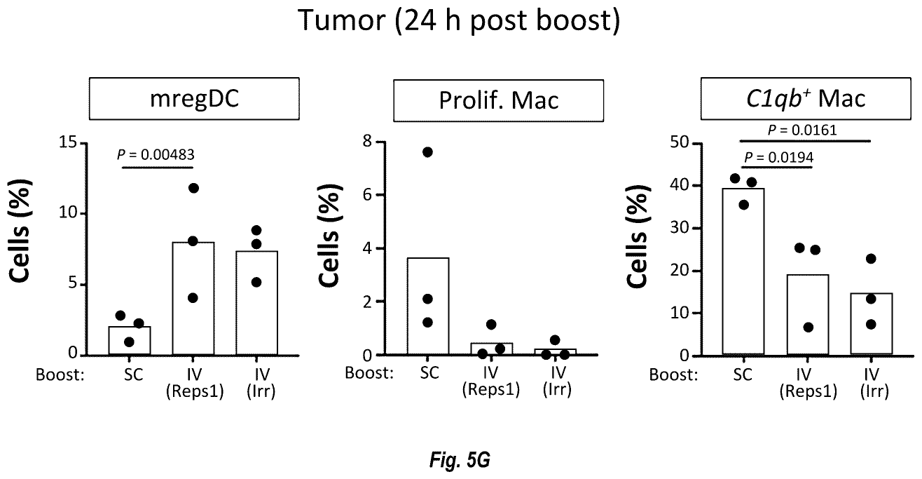

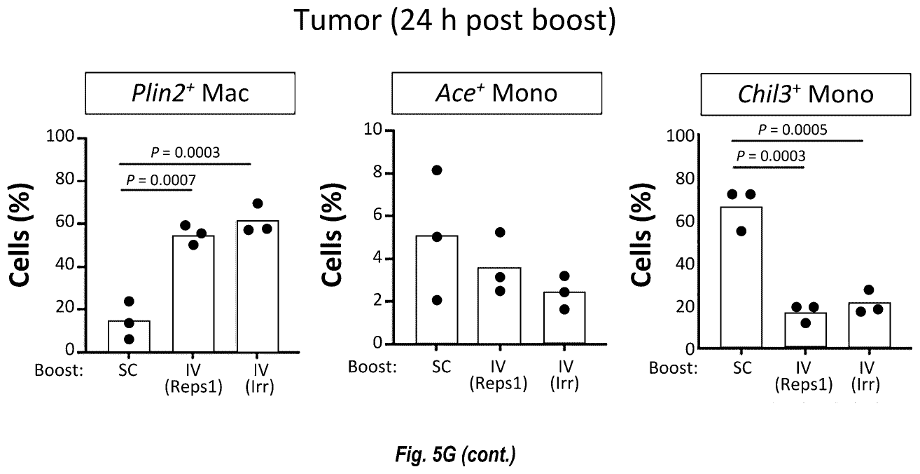

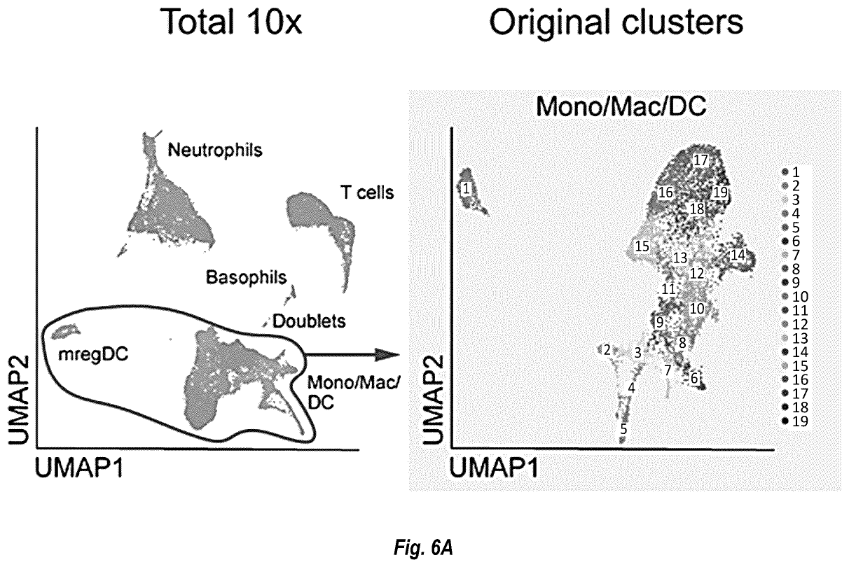

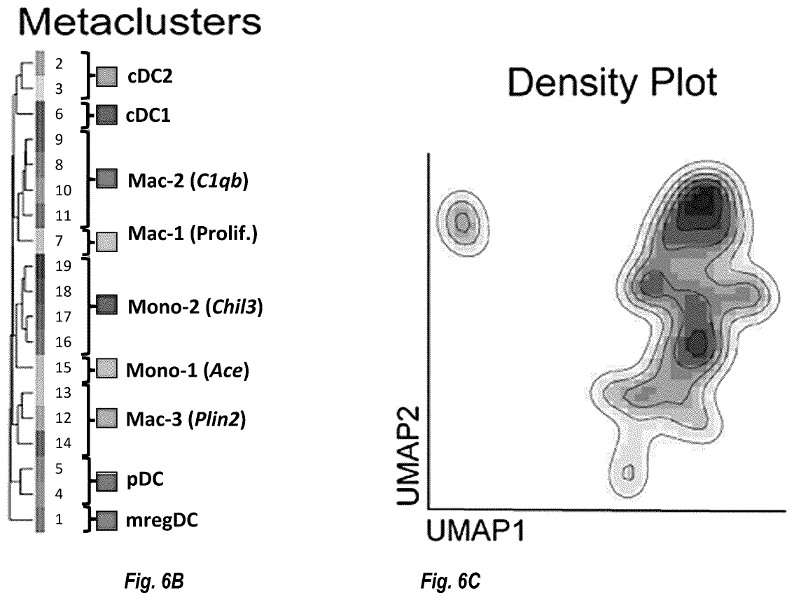

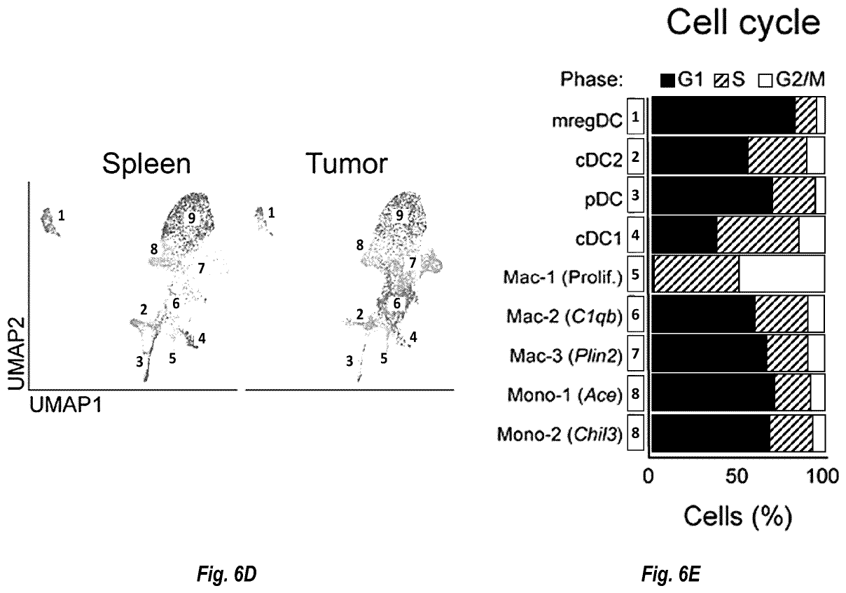

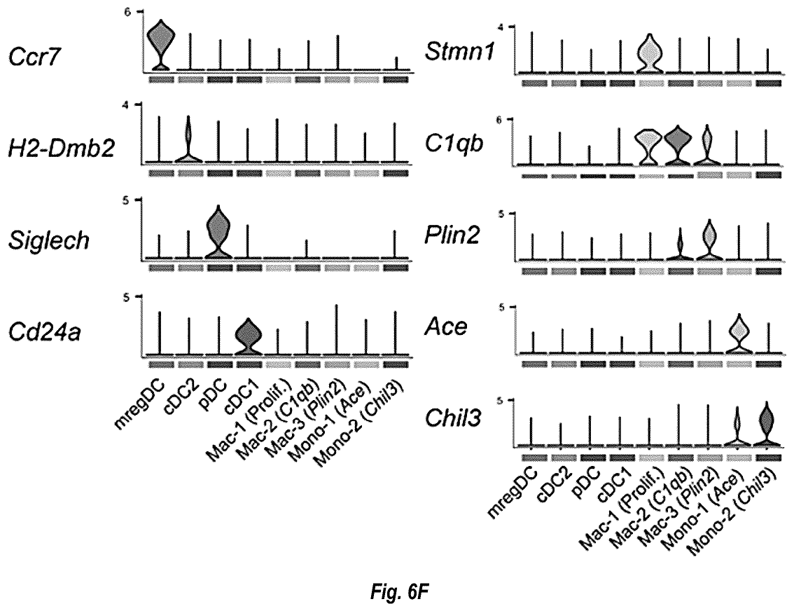

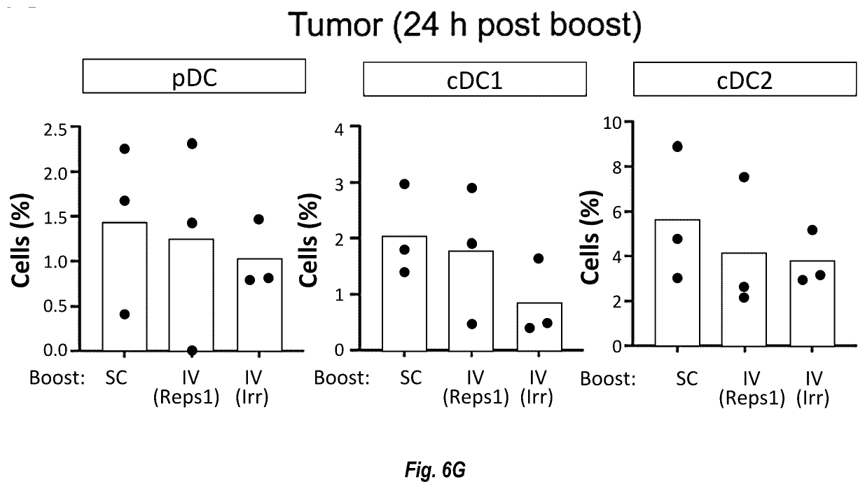

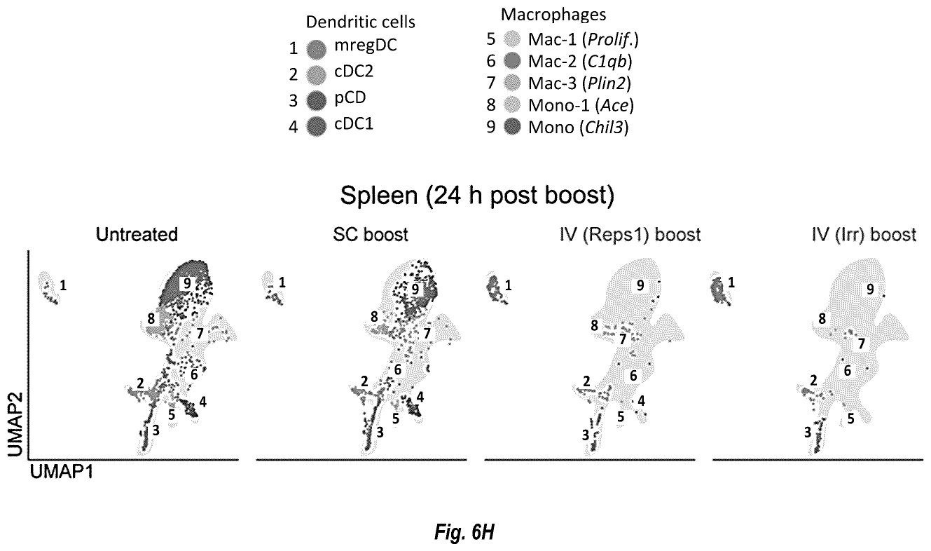

To further characterize the immune responses occurring in the TME, tumors were collected at 24 hours after boosting with SNP-SC or SNP-IV containing the tumor-specific antigen (Reps1) or SNP-IV containing an irrelevant antigen (Trp1). Myeloid cells were sorted by flow cytometry to obtain sufficient cell numbers for downstream analyses based on hematopoietic CD45+ cells expressing CD11b and/or CD11c (FIG. 5A). Spleens were also collected to provide a baseline of major myeloid cell populations. Cells from individual mice were barcoded before scRNA-seq using the droplet-based system of 10× Genomics. Cells were clustered based on gene expression using an unsupervised inference analysis using Seurat (v4) pipeline. Focusing on monocyte, macrophages and DCs, nineteen “original clusters” were identified and visualized by UMAP dimension reduction (FIG. 6A). For simplification, the clusters were organized into 9 “metaclusters” following their hierarchical ordering based on their Euclidian distance (FIG. 6B) as well as formation of stable states as depicted on density plots (FIG. 6C). Based on expression of canonical markers, the 9 metaclusters included 4 DC populations: cDC1 (Batf3, Clec9a, Cd24a), cDC2 (Mgl2, H2-Dmb2, Itgax), pDC (Siglech, Ly6d, Bst2), migratory/regulatory DC (mregDC, Ccr7, Fscn1, Relb), 3 macrophage subpopulations (Cd68, Apo3, Trem2) and 2 monocyte subpopulations (Lyz2, Csf1r, Ccr2) (FIGS. 5B and 5C). Although all 9 metaclusters were present in both spleens and tumors, the TME was primarily composed of monocytes and macrophages (FIGS. 5D and 6D). Based on cell cycle analysis, Mac-1 was identified as a proliferating subset of macrophages due to high expression of genes related to G2/M phase (FIG. 6E). Then, Mac-2, Mac-3, Mono-1 and Mono-2 were annotated based on high expression of C1qb, Plin2, Ace and Chil3 (YM1), respectively (FIGS. 5E and 6F).

Across treatment groups, a pattern was identified corresponding to anti-tumor efficacy: untreated and SNP-SC boosted animals showed enrichment of Chil3+ monocytes in the tumors whereas groups boosted with SNP-IV (Reps1) and SNP-IV (irrelevant antigen) were enriched for Plin2+ macrophages (FIG. 5F). Indeed, the frequency of intratumoral Plin2+ macrophages was significantly increased in SNP-IV boosted animals (FIGS. 5G and 6G). In contrast, the frequencies of Chil3⁺ monocytes and C1qb⁺ macrophages were significantly diminished in SNP-IV boosted animals, both in the tumors and spleens (FIG. 6H). Consistent with flow cytometry data, an enrichment of mregDCs (Maier et al., 2020) was observed in the spleen and tumors after SNP-IV (FIGS. 5G and 6I).

### Example 6. Chil3⁺ Monocytes Express Gene Signatures Associated with Immunoregulatory Responses

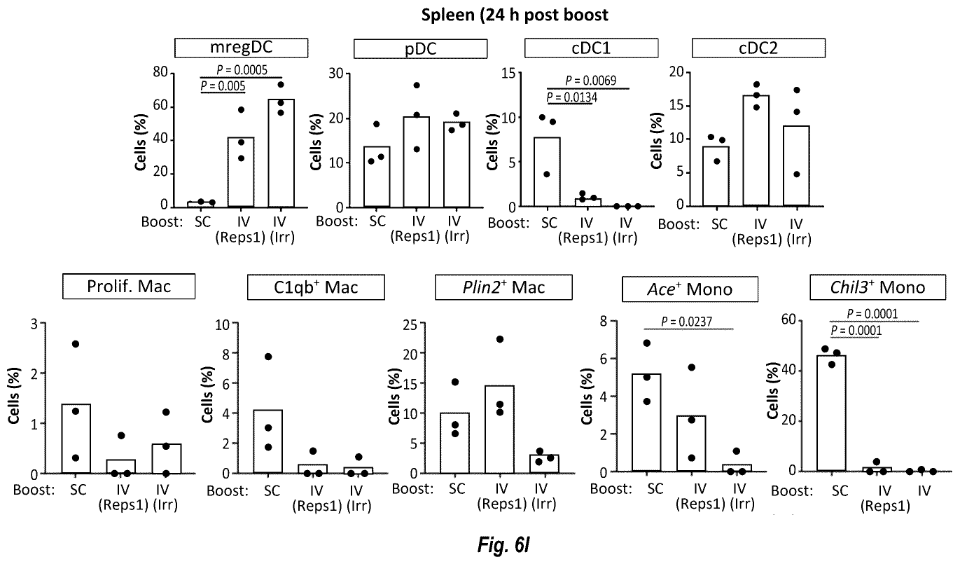

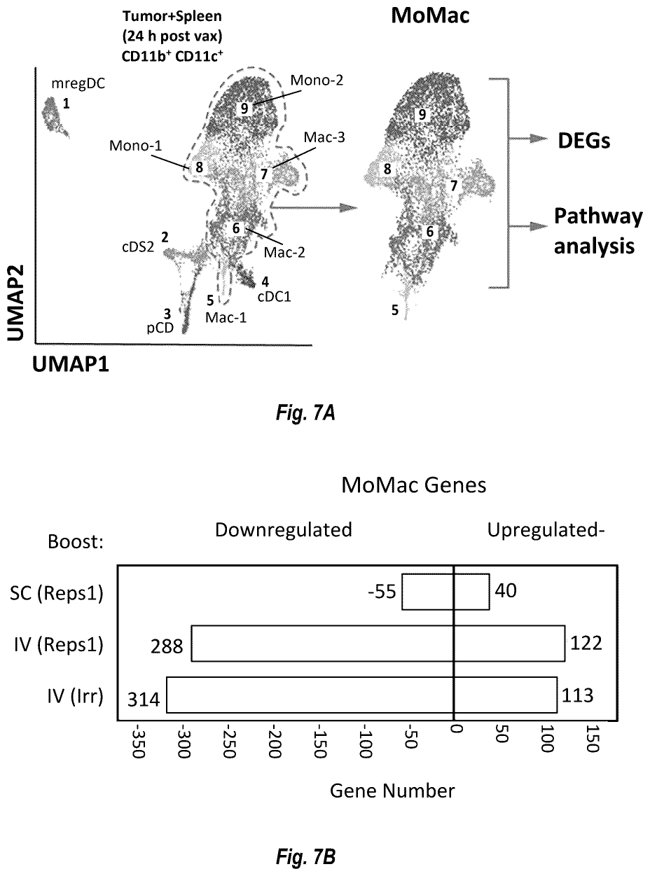

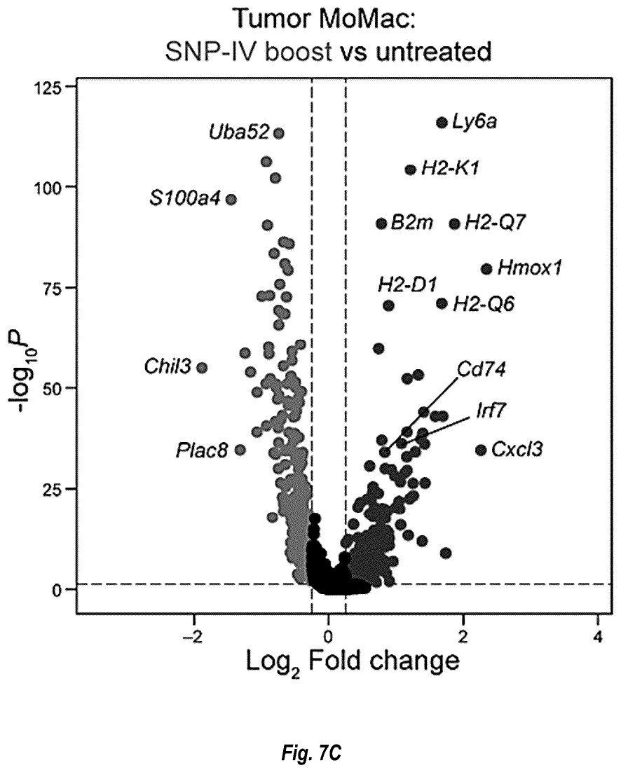

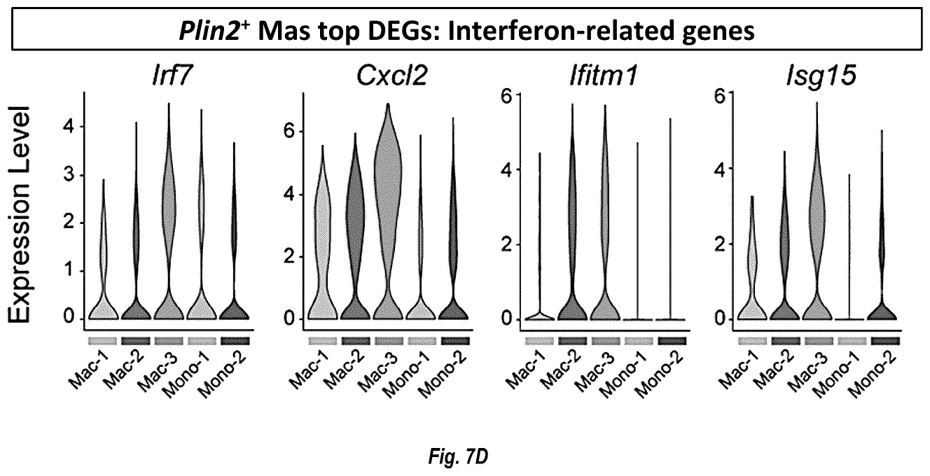

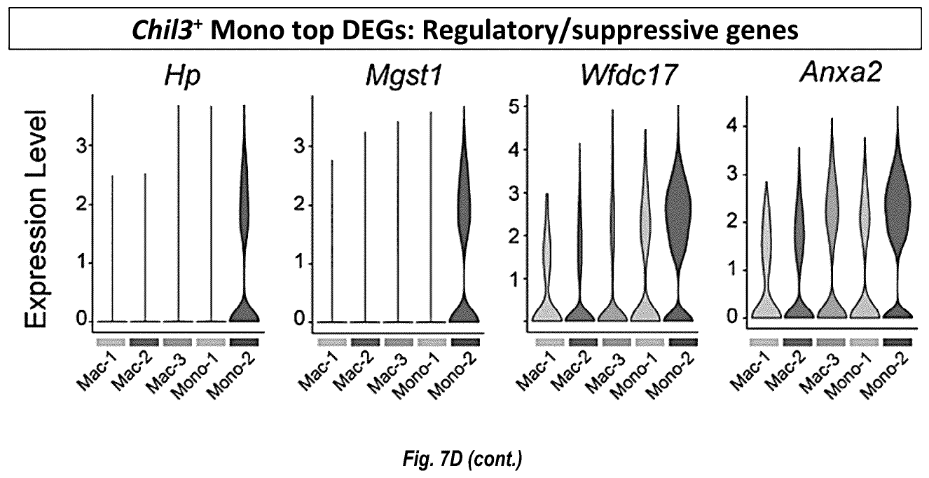

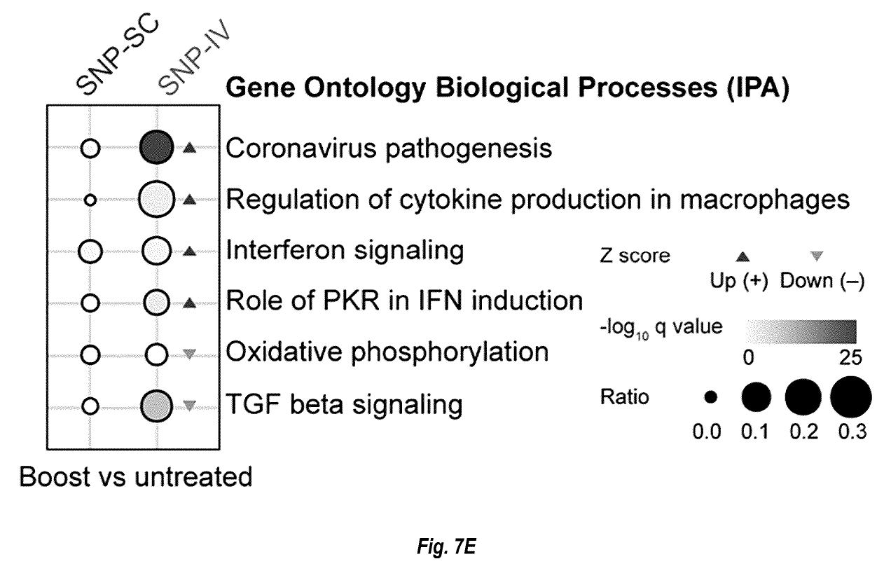

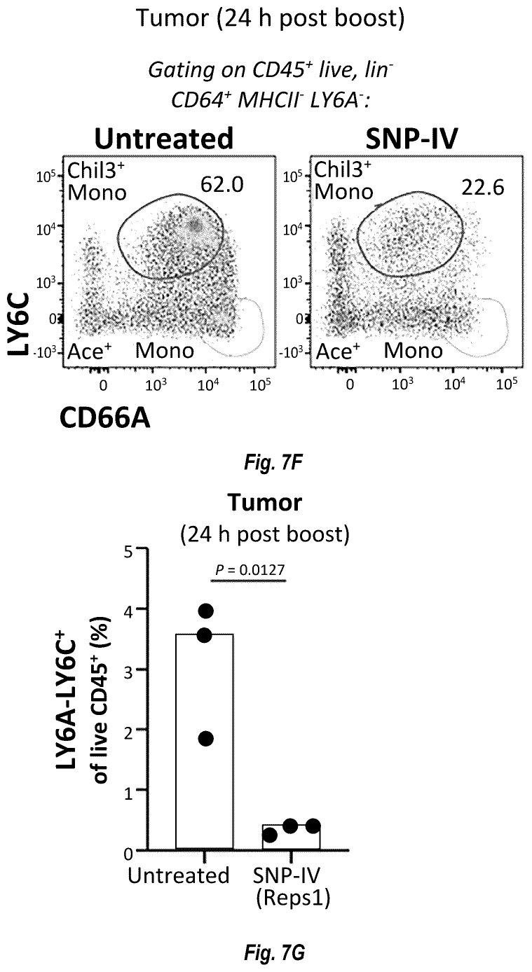

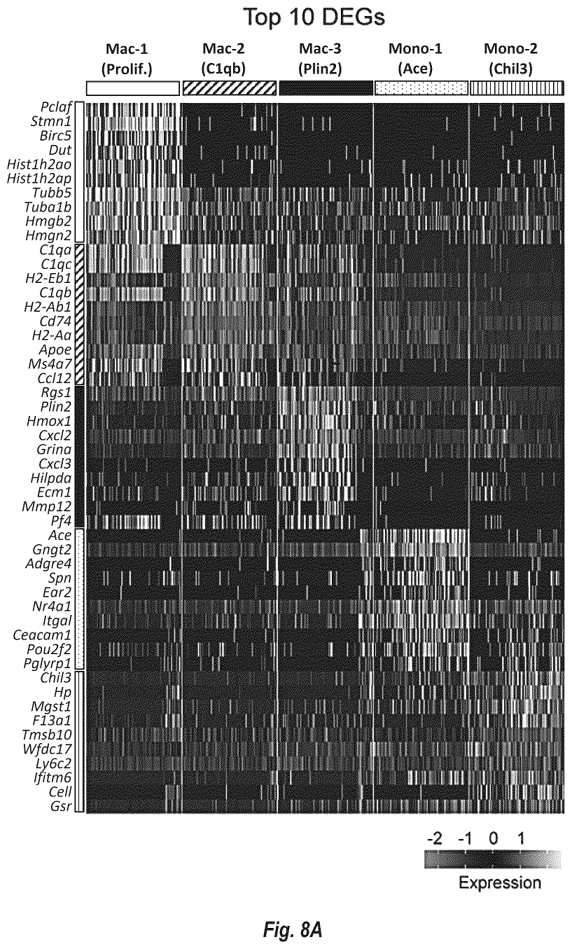

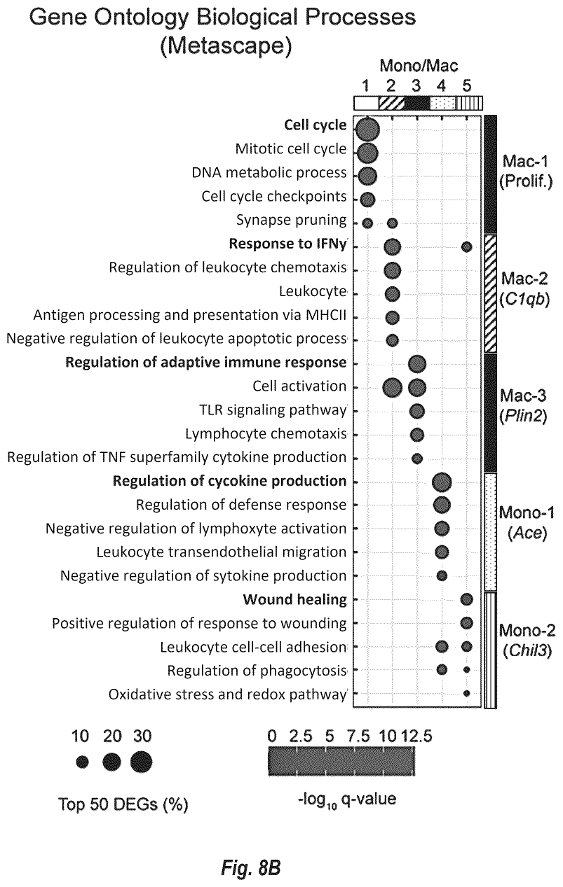

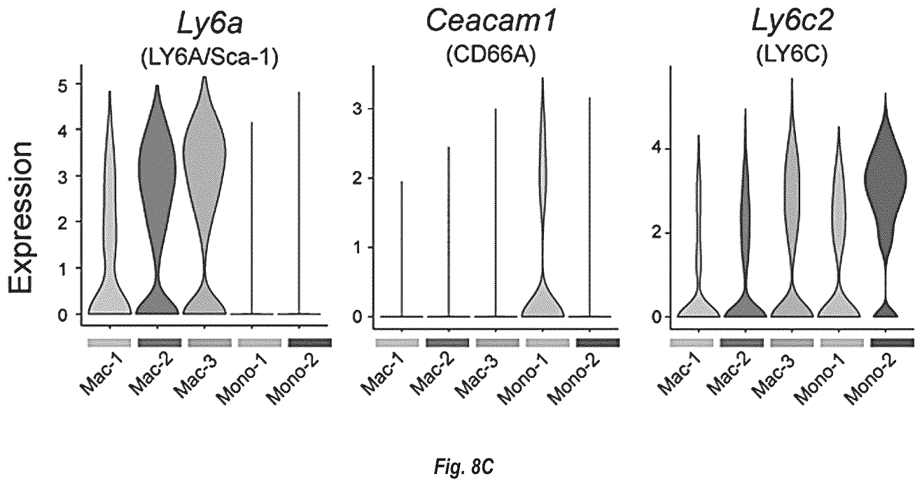

Given the striking differences in the transcription profiles of monocytes and macrophages across groups, further investigation on the intratumoral monocyte/macrophage compartment was performed to understand their unique gene signatures and enriched pathways in each subpopulation (FIG. 7A). More than 100 genes were upregulated following SNP-IV compared to only 40 genes upregulated after SNP-SC (FIG. 7B). Additionally, about 300 genes were downregulated after SNP-IV compared to the 55 genes downregulated after SNP-SC. Each subpopulation can be distinguished by their unique top differentially expressed genes (DEGs) (FIG. 8A and Table S1). Among the top DEGs, Plin2⁺ macrophages upregulated genes related to interferon signaling, including Irf7, Cxcl2, Ifitm1 and Isg15 whereas Chil3⁺ monocytes upregulated genes regulatory or suppressive activity (FIGS. 7C and 7D). For example, Hp encodes haptoglobin that can form complexes with HMGB1 to elicit anti-inflammatory enzymes and cytokines (Yang et al., 2016); Mgst1 encodes microsomal glutathione S-transferase, an enzyme that regulates prostaglandin E2 production, also involved in promoting anti-inflammatory cytokines such as IL-10 (Castoldi et al., 2020; MacKenzie et al., 2013); Wfdc17 encodes a WAP domain protein expressed in MDSCs (Veglia et al., 2021); Anxa2 encodes Annexin A2, a cytoskeletal protein widely implicated in promoting cancer progression (Zhang et al., 2012). Referring to the MetaScape database (Zhou et al., 2019) revealed an enrichment of pathways unique to each monocyte/macrophage population (FIG. 8B). Consistent with a pro-inflammatory response, Plin2⁺ macrophages were enriched in pathways involved in regulation of adaptive immune responses, cell activation, TLR signaling and regulation of TNF production (FIG. 8B). In contrast, Chil3⁺ monocytes were enriched in pathways involved in anti-inflammatory response such as wound healing.

To better interpret the changes in gene expression, patterns of pathway changes were explored using Ingenuity Pathway Analysis (IPA). Genes associated with coronavirus pathogenesis pathway was significantly and highly upregulated after SNP-IV compared to untreated groups in monocytes/macrophages (FIG. 7E). This pathway includes genes involved in IFN-I signaling and inflammasome activation such as Irf7, Ccl5, Oas1 and Pycard, consistent with a viral gene signature. In contrast, genes involved in oxidative phosphorylation, a metabolic process favoring anti-inflammatory phenotypes, was significantly downregulated after SNP-IV. In summary, the observed differences in gene expression across groups further confirm a pattern of macrophages expressing interferon-stimulated genes following SNP-IV and monocytes expressing regulatory genes following SNP-SC.

Next, cell surface markers were analyzed for identification of Chil3⁺ monocytes by flow cytometry. First, LY6A/Sca-1 and MHCII were used as exclusion markers; these are markers are highly expressed on macrophages but not monocytes (FIG. 8C). As Chil3 encodes a secreted protein, antibodies against CD66A (Ceacam1) and LY6C (Ly6c2) that are highly expressed on Ace⁺ monocytes and Chil3⁺ monocytes, respectively (FIG. 8C), were stained with antibodies. Tumors harvested 24 hours post SNP-IV boost showed a three-fold reduction in the frequency of MHCII⁻ LY6A⁻ CD66^{dim} LY6C⁺ monocytes compared to untreated animals (FIGS. 7F and 7G). This finding confirms the identification and reduction of a comparable subpopulation of monocytes in the tumor after SNP-IV that may contribute to the difference in anti-tumor efficacy following vaccination.

### Example 7. Interferon Alpha Required for Mediating Anti-Tumor Efficacy after SNP-IV Treatment

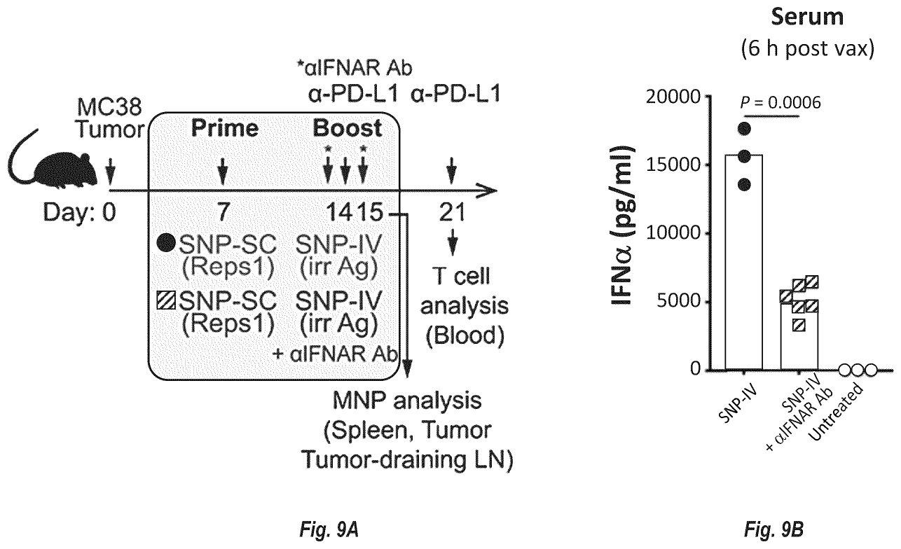

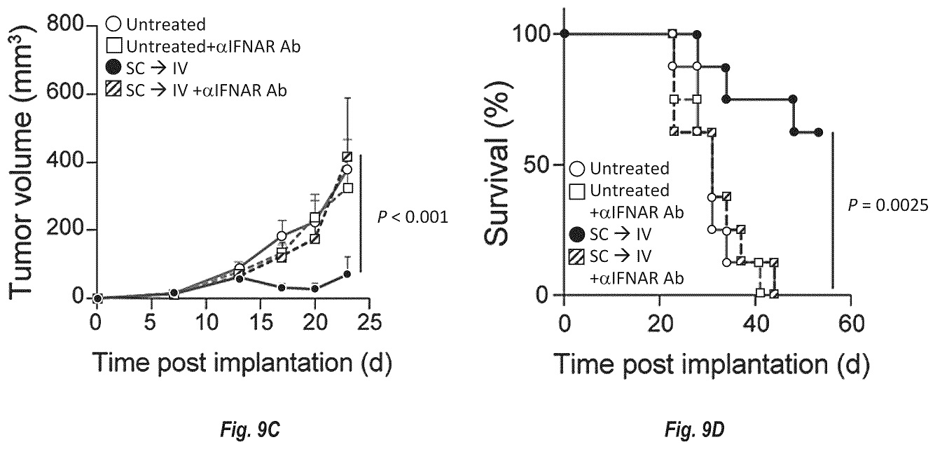

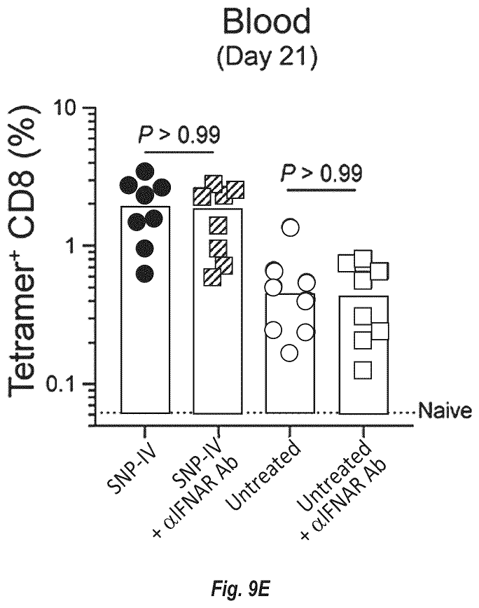

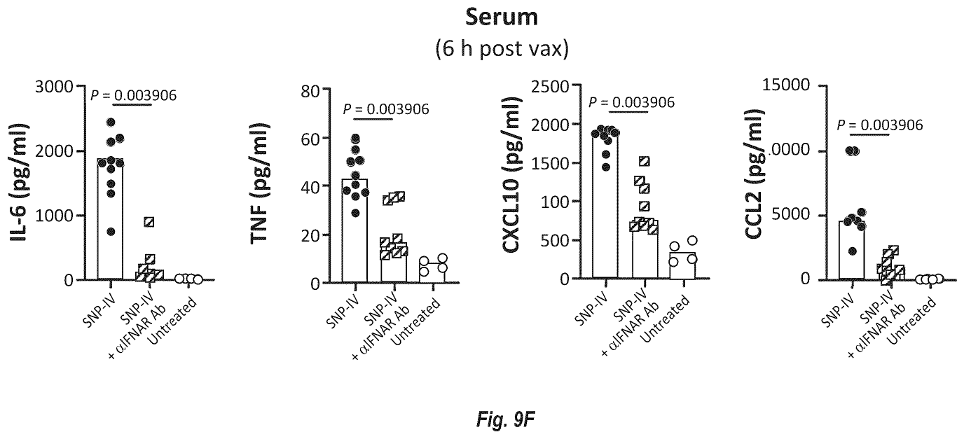

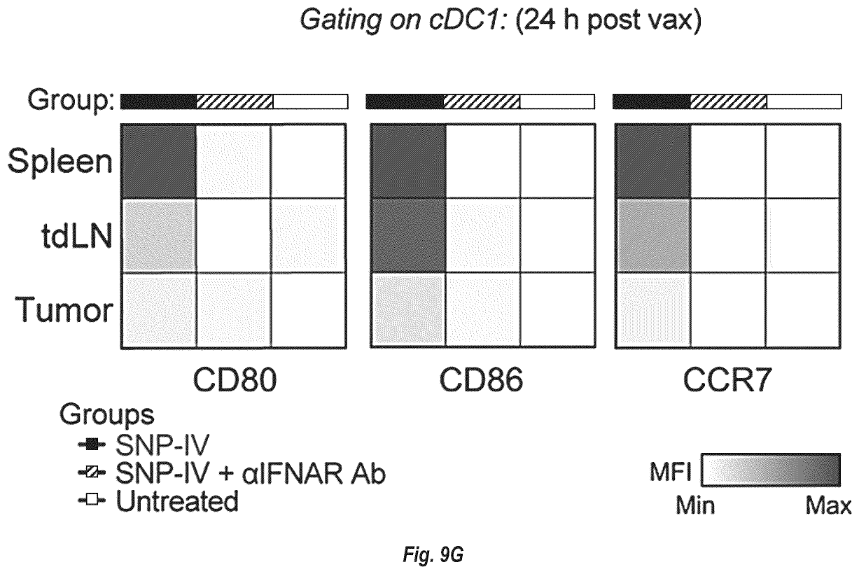

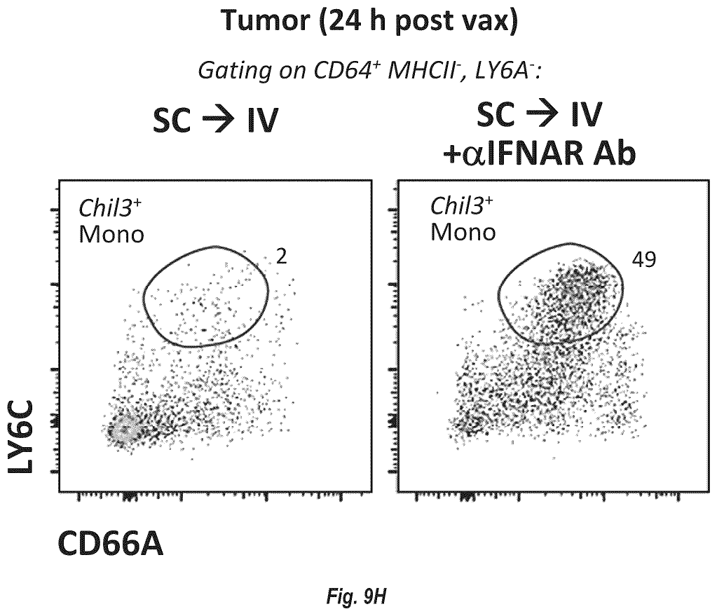

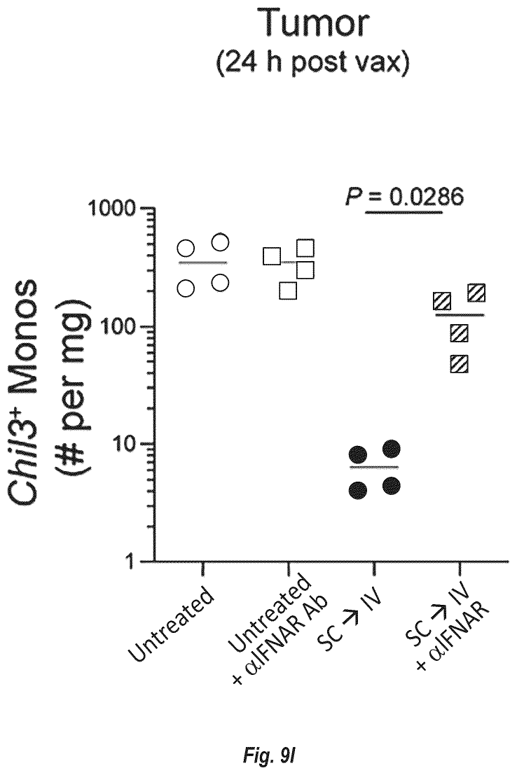

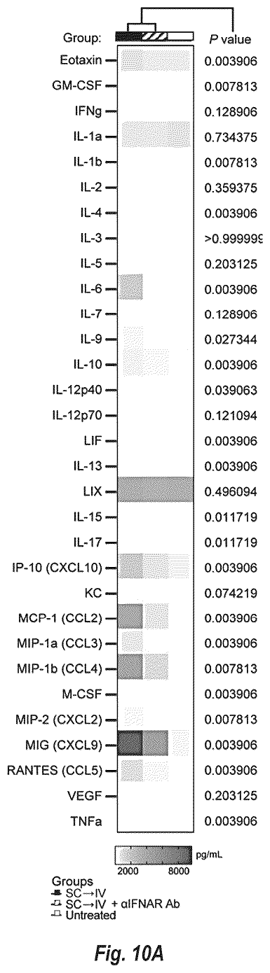

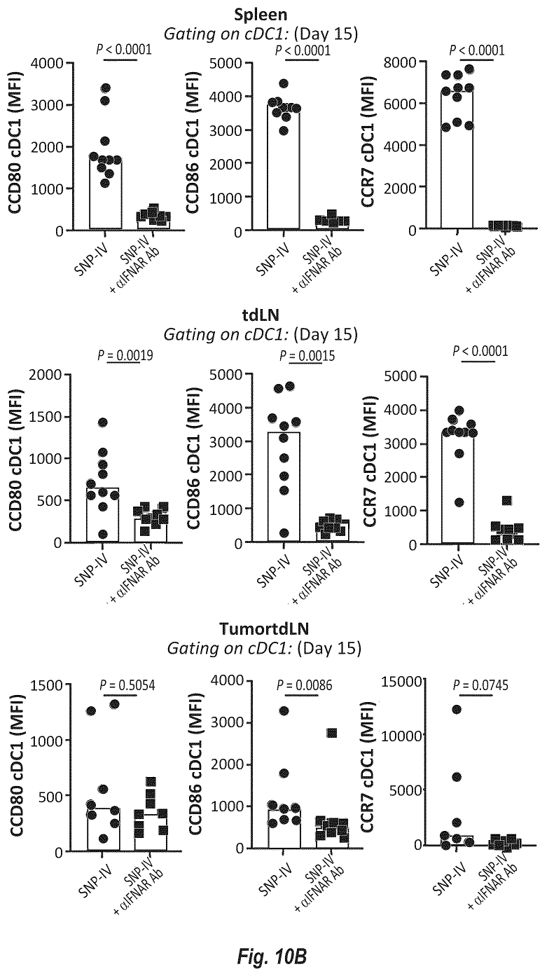

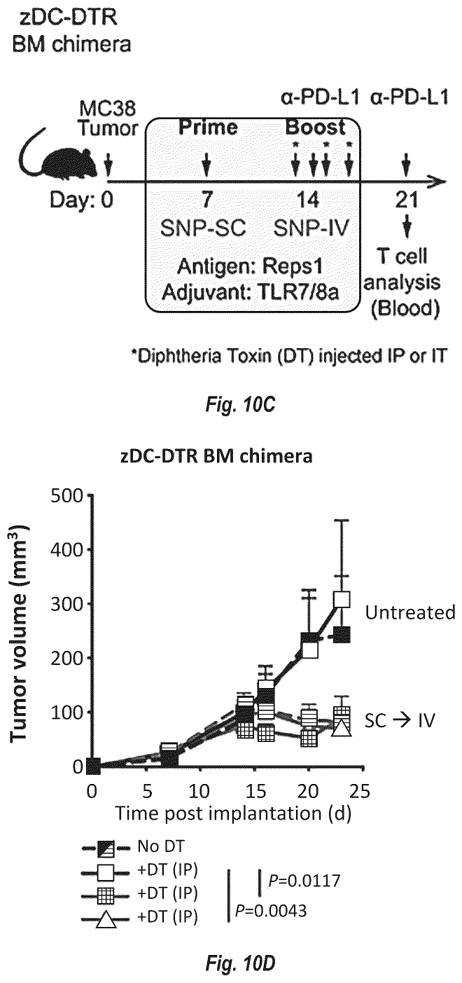

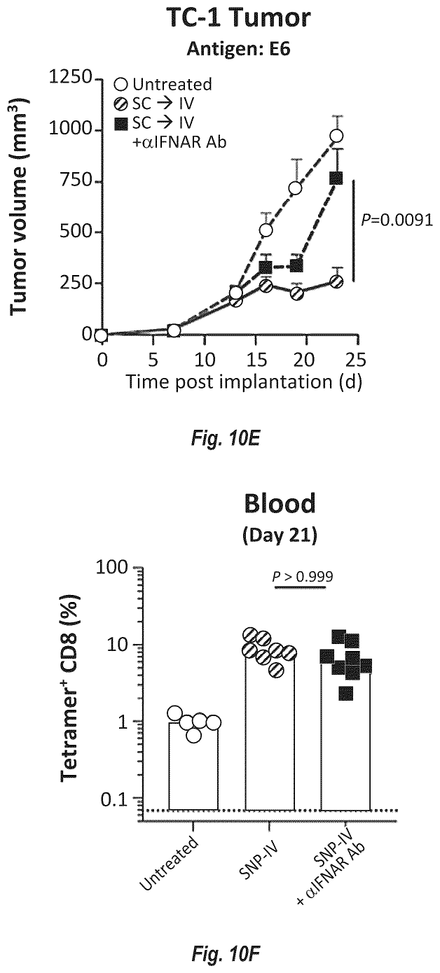

Given that interferon-related genes were highly enriched after SNP-IV, the role of IFN-I in mediating anti-tumor control after SNP-IV was assessed. TLR7/8a and polyIC:LC are both potent inducers of IFN-I with pleiotropic effects across innate and adaptive immune responses (McNab et al., 2015; Sultan et al., 2020). IFN-I signaling plays an important role in promoting anti-tumor function (Duong et al., 2022; Fuertes et al., 2011). It is known that IFN-I signaling is required for CD8+ T cell priming after SNP-7/8a vaccination using Ifnar^{-/-} mice that lack a functional IFNαβ receptor (IFNAR). As boosting T cell responses is not required to mediate anti-tumor effect after SNP-IV given on day 14 (FIGS. 1F and 1H), tumor-bearing mice were injected with control or blocking antibodies against IFNAR on days 13 and 15 (FIG. 9A) to assess its potential role on the innate immune response by SNP-IV. Sera collected 6 hours after SNP-IV confirmed that IFNα was depleted by three-fold after IFNAR blocking although a residual amount of IFNα was detectable (FIG. 9B). Blocking IFNAR signaling had no additional impact on untreated animals but abrogated control of tumor growth following SNP-IV boost (FIG. 9C) as well as decreased survival (FIG. 9D) compared to SNP-IV treated animals without IFNAR blocking. As animals were boosted with SNP-IV containing an irrelevant antigen, blocking IFNAR had no impact on the frequency of neoAg-specific CD8⁺ T cells (FIG. 9E). Other pro-inflammatory cytokines such as IL-6 and TNF as well as chemokines such as CXCL10 and CCL2 were also significantly reduced following IFNAR blocking (FIGS. 9F and 10A).

To investigate the mechanism of how IFN-I promotes T cell-mediated tumor control, the effect of blocking IFNAR DC maturation or impairment was assessed. Blocking IFNAR resulted in lower expression of maturation markers such as CD80, CD86 and CCR7 on cDC1s and cDC2s in the spleen, tumor and tumor-draining LN a day after SNP-IV boost (FIGS. 9G and 10B). To directly test the involvement of DCs in licensing neoAg-specific CD8⁺ T cells, conditional knockouts of cDCs known as zDC-DTR bone marrow chimeric mice were generated (Meredith et al., 2012) (FIG. 10C). Mice were injected with diphtheria toxin (DT) on day 13, day 15 and day 17 i.e., 1 day prior to SNP-IV boost as well as 1 day and 3 days after to sustain the depletion. Groups that received SNP-SC prime followed by SNP-IV boost had significantly smaller tumors compared to untreated controls, regardless of whether the mice received DT injections or not, suggesting that cDCs may be dispensable at the time of boost in promoting tumor regression (FIG. 10D). Next, to test the hypothesis that monocytes and macrophages may be playing an important role in promoting anti-tumor immunity via IFN-I signaling induced by SNP-IV, tumors were harvested 24 h after vaccination with or without blocking IFNAR. Indeed, the depletion of Chil3⁺ monocytes following SNP-IV was reversed when IFNAR was blocked in MC38 tumors (FIGS. 9H and 91) or TC-1 tumors (FIGS. 10E-10G). These data show that anti-tumor efficacy after SNP-IV at the time of boost was dependent on type I IFN leading to depletion of immunoregulatory Chil3⁺ monocytes in the tumor.

### Example 8. Chil3⁺ Monocyte Gene Signature is Enriched in Human Tumor-Associated Monocytes

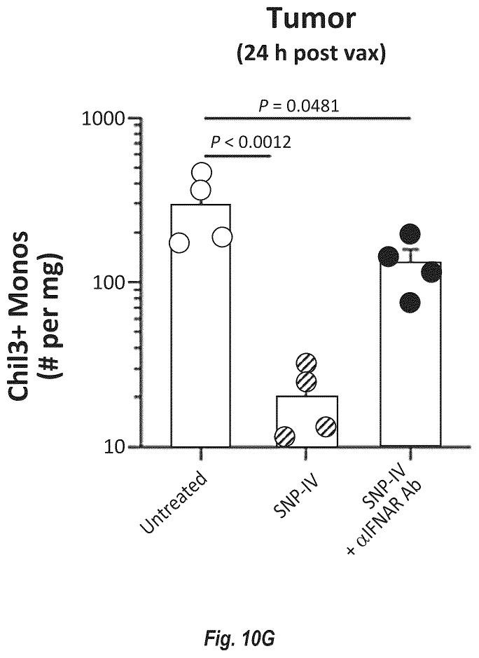

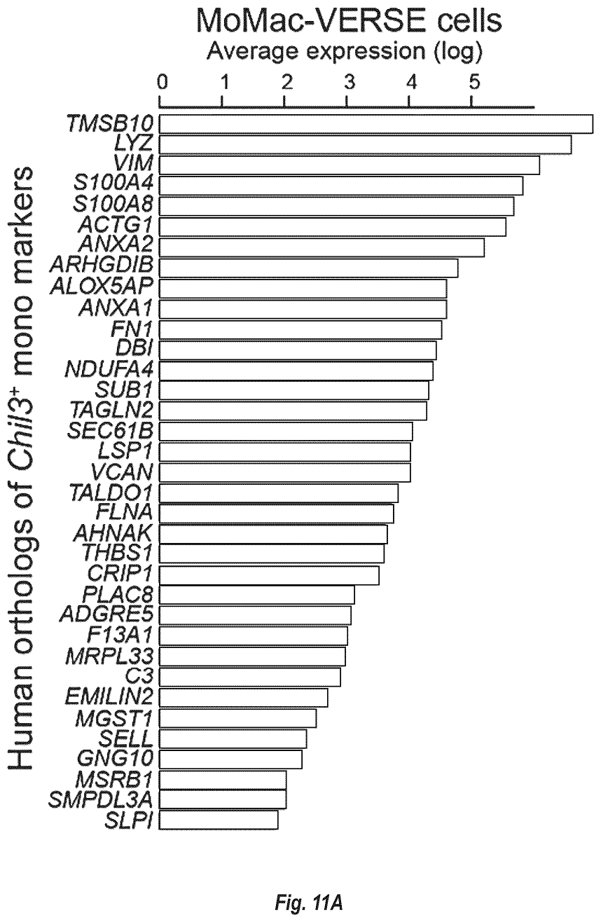

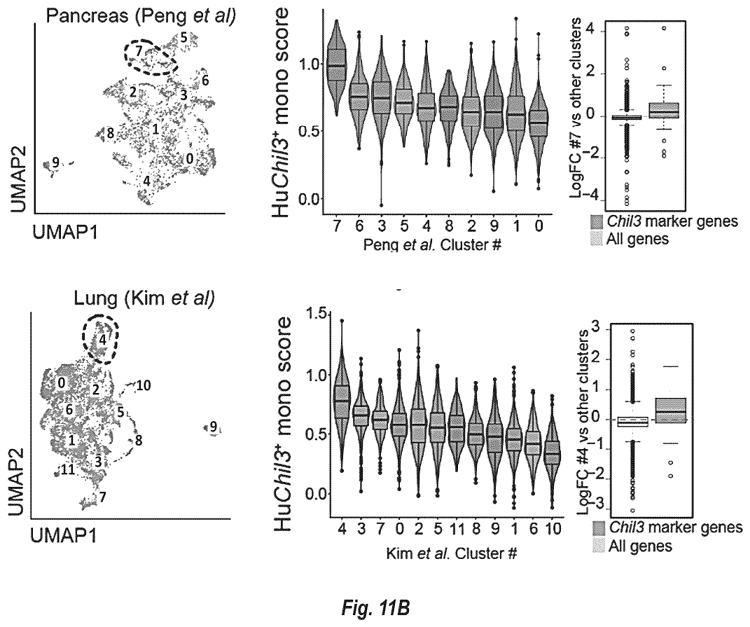

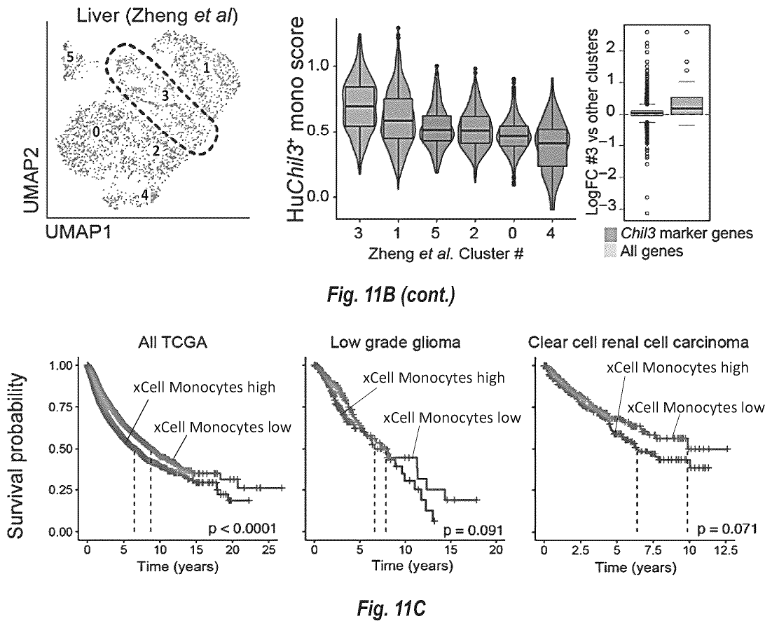

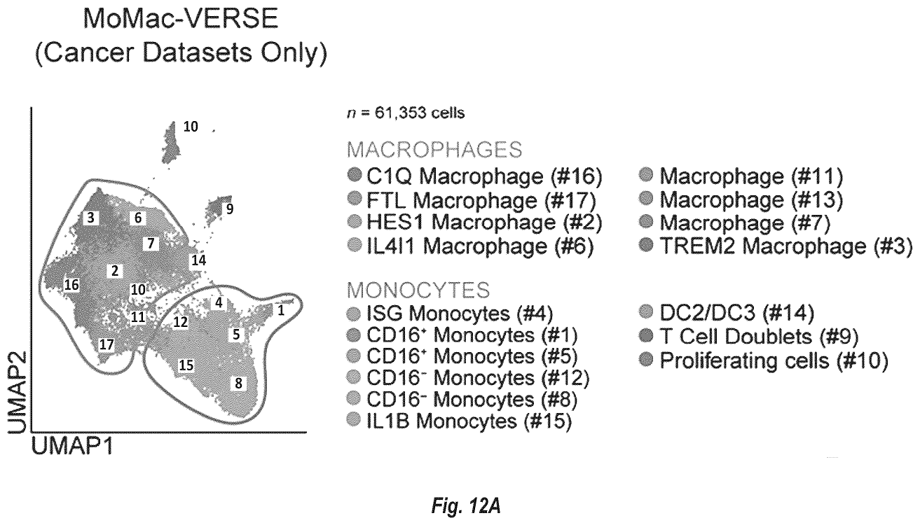

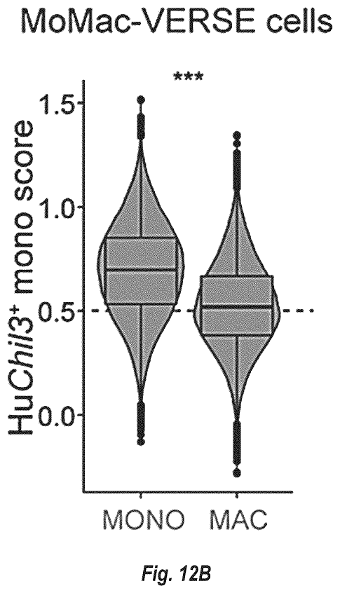

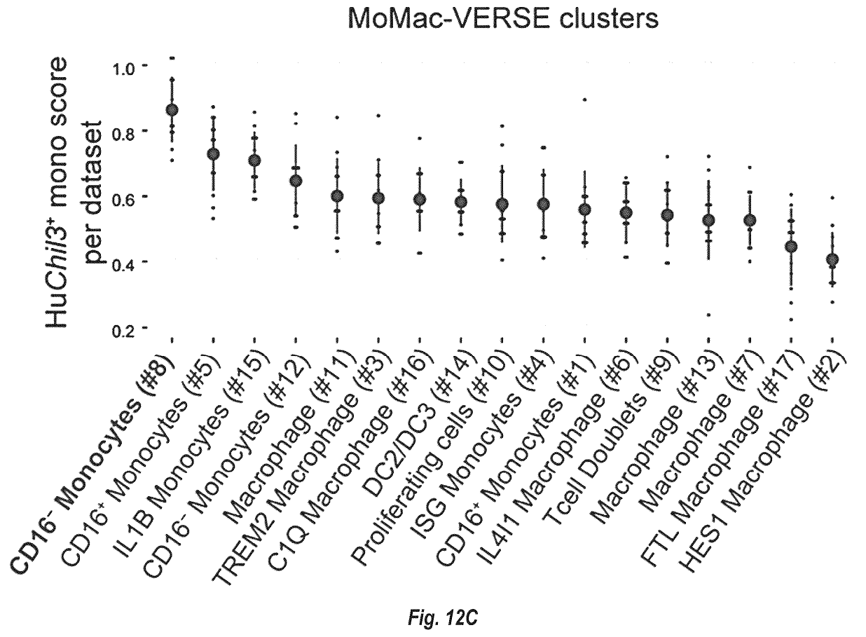

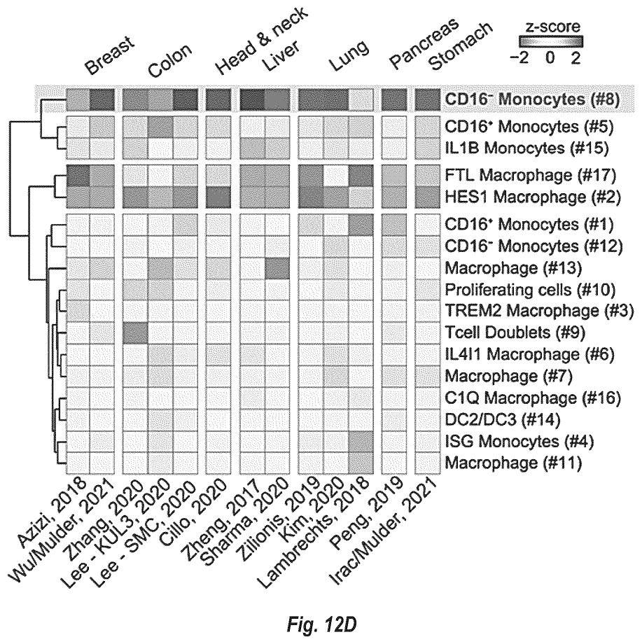

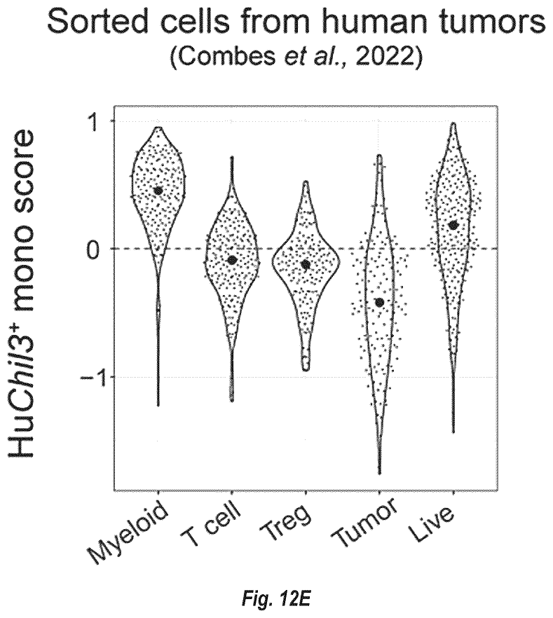

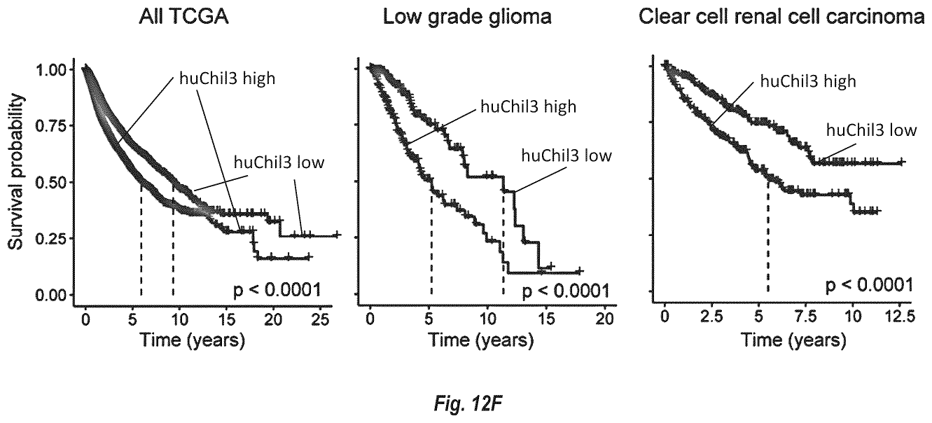

The data in two murine models discussed in Examples 2-7 suggest that altering the TME through IFN-I-mediated modulation of monocytes may be important for T cells to control tumor growth. To determine whether the TME in human cancers may also contain similar monocyte populations, a list of human ortholog markers (referred to as “huChil3 geneset” based on the top 50 differentially expressed genes by Chil3⁺ monocytes was created (FIG. 11A). Droplet-based cancer datasets from the human monocyte macrophage atlas (MoMac-VERSE) (Mulder et al., 2021) consisting of 61,353 cells were analyzed (FIG. 12A). An enrichment of various huChil3 genes in the MoMac-VERSE was discovered with a bias towards monocytes rather than macrophages in line with prior annotations (FIG. 12B). The huChil3 geneset was found to be enriched in CD16⁻ monocytes (cluster #8 of MoMac-VERSE) (FIG. 12C) across various tumors including breast (Azizi et al., 2018; Mulder et al., 2021; Wu et al., 2021), colon (Lee et al., 2020; Zhang et al., 2020), head and neck (Cillo et al., 2020), liver (Sharma et al., 2020; Zheng et al., 2017), lung (Kim et al., 2020; Lambrechts et al., 2018; Zilionis et al., 2019), pancreas (Peng et al., 2019) and stomach cancer (Mulder et al., 2021) (FIG. 12D). To confirm that this observation is not an artifact of large-scale dataset integration, unbiased clustering was performed on individual datasets of pancreatic cancer, lung cancer and liver cancer; huChil3 geneset was enriched in the CD16⁻ monocyte cluster of each study (FIG. 11B).

Finally, these observations were assessed for a possible correlation with disease outcome. Bulk RNA-seq samples analyzed from 364 individual tumors across 12 cancer types (Combes et al., 2022) showed that the huChil3 geneset is enriched in sorted myeloid cells but not T cells, regulatory T cells or tumor cells (FIG. 12E). This enrichment suggests that this geneset allows us to infer h huChil3⁺ monocyte abundance via deconvolution in bulk RNA-seq data. Expression and associated survival data from The Cancer Genome Atlas (TCGA) was used next. Low huChil3 levels were found associated with better survival in a pan-TCGA analysis. This was also true for general monocyte abundance as inferred via xCell (Aran et al., 2017). Notably, a subset of cancer indications including clear cell renal cell carcinoma (ccRCC) and low-grade glioma (LGG) deviated from this overall observation, where high huChil3 expression but not general monocyte expression was associated with worse outcome (FIGS. 12F and 11C). In summary, large-scale datasets from various human tumors support the identification of tissue monocytes that express immunosuppressive genes such as TMSB10 (Thymosin β10, a key regulator of tumorigenesis (Zhang et al., 2017) and ANXA2 (Annexin A2), which are shared with Chil3⁺ monocytes and were identified here as a negative regulator of anti-tumor immunity that may contribute to worse disease outcomes.

### Discussion Regarding Examples 1-8

Efforts in developing therapeutic cancer vaccines have primarily focused on expanding the magnitude or quality of tumor-specific T cell responses in combination with checkpoint inhibitors that can enhance CD8⁺ T cell function (Saxena et al. 2021 Nat Rev Cancer). While generating tumor-specific T cells may be necessary for protection, tumor-induced immune suppression may be a major obstacle in achieving complete tumor regression in patients. The present disclosure provides direct evidence in a preclinical tumor model that systemic induction of IFN-I provided by an immunostimulant delivered intravenously can have a profound effect on remodeling the TME thus enabling improved anti-tumor efficacy of vaccine-generated tumor-specific CD8⁺ T cells.

Previously, modifying the route of administration of the SNP-7/8a vaccines was shown to alter the quality of neoAg⁺ CD8⁺ T cells. SNP-IV generated more stem-like cells compared to SNP-SC that resulted in more terminally differentiated cells. A prime-boost regimen of SNP-IV given twice resulted in significant tumor regression but not SNP-SC given twice, despite high magnitude responses. One explanation for why SNP-IV was effective is that the stem-like cells responded to checkpoint inhibitors by replenishing the pool of effector cells. The present disclosure provides a second explanation as to why SNP-IV may be beneficial: systemic innate immune activation. This paradigm is referred to as “vax-innate” and it emphasizes two immunological events that can lead to effective tumor regression: (1) generation of tumor-specific CD8⁺ T cells and (2) systemic innate immune activation to reprogram the suppressive TME.

The requirement for systemic immunity in the context of cancer immunotherapies have been explored by others, especially in the context of sustaining T cell immunity (Allen et al., 2020; Spitzer et al., 2017). As a large secondary lymphoid organ, the spleen would be the primary site for positive DC-T cell interactions following SNP-IV; stem-like CD8⁺ T cells have been shown to form niches within the spleen (Im et al., 2016). Given that tumors are also vascularized, a systemic delivery of a nanoparticle peptide-based TLR7/8a vaccine can lead to intratumoral distribution. In translating this to humans, IV injection may be the most effective approach for treating metastatic tumors rather than direct intratumoral injections of innate stimulation to one or a few sites. An important caveat will be how IV delivery of an innate stimuli can be clinically tolerated.

Investigations into the TME and how the immune compartment influences tumor progression or regression have led to a focused effort on myeloid cells (Binnewies et al., 2018). Myeloid-targeting therapies have emerged as a promising approach for cancer immunotherapies, given the greater flexibility for tumor-antigen agnostic treatments. Various approaches include antibodies activating CD40 (a co-stimulatory receptor expressed by DCs) or blocking CSF1R (a survival receptor expressed by macrophages) or agonists to innate immune receptors such as stimulator of interferon genes (STING) or toll-like receptors (TLRs) could potentially reprogram suppressive myeloid cells to a pro-inflammatory state. These monotherapies may not be effective in patients that do not have sufficient levels of endogenous tumor-infiltrating T cells (Chaib et al., 2020).

Vaccines that induce CD8⁺ T cells usually require the induction of IFN-I for cross presentation. As disclosed herein, the vaccine-induced innate stimulation can have an added effect of altering the TME when delivered systemically. Other immune modulators that have shown superior anti-tumor efficacy when given systemically include polyIC:LC (an agonist that binds MDA5 and TLR3) (Sultan et al., 2020). Production of IFN-I leads to increased CXCL9 and CXCL10, which are chemokines that may be important in recruiting T cells into the tumor. As disclosed herein, the efficacy of SNP-IV relies on the systemic production of IFN-I. However, a significant difference was not observed in the infiltration of neoAg-specific CD8⁺ T cells measured in digested single cell suspensions. Ongoing efforts are focused on visualizing the TME using spatial profiling technologies to better understand the role of systemic inflammation in dictating the localization of CD8⁺ T cells within the tumor.

RNA-lipoplexes encoding tumor antigens have also been given systemically with the rationale that targeting the spleen offers the highest density of DCs to prime high magnitude T cells (Kranz et al., 2016). Although not directly addressed by Kranz et al., the RNA-lipoplexes themselves may act as an immunostimulant that could have modified the TME. The requirement for positive T cell-myeloid cell interactions in the TME following vaccination has been described by Thoreau et al. where the authors co-delivered E7 peptide (an HPV antigen) together with IFNα (Thoreau et al., 2015). Interestingly, administering systemic IFNα alone was ineffective; antigen-specific T cells still need to be present. This is consistent with observations that while systemic inflammation can modify the TME, there is still a dependency on CD8⁺ T cells to promote tumor regression.

Although antibodies inhibiting CSF1R are often used to deplete TAMs, Sluis et al. showed that peptide-based vaccine-induced tumor regression was lost upon depletion of TAMs (van der Sluis et al., 2015). This points to the anti-tumoral role of macrophages that has not been as well described as their pro-tumoral contributions. Given the heterogeneity of monocytes and macrophages, a better characterization of subpopulations can provide a more defined approach in targeting specific cells rather than broadly depleting all monocytes and macrophages. As disclosed herein, Plin2⁺ macrophages were upregulated in tumors after SNP-IV but not in untreated or SNP-SC treated mice. As PLIN2 is involved in lipid droplet formation, lipid metabolism may play an important role in supporting inflammatory conditions in the TME, similar to classically-activated pro-inflammatory macrophages (M1) (Rosas-Ballina et al., 2020). Lipid-associated macrophages (LAMs) have also been described as having a protective role in maintaining metabolic homeostasis, both in mice and humans (Jaitin et al., 2019). In contrast, Chil3⁺ monocytes and C1qb⁺ macrophages were downregulated in tumors after SNP-IV but highly expressed in untreated or SNP-SC treated mice. Chil3 is a gene also highly expressed in alternatively-activated anti-inflammatory macrophages (M2). Further expression of genes encoding inhibitory molecules such as ANXA2, MGST1 and WFDC17 suggest a mechanism of inhibiting T cells via soluble factors. Although we did not specifically deplete Chil3⁺ monocytes in this study, Shibuya et al. treated Chil3-DTR mice with DT and observed reduced tumor metastases in a systemic B16 tumor model (Shibuya et al., 2021). Their findings in a different tumor model further support the generalizability of our hypothesis that Chil3⁺ monocytes may play a more pro-tumoral role. C1qb⁺ macrophages, which were also found to be modulated by IFN-I, express high levels of Trem2 that encodes TREM2. The high expression of TREM2 on TAMs in both mice and humans (Katzenelenbogen et al., 2020) has made these cells a leading candidate for targeted therapies to overcome the immunosuppressive microenvironment in the tumor (Molgora et al., 2020). Thus, the identification of CD16⁻ monocytes in tumors expressing the human orthologs of Chil3⁺ monocyte geneset offers another potential human candidate for targeted therapies.

While our TLR7/8a-based vaccine triggers a wide range of cytokines, it will be important to identify whether other stimulants of IFNα could also remodel the TME. Similar tumor regression was achieved when combining SNP-7/8a (prime) with viral vaccines (boost) that can also induce systemic innate activation including IFNα production (data not shown). IFNα alone has long been tested as a cancer therapy but its use is dampened by the occurrence of toxic side effects (Sleijfer et al., 2005). The challenge of balancing systemic innate activation with tolerated toxicity will shape the future development of vaccines and immune interventions that can promote efficient T cell-mediated control of the tumor. Finally, the “vax-innate” paradigm can be extended to other approaches of introducing tumor-specific T cells such as adoptive cell therapy and chimeric antigen receptor T cell therapy and then coupling these T cell-based approaches with providing exogenous systemic innate immune activation.

### REFERENCES FOR EXAMPLES 1-8

Allen, B. M., Hiam, K. J., Burnett, C. E., Venida, A., DeBarge, R., Tenvooren, I., Marquez, D. M., Cho, N. W., Carmi, Y., and Spitzer, M. H. (2020). Systemic dysfunction and plasticity of the immune macroenvironment in cancer models. Nat Med 26, 1125-1134.
Aran, D., Hu, Z., and Butte, A. J. (2017). xCell: digitally portraying the tissue cellular heterogeneity landscape. Genome Biol 18, 220.
Azizi, E., Carr, A. J., Plitas, G., Cornish, A. E., Konopacki, C., Prabhakaran, S., Nainys, J., Wu, K., Kiseliovas, V., Setty, M., et al. (2018). Single-Cell Map of Diverse Immune Phenotypes in the Breast Tumor Microenvironment. Cell 174, 1293-1308 e1236.
Baharom, F., Ramirez-Valdez, R. A., Tobin, K. K. S., Yamane, H., Dutertre, C. A., Khalilnezhad, A., Reynoso, G. V., Coble, V. L., Lynn, G. M., Mule, M. P., et al. (2021). Intravenous nanoparticle vaccination generates stem-like TCF1(+) neoantigen-specific CD8(+) T cells. Nat Immunol 22, 41-52.
Binnewies, M., Roberts, E. W., Kersten, K., Chan, V., Fearon, D. F., Merad, M., Coussens, L. M., Gabrilovich, D. I., Ostrand-Rosenberg, S., Hedrick, C. C., et al. (2018). Understanding the tumor immune microenvironment (TIME) for effective therapy. Nat Med 24, 541-550.
Bleriot, C., Chakarov, S., and Ginhoux, F. (2020). Determinants of Resident Tissue Macrophage Identity and Function. Immunity 52, 957-970.
Borden, E. C. (2019). Interferons alpha and beta in cancer: therapeutic opportunities from new insights. Nat Rev Drug Discov 18, 219-234.
Browaeys, R., Saelens, W., and Saeys, Y. (2020). NicheNet: modeling intercellular communication by linking ligands to target genes. Nat Methods 17, 159-162.
Bunis, D. G., Andrews, J., Fragiadakis, G. K., Burt, T. D., and Sirota, M. (2020). dittoSeq: Universal User-Friendly Single-Cell and Bulk RNA Sequencing Visualization Toolkit. Bioinformatics.
Castoldi, A., Monteiro, L. B., van Teijlingen Bakker, N., Sanin, D. E., Rana, N., Corrado, M., Cameron, A. M., Hassler, F., Matsushita, M., Caputa, G., et al. (2020). Triacylglycerol synthesis enhances macrophage inflammatory function. Nat Commun 11, 4107.
Chaib, M., Chauhan, S. C., and Makowski, L. (2020). Friend or Foe?Recent Strategies to Target Myeloid Cells in Cancer. Front Cell Dev Biol 8, 351.
Cheng, S., Li, Z., Gao, R., Xing, B., Gao, Y., Yang, Y., Qin, S., Zhang, L., Ouyang, H., Du, P., et al. (2021). A pan-cancer single-cell transcriptional atlas of tumor infiltrating myeloid cells. Cell 184, 792-809 e723.
Cillo, A. R., Kurten, C. H. L., Tabib, T., Qi, Z., Onkar, S., Wang, T., Liu, A., Duvvuri, U., Kim, S., Soose, R. J., et al. (2020). Immune Landscape of Viral- and Carcinogen-Driven Head and Neck Cancer. Immunity 52, 183-199 e189.
Combes, A. J., Samad, B., Tsui, J., Chew, N. W., Yan, P., Reeder, G. C., Kushnoor, D., Shen, A., Davidson, B., Barczak, A. J., et al. (2022). Discovering dominant tumor immune archetypes in a pan-cancer census. Cell 185, 184-203 el 19.
Dunn, G. P., Koebel, C. M., and Schreiber, R. D. (2006). Interferons, immunity and cancer immunoediting. Nat Rev Immunol 6, 836-848.
Duong, E., Fessenden, T. B., Lutz, E., Dinter, T., Yim, L., Blatt, S., Bhutkar, A., Wittrup, K. D., and Spranger, S. (2022). Type I interferon activates MHC class I-dressed CD11b(+) conventional dendritic cells to promote protective anti-tumor CD8(+) T cell immunity. Immunity 55, 308-323 e309.
Durinck, S., Spellman, P. T., Birney, E., and Huber, W. (2009). Mapping identifiers for the integration of genomic datasets with the R/Bioconductor package biomaRt. Nat Protoc 4, 1184-1191.
Fuertes, M. B., Kacha, A. K., Kline, J., Woo, S. R., Kranz, D. M., Murphy, K. M., and Gajewski, T. F. (2011). Host type I IFN signals are required for antitumor CD8+ T cell responses through CD8{alpha}+ dendritic cells. J Exp Med 208, 2005-2016.
Gubin, M. M., Esaulova, E., Ward, J. P., Malkova, O. N., Runci, D., Wong, P., Noguchi, T., Arthur, C. D., Meng, W., Alspach, E., et al. (2018). High-Dimensional Analysis Delineates Myeloid and Lymphoid Compartment Remodeling during Successful Immune-Checkpoint Cancer Therapy. Cell 175, 1443.
Guilliams, M., Ginhoux, F., Jakubzick, C., Naik, S. H., Onai, N., Schraml, B. U., Segura, E., Tussiwand, R., and Yona, S. (2014). Dendritic cells, monocytes and macrophages: a unified nomenclature based on ontogeny. Nat Rev Immunol 14, 571-578.
Hao, Y., Hao, S., Andersen-Nissen, E., Mauck, W. M., 3rd, Zheng, S., Butler, A., Lee, M. J., Wilk, A. J., Darby, C., Zager, M., et al. (2021). Integrated analysis of multimodal single-cell data. Cell 184, 3573-3587 e3529.
Hegde, P. S., and Chen, D. S. (2020). Top 10 Challenges in Cancer Immunotherapy. Immunity 52, 17-35.
Hegde, S., Leader, A. M., and Merad, M. (2021). MDSC: Markers, development, states, and unaddressed complexity. Immunity 54, 875-884.
Hourani, T., Holden, J. A., Li, W., Lenzo, J. C., Hadjigol, S., and O'Brien-Simpson, N. M. (2021). Tumor Associated Macrophages: Origin, Recruitment, Phenotypic Diversity, and Targeting. Front Oncol 11, 788365.
Im, S. J., Hashimoto, M., Gerner, M. Y., Lee, J., Kissick, H. T., Burger, M. C., Shan, Q., Hale, J. S., Lee, J., Nasti, T. H., et al. (2016). Defining CD8+ T cells that provide the proliferative burst after PD-1 therapy. Nature 537, 417-421.
Jaitin, D. A., Adlung, L., Thaiss, C. A., Weiner, A., Li, B., Descamps, H., Lundgren, P., Bleriot, C., Liu, Z., Deczkowska, A., et al. (2019). Lipid-Associated Macrophages Control Metabolic Homeostasis in a Trem2-Dependent Manner. Cell 178, 686-698 e614.
Katzenelenbogen, Y., Sheban, F., Yalin, A., Yofe, I., Svetlichnyy, D., Jaitin, D. A., Bornstein, C., Moshe, A., Keren-Shaul, H., Cohen, M., et al. (2020). Coupled scRNA-Seq and Intracellular Protein Activity Reveal an Immunosuppressive Role of TREM2 in Cancer. Cell 182, 872-885 e819.
Kim, N., Kim, H. K., Lee, K., Hong, Y., Cho, J. H., Choi, J. W., Lee, J. I., Suh, Y. L., Ku, B. M., Eum, H. H., et al. (2020). Single-cell RNA sequencing demonstrates the molecular and cellular reprogramming of metastatic lung adenocarcinoma. Nat Commun 11, 2285.
Kranz, L. M., Diken, M., Haas, H., Kreiter, S., Loquai, C., Reuter, K. C., Meng, M., Fritz, D., Vascotto, F., Hefesha, H., et al. (2016). Systemic RNA delivery to dendritic cells exploits antiviral defence for cancer immunotherapy. Nature 534, 396-401.
Lam, K. C., Araya, R. E., Huang, A., Chen, Q., Di Modica, M., Rodrigues, R. R., Lopes, A., Johnson, S. B., Schwarz, B., Bohrnsen, E., et al. (2021). Microbiota triggers STING-type I IFN-dependent monocyte reprogramming of the tumor microenvironment. Cell 184, 5338-5356 e5321.
Lambrechts, D., Wauters, E., Boeckx, B., Aibar, S., Nittner, D., Burton, O., Bassez, A., Decaluwe, H., Pircher, A., Van den Eynde, K., et al. (2018). Phenotype molding of stromal cells in the lung tumor microenvironment. Nat Med 24, 1277-1289.
Le Bon, A., Etchart, N., Rossmann, C., Ashton, M., Hou, S., Gewert, D., Borrow, P., and Tough, D. F. (2003). Cross-priming of CD8+ T cells stimulated by virus-induced type I interferon. Nat Immunol 4, 1009-1015.
Lee, H. O., Hong, Y., Etlioglu, H. E., Cho, Y. B., Pomella, V., Van den Bosch, B., Vanhecke, J., Verbandt, S., Hong, H., Min, J. W., et al. (2020). Lineage-dependent gene expression programs influence the immune landscape of colorectal cancer. Nat Genet 52, 594-603.
Lynn, G. M., Sedlik, C., Baharom, F., Zhu, Y., Ramirez-Valdez, R. A., Coble, V. L., Tobin, K., Nichols, S. R., Itzkowitz, Y., Zaidi, N., et al. (2020). Peptide-TLR-7/8a conjugate vaccines chemically programmed for nanoparticle self-assembly enhance CD8 T-cell immunity to tumor antigens. Nat Biotechnol 38, 320-332.
MacKenzie, K. F., Clark, K., Naqvi, S., McGuire, V. A., Noehren, G., Kristariyanto, Y., van den Bosch, M., Mudaliar, M., McCarthy, P. C., Pattison, M. J., et al. (2013). PGE(2) induces macrophage IL-10 production and a regulatory-like phenotype via a protein kinase A-SIK-CRTC3 pathway. J Immunol 190, 565-577.
Maier, B., Leader, A. M., Chen, S. T., Tung, N., Chang, C., LeBerichel, J., Chudnovskiy, A., Maskey, S., Walker, L., Finnigan, J. P., et al. (2020). A conserved dendritic-cell regulatory program limits antitumour immunity. Nature 580, 257-262.
Mariathasan, S., Turley, S. J., Nickles, D., Castiglioni, A., Yuen, K., Wang, Y., Kadel, E. E., III, Koeppen, H., Astarita, J. L., Cubas, R., et al. (2018). TGFbeta attenuates tumour response to PD-L1 blockade by contributing to exclusion of T cells. Nature 554, 544-548.
McGinnis, C. S., Murrow, L. M., and Gartner, Z. J. (2019). DoubletFinder: Doublet Detection in Single-Cell RNA Sequencing Data Using Artificial Nearest Neighbors. Cell Syst 8, 329-337 e324.
McNab, F., Mayer-Barber, K., Sher, A., Wack, A., and O'Garra, A. (2015). Type I interferons in infectious disease. Nat Rev Immunol 15, 87-103.
Melero, I., Gaudernack, G., Gerritsen, W., Huber, C., Parmiani, G., Scholl, S., Thatcher, N., Wagstaff, J., Zielinski, C., Faulkner, I., et al. (2014). Therapeutic vaccines for cancer: an overview of clinical trials. Nat Rev Clin Oncol 11, 509-524.
Meredith, M. M., Liu, K., Darrasse-Jeze, G., Kamphorst, A. O., Schreiber, H. A., Guermonprez, P., Idoyaga, J., Cheong, C., Yao, K. H., Niec, R. E., et al. (2012). Expression of the zinc finger transcription factor zDC (Zbtb46, Btbd4) defines the classical dendritic cell lineage. J Exp Med 209, 1153-1165.
Molgora, M., Esaulova, E., Vermi, W., Hou, J., Chen, Y., Luo, J., Brioschi, S., Bugatti, M., Omodei, A. S., Ricci, B., et al. (2020). TREM2 Modulation Remodels the Tumor Myeloid Landscape Enhancing Anti-PD-1 Immunotherapy. Cell 182, 886-900 e817.
Mosely, S. I., Prime, J. E., Sainson, R. C., Koopmann, J. O., Wang, D. Y., Greenawalt, D. M., Ahdesmaki, M. J., Leyland, R., Mullins, S., Pacelli, L., et al. (2017). Rational Selection of Syngeneic Preclinical Tumor Models for Immunotherapeutic Drug Discovery. Cancer Immunol Res 5, 29-41.
Mulder, K., Patel, A. A., Kong, W. T., Piot, C., Halitzki, E., Dunsmore, G., Khalilnezhad, S., Irac, S. E., Dubuisson, A., Chevrier, M., et al. (2021). Cross-tissue single-cell landscape of human monocytes and macrophages in health and disease. Immunity 54, 1883-1900 e1885.
Peng, J., Sun, B. F., Chen, C. Y., Zhou, J. Y., Chen, Y. S., Chen, H., Liu, L., Huang, D., Jiang, J., Cui, G. S., et al. (2019). Single-cell RNA-seq highlights intra-tumoral heterogeneity and malignant progression in pancreatic ductal adenocarcinoma. Cell Res 29, 725-738.
Romero, P., Banchereau, J., Bhardwaj, N., Cockett, M., Disis, M. L., Dranoff, G., Gilboa, E., Hammond, S. A., Hershberg, R., Korman, A. J., et al. (2016). The Human Vaccines Project: A roadmap for cancer vaccine development. Sci Transl Med 8, 334ps339.
Rosas-Ballina, M., Guan, X. L., Schmidt, A., and Bumann, D. (2020). Classical Activation of Macrophages Leads to Lipid Droplet Formation Without de novo Fatty Acid Synthesis. Front Immunol 11, 131.
Saxena, M., van der Burg, S. H., Melief, C. J. M., and Bhardwaj, N. (2021). Therapeutic cancer vaccines. Nat Rev Cancer 21, 360-378.
Sharma, A., Seow, J. J. W., Dutertre, C. A., Pai, R., Bleriot, C., Mishra, A., Wong, R. M. M., Singh, G. S. N., Sudhagar, S., Khalilnezhad, S., et al. (2020). Onco-fetal Reprogramming of Endothelial Cells Drives Immunosuppressive Macrophages in Hepatocellular Carcinoma. Cell 183, 377-394 e321.
Shibuya, T., Kamiyama, A., Sawada, H., Kikuchi, K., Maruyama, M., Sawado, R., Ikeda, N., Asano, K., Kurotaki, D., Tamura, T., et al. (2021). Immunoregulatory Monocyte Subset Promotes Metastasis Associated With Therapeutic Intervention for Primary Tumor. Front Immunol 12, 663115.
Sleijfer, S., Bannink, M., Van Gool, A. R., Kruit, W. H., and Stoter, G. (2005). Side effects of interferon-alpha therapy. Pharm World Sci 27, 423-431.
Spitzer, M. H., Carmi, Y., Reticker-Flynn, N. E., Kwek, S. S., Madhireddy, D., Martins, M. M., Gherardini, P. F., Prestwood, T. R., Chabon, J., Bendall, S. C., et al. (2017). Systemic Immunity Is Required for Effective Cancer Immunotherapy. Cell 168, 487-502 e415.
Sultan, H., Salazar, A. M., and Celis, E. (2020). Poly-ICLC, a multi-functional immune modulator for treating cancer. Semin Immunol 49, 101414.
Thoreau, M., Penny, H. L., Tan, K., Regnier, F., Weiss, J. M., Lee, B., Johannes, L., Dransart, E., Le Bon, A., Abastado, J. P., et al. (2015). Vaccine-induced tumor regression requires a dynamic cooperation between T cells and myeloid cells at the tumor site. Oncotarget 6, 27832-27846.
Thorsson, V., Gibbs, D. L., Brown, S. D., Wolf, D., Bortone, D. S., Ou Yang, T. H., Porta-Pardo, E., Gao, G. F., Plaisier, C. L., Eddy, J. A., et al. (2018). The Immune Landscape of Cancer. Immunity 48, 812-830 e814.
U'Ren, L., Guth, A., Kamstock, D., and Dow, S. (2010). Type I interferons inhibit the generation of tumor-associated macrophages. Cancer Immunol Immunother 59, 587-598.
van der Sluis, T. C., Sluijter, M., van Duikeren, S., West, B. L., Melief, C. J., Arens, R., van der Burg, S. H., and van Hall, T. (2015). Therapeutic Peptide Vaccine-Induced CD8 T Cells Strongly Modulate Intratumoral Macrophages Required for Tumor Regression. Cancer Immunol Res 3, 1042-1051.
Veglia, F., Sanseviero, E., and Gabrilovich, D. I. (2021). Myeloid-derived suppressor cells in the era of increasing myeloid cell diversity. Nat Rev Immunol 21, 485-498.
Waldman, A. D., Fritz, J. M., and Lenardo, M. J. (2020). A guide to cancer immunotherapy: from T cell basic science to clinical practice. Nat Rev Immunol 20, 651-668.
Wu, S. Z., Al-Eryani, G., Roden, D. L., Junankar, S., Harvey, K., Andersson, A., Thennavan, A., Wang, C., Torpy, J. R., Bartonicek, N., et al. (2021). A single-cell and spatially resolved atlas of human breast cancers. Nat Genet 53, 1334-1347.
Yang, H., Wang, H., Levine, Y. A., Gunasekaran, M. K., Wang, Y., Addorisio, M., Zhu, S., Li, W., Li, J., de Kleijn, D. P., et al. (2016). Identification of CD163 as an antiinflammatory receptor for HMGB1-haptoglobin complexes. JCI Insight 1.
Yang, Y., Guo, J., and Huang, L. (2020). Tackling TAMs for Cancer Immunotherapy: It's Nano Time. Trends Pharmacol Sci 41, 701-714.
Zhang, L., Li, Z., Skrzypczynska, K. M., Fang, Q., Zhang, W., O'Brien, S. A., He, Y., Wang, L., Zhang, Q., Kim, A., et al. (2020). Single-Cell Analyses Inform Mechanisms of Myeloid-Targeted Therapies in Colon Cancer. Cell 181, 442-459 e429.
Zhang, X., Liu, S., Guo, C., Zong, J., and Sun, M. Z. (2012). The association of annexin A2 and cancers. Clin Transl Oncol 14, 634-640.
Zhang, X., Ren, D., Guo, L., Wang, L., Wu, S., Lin, C., Ye, L., Zhu, J., Li, J., Song, L., et al. (2017). Thymosin beta 10 is a key regulator of tumorigenesis and metastasis and a novel serum marker in breast cancer. Breast Cancer Res 19, 15.
Zheng, C., Zheng, L., Yoo, J. K., Guo, H., Zhang, Y., Guo, X., Kang, B., Hu, R., Huang, J. Y., Zhang, Q., et al. (2017). Landscape of Infiltrating T Cells in Liver Cancer Revealed by Single-Cell Sequencing. Cell 169, 1342-1356 e1316.
Zhou, Y., Zhou, B., Pache, L., Chang, M., Khodabakhshi, A. H., Tanaseichuk, O., Benner, C., and Chanda, S. K. (2019). Metascape provides a biologist-oriented resource for the analysis of systems-level datasets. Nat Commun 10, 1523.
Zilionis, R., Engblom, C., Pfirschke, C., Savova, V., Zemmour, D., Saatcioglu, H. D., Krishnan, I., Maroni, G., Meyerovitz, C. V., Kerwin, C. M., et al. (2019). Single-Cell Transcriptomics of Human and Mouse Lung Cancers Reveals Conserved Myeloid Populations across Individuals and Species. Immunity 50, 1317-1334 e1310.
Zitvogel, L., Galluzzi, L., Kepp, O., Smyth, M. J., and Kroemer, G. (2015). Type I interferons in anticancer immunity. Nat Rev Immunol 15, 405-414.

### Example 9. Introduction and Methods for Examples 9-16

### A. Introduction

Patients have benefitted greatly from the advancement of immunotherapies that activate the immune system to promote tumor cell killing. Studies using checkpoint inhibitors (CPIs) that target PD-1 and/or CTLA-4 have revealed an essential role for CD8 T cells in mediating anti-tumor efficacy. In the absence of endogenously primed tumor-antigen specific CD8 T cells, these CPIs fail to induce tumor regression. The goal of cancer vaccines is to prime anti-tumor CD8 T cells in lieu of endogenous priming that many patients' immune systems fail to perform.

Historically, cancer vaccines have failed to induce robust anti-tumor immunity, which may be the result of poor antigen selection, suboptimal vaccine platforms, and the pre-existing immunosuppressive tumor microenvironment (TME). The last decade has seen major improvements in the understanding of these facets, with multiple pre-clinical studies demonstrating the synergistic effects of cancer vaccines administered in combination with CPIs. Of note, peptide-based vaccine strategies have not yet demonstrated robust priming of neoantigen-specific CD8 T cell responses in most patients and though mRNA-based vaccines have been more immunogenic, they appear to preferentially elicit CD4 T cell responses against neoantigens in humans. One solution to the problem of low-magnitude immunogenicity is the use of a heterologous prime-boost strategy, which has been shown to increase immunity relative to homologous vaccination strategies against infectious diseases like SARS-CoV-2 in humans as well as against tumor antigens in pre-clinical mouse models.

The self-assembling nanoparticle (SNP) vaccine that co-delivers a peptide antigen with a TLR-7/8 agonist was described in Examples 1-8. The SNP vaccine was designed to be rapidly manufactured and can enable delivery of ˜98% of possible neoantigen peptides as stable 20-40 nm micelles, features which allow for the production of personalized vaccines targeting patient tumor neoantigens. The SNP vaccine induces high quality anti-tumor antigen-specific CD8 T cells when administered by the intravenous (IV) route, as shown in Examples 1-8, but there are concerns it may suffer from the weak immunogenicity characteristic of peptide vaccines in humans. To reduce the risk of priming sub-efficacious frequencies of anti-tumor antigen-specific CD8 T cell responses, the boosting of IV-SNP primed CD8 T cell responses with the adenovirus ChAdOx1 by administering a ChAdOx1 vaccine vector as a second (i.e. boost) treatment, was tested as adenoviral vectors are known for their ability to prime robust, high magnitude CD8 T cell responses.

The ChAdOx1 vector was developed by identifying a chimp adenovirus with low seroprevalence in humans to avoid pre-existing neutralizing antibodies and modifying ChAdOx1 to express disease target antigens. ChAdOx1 has been successfully used to vaccinate people against SARS-CoV-2, and has demonstrated promising anti-tumor efficacy in multiple pre-clinical mouse tumor models as a heterologous prime boost approach with a modified vaccinia ankara (MVA) boost. Recent clinical trials utilizing IM adenovirus followed by either MVA or a self-amplifying RNA to induce neoantigen-specific CD8 T cell responses have demonstrated the safety and immunogenicity of a heterologous prime-boost approach in humans.

Cancer vaccine studies have focused predominantly on their ability to prime tumor antigen specific CD8 T cell responses, and have largely ignored the TME under the assumption that combination with existing CPIs will enable the efficacy that has previously eluded cancer vaccines. The TME is a complex collection of immune and non-immune cells that can be characterized according to the degree of immune infiltration, spanning a spectrum from ‘immune desert’ to ‘inflamed.’ Typical myeloid cell populations present in colorectal cancer tumors in both mice (i.e., MC38) and humans include monocytes, macrophages, and conventional dendritic cells (cDCs). Whereas cDCs, which come in two major subtypes—cDC1s that cross-present to CD8 T cells and cDC2s that specialize in priming CD4 T cells, generally have anti-tumor functions; monocytes and macrophages can have both pro-tumoral or anti-tumoral functions depending on the microenvironment and associated signals. Indeed, tumor associated macrophages (TAMs), which broadly refers to a heterogeneous population of myeloid cells of embryonic or monocytic origin present in the tumor have emerged as a promising target to promote anti-tumor immunity. The goal of TAM targeting therapeutics is to promote their polarization away from an anti-inflammatory (pro-tumoral) state towards a pro-inflammatory (anti-tumoral) state, also referred to as an M2-phenotype to M1-phenotype polarization, respectively. This effect can be induced by exposing TAMs to type I IFNs (IFNα or IFNβ), which results in the upregulation of interferon-stimulated genes (ISGs) that promote a pro-inflammatory response. Myeloid cell polarization towards a pro-inflammatory state is associated with an improved response to CPIs in mice and may enable CD8 T cell function at the tumor site.

In the subsequent studies, a heterologous prime-boost approach was tested using IV SNP to prime and ChAdOx1 to boost neoantigen specific CD8 T cell responses. In contrast to the aforementioned heterologous prime boost cancer vaccine strategies, we opted to use ChAdOx1 as a boost rather than prime vaccination, due largely to the belief that a personalized ChAdOx1 vector is likely to take longer to manufacture than the SNP vaccine. Further, the effect of the route of ChAdOx1 administration was tested by testing IV administration, as IV vaccination has been shown to provide superior efficacy in pre-clinical mouse tumor models and against TB in non-human primates. These two major differences to standard heterologous prime boost vaccination strategies revealed that IV ChAdOx1 boosting elicits far higher frequencies of antigen-specific cells as compared to intramuscular (IM) vaccination. In addition, IV ChAdOx1 administration causes the release of large amounts of systemic type I IFNs. This appears to increase the number and activation state of cDC1s in the tumor draining lymph node (tdLN). Furthermore, the Type I IFNs reduce the frequency of immunosuppressive M2-like myeloid cells and increases the relative frequency of pro-inflammatory M1-like macrophages at the tumor site. Blockade of IFNAR1 negates these effects on myeloid cells and the associated efficacy of the heterologous prime boost approach, demonstrating the importance of functional IFN-I signalling. Collectively, these data suggest that IV ChAdOx1 boosting not only increases the magnitude of the antigen-specific CD8 T cell response, but also modulates the TME to promote tumor regression.

### B. Materials and Methods

The cells from different mice were barcoded prior to loading samples into the 10× Genomics chromium controller. Single cell cDNA libraries were created, sequenced, and subsequently used to cluster single cells based on their gene expression profiles by using an unsupervised clustering algorithm through the Seurat (v4) pipeline. As part of the data processing, all contaminating cells that were not monocytes, macrophages or DCs were excluded from downstream analysis.

### Example 10. Intravenous ChAdOx1 Vaccination Elicits High Frequencies of Differentiated Neo-Antigen Specific CD8 T Cell Responses

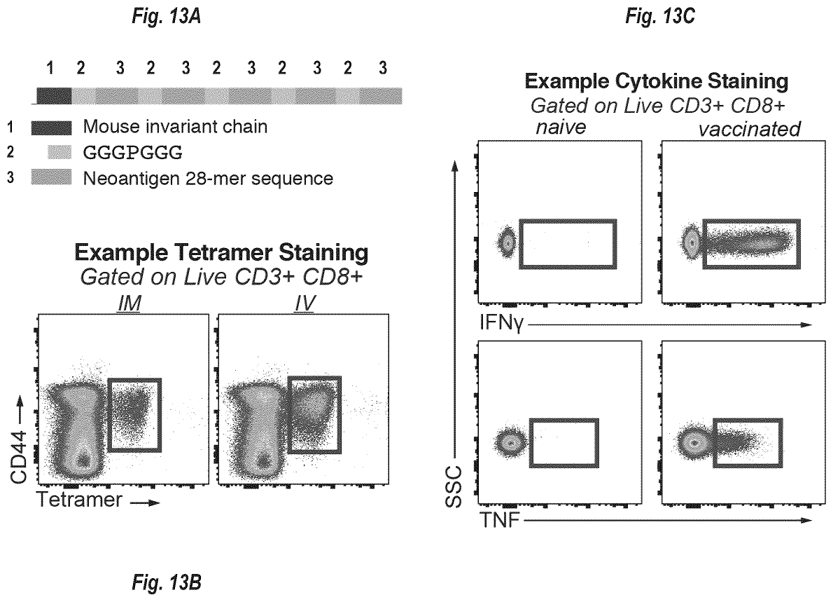

A key goal for therapeutic cancer vaccines is to elicit a high frequency of antigen-specific CD8 T cells. Examples 1-8 show that a peptide based self-assembling nanoparticle vaccine (SNP) elicits neoantigen-specific CD8 T cell immunity and mediates protection in mouse tumor models and that the route of vaccination can influence the magnitude and quality of T cell immunity. It was hypothesized that anti-tumor efficacy could be improved in mouse tumor models by using a heterologous prime-boost approach, administering SNP followed by a polynucleotide vaccine vector, to elicit higher magnitude anti-tumor CD8 T cell responses. To test this, a new ChAdOx1 vaccine (described in Example 9) that encodes the MC38 neoantigen Reps1 (FIG. 13A) was generated to be used in combination with the SNP vaccine.

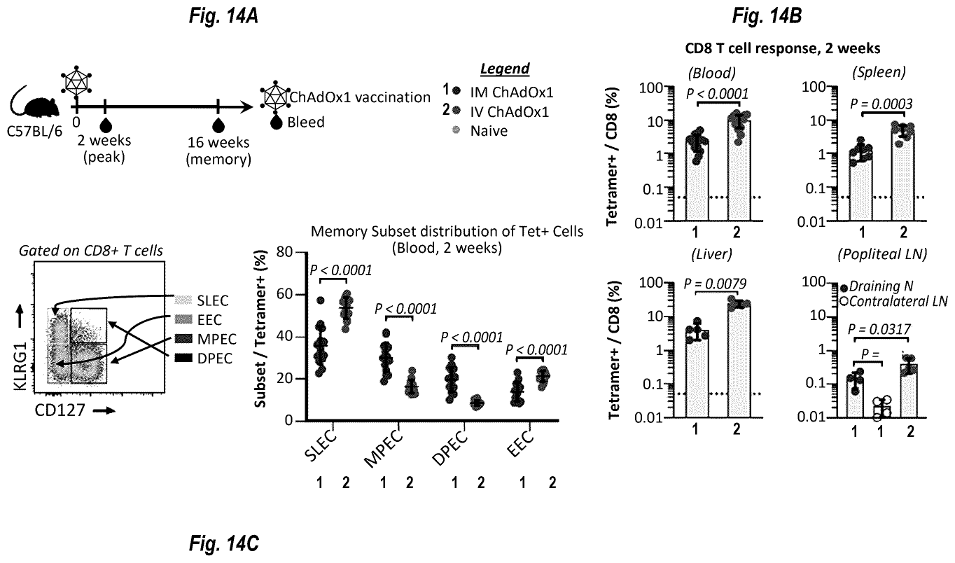

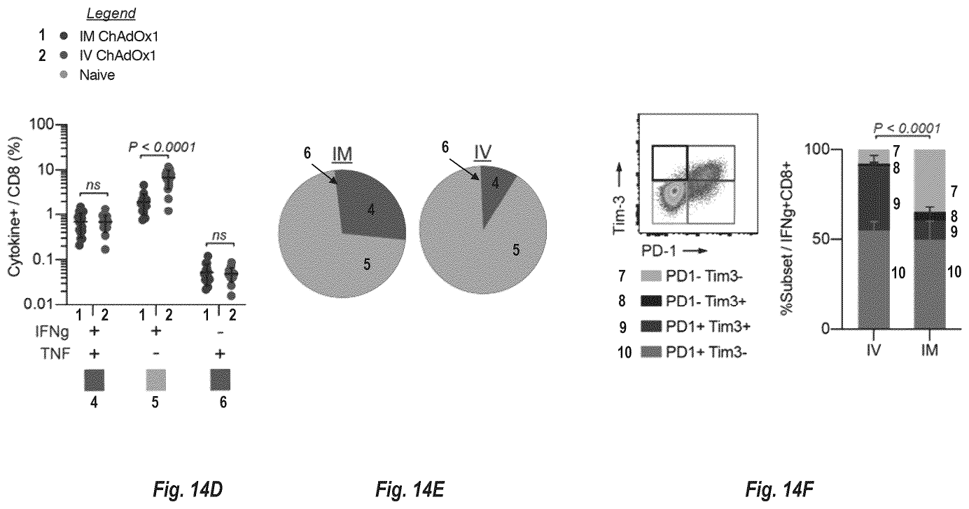

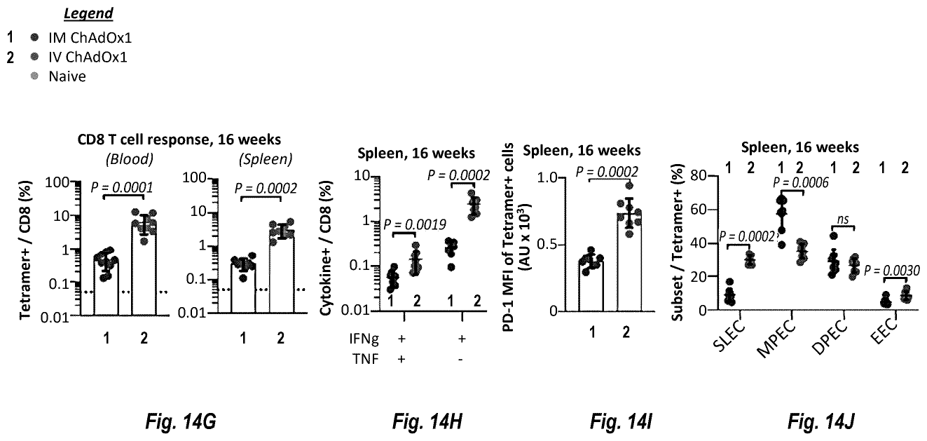

First, this new ChAdOx1 vaccine was assessed for immunogenicity, and it was determined how the route of immunization altered the magnitude, phenotype, and quality of T cell responses. Mice were vaccinated intramuscularly (IM) or intravenously (IV) with Reps1-encoding ChAdOx1 and the frequency of CD8 T cell responses was assessed at peak (2 weeks) and memory (16 weeks) time points in blood and multiple tissue sites by tetramer staining (FIG. 14A). At 2 weeks post immunization, IV ChAdOx1 elicited significantly higher magnitude T cell responses in the blood, spleen, liver, and popliteal lymph node (popLN) than IM ChAdOx1 (FIGS. 13B and 14B). The memory phenotypes of CD8 T cells following ChAdOx1 immunization were assessed using the canonical cell surface markers CD127 (IL-7R) and KLRG1 (FIG. 14C, left panel). Memory precursor effector cells (MPECs) retain CD127 expression whereas short-lived effector cells lose CD127 expression and upregulate KLRG1. Two weeks after IV ChAdOx1 vaccination, there was a higher frequency of SLECs and lower frequency of MPECs as compared to IM ChAdOx1 (FIG. 14C, right panel).

To characterize the functional capacity of the T cell response, the frequency of CD8 T cells producing IFNγ and TNF was determined following peptide restimulation (FIG. 13C). 90% of the total responding cells produced IFNγ alone following IV immunization compared to 75% by IM immunization (FIG. 14D). CD8 T cells producing IFNγ and TNF represented ˜25% of the antigen-specific cells following IM vaccination but only ˜7% following IV vaccination (FIG. 14E). Lastly, IV ChAdOx1 elicited a higher frequency of PD-1 and Tim-3 co-expressing antigen-specific CD8 T cells (FIG. 14F).

Similar analyses were performed 16 weeks post-vaccination to assess the durability and qualitative profile of the CD8 T cell response. The frequency of Reps1-specific CD8 T cells remained significantly higher 16 weeks after IV ChAdOx1 compared to IM immunization in blood and spleen (FIG. 14G), and was still predominantly comprised of solely IFNγ producing cells. (FIG. 14H). The expression of PD-1 remained higher on IV ChAdOx1 elicited CD8 T cells as compared to the IM route (FIG. 14I). The frequency of SLECs decreased and MPECS increased following IV or IM ChAdOx-1 with the relative proportions of MPECs being higher in the IM group compared to week 2 (FIG. 14J). Collectively, these data show that IV vaccination elicits high magnitude, more terminally differentiated T cell responses that are durable over a 16-week period compared to the same dose administered by the IM route.

### Example 11. Intravenous Heterologous Prime-Boost Elicits High Magnitude CD8 T Cell Responses that are Protective Against MC38 Tumor Challenge

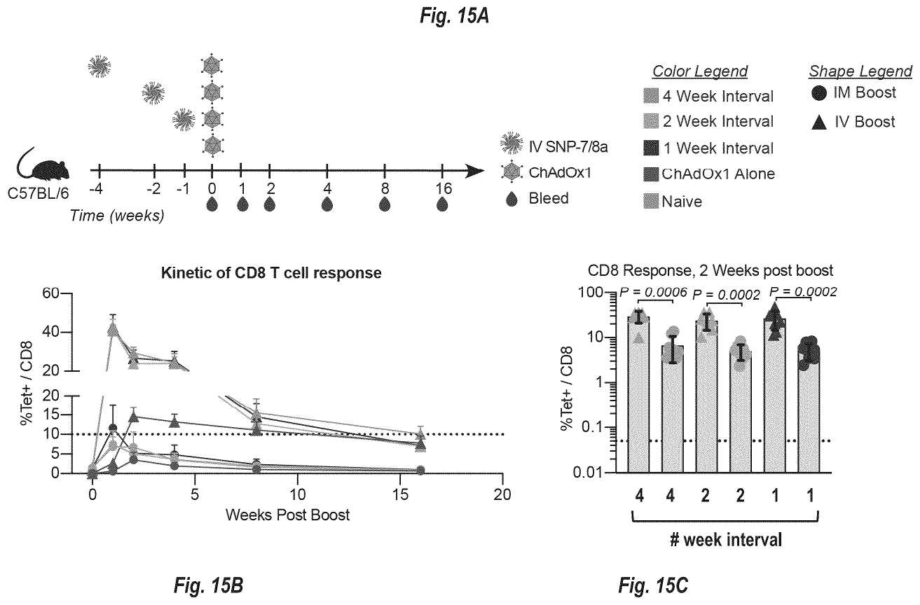

The next series of studies focused on heterologous prime-boost immunization using the SNP vaccine administered IV to prime CD8 T cells followed by ChAdOx1 as a boost. To determine whether the interval between prime and boost affected the magnitude of CD8 T cell responses, groups of mice were primed either 4, 2, or 1 week(s) prior to boosting with ChAdOx1 given IV or IM and tetramer staining was performed through 16 weeks (FIG. 15A). There were no significant differences in magnitude of the CD8 T cell response based on the interval between prime and boost when comparing groups that received ChAdOx1 by the IM or IV route (FIG. 15B). However, IV ChAdOx1 boost elicited significantly higher magnitude CD8 T cell responses than IM, regardless of interval and consistent with the differences seen when using ChAdOx1 alone (FIG. 15C).

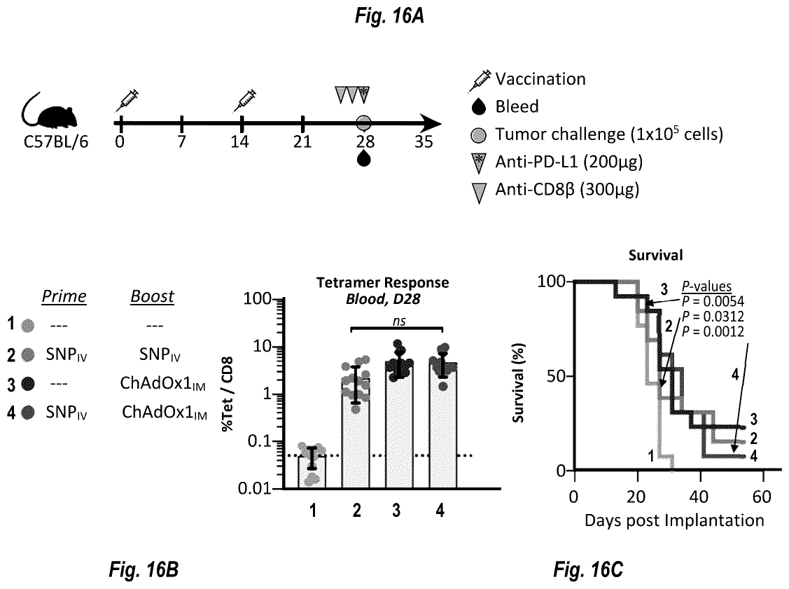

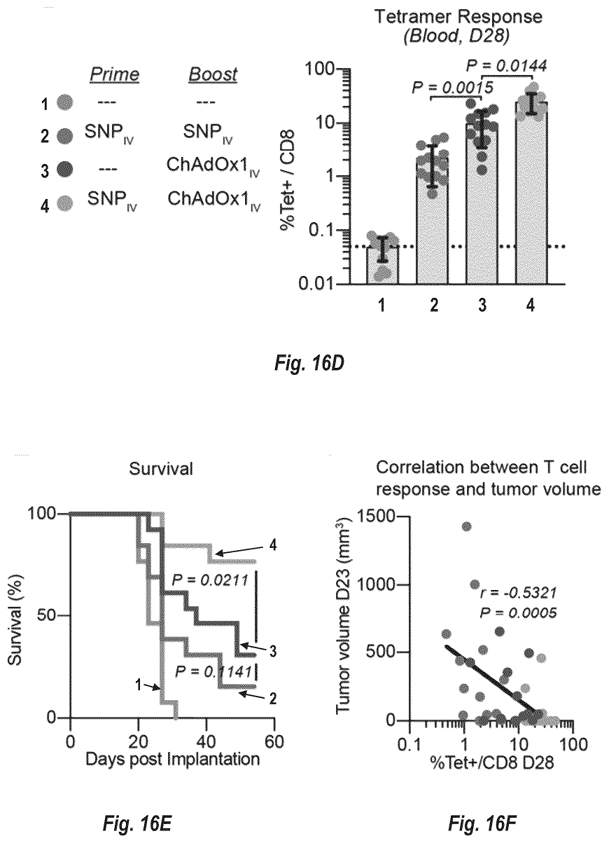

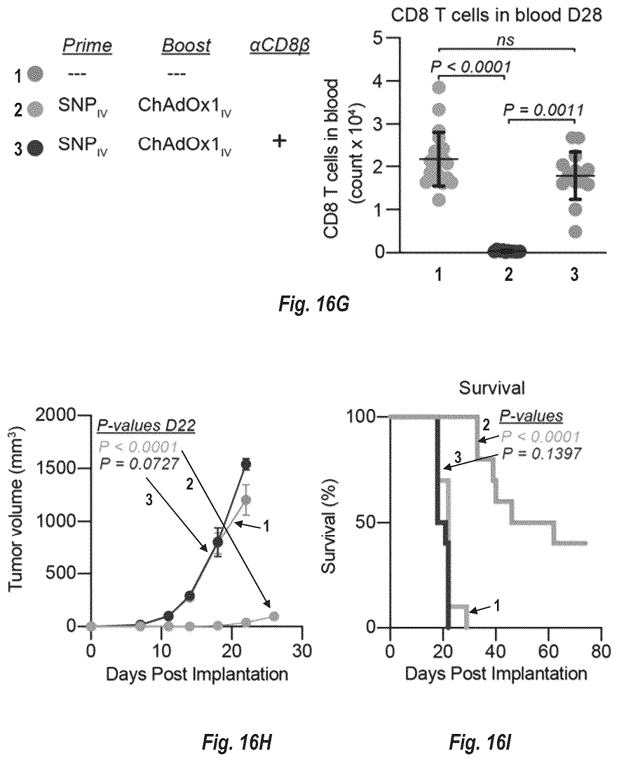

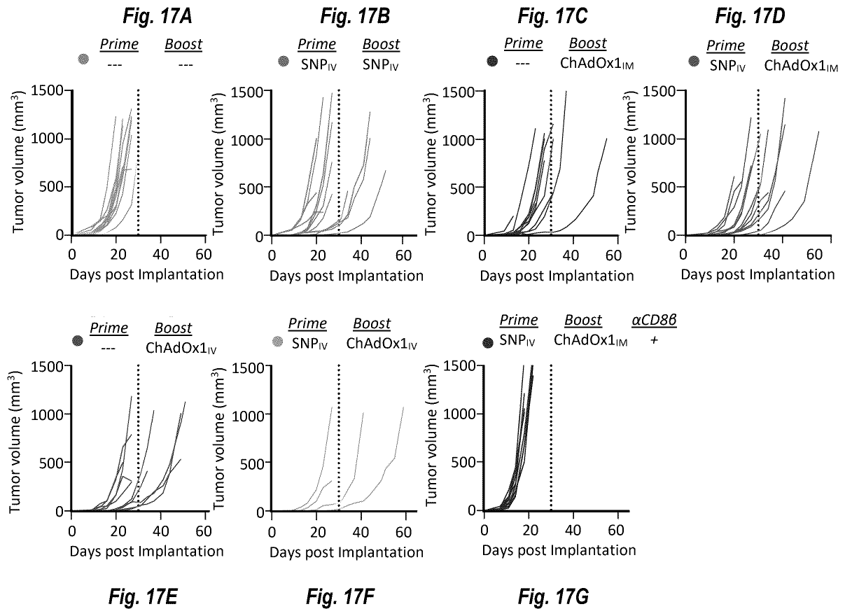

To test the protective efficacy of heterologous prime-boost vaccination in a prophylactic model, a 2-week interval between prime and boost was used. Homologous prime-boost vaccination with SNP (positive control) was compared to heterologous prime-boost with SNP followed by ChAdOx1, and ChAdOx1 alone at the time of boost. Initially, administration of ChAdOx1 by the IM route was explored as this is the standard route used for adenoviral vaccinations in the majority of infectious disease and tumor models. Mice were challenged with MC38 cells 2 weeks post boost and received a single dose of αPD-L1 at this time (FIG. 16A). Heterologous prime-boost with IM ChAdOx1 did not increase the magnitude of the Reps1-specific CD8 T cell response magnitude as compared to either SNP given twice or IM ChAdOx1 alone (FIG. 16B). All vaccinated groups showed reduced tumor growth (FIGS. 17A-17D) and improved survival compared to untreated animals (FIG. 16C). There were no differences in terms of immunogenicity or tumor control between the different vaccine regimens when using the IM route for ChAdOx1 vaccination.

Based on the striking increase in CD8 T cell immunity elicited by the IV route as compared to IM administration with ChAdOx1, a similar study was done using the IV route. Intravenous boosting with ChAdOx1 elicited significantly higher magnitude Reps1-specific CD8 T cell responses than IV ChAdOx1 alone or SNP given twice (FIG. 16D). All these vaccine groups displayed reduced tumor growth rates (FIGS. 17E-17F) and improved survival (FIG. 16E) as compared to untreated mice. There was a moderate negative correlation between the magnitude of the Reps1-specific CD8 T cell responses of individual animals and their tumor volumes 23 days post challenge (FIG. 16F), suggesting that the vaccine elicited CD8 T cell response was mediating the enhanced protection. The data demonstrates that heterologous prime-boost with IV ChAdOx1 elicits the highest CD8 T cell response and best protection.

To confirm that the prophylactic efficacy observed with the IV heterologous prime-boost vaccination regimen was dependent on CD8 T cell responses, an antibody that depletes cells expressing CD80 was administered to mice 3 days and 1 day prior to tumor challenge. Circulating CD8 T cells were effectively depleted by this treatment (FIG. 16G) and resulted in a complete loss of tumor growth control (FIG. 16H), and reduced survival (FIG. 16I). These data demonstrate that protection from MC38 in the prophylactic setting is dependent on the magnitude of pre-existing Reps1-specific CD8 T cell responses.

### Example 12. Therapeutic Heterologous Prime-Boost Vaccination with IV ChAdOx1 Controls Established Tumors

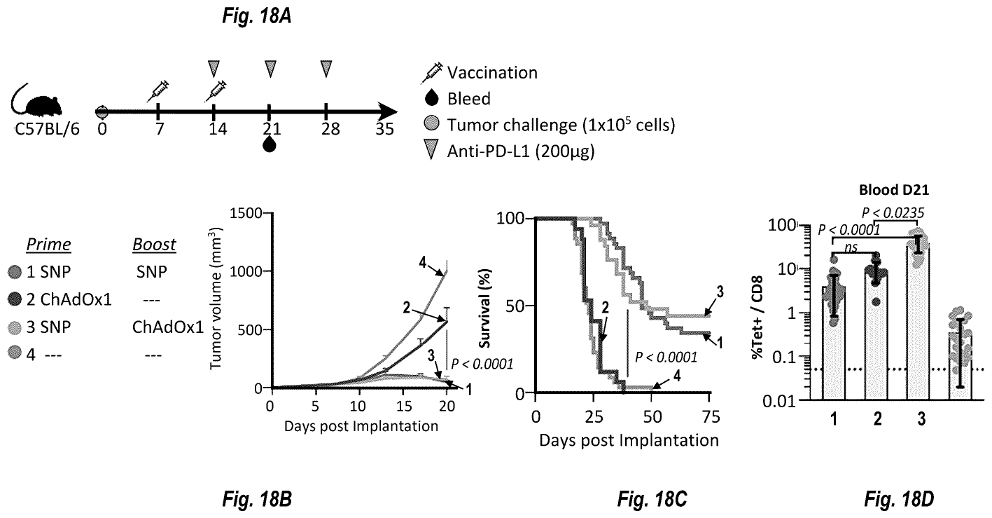

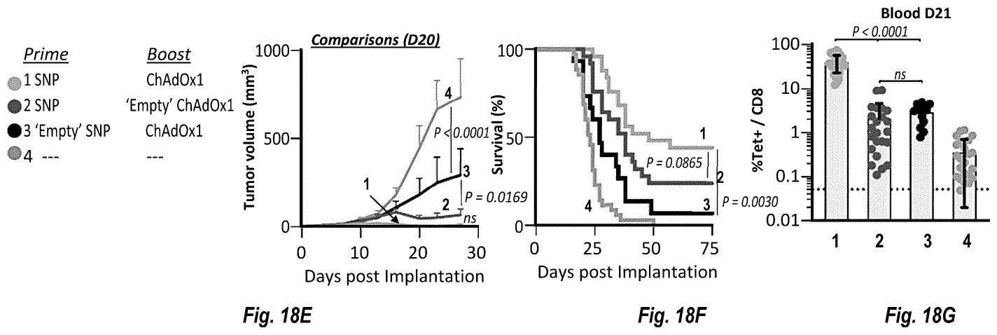

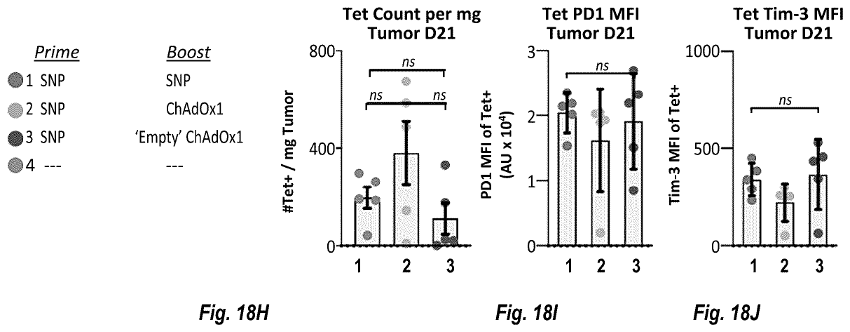

To determine efficacy in a therapeutic setting, heterologous prime-boost immunization was tested for induction of tumor regression of established MC38 tumors. Mice were vaccinated 1 week after tumors were implanted and boosted a week later. The shortened interval between prime and boost was necessary due to the rapid growth of the tumors. All mice received αPD-L1 weekly for a total of 3 doses starting at the time of boost (FIG. 18A). Given the lack of efficacy in the prophylactic setting observed when administering ChAdOx1 IM, all subsequent studies focused on the IV route of administration.

Previously, it was observed that SNP given twice can provide ˜40% protection in the therapeutic setting with MC38 tumor challenge. This observation was used to benchmark our IV heterologous prime-boost group and an IV ChAdOx1 alone group. The IV heterologous prime-boost vaccine group displayed similar control of tumor growth (FIG. 18B) and survival (FIG. 18C) as homologous prime and boost with SNP. Both prime-boost vaccine groups had significantly better tumor control than the ChAdOx1 prime alone group (FIGS. 18B-18C). In contrast to the prophylactic model, the protection across the vaccine groups in this therapeutic model did not correlate with the magnitude of the Reps1-specific CD8 T cell response 1 week post boost. Indeed, mice that received IV ChAdOx1 alone did not exhibit any tumor control despite inducing ˜10% Reps1-specific CD8 T cell responses (FIG. 18D). These data suggest that T cells may be necessary but not sufficient to mediate tumor control and that the homologous or heterologous prime-boost regimen given by the IV route was required for protection.

To delineate the requirement of the antigen versus adjuvant provided by ChAdOx1 in the prime or boost vaccinations, we used a SNP vaccine and a ChAdOx1 vector that encoded an irrelevant antigen referred to as ‘empty’. Reps-1 SNP followed by ‘empty’ ChAdOx1 resulted in significant tumor regression whereas the empty-SNP followed by Reps1-encoding ChAdOx1 only delayed tumor growth (FIG. 18E). These differences were reflected in the survival curves, where there was no statistically significant difference between the IV heterologous prime-boost and SNP followed by ‘empty’ ChAdOx1 (FIG. 18F). However, there was a significant difference between IV heterologous prime-boost and ‘empty’ SNP followed by antigen-encoding ChAdOx1, indicating that priming a CD8 T cell response may be critical for protection (FIG. 18F). As expected, the heterologous prime-boost group elicited the highest magnitude CD8 T cell response, and the groups where only one vaccination delivered Reps1-antigen had equivalent magnitude T cell responses 1 week post boost vaccination (FIG. 18G). These data suggest that two vaccinations are required to attain efficacy and that priming a Reps1-specific CD8 T cell response is required for protection, but boosting the Reps1-specific CD8 T cell response may not be essential.

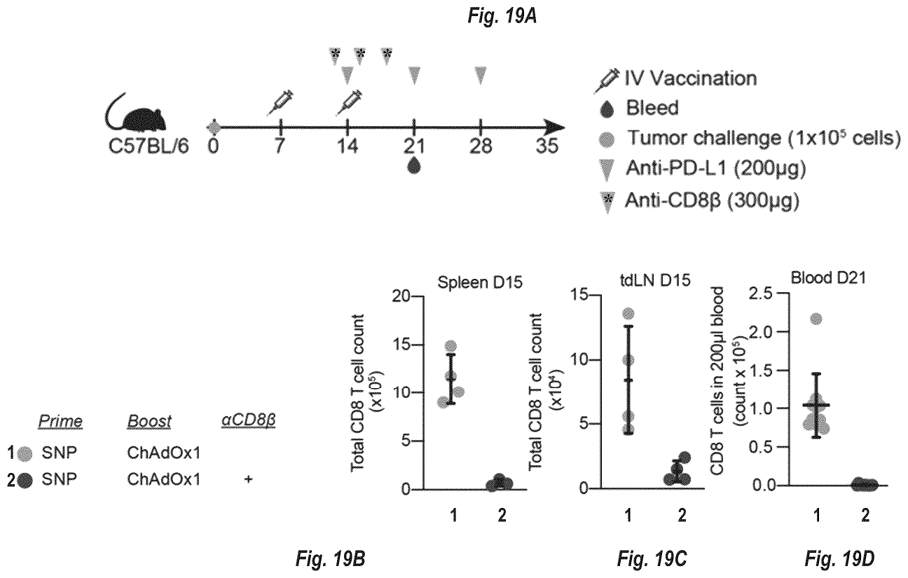

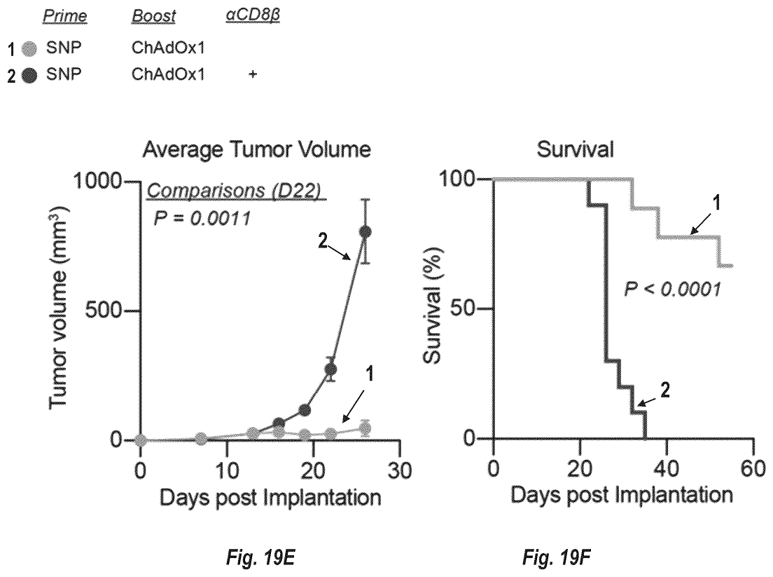

To confirm that protection requires CD8 T cells in the therapeutic setting, anti-CD80 was administered just prior to, and immediately after, the antigen encoding ChAdOx1 boost (FIG. 19A). Depletion of CD8 T cells was verified in the spleen and tumor draining lymph node (tdLN) one day post boost vaccination (FIGS. 19B-19C) and in blood 1 week post boost (FIG. 19D). Depletion of CD8 T cells abrogated control of tumor growth (FIG. 19E) and survival (FIG. 19F). Thus, CD8 T cells are required for therapeutic efficacy after the boost vaccination, even if boosting the response with a vaccine containing the antigen is non-essential.

Finally, the Reps1 specific CD8 T cell response in the tumors of the selected vaccine groups was also assessed at 1 week post boost vaccination. There were no significant differences in the number of Reps1-specific CD8 T cells in the tumor (FIG. 18H) or expression of PD-1 or Tim-3 on these cells (FIGS. 18I-18J). Collectively the data suggest that two vaccinations are required to promote tumor regression in mice challenged with MC38. A CD8 T cell response is required, but the magnitude or quality of that response does not appear to be critical for efficacy, given that the ‘empty’ ChAdOx1 boost also elicits protection.

### Example 13. ChAdOx1 Vaccination Activates STING Dependent Induction of Type 1 IFNα Required for Priming CD8 T Cell Responses

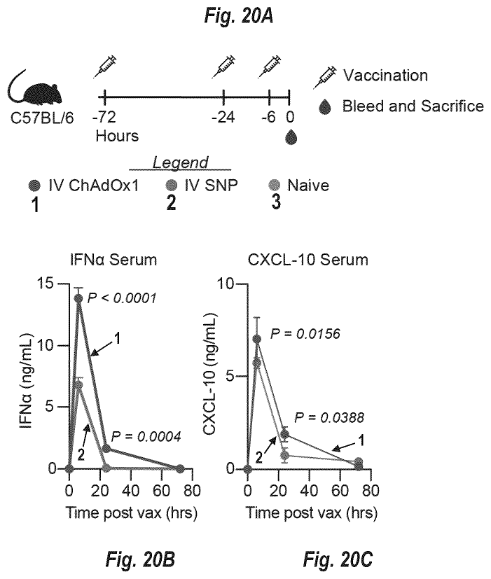

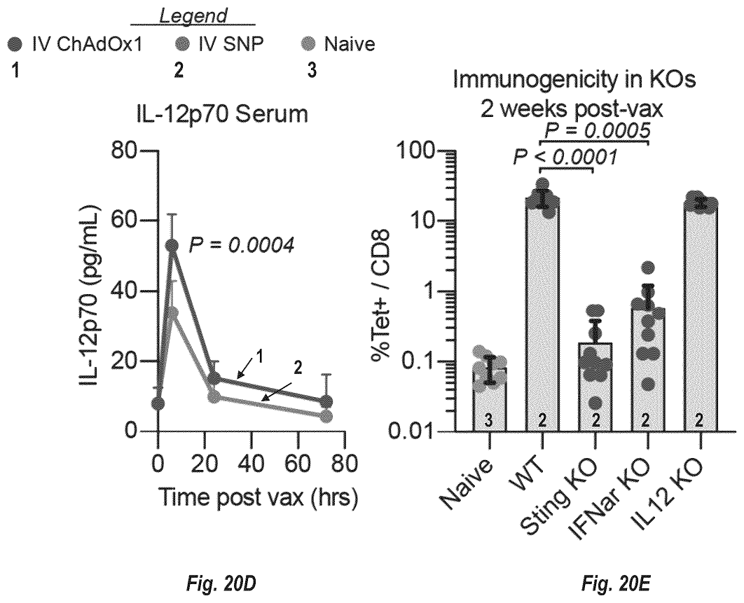

Data disclosed on preceding Examples show that IV ChAdOx1 vaccination induces potent CD8 T cell responses and can also mediate tumor regression when used as a boost that does not contain an antigen. Thus, the next hypothesis was that the ability to prime CD8 T cells and the protective effect of boosting with IV ChAdOx1 without the specific antigen may be due to its ability to enhance systemic innate cytokines. Indeed, IV vaccination with ChAdOx1 induced rapid production of IFNα, IP-10, and IL-12 canonical cytokines important for CD8 T cell priming and also have pleiotropic effects on immune activation and tumor control. As SNP contains the TLR 7/8a, we also showed production of these cytokines following SNP IV (FIGS. 20B-20D).

To assess the role of these cytokines on CD8 T cell priming, IFNα receptor was used with IL-12 KO mice. IFNα receptor KO mice had a significant reduction in the magnitude of CD8 T cell responses (FIG. 20E). However, there was no effect on the responses in IL-12 KO mice (FIG. 20E). To determine the innate pathway by which IFNα was induced, STING deficient mice were used because ChAdOx1 is a DNA virus. Indeed, CD8 T cells were significantly reduced. Together these data show that ChAdOx1 induces Sting dependent type I IFNs required for CD8 T cell priming and potentially an additional role for therapeutic efficacy.

### Example 14. Intravenous ChAdOx1 Vaccination Elicited Type I IFNs are Required for Tumor Regression and Activate cDC1s

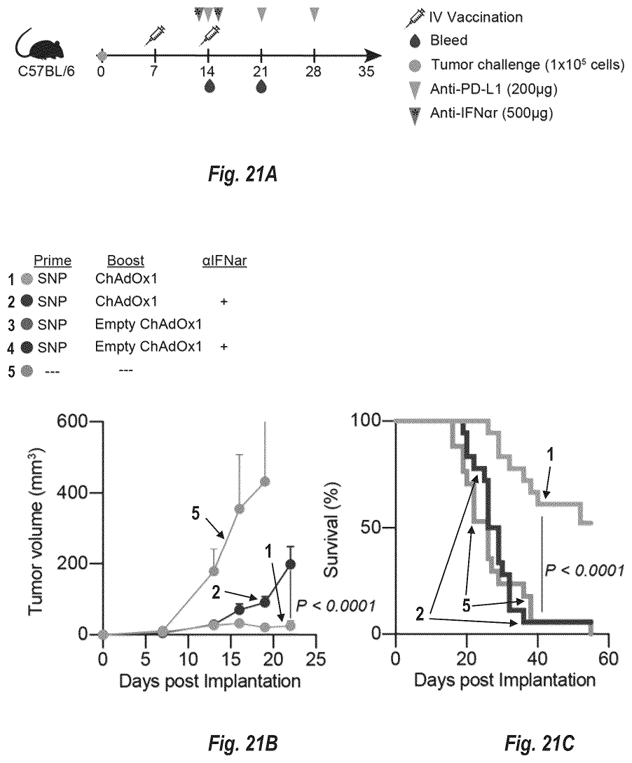

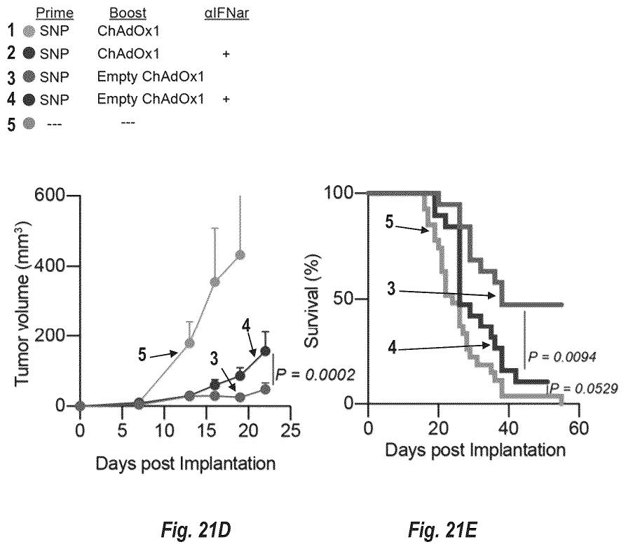

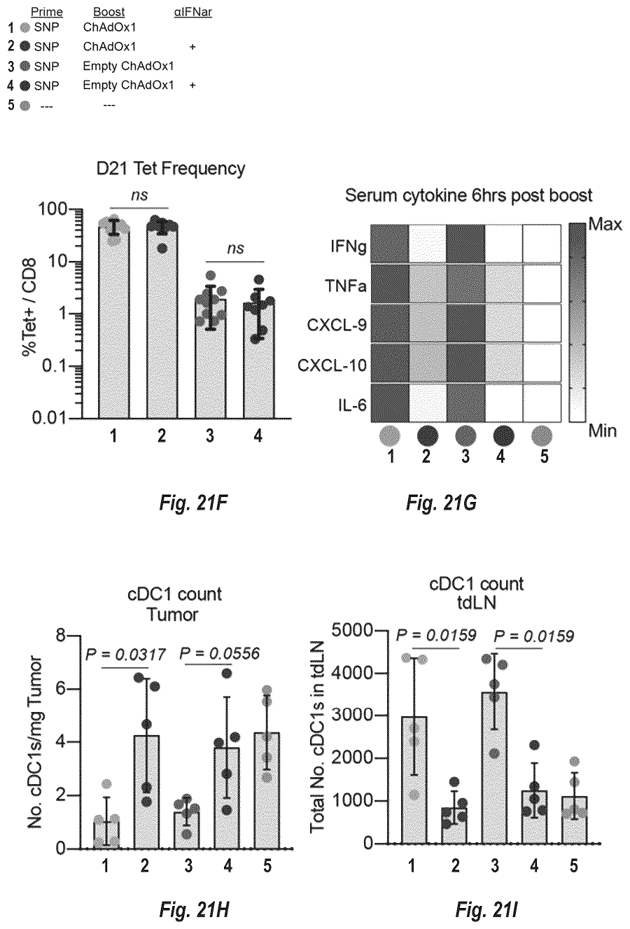

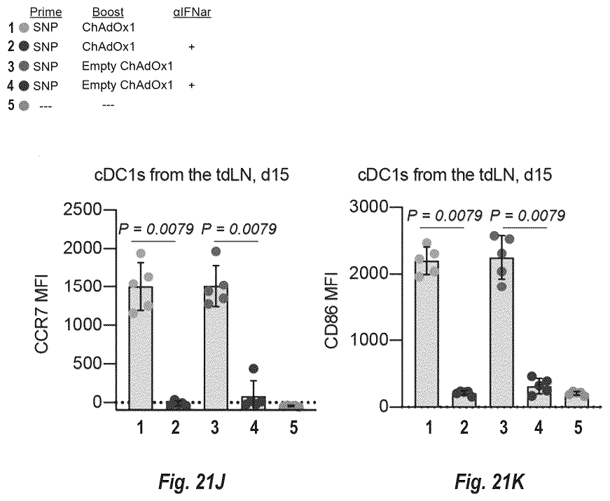

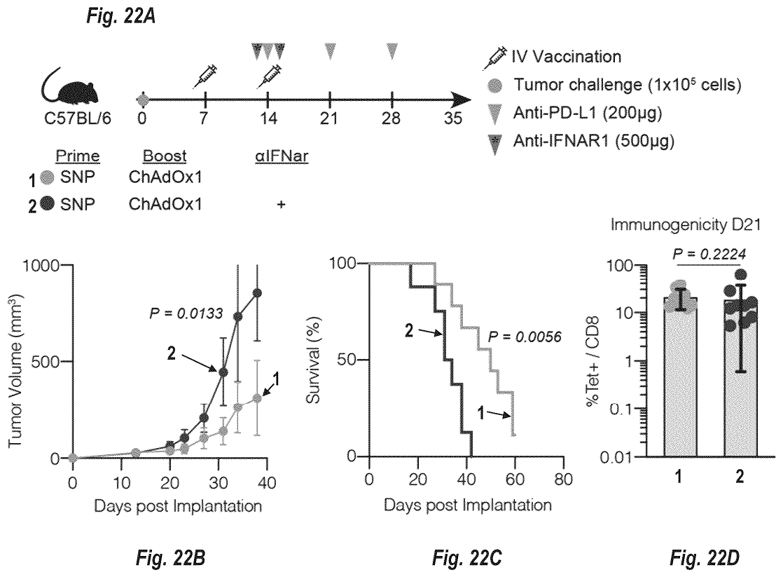

To determine whether type I IFNs are required for tumor control in the therapeutic setting, we used an IFNAR1 blocking antibody administered intraperitoneally (IP) 1 day before and after the boost vaccination (FIG. 21A). The protection elicited by IV ChAdOx1 boosting, with or without Reps-1 encoded, was dependent on functional IFNAR1 signaling. The IFNAR1 blockade resulted in loss of tumor control (FIGS. 21B and 21D) and abrogated any survival benefits of IV ChAdOx1 boosting (FIGS. 21C and 21E). Interestingly, the magnitude of the Reps1-specific CD8 T cell response measured in the blood 1 week post boost was unaffected by IFNAR1 blockade (FIG. 21F). The lack of tumor control, in the presence of high magnitude Reps-1 specific CD8 T cell responses elicited by the IV heterologous prime-boost regimen with IFNAR1 blockade, further demonstrates the critical requirement of functional IFNAR signaling for anti-tumor efficacy. The same dependence on IFNAR1 signaling was observed using an IV heterologous prime-boost vaccination strategy in the therapeutic setting with mice challenged with B16-F10 expressing the neoantigen Adpgk (FIGS. 22A-22D).

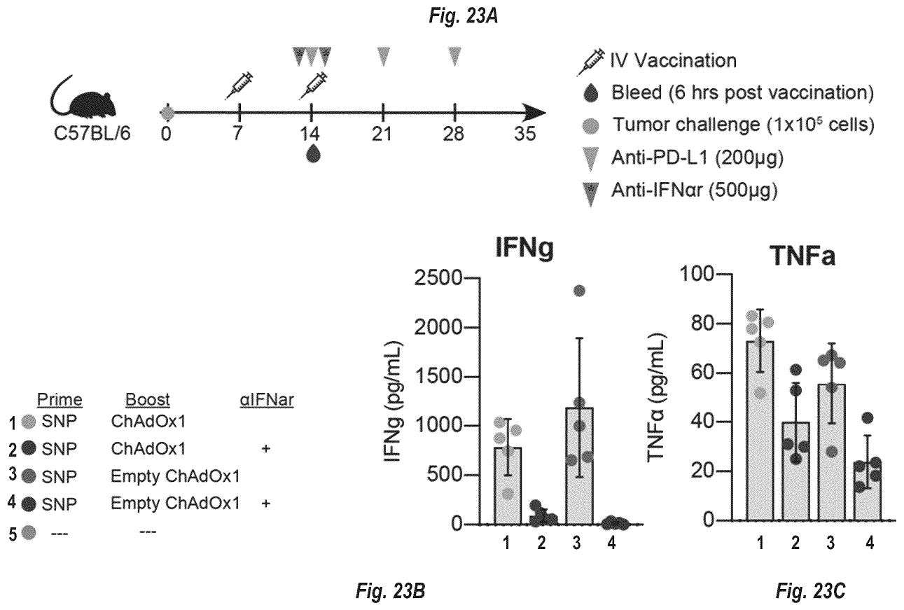

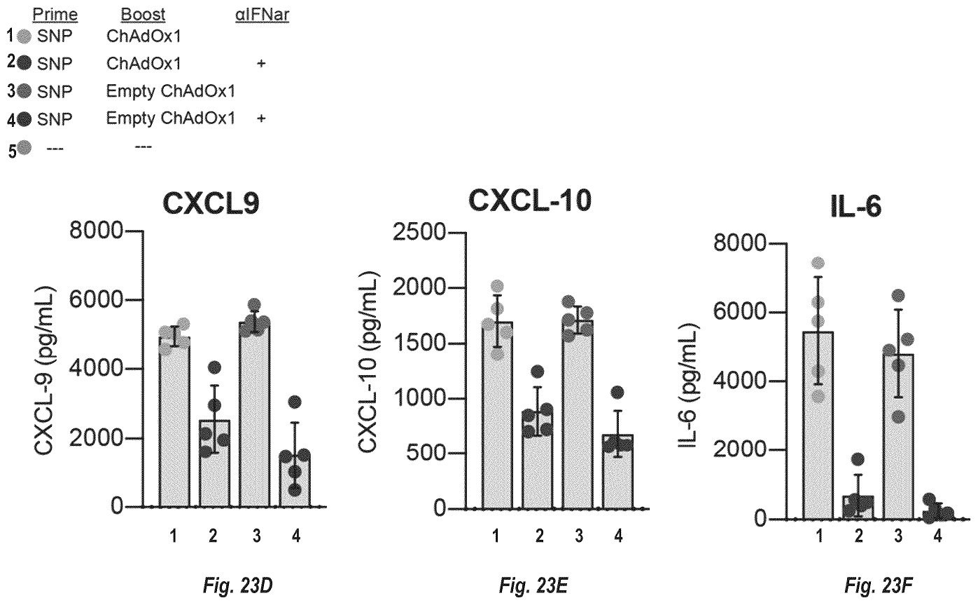

Type I IFNs have pleotropic effects on cytokines, chemokines and antigen presenting cell activation. IFNAR1 blockade around the time of boost resulted in a decrease of multiple cytokines including the proinflammatory cytokines IFNγ, TNFα, and IL-6, as well as the chemokines CXCL-9 and CXCL-10, which are produced by cDC1s to promote T cell infiltration into the tumor (FIGS. 21G and 23B-23F). It was hypothesized that cDC1s may be promoting tumor control following IV ChAdOx1 vaccination because: 1) their specialized function is to cross-present antigen to CD8 T cells, 2) IFNα is required to properly activate cDC1s and induce upregulation of co-stimulatory molecules involved in activating CD8 T cells, and 3) cDC1s secrete cytokines to promote T cell infiltration into tumors.

Therefore, the effects of IFNAR1 blockade on cDC1s in the tumor and the tumor-draining LN (tdLN) were assessed at 1 day post boost. In the tumor, IV ChAdOx1 vaccination was associated with a reduction in the number of cDC1s, and this was dependent on IFNα receptor signaling (FIG. 21H). In contrast, the number of cDC1s in the tdLN increased in the IV heterologous prime-boost groups and this was also dependent on IFNα receptor signaling (FIG. 21I). This was associated with increased expression of CCR7 on these cDC1s (FIG. 21J), which is known to be required for migration of cDC1s from the tumor to the tdLN to prime anti-tumor CD8 T cell responses. In addition, IFN-Is were also required for the upregulation of the co-stimulatory molecule CD86 on cDC1s (FIG. 21K). Collectively, these data suggest that IFN-Is induced by IV vaccination with ChAdOx1 promote the activation, maturation, and migration of cDC1s to the tdLN, which may support the anti-tumor efficacy of intravenous vaccination.

### Example 15. Intravenous Vaccination with ChAdOx1 Remodels the TME by Increasing the Frequency of Pro-Inflammatory Macrophages and Reducing the Frequency of Immunosuppressive Chil3 Monocytes

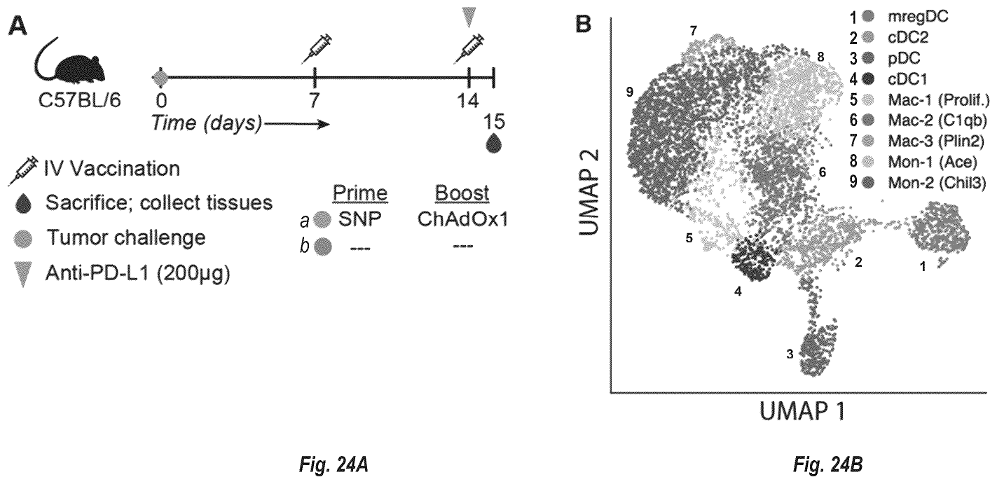

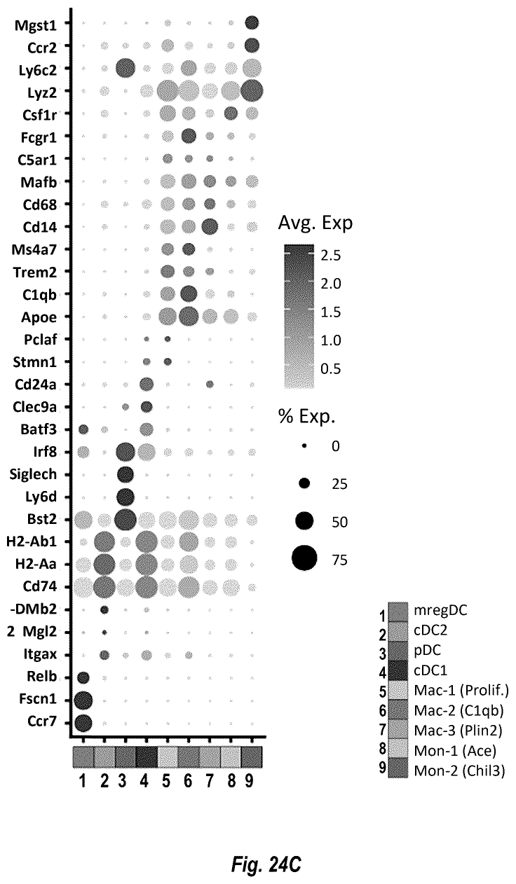

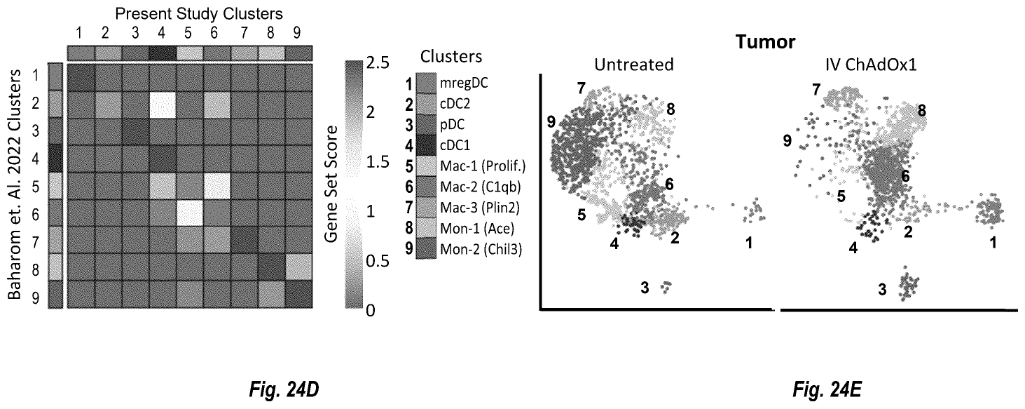

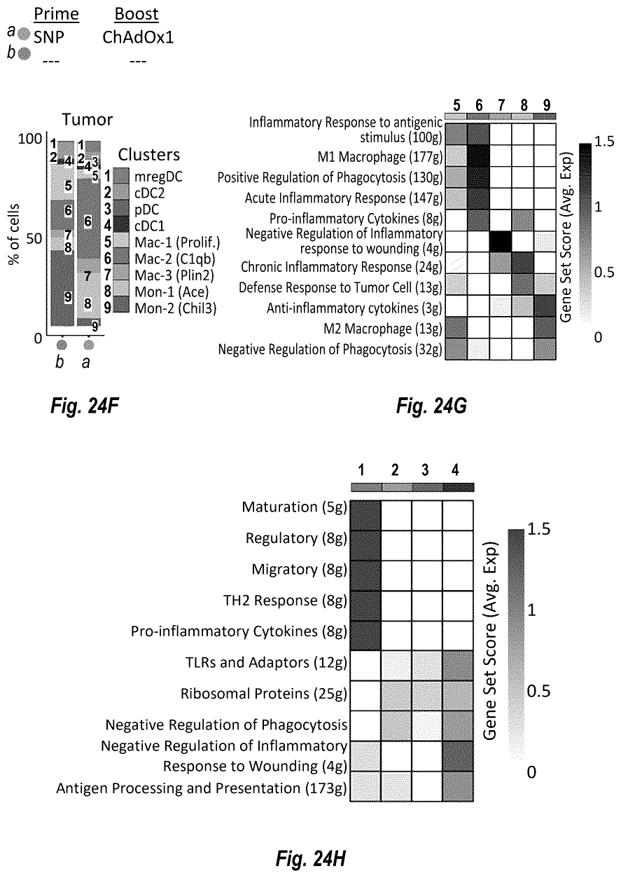

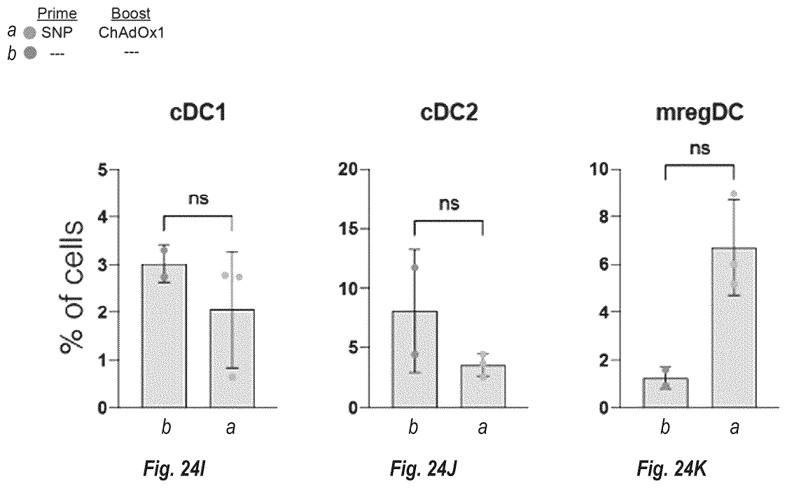

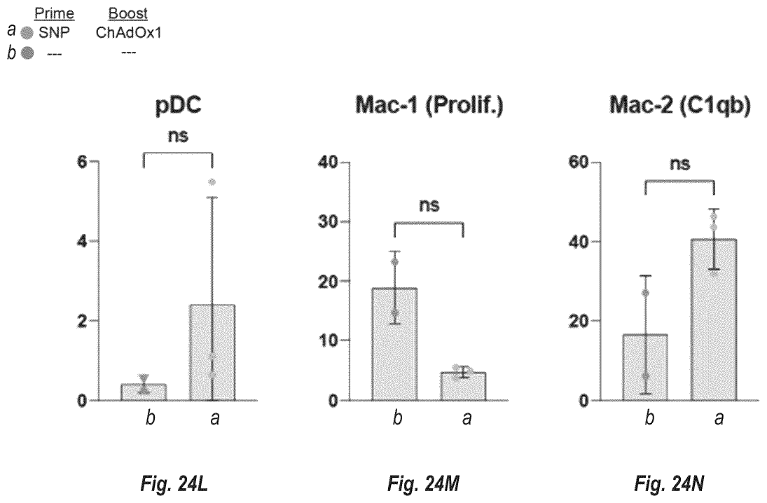

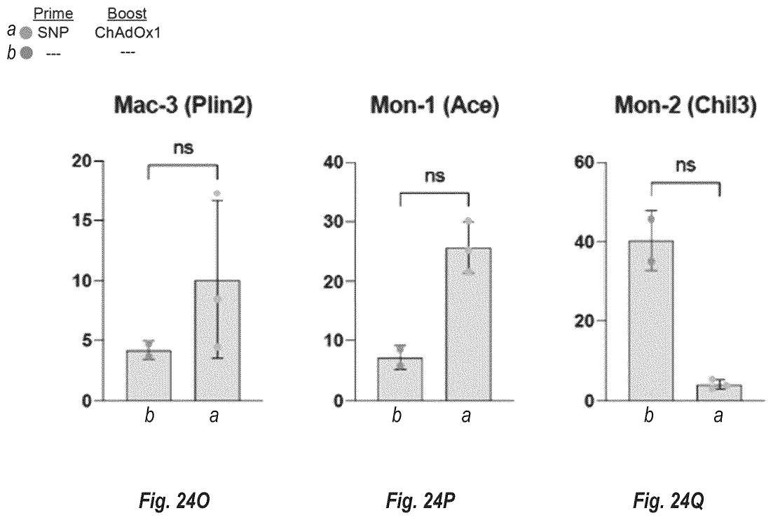

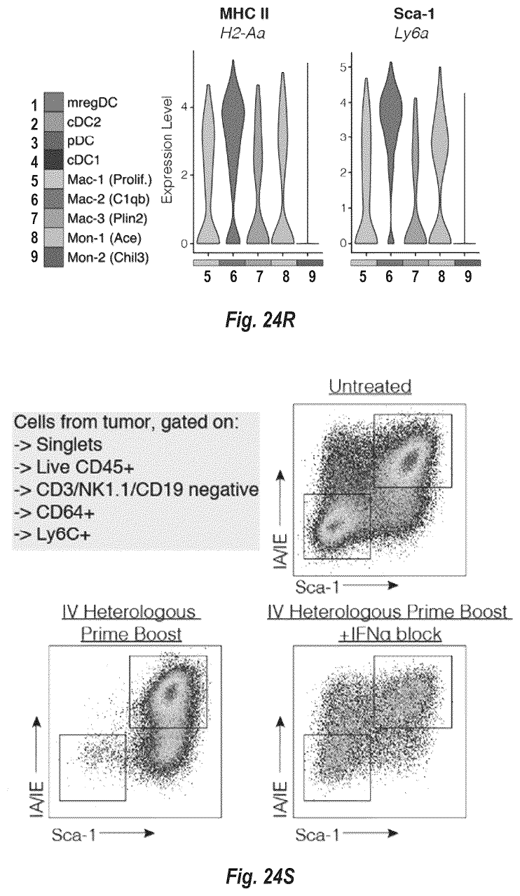

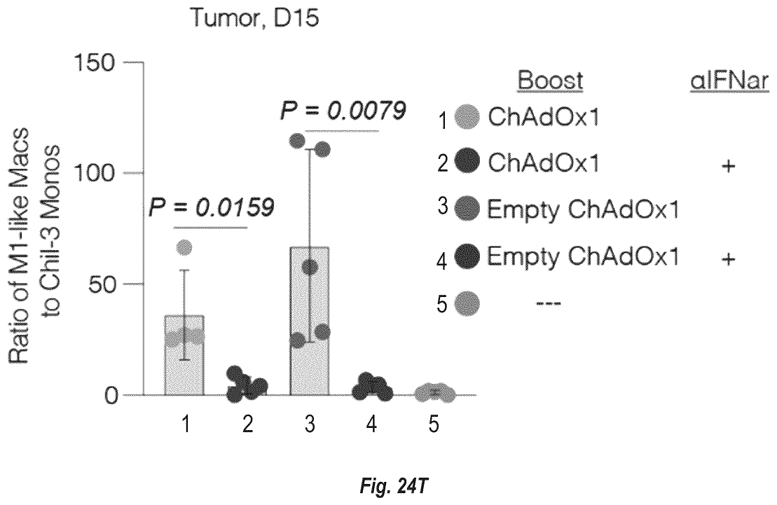

The TME is a heterogenous mixture of antigen presenting and myeloid cells. Thus, to further determine how IV vaccination by ChAdOx1 may be affecting the TME in an unbiased manner, a scRNA sequencing experiment was performed on myeloid cells from tumors and spleens collected 24 hours post boost with ChAdOx1 and compared these with cells from untreated mice (FIG. 24A). To obtain sufficient cells for this analysis from each tissue, CD45+, CD11b+ and/or CD11c+ were stained, sorted, and processed using the 10× genomics 5′ sequencing protocol.

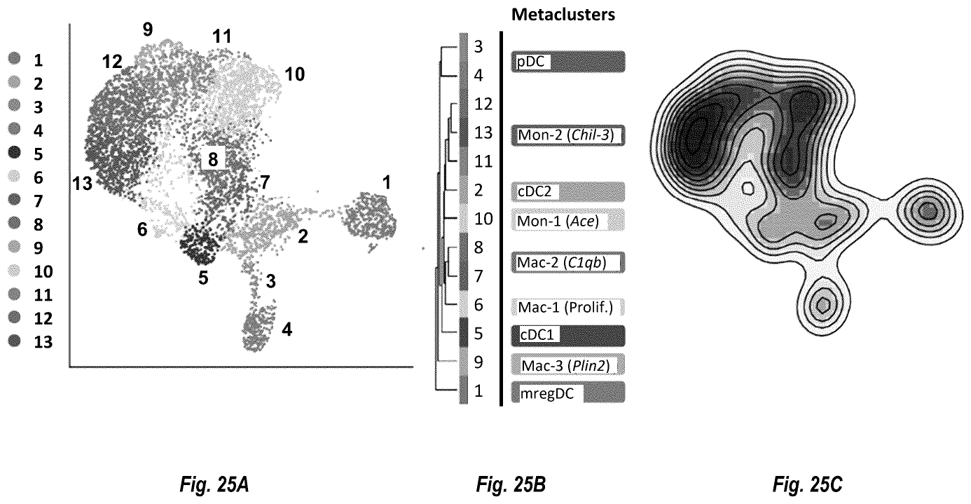

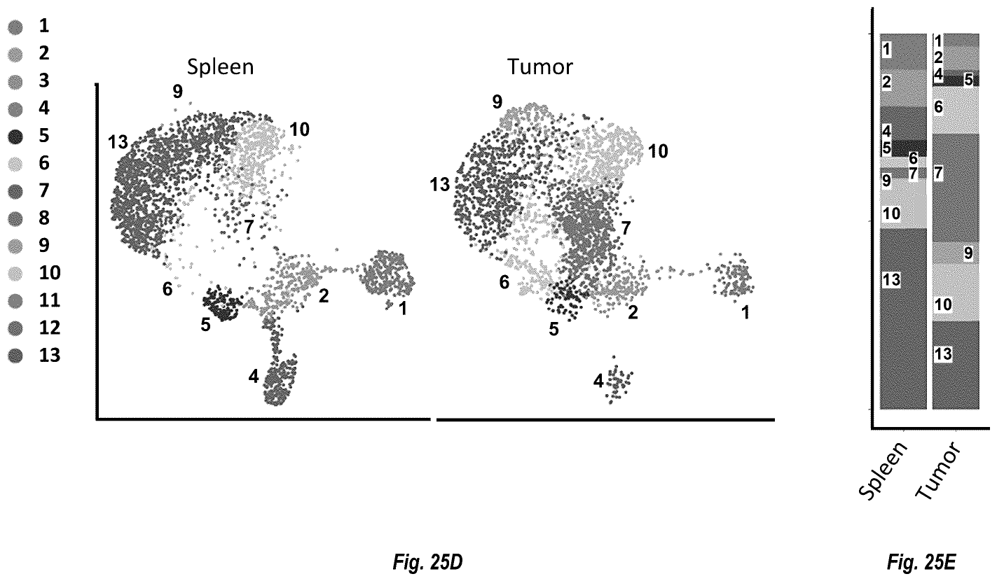

Clustering of the myeloid cells revealed 13 unique cell clusters visualized by UMAP dimension reduction (FIG. 25A). These clusters were reclassified into 9 ‘metaclusters’ through the use of an algorithm that calculates stable states depicted by a density plot (FIG. 25C). The Euclidean distance between original clusters and the focal point of a stable state was used to merge clusters with short Euclidean distances, and therefore greatest similarity (FIG. 25B). The identities of the new metaclusters visualized by UMAP (FIG. 24B) were assigned by assessing the expression levels of canonical markers. The 9 metaclusters included 4 DC populations: migratory/regulatory DC (mregDC, Ccr7, Fscn1, Relb), pDC (Siglech, Ly6d, Irf8), cDC1 (Batf3, Clec9a, Cd24a), cDC2 (Mgl2, H₂-Dmb2, Itgax), 3 macrophage subpopulations (Apoe, C1qb, Trem2), and 2 monocyte populations (Lyz2, Csf1r, Ccr2) (FIG. 24C). All 9 metaclusters were present in both the spleen and tumor (FIGS. 25D and 25E). The monocyte and macrophage clusters were annotated based on their expression of specific markers or transcriptional profiles, C1qb, Plin2, and proliferating for the macrophages and Ace or Chil3 for the monocytes (FIG. 25E). These metaclusters were very similar to clusters identified in Example 5 (see FIGS. 5-6), as demonstrated by the correlation heatmap comparing the expression of the top 10 expressed genes for each meta-cluster from this data set and the dataset of Example 5 (FIG. 24D).

The most striking finding was the disappearance of the Chil3 monocytes in the tumor following IV vaccination with ChAdOx1 (FIGS. 24E and 24F). There was a trend towards an increase in both Ace monocytes and C1qb macrophages in the tumor of ChAdOx1 vaccinated mice as compared to the untreated mice (FIGS. 24F and 24I-24Q). Gene set analysis using published data sets of genes associated with specific functions revealed that the C1qb macrophages expressed pro-inflammatory cytokines and an M1-macrophage like transcriptional profile, which suggests an anti-tumoral function (FIG. 24G). In contrast, the Chil3 monocytes are highly expressed anti-inflammatory cytokines and were characterized by an M2-macrophage like transcriptional profile, indicating that they are likely immunosuppressive and therefore pro-tumoral. Thus, IV ChAdOx1 vaccination appears to decrease the frequency of immunosuppressive Chil3 monocytes and increases the frequency of pro-inflammatory C1qb macrophages.

In addition, analysis of the DCs in the tumor revealed an increase in the frequency of mregDCs in the tumor of IV ChAdOx1 vaccinated mice (FIGS. 24I-24Q). These cells appear to be activated and express high levels of maturation genes and pro-inflammatory cytokines (FIG. 24H). Collectively, these data suggest that IV vaccination increases the presence of pro-inflammatory myeloid cells in the tumor that may promote tumor regression.

### Example 16. Intravenous Vaccination with ChAdOx1 Results in Loss of Chil-3+ Monocytes at the Tumor Site Through a Type I IFN Dependent Mechanism

The differentially expressed genes between the different monocyte/macrophage metaclusters were assessed and two genes encoding cell surface proteins were identified that could be used to differentiate Chil-3 monocytes from the other monocyte and macrophage subsets. These were MHC class II (H₂-2a) and stem-cell antigen gene 1 (SCA-1, Ly6A), an interferon-stimulated gene (ISG) (FIG. 24R). By sequential gating of APCs, we were able to distinguish cell that are likely the M2-like Chil-3 monocytes (Sca-1−, MHC II−) and activated M1-like macrophages (Sca-1+, MHC-II+) (FIG. 24S).

This staining panel was then used to assess monocyte populations in tumors 1 day post boost. Using this staining panel as a surrogate to identify Chil3 monocytes, there is a striking reduction in the frequency of Chil3 monocytes in the IV heterologous prime-boost as compared to the untreated mice or those that received both vaccinations and the IFNAR1 blocking antibody (FIG. 24S). This was quantified by calculating the ratio of activated macrophages to Chil3 monocytes and found that IFNα signaling shifts this ratio in favor of the activated and likely pro-inflammatory M1-like macrophages (FIG. 24T). These data suggest that type I IFNs elicited by IV ChAdOx1 vaccination are altering the balance of myeloid cells at the tumor site in favor of pro-inflammatory states that may enable anti-tumor immunity.

### Discussion Regarding Examples 9-16

The goal of cancer vaccines is to prime and expand high frequencies of tumor-specific T cell responses that can mediate tumor regression. =By utilizing the ChAdOx1 as an intravenous boost (i.e., an IV second treatment), Examples 9-16 demonstrate an increase in the attainable magnitude of CD8 T cell responses that is ˜4× fold higher than through IM. These responses also remain elevated over a prolonged period, remaining at ˜10% of the T cells in blood 16 weeks post vaccination in mice. This improvement in magnitude and durability as a result of IV vaccination may be a broadly applicable observation with ramifications for other groups developing adenoviral vaccines to induce T cell immunity.

Though CD8 T cells are required for efficacy of CPIs, in many instances this may be insufficient as the newly primed CD8 T cells may encounter an immunosuppressive TME that inhibits their function through multiple mechanisms beyond checkpoints. As disclosed herein, IV vaccination had a secondary effect in reducing the frequency of immunosuppressive Chil3 monocytes with a concomitant increase in the frequencies of M1 macrophages (pro-inflammatory) through an IFNAR1 signaling dependent mechanism. Also, systemic type I IFNs were found to be associated with an increase in the number of cDC1s in the tdLN expressing the migratory marker CCR7 and co-stimulatory molecule CD86. It was hypothesized that these may be priming de novo anti-tumor T cell responses to support anti-tumor immunity.

The anti-tumoral effect of type I IFNs has been known for decades, as IFNα2 was used in the 80's as a treatment for cancer. This was associated with toxicity and subsequently discontinued with the advent of more well-tolerated cancer therapeutics whose mechanism of action was better understood. The mechanisms underpinning the anti-tumor effects of IFNs on myeloid, lymphoid, and tumor cells have only begun to be understood more recently. Examples 9-16 focus predominantly on the effects of systemic type I IFNs on cDC1s, monocytes, and macrophages as these were the most readily detectable. However, IFNs can also increase the expression of tumor cell antigen processing and presentation machinery, increase proliferation and activation of NK cells, reduce Treg activity, and increase T cell effector function, among other effects. It is possible that the vaccine elicited Type I IFNs are orchestrating a systemic revitalization of the immune system through the activation of many of these pathways to promote anti-tumor immunity.

As disclosed in Examples 9-16 herein, IV ChAdOx1 boosting provided a two-fold therapeutic benefit by not only increasing the magnitude of the antigen-specific CD8 T cell response, but also modulating the TME towards a more pro-inflammatory state.

The foregoing written specification is considered to be sufficient to enable one skilled in the art to practice the embodiments. The foregoing description and Examples detail certain embodiments contemplated by the inventors. It will be appreciated, however, that no matter how detailed the foregoing may appear in text, the embodiment may be practiced in many ways and should be construed in accordance with the appended claims and any equivalents thereof. All cited publications and patent applications are incorporated by reference herein in their entirety.

1. A method for treating a cancer in a subject in need thereof, comprising (a) administering a first treatment comprising an antigen that provides antigen-specific CD4+ and/or CD8+ T cells in the subject, and (b) following a time interval (T), administering a second treatment that induces systemic and/or tumor specific inflammation in the subject.

2. (canceled)

3. The method of claim 1, wherein the first treatment provides antigen-specific CD8+ T cells in the subject; and/or the second treatment induces type-1 interferon (IFN-1) signaling in the subject, and/or increases the level of IL12 or IFN-alpha in a blood sample from the subject, and/or induces tumor specific inflammation in the subject.

4. (canceled)

5. The method of claim 1 wherein the first treatment comprises administering a vaccine to the subject,
optionally wherein the vaccine comprises a polynucleotide encoding an antigen, wherein the polynucleotide is optionally an adenovirus, adeno-associated virus, rhabdovirus, ChAdOx, MVA virus, DNA vector, or RNA vector; or the vaccine comprises a peptide antigen, optionally comprised within a chimeric protein or a peptide-antigen conjugate.

6.-7. (canceled)

8. The method of claim 5, wherein the vaccine comprises a peptide antigen conjugate of formula S-[E1]-A-[E2]-[U]-H-[D] or of formula PEG-[E1]-A-[E2]-[U]-H-[D], wherein:
A is a peptide antigen,
H is a hydrophobic molecule, optionally wherein the hydrophobic molecule (H) is water insoluble at pH 7.4,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage.

9. (canceled)

10. The method of claim 8, wherein the vaccine further comprises an amphiphile, such as an amphiphile of formula S-[B]-[U]-H-[D], wherein:
S is a solubilizing block,
H is a hydrophobic block,
B is an extension,
U is a linker,
D is a drug molecule,
[ ] denotes that the group is optional, and
a dash (-) indicates a covalent linkage;
and/or wherein the vaccine further comprises or is administered together with an immunostimulant, such as one or more of a TLR-3, TLR-7, TLR-8, TLR-7/8, TLR-9, MDA5, RIG1, or STING agonist.

11.-14. (canceled)

15. The method of claim 1, wherein the second treatment comprises (a) one or more of a TLR-3, TLR-7, TLR-8, TLR-7/8, TLR-9, MDA5, RIG1, or STING agonist, and/or (b) one or more molecules that induce Flt3, IL-12, and/or type-I IFN signaling, and/or (c) one or more amphiphiles, such as an amphiphile of formula S-[B]-[U]-H-[D], wherein:
S is a solubilizing block,
H is a hydrophobic block,
B is an extension,
U is a linker,
D is a drug molecule, such as an immunostimulant drug molecule,
[ ] denotes that the group is optional, and
a dash (-) indicates a covalent linkage.

16. The method of claim 1, wherein the second treatment comprises a vaccine
optionally wherein the first treatment comprises a vaccine of formula S-[E1]-A-[E2]-[U1-H-D], and/or the second treatment comprises a vaccine of formula PEG-[E1]-A-[E2]-[U]-H-[D].

17.-19. (canceled)

20. The method of claim 16, wherein the first and second treatment comprise the same vaccine or wherein the first and second treatment comprise or encode the same antigen.

21. (canceled)

22. A method for treating cancer in a subject in need thereof, comprising (a) administering a first treatment by IM or IV and (b) following a time interval, administering a second treatment by IV, wherein the first treatment comprises a first peptide antigen conjugate of formula S-[E1]-A-[E2]-[U]-H-[D] or of formula PEG-[E1]-A-[E2]-[U]-H-[D], wherein:
A is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage,
wherein the second treatment comprises a second peptide antigen conjugate of formula PEG-[E1]-A-[E2]-[U]-H-[D], and wherein the second peptide antigen conjugate comprises or is administered together with (a) one or more of a TLR-3, TLR-7, TLR-8, TLR-7/8, TLR-9, MDA5, RIG1, or STING agonist, and/or (b) a molecule that induces Flt3, IL-12, and/or type-I IFN signaling; and
optionally wherein the first treatment provides antigen-specific CD4+ and/or CD8+ T cells in the subject and the second treatment induces systemic and/or tumor specific inflammation in the subject.

23. The method of claim 22, wherein the first treatment and the second treatment comprise the same peptide antigen.

24. (canceled)

25. The method of claim 22, wherein the first and/or second treatment further comprises an amphiphile, such as an amphiphile of formula S-[B]-[U]-H-[D], wherein:
S is a solubilizing block,
H is a hydrophobic block,
B is an extension,
U is a linker,
D is a drug molecule, optionally wherein D is an immunostimulant drug,
[ ] denotes that the group is optional, and
a dash (-) indicates a covalent linkage.

26. A method for treating cancer in a subject in need thereof, comprising (a) administering a first treatment by IV or IM, and (b) following a time interval, administering a second treatment by IV, wherein the first treatment comprises a first peptide antigen conjugate of formula S-[E1]-A-[E2]-[U]-H-[D] or of formula PEG-[E1]-A-[E2]-[U]-H-[D], wherein:
A is a peptide antigen,
H is a hydrophobic molecule,
S is a solubilizing block,
PEG is polyethylene glycol,
E1 is an N-terminal extension,
E2 is a C-terminal extension,
U is a linker,
D is a drug molecule,
[ ] denotes that the group is optional, and
a dash (-) indicates a covalent linkage,
optionally wherein the peptide antigen conjugate has a net electrostatic charge greater than or equal to +3 or less than or equal to −3 in an aqueous buffer at a pH of 7.4, and optionally wherein the hydrophobic molecule (H) is water insoluble at pH 7.4; and
wherein the second treatment comprises a polynucleotide optionally encoding an antigen, wherein the polynucleotide is optionally an adenovirus, adeno-associated virus, rhabdovirus, ChAdOx, MVA virus, DNA vector, or RNA vector; and
optionally wherein the first treatment provides antigen-specific CD4+ and/or CD8+ T cells in the subject and the second treatment induces systemic and/or tumor specific inflammation in the subject.

27. (canceled)

28. The method of claim 26, wherein the polynucleotide encodes an antigen that is the same as the antigen of the first treatment.

29.-31. (canceled)

32. The method of claim 8, wherein the dose of the peptide antigen conjugate is 250 nmol-40,000 nmol total conjugate, 500-20,000 nmol total conjugate, or 1000-10,000 nmol total conjugate.

33. The method of claim 1, wherein the time interval (T) is at least 3 days, at least 5 days, 5 to 90 days, 5 to 60 days, 5 to 30 days, 5 days to three weeks, one week to three weeks, one week to two weeks, 3 to 28 days, 5 to 28 days, 5 to 14 days, 7 to 28 days, 3 to 21 days, 5 to 21 days, 7 to 21 days, 3 to 14 days, 5 to 14 days, 7 to 14 days, 14 to 28 days, or 14 to 21 days.

34. (canceled)

35. The method of claim 1, wherein the second treatment comprises a TLR 7/8 agonist, optionally wherein the TLR 7/8 agonist is administered at a dose of 750 to 120,000 nmol, or 3000 to 30,000 nmol.

36. A method for treating cancer in a subject in need thereof, comprising administering an adoptive cell therapy (ACT) and intravenously administering a vaccine, wherein the ACT and the vaccine are administered sequentially such that the vaccine is administered from 3 days prior to administration of the ACT to 14 days following administration of the ACT.

37.-39. (canceled)

40. A method for treating cancer in a subject in need thereof, comprising administering an adoptive cell therapy (ACT) and intravenously administering an immunostimulant, wherein the ACT and the immunostimulant are administered sequentially such that the immunostimulant is administered from 3 days prior to administration of the ACT to 14 days following administration of the ACT, wherein the immunostimulant comprises: (a) one or more of a TLR-3, TLR-7, TLR-8, TLR-7/8, TLR-9, MDA5, RIG1, or STING agonist, (b) a molecule that induces Flt3, IL-12, and/or type-I IFN signaling, and/or (c) an amphiphile, such as such as an amphiphile of formula S-[B]-[U]-H-[D], wherein:
S is a solubilizing block,
H is a hydrophobic block,
B is an extension,
U is a linker
D is a drug molecule,
[ ] denotes that the group is optional, and
a dash (-) indicates a covalent linkage.

41. (canceled)

42. The method of claim 36, wherein the ACT comprises administration of a TIL or CAR-T therapy.

43.-46. (canceled)

47. A kit comprising a first and a second treatment of claim 1, and optionally further comprising instructions for use.
